SEQUENCE LISTING (99 sequences)

SEQ ID NO: 1 - DNA, 11141 bp, Clostridium hiranonis
  source 1..11141: mol_type = unassigned DNA
gcaaattgattttgattggtatttctttcattcaaaatatctcctttcctttatttagctgtattaaaatttataaaaaattttcattgttaataaaaaaatattctttgttagtattatagcataatttataaaaataatgataatgttttaatattgaaataataaatatgtaaaaaggttggaaatttatttaaaaatgaccagagataaaaagctcaggtcattttttttattattacaagtaatttgaaaaaaatatatgaaatgaatggagaaaatataactgagatacatttgataatgaaaaaaacatttatcgaaattgtaaatagactcattgttataattaataaatatttattatggcatagttgttaaaattataccctaaagaaacgtttcctcaaaaagtgggttataaaataaatgttttttgacgaaagatgtgattttatttgtaccccttttgtataaagattaaacagtatttttgtataaatatattgtatacagtatagagaatgtcgatgtaaaaaagtatataaaagtaaataataatcaaaaaaactagttttaattattaaaaatgataaaaaatattaataaaataaagagtcaaaaatacttgttagttaaatcacagattttgtctaagtatagattaggttttgtatttgaaaaggtcatctatagtgttgtaagaaagcgagttattagcacatattgtatctcaaaaaaatgttaagataatatcaagatagggcgataaagaaaaaagcaaattgaaaaagaaaaaagtaactataagtttttacaataaatcaaaagagaattgattttaaaagagggaggcaaaataccgatatgaatgatgtgaaatgtaaatattttaataaatttaatacaggaatgtcagattttgttactccaggaaaacagttagaatatgtagcaaaatgcaagccagatgaaaaagctatcatatatatagataaagaagacaatgtgagagatatcacttggaaggaacttcacatagcttcaaataaactagcttggcatttaatgaaaaagggatttggaaaaggtcaggtagcaatggtatctttcccaaatggtatagaacatatattagcaacattagctgtttggaaaacaggaggttgctacatgccagtttcttgtaagataacagatacagagcttggtgatatatgcagaataataaaaccaacagtttcttttacagataaagaaatgccttgtagaacagaaagtataaaaataggatcagtattcgatgtttgtaaagacgaatcagaagaaatgccagaagatatagctgcaaatccaaatatgatttctccatctggaggaacaacaggagagcctaagttcataaaacagaatgtggcaagtggcttatctgatgaaattataaaaagctggtttgaaatgtcaggtatggaatttgaacaaagacaattattagtaggaccacttttccatggtgctcctcatacagcagcatttaatggattatttgtaggaaatacattgataatacctagaaatttaagacctgaaagtatagttagatatataaaagaatacaaaatagaatttatacagatgatcccaacattaatgaatagaataataaaattagctgatgttgataaagaagattttaaatcaataaaagcactacaccatactggtggatattgttctccatatttaaaagaaaagtggatcgatataataggagctgaaaaagttcacgaaatgtactctatgacagaggcaatcggtatcacttgtataagaggagatgaatggcttaaacactatggaagcgtaggacttccactaggaggaagcagaatatcaataagagatgaagaaggaaatgaattaggaccacatgaggttggagaaattcatatgacttcaccaagtgcttgttgcatgacagaatacataaaccataaaccacttgaaactaaagatggtggatttagaagtgttggtgatttcggttatgtagatgaagatggatacctttacttctcagatagaagaagcgacatgcttgttataggtggagaaaacgtatttgcgactgaagttgaaccagtactaccagcttatgaaaaagtagttgatgctgtggtagttggaatacctgatgaagagtggggaagaagattacacgcaatagtacagaagaaagaagaagtttcagcagaagaattaatcgagtacttaggaaaacacttattaccatataaagttccaaagagctttacatttgttccttgcataccaagaggtgacaatggaaaggtaaacagagataagatgctaaaaggcttaatagaaaaaaatctagttaataaagtttgctaggatataaattcagttaactatctgcaccaagtgcagtggaaaataaatcaaaattaataaaataaattaataaggtaaatttaggaggtctaaaatgagttacgacgcacttttttcaccatttaaaatcagaggattagaacttaaaaacagaatagttctaccaggtatgaatacaaaaatggcaaaaaataaacatgatttaagcgatgatatgatagcttaccatgttgcaagagcaaaagcaggttgtgcattaaatatatttgaatgtgttgcgctatgtccagcacctcatgcatatatgtacatgggattatacaatgacaatcatgtagctcagttaaaaaaattaacagatgctgttcacgaagttggcggtaaaatggctgttcagttatggcatggtggtttcagcccacagatgttctttgataaaacaaatacattagaaacaccagatactataacagttgaacgtattcatgaaatagttaaagagtttggagaaggtgcaagaagagctgttgaagctggattcgatgcagttgaattccatgcagcacacagttacttacctcacgaattcctaagtccaggaatgaacaaaagaactgacgaatatggtggaaacttcgaaaatcgttgcagattctgcttcgaagtagttgaagctatacgtgcaaatataccagaagatatgccattcttcatgagagttgactgcatagatgagttaatggatgaagtaatgacagaagaagaaatagtagaattcataaatagatgtgctgatctaggagtagacgtagctgacttatcaagaggtaatgctcagtcattcgcaacagtttacgaagttcctcctttcaacttacagcacggtttcaatatagaaaacatatacaacatcaaaaaacagataaaaataccagtaatgggtgttggacgtataaacacaggagaaatggctaaccaggtaatagcagatggaaaatttgacttagttggtataggtcgtgctcagttagcagatcaggattgggttgctaaagttagagaaggtaaagaagatttaatacgtcattgtataggatgtgaccagggatgctacgatgcagttataaaccctcagatgactcatataacttgtacaagaaaccctcacttatgcttagaatacaaaggtatgccaaaaactgatgaacctaaaaaagttatgataatcggtggtggtatggctggtatattagcagctgaagtacttaaaaaacgtggacatgaaccagttatattcgaagcttctgatcacttagcaggacagttcgtattagcaggtaaagctccaatgaaagaagactgggcagctgcagctaaatgggaagctgaagaagtagctcgtttaggaatagaagttagatacaatacaaaagttactccagaattaatagaagaattcgctccagaccacgttgttatagctataggatctgattacgtagctccagctataccaggtatagatagtgacaaagtttacactcagtatcaggtattaaaaggtgaagtagaaccaaaaggacatgtagcagtagttggttgtggattagttggtacagaagttgctcagtacttagcagctagaggagctcaggtaacagctatagaaagaaaaggtgttggtacaggtctaagcatgcttagaagaatgttcatgaacccagaattcaaatactacaaaataaacaaaatgtctggaactaacatagttggtatagaaccaggaaaacttcactacataatgactaacaagaaaactcaggaagttactgaaggtgtgttagaatgtgatgcagcagtaatctgtacaggtataactgctagaccaagtgaagatttacaggaaaaatgtaaagaattaggtgttccattcaacgtaataggtgacgcagctggtgctagagatgctagaatagctactcaggaaggttacgaagtaggtatgagtatataatttaaaaattatataattatataaattaaaagttattaaattacaagaaagaggcgaataaaatgactttagaagcaagaatagaagcattagaaaaagaaatacagagattaaacgatatagaagctataaaacagttaaaagctaaatatttccgttgcctagatggaaaattatgggatgaattagaaactactctttctcctaacatagaaacttcttactctgatggaaaattagtattccacagcccaaaagaagtaactgaatatttagcagcagcaatgcctaaagaagaaataagtatgcacatgggacatactccagaaataactatagacagcgaaaatactgctacaggaagatggtacttagaagataacctaatattcacagacggaaaatacaaaaacgttggaataaacggtggagcattctacacagataaatatgaaaaaatagacggacagtggtacataaaagaaactggatatgttcgtatatttgaagaacatttcatgagagatccaaaaatacatataactagcaacatgcataaagaaaaataataactgattgctaataaacaagatataaacagggggctggtaaacagccagccctctgaaaaataaactaaaaaactataatcttttaaaatcttaattaaagtagaaggagataagacaatgaacttagtacaggacaaaatagttataataacaggtggaacaagtggtataggtctttgcgcagcaaaaatattcatggataacggtgcaacagtttctatattcggaaaaactcaggaagaagtagatgctgctaaagcagaattaaaagaaactcacccagataaagaagtattaggatttgctccagatttaactaatagagatgaagttatggctgcagttggtgcagtagctgaaaaatacggaagattagacgttatgataaacaatgctggtgttactagctcaaacgtattctcaagagttagcccagaagaattcacatatttaatggatataaacgttacaggtgtattccatggtgcttgggctgcttaccactgcctgaaaggtgaaaagaagattataataaatactgcttcagtaacaggaatacacggatcattatcaggagttggatacccaacaagtaaatcagctgttgtaggattcactcaggctcttggtagagaaataatacgtaaaaacataagagttgttggtgttgcaccaggtgttgttaacactccaatggttggtaatataccagatgaaatattagatggatacctaagctcattcccaatgaagagaatgttagaaccagaagaaatagctaacacttacttattcttagcttctgacttagctagtggtataacagctacaactgtaagcgttgacggtgcttatagaccatcataagatttactttaatttaaaactgtaattagatagataatacgacgattaatataaaaaatgttctttaaaagaaaaggagaaataaaatggctggattaaaagattttcctaaatttggtgcactttctggattaaaaatattagatagtggatctaacatagctggacctctaggtggtggacttttagcagaatgtggtgctacagttatacacttcgaaggacctaaaaaacctgacaaccagagaggttggtatggataccctcagaaccacagaaaccagttatcaatggttgctgatataaaatctgaagaaggtagaaaaatattcttagacttaataaaatgggctgacatatgggttgaatcatcaaaaggtggacagtacgacagactaagtctttctgatgaagttatatggtcagtaaaccctaaaatagctatagttcacgtttctggatacggacaggttggagatccatcatacgtaacaaaagcttcttatgatgctgttggacaggcattcagtggatacatgtcattaaatggtgttaatgaagcattaaaaataaatccttacctaagtgacttcgtatgtgttcttactacttgctgggcaatgttagcatgctacgtaagtactcagttaactggaaaaggagaatctgtagacgttgctcagtacgaagcattagctcgtataatggacggacgtatgatacagtacgctactgatggtgtaagtgttccaaaaactggtaacaaagatgctcaggcagctctattcagcttctatacttgtaaagatggaagaactatattcataggtatgactggtgctgaagtatgtaagagaggattccctgtaatagggcttccagttcctggtacaggtgaccctgacttcccagaaggattcacaggatggatgataaatactccagttggacagagaatggaaaaagctatggaagcattcgttgctgaaagaactatgccagaagttgaaaaagctatgatagatgctcagataccatgccagagagtttatgatcttgaagactgcttaaacgaccctcactggaatgctcgtggaactataatggaatgggatgacccaatgatgggacacataaaaggtcttggattaataaacaaattcaaaaacaacccttctgaaatatggagaggtgctccattattcggtatggacaacagagacataattagagaccttggatattctgaggaggaagttaacgatttatacgctaaaggtattgtaaacgaattcgaccttgaaacaactataaaacgttacaaacttgatcaggttatacctcacatggctaaaaaagataaataagaaacgtattaaataataaaatataaatgtcgagcctgccagaatgagaattttgacaggcttgatattataacgaaatgttataaaaaaaacaaaataaaaattgcttaaattttatacaaggagaattgaaatgacagcaacaaacgcaaactataaaaaaggctttatcccatttgctatagcagcgttactagtaggtcttataggtggtttcacagccgttctagcacctgcattcgtagcagatatgggtcttaacgataacaatactacatggatagcactagcgcttgcaatgtctacagctgcatgtgctccaatacttggtaaattaggtgacgtacttggacgtcgtaaaactttattattaggaatcatagtattcacaataggtaacgtattaacagcaatagcatcttcattaatattcatgctaggtgcaagatttatagttggggttggtacagcggctatagctccagttataatggcttacatagttacagaatatccaccagaagaaactggtaagggattcgctctttatatgttaatatcaagtgctgcagttgttgttggtccaacttgtggtggattaataatgcaggcatttggatggagaatgatgatgtgggtttgtgttgccctttgtgtagtaacattcttcatatgttcagtaatgattaagaaaacagactttgaaaagaaaagtcttgataacttcgataaaaaaggtgcagtatgcgtactaatattcttcagtttagtattatgtataccatcatttggacagaatataggttggacatcagcgccattcctaggtgttacagcagtagctttagtaacattattcttattaataaaagctgaaagcagtgcagaaaacccaatattaagtggtaaatttatgaaacgtaaagaattcatattaccagtattaatattattccttactcagggattaatgcaggctaacatgactaacgtaatattattcgttagagctactcagccagaaaatacaataatatcaagtttcgcaatatcaatcctttacataggtatgtctttaggttcagtattcataggacctatggcagataaaaaagaaccaaaaactgtacttacaggatcacttctattcactggtataggttgtgcaatgatgtacttcttcacagaaactgcaccattcgcaatgttagctggatctctaggaatgttaggtataggacttggaggaaatgctacaatactaatgaaagtttcattatctggattatctcaggcagaagctggatcaggaacaggaacatacggattattcagagatatatcagctccatttggtgttgcggtattcgtaccactatttgcaaacacagttacaacaagaatggctggagtaatggctaacggaactgcagaagctgctgctaaatcattagcatctgtttcttctatacatacattagcattagttgaagtatgctgtgtaatattagcaatagttgcagttagaatgctaccaaaaatacacaataaataatttaaaaataataacagagttgaaaaaacactcaattaaaagaggggccttgagccccttttttagtgtaaaaatgacaaaatactatcaatttatataaatgataattaaactcgtcaaccaaagaaatattcacaaagtagataataatagatattcaaaaagtgatatattattaggcaaaaagtgcaagaaattagcgagtattcgacaactttttgtccaatggtagaaaagaatatttgttatcataaatatagacaaagggctttgaccaaaactaaggaaaaagtttgcataatataaaaaataaaataaaataaaaaaataaaaataaaataaaagcgaaaggaaaaaacaacatcatggatatgaaaaattctaaactattctcacctttaacaataggatcattaacattaaacaacagagttggtatggcaccaatgagtatggactacgaagctgctgacggaacagttccaaaaagattagcagatatatttgttcgtagagctgaaggtggaacaggatatgtaacaatagacgcggtaacaatagatagtaaatataaatatatgggtaatacaactgctttagattctgatgatttagttactcagttcaaagaatttgcaacaagagttagagaagcaggaagcacattaatacctcaggttatacatccaggaccagaatcaatatgtggatacagacacatagcaccacttggaccatcagttaatacaaatgctaactgccacgtgagccgtgctataagtgtagatgaaatacatgaaataataaaacagtttggacaggctgctagaagagttgaagaagcaggatgcggtggtataggattacactgtgcacatgcttacatgctaccaggttcattcttatctccattaagaaacaaaagaatggatgaatacggcggatgtctagataacagagcaagattcgtaatagaaatgatagaagaagttcgtagaaatgtaagtcctgatttcccaataatgcttagaatatctggggatgaaagaatgataggaggaaactctttagaagatatgttatacttagctccaaaatttgttgaagctggtgtaaatatgtttgaagtttctggaggtactcagtacgaaggattagaacacataataccaagtcagaacaaaagcataggtgtaaacgtacacgaagcatctgaaatcaaaaaagttgtagatgttccagtttacgctgttggtaaaataaatgacataagatacgctgctgaaatagttgaaagaggactagttgatggggtatcaataggtagaccattattagcagatccagacttatgtaataaagcaaaagaaaacttatttgatgaaataactccatgtgcaagctgtggaggaagctgtataagccgtactgcagatagacctcagtgtcgttgccatataaacccaagagttggattcgaatatgattatccagaagttccagctgaaaaatctaaaaaagttctagttgtaggtgctggacctggtggtatgatggcagcagttacagcagctgaaagaggacatgatgtaacactttgggaagctgacactcagataggtggacagataaacttagcagtagtagctccaggtaaacaggaaatgactaaatggttatctcacttaaactacagagctaaaaaagctggagttaaaatggtattaggaaaagaagctacagtagaaaacataaaagaatttgctccagaagcagttatagttgcaacaggtgctagaccattagttccaccaataaaaggaactcaggactacccagttcttacagctcatgacttcttaagaggaaaattcgttataccaaaaggaaaagtttgtgtactaggtggaggagctgttgcttgtgaaactgcagaaacagtattagaaaacgctagaccaaacgcattcactagaggatttgatgctagtatcggtgatgtagatgttacattagtagaaatgttaccacagttattaacaggagtatgtgctccaaatagaactccattaataagaaaacttaaaaacaaaggtgttcatataaatgtaaatactaaaatattagaagtaactgaccacgacgttaaagttcagagagctgacggtgcagaagaatggttaaaaggattcgactacatactattcggacttggttctagaaactacgatccaatatctgaacagataaaagaattcgttccagaagtacacgttgttggggatgctaagagagctagacaggcaagctttgcaatgtgggaagctttcgaagcagcatacagcttataa

SEQ ID NO: 2 - DNA, 1393 bp, Clostridium hiranonis
  source 1..1393: mol_type = unassigned DNA
acatgcaagtcgagcgattctcttcggagaagagcggcggacgggtgagtaacgcgtgggtaacctgccctgtacacacggataacataccgaaaggtatgctaatacgggataatatataagagtcgcatgacttttatatcaaagatttttcggtacaggatggacccgcgtctgattagcttgttggcggggtaacggcccaccaaggcgacgatcagtagccgacctgagagggtgatcggccacattggaactgagacacggtccaaactcctacgggaggcagcagtggggaatattgcacaatgggcgcaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggatttactgggcgtaaagggtgcgtaggcggtctttcaagtcaggagttaaaggctacggctcaaccgtagtaagctcctgatactgtctgacttgagtgcaggagaggaaagcggaattcccagtgtagcggtgaaatgcgtagatattgggaggaacaccagtagcgaaggcggctttctggactgtaactgacgctgaggcacgaaagcgtggggagcaaacaggattagataccctggtagtccacgctgtaaacgatgagtactagttgtcggaggttaccccttcggtgccgcagctaacgcattaagtactccgcctggggagtacgcacgcaagtgtgaaactcaaaggaattgacggggacccgcacaagtagcggagcatgtggtttaattcgaagcaacgcgaagaaccttacctaggcttgacatccttctgaccgaggactaatctcctctttccctccggggacagaagtgacaggtggtgcatggttgtcgtcagctcgtgtcgtgagatgttgggttaagtcccgcaacgagcgcaacccttgtctttagttgccatcattaagttgggcactctagagagactgccagggataacctggaggaaggtggggatgacgtcaaatcatcatgccccttatgcctagggctacacacgtgctacaatgggtggtacagagggcagccaagccgtgaggtggagcaaatcccttaaagccattctcagttcggattgtaggctgaaactcgcctacatgaagctggagttactagtaatcgcagatcagaatgctgcggtgaatgcgttcccgggtcttgtacacaccgcccgtcacaccatgggagttggagacacccgaagccgactatctaaccttttgggagaagtcgtccccctcgaatcaatacccc

SEQ ID NO: 3 - DNA, 9743 bp, Clostridium scindens
  source 1..9743: mol_type = unassigned DNA
aaaagatattaagcattaagaaaatgcacaaaaaatcagcgtgtgagagggagggcaaggagttgaagcgtgacttttttaacaagtttaatttggggacatcgaactttgtcacgccgggaaaacagttggaatacgtttcggaatgcaagccagattctactgcggtcatttgcttagataaagaacagaactgttccgttattacttggcatcagctgcacgtctattccagccagctggcatggtaccttatagaaaatgagattggcccggggtcgatcgtacttacaatgtttccgaacagcatcgagcacattattgcggtatttgcaatctggaaggcgggcgcctgctatatgcccatgtcctataaggcggcggaatccgagatcagggaggcctgcgataccatccacccgaatgcggcttttgcggaatgcaagattccaggattaaaattctgccttagcgcagacgagatatatgaggcgatggaaggaagatccaaggagatgccttcggaccgtctggccaatccgaacatgatatccttatcaggcggaaccagcggaaagatgaagttcatccgtcagaaccttccatgcgggctggacgatgagacgatcagaagctggtctttgatgtctggaatgggatttgagcagcgccagctgctggtaggcccgctgtttcatggcgcgcctcactccgcggcgtttaatggactgttcatgggcaacaccctggtactgaccaggaacctttgcccgggaaatatcctgaacatgattaagaaatataagattgaatttatacagatggtgccgaccctgatgaaccggcttgccaaactggagggagtcggaaaagaagactttgcatccctgaaggcgctgtgccatacagggggcgtctgttctccctggcttaagcagatctggatcgacctgctggggcctgaaaagatctatgagatgtattccatgacggaatgcatcggccttacctgcatccggggagacgagtgggtgaagcatccgggaagcatcggacggccagtgggcgatagcaaggtgtctatccgggatgagaatggcaaggaagttgcgccttttgagattggcgagatctatatgacagcgccggcctcctatctggttaccgagtacatcaattgggaaccgctggaagtgaaagagggaggcttccgaagcgtaggggatatcggctacgtggatgagcagggctatctgtacttttctgaccggcgcagcgacatgctggtatcaggcggagaaaacgtgttcgccaccgaagtcgagacggcgcttttgagatataaggatatcctggacgctgtagtggtagggataccggatgaagatctggggcgaaggctccatgcggtcattgagacagggaaagagataccggcagaggaactgaaaacattcctgagaaagtatctgactccatataagataccaaagacgttcgagttcgtaaggagcatacgaaggggagacaatggaaaggccgacaggaagcggatcctggaagattgtattgcccgcgggggatgattctataaatgcaaagaaaacaaattatataaaggaggagtaacaaaatgagttacgaagcacttttttcaccattcaaggtcagaggactggaacttaaaaaccgtatcgtcctgcctggaatgaacaccaagatggcaaagaacaagcacgacataggcgaggatatgatagcctaccatgttgccagggcaaaagcgggatgcgcgttaaatatatttgaatgcgtagcattatgtccggcgcctcacgcttatatgtatatggggctttatacggaccatcatgtagaacagcttaagaaattgacggatgcagtccatgaagcaggcggcaagatgggcatccagctgtggcatggaggattcagcccgcagatgttctttgacgagaccaacaccctggaaactccggacactcttacggtagagaggattcatgagatcgtagaagaattcggacgcggcgcaaggatggctgttcaggctggatttgacgcagtagaattccatgcggctcacagttatctgcctcacgagttcttaagccctggaatgaacaaacgtacggatgagtacggcggaagttttgagaaccgctgcagattctgttatgaagtcgttcaggcaatccgttccaatatcccggatgacatgccattctttatgcgtgcagactgcatcgacgaattaatggaacagaccatgacagaggaagagatcgttacatttatcaataagtgcgcagaacttggcgtggatgtggcagacctttcccgtggaaacgcgacttcattcgcaaccgtatatgaagttccgccattcaacctggctcatggcttcaacatagagaatatttacaacatcaaaaagcagatcaatatcccggttatgggagttggccgtatcaatacaggagagatggcaaacaaggtcattgaagaaggcaagtttgacctggtaggcatcggacgcgcccagcttgcagatccaaactggatcaccaaagtaagagaaggcaaagaagacctgatccgccactgtatcggatgtgaccagggatgctatgacgcagtcatcaatccaaagatgaagcatatcacctgcacccacaatccaggattgtgcttagagtatcagggaatgccaaagacagacgctcctaagaaagtcatgatcgtaggaggcggaatggcaggcatgatcgctgcggaagtattaaagaccagaggccataacccggtaatcttcgaggcatccgacaagcttgcaggacagttcaggctggcaggcgtagcgccgatgaagcaggattgggcagatgttgcagaatgggaagcaaaagaagtagagcgccttggaatcgaagtacgtctgaataccgaagtgactgcagagaccatcaaggaattcaatccggataatgtcatcatcgcagtaggctctacctatgcgctgcctgagattccgggaatcgacagcccaagcgtatactcccagtatcaggtactgaaaggggaagtaaatccgacaggccgtgtagccgttatcggatgcggactggttggtacggaagtcgcagaacttctggcatccagaggcgcacaggtaatcgcgatcgagaggaagggcgtaggtaccggccttagcatgcttcgcagaatgttcatgaacccggaattcaaatattacaagatcgccaagatgtccggaacaaatgtcaccgctttagagcagggcaaggttcactacatcatgacagacaagaagaccaaagaagtgacgcagggagtcctggaatgcgacgctaccgttatctgtacaggaattaccgcacgtccaagcgatgggcttaaggcaagatgcgaagaacttggaatcccggttgaggtgatcggagacgctgctggcgcaagagactgcacgatcgcgacacgcgaaggctatgacgcaggaatggcaatctagaaaatcagaacttatcaatcttacatatagaaaggatgatacatatgacattagaagagagagttgaagcattagaaaaagaattgcaggagatgaaggatattgaggcaatcaaggaactgaaaggaaagtatttccgctgcctggacggaaagatgtgggatgagctggagaccaccctgtcaccaaatatcgtaacctcttattccaacgggaaactggtattccatagcccgaaggaagttaccgattacttaaagagctcgatgccaaaagaagagatcagcatgcatatgggccacacgccggagatcaccattgacagcgagactacggctacgggcagatggtatctggaagatagactgatctttacggacggtaagtacaaagacgtaggaatcaatggcggcgcgttctatacagacaaatatgagaagatagacggccagtggtacatccttgaaaccggctatgtacgaatctatgaagaacatttcatgcgtgatccaaagatccatatcacgatgaacatgcacaaataagaatattgtaaaagaaaggcaggagtaagagtatgaatctcgtacaagacaaagttacgatcatcacaggcggcacaagaggtattggattcgccgctgccaaaatatttatcgacaatggcgcaaaagtatccatcttcggagagacgcaggaagaagtagatacagcgcttgcacagttaaaagaactttatccggaagaagaggttctgggattcgcgccggatcttacatccagagacgcagttatggcagcggtaggccaggtagcacagaaatatggcagactggatgtcatgatcaacaatgcaggaattaccagcaacaacgtattctccagagtgtctgaagaagagttcaagcatattatggacatcaacgtaacaggcgtattcaacggcgcatggtgcgcataccagtgcatgaaggatgccaaaaagggcgttatcatcaacacggcatccgttacaggcatcttcggatcactctcaggcgtaggatatccggccagcaaggcaagcgtgatcggactcacccatggacttggaagagagatcatccgcaagaatatccgtgtagtaggagtggctcctggagttgtgaacacggatatgaccaatggcaatcctccggagatcatggaaggatatctgaaggcgcttccgatgaagagaatgcttgagccggaagagatcgctaatgtatacctgttcctggcatctgacttggcaagcggcattacggctactacggtcagcgtagacggggcttacagaccataattttaatttttactaagtagaatatgtgatatagaaaaggagatataaaaacatggctggaataaaagattttccaaaattcggagctcttgcagggcttaagatacttgacagcggatctaacatcgccggacctttaggcggaggccttctggcagaatgcggagcaacggtcatccattttgaaggaccaaagaaacctgataaccagagaggatggtacggctatccacagaatcaccgtaatcagctgtctatggtagcagacatcaaatctgaagaaggaagaaagatcttccttgatctgatcaaatgggcagatatctgggtagagtcatccaaaggcggacagtatgacaggctgggactttccgatgaagtcatctgggaagtaaatcctaagattgccatcgtgcacgtatccggatatggacagacaggagacccgtcttacgttacacgtgcatcctatgacgcagtaggccaggcattcagcggctatatgtcactgaacggaacaacggaagcgctgaagatcaatccttatctgagcgatttcgtatgcggacttaccacatgctgggctatgcttgcctgctatgtaagcaccattcttaccggaaaaggcgaatctgttgacgttgcacagtacgaagcgctggcacgtatcatggacggacgtatgatccagtacgctacagacggcgtgaagatgccaagaaccggcaataaggatgcgcaggctgccctgttcagcttctacacctgtaaagacggacgtacgatctttatcggaatgactggcgcggaagtatgtaagagaggcttcccgatcatcggacttccggtacctggaaccggagacccggacttcccggaaggcttcacaggctggatgatctatactcctgtaggacagagaatggaaaaggctatggagaagtatgtatctgagcatacgatggaagaagtagaggctgagatgcaggcacaccagattccatgccagagagtatacgagctggaagactgcctgaacgatcctcactggaaagcacgtggaactattacggagtgggatgacccgatgatgggacatatcacaggccttggactgatcaacaagttcaagagaaatccttccgaaatctggagaggcgctccgctgttcggtatggataaccgcgatatcctgaaagacctgggatatgacgatgcaaagatcgatgaactctatgagcagggcatcgtcaatgaattcgaccttgacactactatcaaacgctatagactggatgaagtaattccacatatgagaaagaaagaggagtaagagtatgagcaccgtagccaatccaaattataagaaaggttttgtcccctttgcaattgcagcactcctggtgagcctgatcggcggttttaccgccgttctcggcccggccttcgtggcggaccaggggattgactataataataccacatggatttccctggcgctggcgatgtcttccgccgcatgcgctccaatccttggaaaactgggagacgtgctaggacgcaggacgacgctgcttctgggtattgtgatctttgcggccggcaatgtgctgacagccgtagccacgtccctgatattcatgctggcagcccgttttatcgtaggtatcggaacagcagcgatctcaccgatcgttatggcctatatcgtaaccgagtatccgcaggaggagacaggaaaggcctttggcctgtatatgctgatctccagcggcgccgtcgtggtaggacctacctgtggcggcctgatcatgaatgcggctggctggagagtcatgatgtgggtatgcgtcgctctgtgcgtcgttgtattcctgatctgcacattctccatcaagaagactgcatttgagaagaagagcatggcaggatttgacaagccgggcgcagccctggtagtcgtattcttcagtttgttcctgtgcatcccatccttcggacagaatatcggatggtcttccacagcatttatcgcagcagcggcagtagcgctggtagcacttttcatcctggtaatggtagaaaagaaagcgaagagtccgatcatgaacggcaagtttatggcacgcaaggaattcgtgcttccagtattgatcctgttccttacacagggacttatgatggcaaatatgaccaatgtcatcgtgttcgtgcgctatacgcagccggacaatgtcattatatcaagttttgcgatctccatcatgtacataggaatgtccttaggctccgttatcattggacctgttgcagataagaaagagccaaagacggttctgacattctctctggtactgacagccatcggctgtgcgctgatgtatctgttcaaggcagattcctccgtcgctatctttgcggcatccttgggaatccttggatttggccttggaggaaatgcaaccatcttcatgaaggtagcgctttccggcctgtccagcgaagtagctggctctggtactggaacctatggcctgttcagagatatctcggcaccattcggcgtggcagtgttcgtgcctatgtttgccaacggcgtaacagcgaatattgcgaaatacgcgtcaggcggcatggaagaaggcgccgctacggtaaaagcagccatctcatccatccagacgctgacactggttgaacttggatgtatcgttgtgggaatcatccttgtgagaatgctgccaagaatctatcagaagaaagaggcataaataagttaagaaaagaggtaattataaatggatatgaaacattccagattattttcgccgcttcagatcggatccctgacactgtctaaccgtgtcggcatggctcccatgagcatggactatgaagcagcagacggaactgtgcccaagaggctggcggacgtatttgtccgccgcgccgagggaggcacaggctacgtcatgatcgacgcggtgacgatagacagcaagtatccttatatgggaaatacaacggcccttgaccgtgatgaactggttccccagtttaaggaatttgctgacagagtaaaagaagcaggcagcacgctggtgccgcagatcattcatccgggtccggaatccgtatgcggctaccggcatatcgctccgcttggaccttctgccaacaccaatgcaaactgccacgtgagcagatcgatcagcatagatgagatccatgacatcattaagcagttcggccaggcggcacgccgcgccgaagaagcaggatgcggggcaatctccctgcactgcgcgcatgcgtatatgctgccaggatccttcctgtcaccgcttcgcaacaagcgcatggatgaatatggcggaagccttgacaaccgtgcccgtttcgtgatcgagatgattgaggaggcccgcaggaatgtgagtcctgatttcccgatcttccttcgtatctccggagacgagagaatggtaggaggcaacagccttgaagatatgctctacctggcaccgaagttcgaggctgccggcgtaagcatgctggaagtatccggcggaacccagtatgaaggcctggaacatatcattccttgccagaataagagcaggggcgtcaatgtatatgaagcttctgagatcaagaaagtagtgggcatcccggtatacgcagtaggaaagatcaacgatatacgctatgcggcagagatcgtagaacgcggcctggtagacggcgtggctatgggacgtccgcttctggcagatccggacctttgcaagaaggcagtggaaggccagtttgacgagatcactccatgcgcaagctgcggcggaagctgcatcagccgttctgaggcagcgcctgagtgccattgccatattaatccaaggcttggccgggagtatgaattcccggatgtgcctgccgagaagtccaagaaggtactggttatcggcgcaggccctggaggaatgatggctgccgtgacagctgcggaacgcggccatgatgttacggtatgggaggctgacgacaagatcggcggccagctgaacctggcagtagtggctcctggcaagcaggagatgacccagtggatggtacatctgaactatcgcgcgaagaaagcaggcgtgaagtttgaattcaataaagaagcgacggcagaagatgtcaaggcgctggcgccggaagcagtgatcgttgctacaggcgcgaagccgctggttcctccgattaaaggaacacaggattatccggtgcttactgcccatgatttccttcgcggcaagttcgtgattccgaagggacgcgtctgcgtgctgggaggaggcgcggttgcctgcgagactgccgagacagccctggagaatgcacgtccgaattcttataccagaggatacgatgcaagcatcggagatatcgatgtcacgcttgtggagatgcttccgcagctccttaccggcgtatgcgcgccgaaccgcgagcctttgatccgcaagttaaagagcaagggcgtacacatcaacgtcaataccaagatcatggaagtaacagaccatgaagtaaaggttcagagacaggatggaacgcaggaatggctggaaggatttgactatgtcctctttggccttggttccagaaattacgatccgctttcagagaccctcaaggaattcgttccggaagtacatgtcatcggcgatgccgtaagggcgcgccaggcaagctacgcaatgtgggaaggatttgagaaggcatacagcctgtaaaagcggtttgagtaaaaggaggcttaagaaatggcagtgaaggcaatctcaggctgcgacaaggatcaggaactgatca

SEQ ID NO: 4 - DNA, 1531 bp, Clostridium scindens
  source 1..1531: mol_type = unassigned DNA
gagagtttgatcctggctcaggatgaacgctggcggcgtgcctaacacatgcaagtcgaacgaagcgcttccgctagattttcttcggagatgaaggcggctgcgactgagtggcggacgggtgagtaacgcgtgggcaacctgccttgcactgggggataacagccagaaatggctgctaataccgcataagaccgaagcgccgcatggcgcagcggccaaagccccggcggtgcaagatgggcccgcgtctgattaggtagttggcggggtaacggcccaccaagccgacgatcagtagccgacctgagagggtgaccggccacattgggactgagacacggcccagactcctacgggaggcagcagtggggaatattgcacaatgggggaaaccctgatgcagcgacgccgcgtgaaggatgaagtatttcggtatgtaaacttctatcagcagggaagaagatgacggtacctgactaagaagccccggctaactacgtgccagcagccgcggtaatacgtagggggcaagcgttatccggatttactgggtgtaaagggagcgtagacggcgatgcaagccagatgtgaaagcccggggctcaaccccgggactgcatttggaactgcgtggctggagtgtcggagaggcaggcggaattcctagtgtagcggtgaaatgcgtagatattaggaggaacaccagtggcgaaggcggcctgctggacgatgactgacgttgaggctcgaaagcgtggggagcaaacaggattagataccctggtagtccacgccgtaaacgatgactactaggtgtcgggtggcaaggccattcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgatcttgacatcccgatgccaaagcgcgtaacgcgctctttcttcggaacatcggtgacaggtggtgcatggttgtcgtcagctcgtgtcgtgaggtgttgggttaagtcccgcaacgagcgcaacccctatcttcagtagccagcatttcggatgggcactctggagagactgccagggacaacctggaggaaggtggggatgacgtcaaatcatcatgccccttatgaccagggctacacacgtgctacaatggcgtaaacaaagggaggcgaacccgcgagggtgggcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagccggtgacccaacccgcaagggagggagccgtcgaaggtgggaccgataactggggtgaagtcgtaacaaggtagccgtatcggaaggtgcggctggatcacctccttc

SEQ ID NO: 5 - DNA, 20 bp, synthetic construct
  misc_feature 1..20: note = Synthetic primer
  source 1..20: mol_type = other DNA
agagtttgatcmtggctcag

SEQ ID NO: 6 - DNA, 19 bp, synthetic construct
  misc_feature 1..19: note = Synthetic primer
  source 1..19: mol_type = other DNA
gtgccagcmgccgcggtaa

SEQ ID NO: 7 - DNA, 20 bp, synthetic construct
  misc_feature 1..20: note = Synthetic primer
  source 1..20: mol_type = other DNA
cggttaccttgttacgactt

SEQ ID NO: 8 - DNA, 1657 bp, Helicobacter sp.
  source 1..1657: mol_type = unassigned DNA
agagtttgatcctggctcagagtgaacgctggcggcgtgcctaatacatgcaagtcgaacgatgaaacttctagcttgctagaagtggattagtggcgcacgggtgagtaatgcataggtaacatgccctttagtctgggatagccactggaaacggtgattaatactggatactccctacgggggaaaggggctttcaataaagaatttctctttttagtgttttgtgttgttggcacaaaattctagtatttggaatgagaaattggtgttgtgaagcaatttgtgcggagattagacttagtgtctgtcgtgtcagcaaattgcgaactcatcgatttatcatccaaagacgaattttttattgaaagccttcgctaaaggattggcctatgtcctatcagcttgttggtgaggtaatggctcaccaaggctatgacgggtatccggcctgagagggtgatcggacacactggaactgagacacggtccagactcctacgggaggcagcagtagggaatattgctcaatgggggaaaccctgaagcagcaacgccgcgtggaggatgaaggttttaggattgtaaactccttttgtaagagaagattatgacggtatcttacgaataagcaccggctaactccgtgccagcagccgcggtaatacggagggtgcaagcgttactcggaatcactgggcgtaaagagcgcgtaggcgggtggtcaagtcagatgtgaaatcctgtagcttaactacagaactgcatttgaaactgaccatctagagtatgggagaggtaggtggaattcttggtgtaggggtaaaatccgtagagatcaagaggaatactcattgcgaaggcgacctgctggaacattactgacgctgatgcgcgaaagcgtggggagcaaacaggattagataccctggtagtccacgccctaaacgatgaatgctagttgttgtgaggcttgtccttgcagtaatgcagctaacgcattaagcattccgcctggggagtacggtcgcaagattaaaactcaaaggaatagacggggacccgcacaagcggtggagcatgtggtttaattcgatgatacgcgaagaaccttacctaggcttgacattgatagaatctactagagatagtggagtgcccttttagggagcttgaaaacaggtgctgcacggctgtcgtcagctcgtgtcgtgagatgttgggttaagtcccgcaacgagcgcaaccctcgtccttagttgctagcagtttggctgagcactctaaggagactgccttcgtaaggaggaggaaggtgaggacgacgtcaagtcatcatggcccttacgcctagggctacacacgtgctacaatggggtgcacaaagagatgcaatagtgtgagctggagccaatctctaaaacatctctcagttcggattgtagtctgcaactcgactacatgaagctggaatcgctagtaatcgcaaatcagcaatgttgcggtgaatacgttcccgggtcttgtactcaccgcccgtcacaccatgggagttgtatttgccttaagtcggaatgctaaattggctaccgcccacggcagatgcagcgactggggtgaagtcgtaacaaggtaaccgtaggtgaacctgcggttg

SEQ ID NO: 9 - DNA, 1340 bp, Campylobacter sp.
  source 1..1340: mol_type = unassigned DNA
agtgaacgctggcggcgtgcctaatacatgcaagtcgaacgatgaagcttctagcttgctagaagtggattagtggcgcacgggtgagtaaggtatagttaatctgccctacacaagaggacaacacctagaaatgggtgctaatactctatactcctgcttaacacaagttgagtagggaaagtttttcggtgtaggatgagactatatagtatcagctagttggtaaggtaaaggcttaccaaggctatgacgcttaagaggtctgagaggatgatctctcacactggaactgagacacggtccagactcctacgggaggcagcagtagggaatattgcgcaatgggcgaaagcctgacgcagcaacgccgcgtggaggatgacacttttaggagcgtaaactccttttcttagggaagaattctgacggtacctaaggaataagcaccggctaactccgtgccagcagccgcggtaatacggagggtgcaagcgttactcggaatcactgggcgtaaagggcgcgtaggcggattatcaagtctcttgtgaaatctaatggcttaaccattaaactgcttgggaaactgatagtctagagtgagggagaggcagatggaattggtggtgtaggggtaaaatccgtagatatcaccaagaatacccattgcgaaggcgatctgctggaactcaactgacgctaaggcgcgaaagcgtggggagcaaacaggattagataccctggtagtccacgccctaaacgatgtatgctagttgttgggctgctagtcagctcagtaatgcagctaacgcattaagcataccgcctggggagtacggtcgcaagattaaaactcaaaggaatagacggggacccgcacaagcggtggagcatgtggtttaattcgaagatacgcgaagaaccttacctaggcttgatatccaacaaagcttctagagatagaagtgtgctagcttgctagaatgttgagacaggtgctgcacggctgtcgtcagctcgtgtcgtgagatgttgggttaagtcccgcaacgagcgcaacccacgtatttagttgctaacacttcgggtgagcactctaaatagactgccttcgtaaggaggaggaaggtgtggacgacgtcaagtcatcatggcccttatgcctagggcgacacacgtgctacaatggcatatacaatgagacgcaataccgcgaggtggagcaaatctataaaatatgtcccagttcggattgttctctgcaactcgagagcatgaagccggaatcgctagtaatcgcaaatcagccatgttgcggtgaatacgttcccgggtct

SEQ ID NO: 10 - DNA, 1378 bp, Succinivibrio sp.
  source 1..1378: mol_type = unassigned DNA
catgcaagtcgaacggtaacatagaggaagcttgctttctctgatgacgagtggcggacgggtgagtaaggtctgggaaactgcctgacagagggggacaacaactggaaacggttgctaataccgcatacaccctgagggggaaagtcgaaagacgctgtcagatgtgcccagatgggattagctagtaggtgaggtaaaggctcacctaggcgacgatctctagctggtctgagaggatgatcagccacattgggactgagacacggcccagactcctacgggaggcagcagtagggaatattgcacaatggggggaaccctgatgcagccatgccgcgtgtgtgaagaaggccttcgggttgtaaagcactttcagaggggaggaaaatgacgttaccctcagaagaagcaccggctaactccgtgccagcagccgcggtaatacggagggtgcaagcgttaatcggaataactgggcgtaaagggcatgcaggcggttctgcaagtagggtgtgaaagcccggggctcaacctcggaattgcactctaaactgtgggactagagtattgcagggggagacggaattccaggtgtagcggtggaatgcgtagagatctggaagaacaccaaaggcgaaggcagtctcctgggcaaatactgacgctcatatgcgaaagcgtgggtagcaaacaggattagataccctggtagtccacgccgtaaacgatgttgattagaagcttgcttgtaagagtgggtttcgcagctaacgcgataaatcaaccgcctggggagtacggccgcaaggttaaaactcaaatgaattgacgggggcccgcacaagcggtggagcatgtggtttaattcgacgcaacgcgatgaaccttacctgatcttgacatcgcgagaattacttgtaatgagtaagtgccttcgggaactcgcagacaggtgctgcatggctgtcgtcagctcgtgtcgtgagatgttgggttaagtcccgcaacgagcgcaacccttgtcctttgttgccagcgggtagagccgggaactcaaaggagactgccagtgataaactggaggaaggtagggatgacgtcaagtcatcatggcccttacggtcagggctacacacgtgctacaatggggcgtacagagggaaacgaaactgcgaggtggagtggaacccagaaagcgtccctaagttcggattggagtctgcaactcgactccatgaagtcggaatcgctagtaatcgcaaatcagaatgttgcggtgaatacgttcccgggccttgtacacaccgcccgtcacaccatgggagtggattgcaccagaagtggccagcctaactgcaaagagggcggtaccacg

SEQ ID NO: 11 - DNA, 253 bp, Anaerobiospirillum sp.
  source 1..253: mol_type = unassigned DNA
tacggagggtgcaagcgttaatcggaataactgggcgtaaagggcatgtaggcggaaaggcaagcaagatgtgaaagacctgggctcaacctgggttggtcattttgaactacctttctagagtattgcagagggagatggaatttcaggtgtagcggtggaatgcgtagatatctgaaagaacaccagaggcgaaggcggtctcctgggcaaatactgacgctgaggtgcgaaagcgtggggagcaaacagg

SEQ ID NO: 12 - DNA, 1357 bp, Collinsella sp.
  misc_difference 766: note = a, c, t, g, unknown or other
  source 1..1357: mol_type = unassigned DNA
acggcacccctctccggagggaagcgagtggcgaacggctgagtaacacgtggagaacctgccccctcccccgggatagccgcccgaaaggacgggtaataccggatacccccgggcgccgcatggcgcccgggctaaagccccgacgggaggggatggctccgcggcccatcaggtagacggcggggtgacggcccaccgtgccgacaacgggtagccgggttgagagaccgaccggccagattgggactgagacacggcccagactcctacgggaggcagcagtggggaatcttgcgcaatggggggaaccctgacgcagcgacgccgcgtgcgggacggaggccttcgggtcgtaaaccgctttcagcagggaagagtcaagactgtacctgcagaagaagccccggctaactacgtgccagcagccgcggtaatacgtagggggcgagcgttatccggattcattgggcgtaaagcgcgcgtaggcggcccggcaggccgggggtcgaagcggggggctcaaccccccgaagcccccggaacctccgcggcttgggtccggtaggggagggtggaacacccggtgtagcggtggaatgcgcagatatcgggtggaacaccggtggcgaaggcggccctctgggccgagaccgacgctgaggcgcgaaagctgggggagcgaacaggattagataccctggtagtcccagccgtaaacgatggacgctgggtgtggggggacgatccccccgtgccgcagccnacgcattaagcgtcccgcctggggagtacggccgcaaggctaaaactcaaaggaattgacgggggcccgcacaagcagcggagcatgtggcttaattcgaagcaacgcgaagaaccttacggcgcatccccccgaggcccacggggggtccgccgcgtgggtcagaggagcgcatacgggaggtgcatggttgtcgtcagctcgtgtcgtgagatgttgggttaagtcccgcaacgagcgcaacccccgccgcgtgttgccatcgggtgatgccgggaacccacgcgggaccgccgccgtcaaggcggaggagggcggggacgacgtcaagtcatcatgccccttatgccctgggctgcacacgtgctacaatggccggtacagagggatgccaccccgcgagggggagcggatcccggaaagccggccccagttcggattgggggctgcaacccgcccccatgaagtcggagttgctagtaatcgcggatcagcatgccgcggtgaatgcgttcccgggccttgtacacaccgcccgtcacaccacccgagtcgtctgcacccgaagtcgccggcccaaccgcaaggggg

SEQ ID NO: 13 - DNA, 1454 bp, Clostridium sp.
  misc_difference 635: note = a, c, t, g, unknown or other
  misc_difference 704: note = a, c, t, g, unknown or other
  source 1..1454: mol_type = unassigned DNA
tgagtttgatcatggctcaggacgaacgctggcggcgtgcctaacacatgcaagtcgagcgatgaaattttcttcggaaaatggattagcggcggacgggtgagtaacacgtgggtaacctgccctatagagagggatagccttccgaaagggagattaatacctcataatatcctagtatcgcatgatacatggattaaaggagcaatccgctataggatggacccgcggcgcattagctagttggtgaggtaacggctcaccaaggcgacgatgcgtagccgacctgagagggtgatcggccacattgggactgagacacggcccagactcctacgggaggcagcagtggggaatattgcacaatgggggaaaccctgatgcagcaacgccgcgtgagtgatgacggtcttcggattgtaaagctctgtctttagggacgataatgacggtacctaaggaggaagccacggctaactacgtgccagcagccgcggtaatacgtaggtggcaagcgttgtccggatttactgggcgtaaagggagcgtaggcggatctttaagtgggatgtgaaatactcgggctcaacctgggggctgcattccaaactggggatctagagtacaggaggggngagtggaattcctagtgtagcggtgaaatgcgtagagattaggaagaacaccagtggcgaaggcgactntctggactgtaactgacgctgaggctcgaaagcgtggggagcaaacaggattagataccctggtagtccacgccgtaaacgatgaatactaggtgtagggggtgtcaactccccctgtgccgccgctaacgcattaagtattccgcctggggagtacggtcgcaagattaaaactcaaaggaattgacgggggcccgcacaagtagcggagcatgtggtttaattcgacgcaacgcgaagaaccttacctagacttgacatcttctgcattacccttaatcggggaagttccttcggggacagaatgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgtctagggctacacacgtgctacaatggcaagtacaaagagaagcaatactgtgaagtggagcaaaactcaaaaacttgtctcagttcggattgtaggctgaaactcgcctacatgaagctggagttgctagtaatcgcgaatcagaatgtcgcggtgaatacgttcccgggtcttgtacacaccgcccgtcacaccatgagagttggcaatacccgaagtccgtaagctaaccgtaaggaggcagcggccgaaggtagggtcagcgatgggg

SEQ ID NO: 14 - DNA, 253 bp, Clostridium sp.
  source 1..253: mol_type = unassigned DNA
tacgtaggtggcgagcgttatccggatttactgggcgtaaagggagcgtaggcggatgattaagtgggatgtgaaatacccgggctcaacttgggtgctgcattccaaactggttatctagagtgcaggagaggagagtggaattcctagtgtagcggtgaaatgcgtagagattaggaagaacaccagtggcgaaggcgactctctggactgtaactgacgctgaggctcgaaagcgtggggagcaaacagg

SEQ ID NO: 15 - DNA, 1403 bp, Allisonella sp.
  source 1..1403: mol_type = unassigned DNA
agagtttgatcctggctcaggacgaacgctggcggcatgcttaacacatgcaagtcgaacggactgattccttcgggatgaaagttagtggcgaacgggtgagtaatgtatgagcaacctgcctctgtcaacgggataacagttggaaacgactgctaatacggtatatgaccacggcaccgcatggtgcagcggtaaaagattttatcggacagagatgggctcatatcccattaggtagttggtgagataacagcccaccaagccgacgatcagtagccggtctgagaggatgaacggccacactggaactgagacacggtccagactcctacgggaggcagcagtggggaatcttccgcaatggacgaaagtctgacggagcaacgccgcgtgaacgatgaaggtcttcggattgtaaagttctgtgatccgggacgaaggcattgattgagaacattgattgatgttgacggtaccggaaaagcaagccacggctaactacgtgccagcagccgcggtaatacgtaggtggcaagcgttgtccggaattattgggcgtaaagcgcgcgcaggcggccgtgcaagtccatcttaaaagcgtggggcttaaccccatgaggggatggaaactgcagggctggagtgtcggaggggaaagtggaattcctagtgtagcggtgaaatgcgtagagattaggaagaacaccggtggcgaaggcgactttctagacgacaactgacgctgaggcgcgaaagcgtggggagcaaacaggattagataccctggtagtccacgccgtaaacgatggatactaggtgtaggaggtatcgaccccttctgtgccggagttaacgcaataagtatcccgcctgggaagtacgatcgcaagattaaaactcaaaggaattgacgggggcccgcacaagcggtggagtatgtggtttaattcgacgcaacgcgaagaaccttaccaagccttgacattgatcgcaatccgcagaaatgcggagttcctcttcggaggacgagaaaacaggtggtgcacggctgtcgtcagctcgtgtcgtgagatgttgggttaagtcccgcaacgagcgcaacccctatcttctgttgccagcacgtaaaggtgggaactcaggagagaccgccgcggacaacgcggaggaaggcggggatgacgtcaagtcatcatgccccttatggcttgggctacacacgtactacaatgggtgcaaacaaagagaagcgaagtcgcgagatggagcggacctcataaacgcactcccagttcagattgcaggctgcaacccgcctgcatgaagtaggaatcgctagtaatcgcgggtcagcataccgcggtgaatacgttcccgggccttgtacacaccgcccgtca

SEQ ID NO: 16 - DNA, 1375 bp, Tyzzerella sp.
  source 1..1375: mol_type = unassigned DNA
ctggctcaggatgaacgctggcggcgtgcttaacacatgcaagtcgaacgaagagggttagaatgagagcttcggcaggatttctttccatcttagtggcggacgggtgagtaacgtgtgggcaacctgccctgtactgggggataatcattggaaacgatgactaataccgcatgtggttctcggaaggcatcttctgaggaagaaaggatttattcggtacaggatgggcccgcatctgattagctagttggtgagataacagcccaccaaggcgacgatcagtagccgacctgagagggtgatcggccacattgggactgagacacggcccaaactcctacgggaggcagcagtggggaatattgcacaatgggcgaaagcctgatgcagcaacgccgcgtgaaggatgaagggtttcggctcgtaaacttctatcaatagggaagaaacaaatgacggtacctaaataagaagccccggctaactacgtgccagcagccgcggtaatacgtagggggcaagcgttatccggaattactgggtgtaaagggagcgtaggcggcatggtaagccagatgtgaaagccttgggcttaacccgaggattgcatttggaactatcaagctagagtacaggagaggaaagcggaattcctagtgtagcggtgaaatgcgtagatattaggaagaacaccagtggcgaaggcggctttctggactgaaactgacgctgaggctcgaaagcgtggggagcaaacaggattagataccctggtagtccacgccgtaaacgatgagtgctaggtgtcggggaggaatcctcggtgccgtagctaacgcaataagcactccacctggggagtacgaccgcaaggttgaaactcaaaggaattgacgggggcccgcacaagcggtggagcatgtggtttaattcgaagcaacgcgaagaaccttaccaaggcttgacatcccgatgaccgtcctagagataggacttctcttcggagcatcggtgacaggtggtgcatggttgtcgtcagctcgtgtcgtgagatgttgggttaagtcccgcaacgagcgcaacccttgtcactagttgctacgaaagggcactctagtgagactgccggtgacaaaccggaggaaggtggggatgacgtcaagtcctcatggcccttatgggtagggcttcacacgtcatacaatggtcggaacagagggcagcgaagccgtgaggcggagccaatcccagaaaaccgatcgtagtccggattgcagtctgcaactcgactgcatgaagtcggaatcgctagtaatcgcggatcagcatgccgcggtgaatacgttcccgggtcttgtacacaccgcccgta

SEQ ID NO: 17 - DNA, 1410 bp, Bacteroides sp.
  source 1..1410: mol_type = unassigned DNA
ggcgcacgggtgagtaacacgtatccaacctgccgataactcggggatagcctttcgaaagaaagattaatacccgatggcatgtaaagacctcctggtctttacattaaagaatttcggttatcgatggggatgcgttccattagatagtaggcggggtaacggcccacctagtccacgatggataggggttctgagaggaaggtcccccacattggaactgagacacggtccaaactcctacgggaggcagcagtgaggaatattggtcaatggacgcgagtctgaaccagccaagtagcgtgaaggaagactgccctatgggttgtaaacttcttttatacgggaataaagtattccacgtgtgggattttgtatgtaccgtatgaataaggatcggctaactccgtgccagcagccgcggtaatacggaggatccgagcgttatccggatttattgggtttaaagggagcgtaggtggaagattaagtcagcctgtgaaagtttgcggcttaaccgtaaaattgcagttgatactggttttcttgagtgcagtagaggtgggcggaattcgtggtgtagcggtgaaatgcttagatatcacgaagaactccgattgcgaaggcagctcactggactgtaactgacactgatgctcgaaagtgtgggtatcaaacaggattagataccctggtagtccacacagtaaacgatgaatactcgctgtttgcgatatacagtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattacacctgaatagattggaaacattttagccgcaaggcaggtgtgaaggtgctgcatggttgtcgtcagctcgtgccgtgaggtgtcggcttaagtgccataacgagcgcaacccttatcttcagttactaacagttatagctgaggactctgaagagactgccgtcgtaagatgtgaggaaggtggggatgacgtcaaatcagcacggcccttacgtccggggctacacacgtgttacaatggggggtacagaaggctgctacctggcgacaggatgccaatccttaaatcctctctcagttcggactggagtctgcaacccgactccacgaagctggattcgctagtaatcgcgcatcagccatggcgcggtgaatacgttcccgggccttgtacacaccgcccgtcaagccatgaaagccgggggtacctgaagtgcgtaaccgcaaggagcgtcctagggtaaaactggtaattggggctaagtcgtaacaaggtagccgtaccggaaggtgcggctg

SEQ ID NO: 18 - DNA, 1394 bp, Parabacteroides sp.
  source 1..1394: mol_type = unassigned DNA
agagtttgatcctggctcaggatgaacgctagcgacaggcttaacacatgcaagtcgaggggcagcatgatttgtagcaatacagattgatggcgaccggcgcacgggtgagtaacgcgtatgcaacttacctatcagagggggatagcccggcgaaagtcggattaataccccataaaacaggggtcccgcatgggaatatttgttaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgaaggaagaaggatctatggtctgtaaacttcttttataggggaataaagtggaggacgtgtccttttttgtatgtaccctatgaataagcatcggctaactccgtgccagcagccgcggtaatacggaggatgcgagcgttatccggatttattgggtttaaagggtgcgtaggtggtgatttaagtcagcggtgaaagtttgtggctcaaccataaaattgccgttgaaactgggttacttgagtgtgtttgaggtaggcggaatgcgtggtgtagcggtgaaatgcatagatatcacgcagaactccgattgcgaaggcagcttactaaaccataactgacactgaagcacgaaagcgtggggatcaaacaggattagataccctggtagtccacgcagtaaacgatgattactaggagtttgcgatacaatgtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgtagtctgaccggaatggaaacactccttctagcaatagcagattacaaggtgctgcatggttgcctcaactccggcccggaaggtccggcttaattgccataacaagcgcacccttttaccaaggttcaaacaggtgaagcttgaagactctgtggaacctcccccctaacctgtgagaagaagtggggatacactcaataaaccacggcccttaatcccggggggaacactggttacaatgggttgggaaagggggcttcctggcgacaggatgctaatctccaaaccatgtctcagttcggatcggagtctgcaactcgactccgtgaagctggattcgctagtaatcgcgcatcagccatggcgcggtgaatacgttcccgggccttgtacacaccgcccgtc

SEQ ID NO: 19 - DNA, 1451 bp, Bacteroides sp.
  source 1..1451: mol_type = unassigned DNA
gatgaacgctagctacaggcttaacacatgcaagtcgaggggcagcatggtcttagcttgctaaggctgatggcgaccggcgcacgggtgagtaacacgtatccaacctgccgtctactcttggccagccttctgaaaggaagattaatccaggatgggatcatgagttcacatgtccgcatgattaaaggtattttccggtagacgatggggatgcgttccattagatagtaggcggggtaacggcccacctagtcaacgatggataggggttctgagaggaaggtcccccacattggaactgagacacggtccaaactcctacgggaggcagcagtgaggaatattggtcaatgggcgcgagcctgaaccagccaagtagcgtgaaggatgactgccctatgggttgtaaacttcttttgtccgggaataaaaccgcctacgtgtaggcgcttgtatgtaccggtacgaataagcatcggctaactccgtgccagcagccgcggtaatacggaggatgcgagcgttatccggatttattgggtttaaagggagcgcagacgggtttttaagtcagctgtgaaagtttggggctcaaccttaaaattgcagttgatactggagaccttgagtgcagttgaggcaggcggaattcgtggtgtagcggtgaaatgcttagatatcacgaagaactccgattgcgaaggtagcttgctaaagtgtaactgacgttcatgctcgaaagtgtgggtatcaaacaggattagataccctggtagtccacacggtaaacgatggatactcgctgttggcgatatacggtcagcggcttagcgaaagcgttaagtatcccacctggggagtacgccggcaacggtgaaactcaaaggaattgacgggggcccgcacaagcggaggaacatgtggtttaattcgatgatacgcgaggaaccttacccgggcttaaattgcactggactattctggaaacaggatattcttcggaccagtgtgaaggtgctgcatggttgtcgtcagctcgtgccgtgaggtgtcggcttaagtgccataacgagcgcaacccttgctgccagttactaacaggtaatgctgaggactctggcgggactgccatcgtaagatgcgaggaaggtggggatgacgtcaaatcagcacggcccttacgtccggagctacacacgtgttacaatggtaggtacagagggtagctacccagcgatgggatgcgaatctcgaaagcctatctcagttcggattggaggctgaaacccgcctccatgaagttggattcgctagtaatcgcgcatcagccatggcgcggtgaatacgttcccgggccttgtacacaccgcccgtcaagccatgggagccgggggtacctgaagtacgtaaccgcaaggatcgtcctagggtaaaactggtgactgggg

SEQ ID NO: 20 - DNA, 1450 bp, Bacteroides sp.
  source 1..1450: mol_type = unassigned DNA
gatgaacgctagctacaggcttaacacatgcaagtcgaggggcagcatgaagtttgcttgcaaactttgatggcgaccggcgcacgggtgagtaacgcgtatccaacctcccgcatactcggggatagccttctgaaaggaagattaatacccgatggtatcttaagcgcacatgcaattaagattaaagaatttcggtatgcgatggggatgcgttccattaggtagtaggcggggtaacggcccacctagccatcgatggataggggttctgagaggaaggtcccccacattggaactgagacacggtccaaactcctacgggaggcagcagtgaggaatattggtcaatgggcgcgagcctgaaccagccaagtagcgtgaaggatgactgccctatgggttgtaaacttcttttgtccgggaataaaaccgcctacgtgtaggcgcttgtatgtaccggtacgaataagcatcggctaactccgtgccagcagccgcggtaatacggaggatgcgagcgttatccggatttattgggtttaaagggagcgcagacgggtttttaagtcagctgtgaaagtttggggctcaaccttaaaattgcagttgatactggagaccttgagtgcagttgaggcaggcggaattcgtggtgtagcggtgaaatgcttagatatcacgaagaactccgattgcgaaggcagcttgctaaagtgtaactgacgttcatgctcgaaagtgtgggtatcaaacaggattagataccctggtagtccacacggtaaacgatggatactcgctgttggcgatatacggtcagcggcttagcgaaagcgttaagtatcccacctggggagtacgccggcaacggtgaaactcaaaggaattgacgggggcccgcacaagcggaggaacatgtggtttaattcgatgatacgcgaggaaccttacccgggcttaaattgcactggactttcccggaaacgggattttcttcggaccagtgtgaaggtgctgcatggttgtcgtcagctcgtgccgtgaggtgtcggcttaagtgccataacgagcgcaacccttgctgccagttactaacaggtaatgctgaggactctggcgggactgccatcgtaagatgcgaggaaggtggggatgacgtcaaatcagcacggcccttacgtccggggctacacacgtgttacaatggggggtacagaaggccgctacccggcaacgggatgccaatctccaaaacccctctcagttcggactggagtctgcaacccgactccacgaagctggattcgctagtaatcgcgcatcagccacggcgcggtgaatacgttcccgggccttgtacacaccgcccgtcaagccatgaaagccgggggtacctgaagtgcgtaaccgcaaggagcgccctagggtaaaactggtaattggggct

SEQ ID NO: 21 - DNA, 1511 bp, Bacteroides sp.
  source 1..1511: mol_type = unassigned DNA
agagtttgatcctggctcaggatgaacgctagctacaggcttaacacatgcaagtcgaggggcagcgggattgaagcttgcttcaattgccggcgaccggcgcacgggtgagtaacgcgtatccaaccttccgcttactcggggatagcctttcgaaagaaagattaatacccgatggtatcttaagcacgcatgagattaagattaaagatttatcggtaagcgatggggatgcgttccattaggcagttggcggggtaacggcccaccaaacctacgatggataggggttctgagaggaaggtcccccacattggaactgagacacggtccaaactcctacgggaggcagcagtgaggaatattggtcaatgggcgagagcctgaaccagccaagtagcgtgaaggatgacggccctacgggttgtaaacttcttttgtgcgggaataaaggaacctacgtgtaggtttttgcatgtaccgtaacgaataagcatcggctaactccgtgccagcagccgcggtaatacggaggatgcgagcgttatccggatttattgggtttaaagggagcgtagacgggtttttaagtcagctgtgaaagtttggggctcaaccttaaaattgcagttgaaactggagaccttgagtacggttgaggcaggcggaattcgtggtgtagcggtgaaatgcttagatatcacgaagaaccccgattgcgaaggcagcctgctaagccgccactgacgttgaggctcgaaagtgcgggtatcaaacaggattagataccctggtagtccgcacggtaaacgatggatactcgctgttggcgatagacagtcagcggccaagcgaaagcgttaagtatcccacctggggagtacgccggcaacggtgaaactcaaaggaattgacgggggcccgcacaagcggaggaacatgtggtttaattcgatgatacgcgaggaaccttacccgggcttgaactgcagtggaattatccggaaacggataagcgagcaatcgccgctgtggaggtgctgcatggttgtcgtcagctcgtgccgtgaggtgtcggcttaagtgccataacgagcgcaacccttgctgccagttactaacaggtcatgctgaggactctggcaggactgccatcgtaagatgcgaggaaggtggggatgacgtcaaatcagcacggcccttacgtccggggctacacacgtgttacaatggggagtacagagggcagctaccgggcgaccggatgcgaatcccgaaagctcctctcagttcggactggagtctgcaacccgactccacgaagctggattcgctagtaatcgcgcatcagccacggcgcggtgaatacgttcccgggccttgtacacaccgcccgtcaagccatgaaagccgggggtacctgaagtacgtaaccgcgaggatcgtcctagggtaaaaccggtaattggggctaagtcgtaacaaggtagccgtaccggaaggtgcggctg

SEQ ID NO: 22 - DNA, 1491 bp, Blautia sp.
  source 1..1491: mol_type = unassigned DNA
agagtttgatcctggctcaggatgaacgctggcggcgtgcttaacacatgcaagtcgaacgagaaacattttaatgaagcttcggcagatttagtttgtttctagtggcggacgggtgagtaacgcgtgggtaacctgcctcacactgggggataacagtcagaaatgactgctaataccgcataagcgcacggaaccgcatggttttgtgtgaaaaactccggtggtgtgagatggacccgcgttggattagccagttggcagggtaacggcctaccaaagcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagataatgacggtacctgactaagaagccccggctaactacgtgccagcagccgcggtaatacgtagggggcaagcgttatccggatttactgggtgtaaagggagcgtagacggagcagcaagtctgatgtgaaaggcaggggctcaacccctggactgcattggaaactgttgatcttgagtaccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcagagccattcggtgccgcagcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccctctgaccggtccttaaccggacctttccttcgggacagaggagacaggtggtgcatggttgtcgtcagctcgtgtcgtgagatgttgggttaagtcccgcaacgagcgcaacccctatccccagtagccagcatttaaggtgggcactctgaggagactgccagggataacctggaggaaggcggggatgacgtcaaatcatcatgccccttatgatttgggctacacacgtgctacaatggcgtaaacaaagggaagcagagcggtgacgccgagcaaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgaaagggaggagctgccgaaggcgggacggatgactggggtgaagtcgtaacaaggtaacc

SEQ ID NO: 23 - DNA, 1336 bp, Fusobacterium sp.
  source 1..1336: mol_type = unassigned DNA
gatgaacgctgacagaatgcttaacacatgcaagtctacttgaattcacttcggtgatagtaaggtggcggacgggtgagtaacacgtaaagaacttgccttacagtctgggacaactattggaaacgatagctaataccggatattatgcgagagtcgcatgactcttgtatgaaagctatatgcgctgtaagagagctttgcgtcccattagctagttggtgaggtaacggctcaccaaggccacgatgggtagccggcctgagagggtgaacggccacaaggggactgagacacggcccttactcctacgggaggcagcagtggggaatattggacaatggaccaaaagtctgatccagcaattctgtgtgcacgatgacggtcttaggattgtaaagtgctttcaattgggaagaaaaaaatgacggtaccaatagaagaagcgacggctaaatacgtgccagcagccgcggtaatacgtatgtcgcaagcgttatccggatttattgggcgtaaagcgcgtctaggtggtttggtaagtctgatgtgaaaatgcggggctcaactccgtattgcgttggaaactgcctaactagagtatcggagaggtgggcggaactacaagtgtagaggtgaaattcgtagatatttgtaggaatgccgatagagaagtcagctcactggacgaatactgacactgaagcgcgaaagcatggggagcaaacaggattagataccctggtagtccatgctgtaaacgatgattactaagcgtcgggggtcgaacctcggcactcaagctaacgcgataagtaatccgcctggggagtacgtacgcaagtatgaaactcaaaggaattgacggggacccgcacaagtggtggagcatgtggtttaattcgacgcaacgcgaggaaccttaccagcgtttgacatcctaggaatgagaaagagatttcttagtgctccttcgggagaacctagagacaggtggtgcatggctgtcgtcagctcgtgtcgtgagatgttgggttaagtcccgcaacgagcgcaacccctattgtatgttgccatcattaagttgggcactcatgcgatactgcctgcgatgagcaggaggaaggtggggatgacgtcaagtcatcatgccccttatacgctgggctacacacgtgctacaatgggcagtacagagagaagcaaatctgcgaggaggagcaaatctcacaaagctgttcgtagttcggattgtactctgcaactcgagtacatgaagttggaatcactagtaatcgcaaatcagctatgttgcggtgaatacgttctcgggtct

SEQ ID NO: 24 - DNA, 1359 bp, unidentified
  misc_feature 1..1359: note = Bacteria
  source 1..1359: mol_type = unassigned DNA; note = Lachnospiraceae sequence
gatgaacgctggcggcgtgcttaacacatgcaagtcgaacgggaaacattttaatgaagcttcggcagatttagcttgtttctagtggcggacgggtgagtaacgcgtgggtaacctgcctcacactgggggataacagtcagaaatgactgctaataccgcataagcgcacggaaccgcatggttttgtgtgaaaaactccggtggtgtgagatggacccgcgttggattagccagttggcagggtaacggcctaccaaagcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaatggcaagtctgatgtgaaaggcaggggctcaacccctggactgcattggaaactgtcagtcttgagtaccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatacgaggtgtcgggtgggcaaagccattcggtgccgcagcaaacgcaaaaagtaatcccacctgggggagtacgttcccaagaatgaaactcaaaggaaatagcggggacccgcacaagcggtggagcatgtggtgtatttgaagcaacgcgaagaaccttaccaagtcttgacatccctctgaccggtccttaaccggacctctccttcgggacaggggagacaggtggtgcatggttgtcgtcagctcgtgtcgtgagatgttgggttaagtcccgcaacgagcgcaacccctatccttagtagccagcatctgaggtgggcactctgaggagactgccagggataacctggaggaaggcggggaggacgtcaaatcatcatgccccctatgatttgggctacacacgtgctacaatggcgtaaacaaagggaagcagagcggtgacgccgagcaaatcccaaaaataacgtcccagttcggactgcagtctgcaactcgactgcacgaagctggaatcgctagtaatcgcggatcagaatgccgcggtgaataaaagcccgggtcttgcact

SEQ ID NO: 25 - DNA, 1387 bp, Blautia sp.
  source 1..1387: mol_type = unassigned DNA
agagtttgatcatggctcaggatgaacgctggcggcgtgcttaacacatgcaagtcgaacgggaattactttattgaagctttggtcgatttaatttaattatagtggcggacgggtgagtaacgcgtgggtaacctgccttatacagggggataacagtcagaaatggctgctaataccgcataagcgcacagagctgcatggctcagtgtgaaaaactccggtggtataagatggacccgcgttggattagttggttggtggggtaacggcccaccaaggcgacgatccatagccggcctgagagggtgaacggccacattgggactgagacacggcccagactcatacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacctgtggactgcattggaaactgtcatacttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccgcctgaccgatccttaaccggatctttccttcgggacaggcgagacaggtggtgcatggttgtcgtcagctcgtgtcgtgagatgttgggttaagtcccgcaacgagcgcaacccctatcctcagtagccagcattaagttgggcactcatgcgatactgcctgcgatgagcaggaggaaggtggggatgacgtcaagtcatcatgccccttatacgctgggctacacacgtgctacaatgggtagtacagagagtcgcaaacctgcgagggggagctaatctcagaaaactattctcagttcggattgtactctgcaactcgagtacatgaagttggaatcgctagtaatcgcaaatcagctatgttgcggtgaatacgttctcgggtcttgcactcaccgcccgt

SEQ ID NO: 26 - DNA, 1332 bp, Blautia sp.
  source 1..1332: mol_type = unassigned DNA
gctcaggatgaacgctggcggcgtgcttaacacatgcaagtcgagcgaagcacttgccattgactcttcggaagatttggcatttgactgagcggcggacgggtgagtaacgcgtgggtaacctgcctcatacgggggaataacagttagaaatggctgctaatgccgcataaccgcacaggaccgcatggactggtgtgaaaaactgaggtggtatgagatgggcccgcgtctgattaggttagttggcggggtaacggcccaccaagccgacgatcagtagccgacctgagagggaccggccacattgggactgagacatggcccagactcctacgggaggcagcagtggggaatattgcacaatggaggaaactctgatgcagcgacgccgcatgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggacgggcaagtctgatgtgaaagcccggggcttaaccccgggactgcattggaaactgtccatcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatcaataatgggtgtcgggttgcaaagcaatccggtgccgcagcaaacgcagtaagtattccccctcgggagtacgttcgcaagaatgaaactcaaaggaagggacggggatccgcacaagcggcggagcatgtggtttaattagaagcaacgcgaagaaccttaccaagtcttgacatctgcctgaccgttccttaaccggaactatctttcgggacaggcaagacaggtggtgcatggttgtcgtcagctcgtgtcgtgagatgttgggttaagtcccgcaacgagcgcaacccctgtccttagtagccagcagtccggctgggcactctagggagactgccgggggtaacccggaggaaggcggggaggaggtcaaatcatcatgccccccctgatttgggctacacacgtggtacaatggcgtaaacaaagggaagcggagtggtgacgctgagcaaatctcaaaaataacgtcccacttcggactgcagtctgcaactcgactgcacgaagctggaatcgctagtaatcgcgaatcagaatg

SEQ ID NO: 27 - DNA, 253 bp, Blautia sp.
  source 1..253: mol_type = unassigned DNA
tacgtagggggcaagcgttatccggatttactgggtgtaaagggagcgtagacggcatggcaagtctgatgtgaaaggcaggggctcaactcctggactgcattggaaactgccaggcttgagtgccggaggggtaagcggaattcctagtgtagcggtgaaatgcgtagatattaggaggaacaccagtggcgaaggcggcttactggacggtaactgacgttgaggctcgaaagcgtggggagcaaacagg

SEQ ID NO: 28 - DNA, 1324 bp, Intestinibacter sp.
  source 1..1324: mol_type = unassigned DNA
cgtaagtaacctgccctgtacacacggataacataccgaaaggtatgctaatacgggataatatattttgatcgcatggtcgagatatcaaagctccggcggtacaccagggacccccgacagaggagctagttggtagtaatgtcaccaaggcgacgatcagaagccgaactgagagggggatccgcacatgactgagacacggtcaaactcctacgggaggcagcagtggggaatatgccaatgggcgaaagctgatgcagcacgcgcgtgagcgatgaggctcgggtcgtaaagctcgtctcaaggaagataatgacggtacttgaggaggaagccccggctaactacgtgccagcagccgcggtaatacgtagggggctagcgttatccggaattactgggcgtaaagggtgcgtaggcggtctttcaagtcaggagtgaaaggctacggctcaaccgtagtaagctcttgaaactgtaagacttgagtgcaggagaggagagtggaattcctagtgtagcggtgaaatgcgtagatattaggaggaacaccagttgcgaaggcggctctctggactgtaactgacgctgaggcacgaaagcgtggggagcaaacaggattagataccctggtagtccacgccgtaaacgatgagtactagctgtcggaggttacccccttcggtggcgcagctaacgcattaagtactccgcctgggaagtacgctcgcaagagtgaaactcaaaggaattgacggggacccgcacaagtagcggagcatgtggtttaattcgaagcaacgcgaagaaccttacctaagcttgacatcccactgacccttccctaatcggaagcttcccttcgggacagtggtgacaggtggtgcatggttgtcgtcagctcgtgtcgtgagatgttgggttaagtcccgcaacgagcgcaacccttgcctttagttgccagcattaagttgggcactctagagggactgccagggataacccggaggagtggggatgacgtcaaatcatcatgcccttatgctaggctacacacgtgctacaatgggtggtcagaggccagccagtcgtgaggccgagctatcccataagccattctcgtccggattgtaggctgaactcgcctacatgagctggaattacaagtatgcgatcgatgctgcgtgatgcgtccgggtcttgtacacaccgcccgtcacaccatgggagttgggggcgcccgaagccggattgctaaccttttggaagcgtccgtcgaaggtgaaaccaataactggggtgaagtcgtaacaaggtaacc

SEQ ID NO: 29 - DNA, 1342 bp, Corynebacterium sp.
  source 1..1342: mol_type = unassigned DNA
gacgaacgctggcggcgtgcttaacacatgcaagtcgaacggaaaggccccagctcgctggggtactcgagtggcgaacgggtgagtaacacgtgggtgatctgccttgcactctgggataagcttgggaaactgggtctaataccggatatgaactgcctttagtgtggtggttggaaagttttttcggtgcaagatgagctcgcggcctatcagcttgttggtggggtaatggcctaccaaggcgtcgacgggtagccggcctgagagggtgtacggccacattgggactgagatacggcccagactcctacgggaggcagcagtggggaatattgcacaatgggcggaagcctgatgcagcgacgccgcgtgggggatgacggccttcgggttgtaaactcctttcgacagggacgaagctttttgtgacggtacctgtataagaagcaccggctaactacgtgccagcagccgcggtaatacgtagggtgcgagcgttgtccggaattactgggcgtaaagagctcgtaggtggtttgtcgcgtcgtctgtgaaattccggggcttaactccgggcgtgcaggcgatacgggcataacttgagtactgtaggggagactggaattcctggtgtagcggtgaaatgcgcagatatcaggaggaacaccggtggcgaaggcgggtctctgggcagtaactgacgctgaggagcgaaagcatggggagcgaacaggattagataccctggtagtccatgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggcttgacatacactggatcgggctagagatagtctttccctttgtggctggtgtacaggtggtgcatggttgtcgtcagctcgtgtcgtgagatgttgggttaagtcccgcaacgagcgcaacccttgtcttatgttgccagcatttggttggggactcatgagagactgccggggtcaactcggaggaaggtggggatgacgtcaaatcatcatgccccttatgtccagggcttcacacatgctacaatggtcggtacaacgcgcagcgacactgtgaggtggagcgaatcgctgaaagccggccttagttcggattggggtctgcaactcgaccccatgaagtcggagtcgctagtaatcgcagatcagcaatgctgcggtgaatacgttcccgggcct

SEQ ID NO: 30 - DNA, 253 bp, Clostridium sp.; identical to SEQ ID NO: 14
  source 1..253: mol_type = unassigned DNA

SEQ ID NO: 31 - DNA, 1399 bp, Enterococcus sp.
  source 1..1399: mol_type = unassigned DNA
cttgtgtcaccaaccatagggagggggaaaacatggaaacggggttcataccgcataacttttttagcccaatgcataagaagaaaggcctttcgggtttcggtaaaggaggcccccgcggctcttatagtgtgtgtggaagtaaccgcttccacaaggcccaggtttcatacccgactggagagtgtgttcgccacactggggaaaggacccccggcccagtctctctaggggaggcagcagtaggaattttcggcaaaggaaaaaatttctgaccgaacaacgccggttgaatgaagaagtttttcggatcgaaaaactctgttgttagagaagaacaaggacgttagtaactgaacgtcccctgacggtatctaaccagaaagccacggctaattacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagcaaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagctttcccttcggggacaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgctagaccgcgaggtcatgcaaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggtaacctttttggagccagccgcctaaggtgggatagatgattggggtgaagtcgtaaccaacgtatgcc

SEQ ID NO: 32 - DNA, 1393 bp, Streptococcus sp.
  misc_difference 1381: note = a, c, t, g, unknown or other
  source 1..1393: mol_type = unassigned DNA
agactttagcttgctaaagttggaagagttgcgaacgggtgagtaacgcgtaggtaacctgcctactagcgggggataactattggaaacgatagctaataccgcataacagcatttaacccatgttagatgcttgaaaggagcaattgcttcactagtagatggacctgcgttgtattagctagttggtgaggtaacggctcaccaaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagagaagaacgtgtgtgagagtggaaagttcacacagtgacggtaacttaccagaaagggacggctaactacgtgccagcagccgcggtaatacgtaggtcccgagcgttgtccggatttattgggcgtaaagcgagcgcaggcggtttaataagtctgaagttaaaggcagtggcttaaccattgttcgctttggaaactgttagacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttaggccctttccggggcttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgctattcctagagataggaagtttcttcggaactgtgagacttgagggcagaagggtagagtgcacttgtatggggagctgtggaatgcgttcccgcaacgagcgcaacccctattgttagttgccatcattaagttgggcactctagcgagactgccggtaataaaccggaggaaggtggggatgacgtcaaatcatcatgccccttatgacctgggctacacacgtgctacaatggttggtacaacgagtcgcgagtcggtgacggcaagcaaatctcttaaagccaatctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtanccttttaggagc

SEQ ID NO: 33 - DNA, 1418 bp, Megasphaera sp.
  source 1..1418: mol_type = unassigned DNA
agagtttgatcatggctcaggacgaacgctggcggcgtgcttaacacatgcaagtcgaacgagaagagatgagaagcttgcttcttatctcttcgagtggcaaacgggtgagtaacgcgtaagcaacctgcccttcagatggggacaacagctggaaacggctgctaataccgaatacgttctttttgtcgcatggcagagggaagaaagggaggctcttcggagctttcgctgaaggaggggcttgcgtctgattagctagttggaggggtaacggcccaccaaggcgacgatcagtagccggtctgagaggatgaacggccacattgggactgagacacggcccagactcctacgggaggcagcagtggggaatcttccgcaatggacgaaagtctgacggagcaacgccgcgtgaacgatgacggccttcgggttgtaaagttctgttatacgggacgaatggcgtagcggtcaatacccgttacgagtgacggtaccgtaagagaaagccacggctaactacgtgccagcagccgcggtaatacgtaggtggcaagcgttgtccggaattattgggcgtaaagggcgcgcaggcggcgtcgtaagtcggtcttaaaagtgcggggcttaaccccgtgaggggaccgaaactgcgatgctagagtatcggagaggaaagcggaattcctagtgtagcggtgaaatgcgtagatattaggaggaacaccagtggcgaaagcggctttctggacgacaactgacgctgaggcgcgaaagccaggggagcaaacgggattagataccccggtagtcctggccgtaaacgatggatactaggtgtaggaggtatcgaccccttctgtgccggagttaacgcaataagtatcccgcctggggagtacggccgcaaggctgaaactcaaaggaattgacgggggcccgcacaagcggtggaacatgtggtttaattcgatgatacgcgaggaaccttacccgggcttaaattgcagtggaatgatgtggaaacatgtcagtgagcaatcaccgctgtgaaggtgctgcatggttgtcgtcagctcgtgccgtgaggtgtcggcttaagtgccataacgagcgcaacccttatcttcagttactaacaggtcatgctgaggactctggagagactgccgtcgtaagatgtgaggaaggtggggatgacgtcaaatcagcacggcccttacgtccggggctacacacgtgttacaatggggggtacagagggccgctaccacgcgagtggatgccaatcccaaaaacctctctcagttcggactggagtctgcaacccgactccacgaagctggattcgctagtaatcgcgcatcagccacggcgcggtgaatacgttcccgggccttgcactcaccgcccgt

SEQ ID NO: 34 - DNA, 1394 bp, Parabacteroides sp.; identical to SEQ ID NO: 18
  source 1..1394: mol_type = unassigned DNA

SEQ ID NO: 35 - DNA, 1472 bp, unidentified
  misc_feature 1..1472: note = Bacteria
  source 1..1472: mol_type = unassigned DNA; note = Escherichia-Shigella sequence
cgccctgattgacggctatacacatgcaagtcgaacggtaacaggaaacagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctccatccctaggcgagccgaatccttagcctggtctgagaggaatgaccagccacactgggactgagaacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaataccctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacgggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtccaggtcatcatggcccttacgaaccagggctacacacgtgcctacaatggacgcatccaaagagagagcgaaccctgcccgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttggatgcgagg

SEQ ID NO: 36 - DNA, 1440 bp, Tyzzerella sp.
  source 1..1440: mol_type = unassigned DNA
taagatgaacgctggcggcgtgcttaacacatgcaagtcctatgaagcgcttaaacggatttcttcggattgaagtttttgtgactgagtggcggacgggtgagtaacgcgtgggtaacttgcctcatacagggggataacagttagaaatgactgctaataccgcataagcgcacagtgctgcatggcacagtgtgaaaaactccggtggtatgagatggacccgcgtctgattagctagttggtggggtaacggcctaccaaggcgacgatcagtagccggcctgagagggtgaacggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggttgtgtaagtctgatgtgaaagcccggggctcaaccccgggactgcattggaaactatgtaactagagtgtcggagaggtaagcggaattcctagtgtagcggtgaaatgcgtagatattaggaggaacaccagtggcgaaggcggcttactggacgatcactgacgttgaggctcgaaagcgtggggagcaaacaggattagataccctggtagtccacgccgtaaacgatgactactaggtgtcggggcccataagggcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttacctggtcttgacatcccactgaccggacagtaatgtgtcctttcctccgggacagtggagacaggtggtgcatggttgtcgtcagctcgtgtcgtgagatgttgggttaagtcccgcaacgagcgcaacccctatccttagtagccagcagtaagatgggcactctagggagactgccagggataacctggaggaaggtggggatgacgtcaaatcatcatgccccttatgacttgggctacacacgtgctacaatggcgtaaacaaagtgaagcgaagtcgtgaggccaagcaaatcacaaaaataacgtctcagttcggattgtagtctgcaactcgactacaagaagctggaatcgctagtaatcgcagatcagaatgctgcggtgaatacgttcccgggtcttgtacacaccgcccgtcacaccatgggagtcgaaaatgcccgaagtcggtgacctaacgaaagaaggagccgccgaaggcaggtt

SEQ ID NO: 37 - DNA, 1366 bp, Fusobacterium sp.
  source 1..1366: mol_type = unassigned DNA
agagtttgatcctggctcaggatgaacgctgacagaatgcttaacacatgcaagtatacttgatccttcgggtgatggtggcggacgggtgagtaacgcgtaaagaacttgccctgcagtctgggacaacatttggaaacgaatgctaatcccgcataagcccacagctcggcatcgagcagagggaaaaggagtgatctgctttgagatggcctcgcgtccgattagctggttggtgaggtgacggcccatcaaggcaacgatcggtagccggactgagaggttgaacggccacattgggattgagacacggcccttactcctacgggaggcagcagtggggaatattggacaatggaccaaaagtctgatccagcaattctgtgtgcacgatgaagtttttcggaatgtaaagtgctttcagttgggacgaagtaagtgacggtaccaacagaagaagcgacggctaaatacgtgccagcagccgcggtaatacgtatgtcgcaagcgttatccggatttattgggcgtaaagcgcgtctaggcggtttggtaagtctgatgtgaaaatgcggggctcaactccgtattgcgttggaaactgccaaactagagtactggagaggtgggcggaactacaagtgtagaggtgaaattcgtagatatttgtaggaatgccgatggggaagccagcccactggacagatactgacgctaaagcgcgaaagcgtgggtagcaaacaggattagataccctggtagtccacgccgtaaacgatgattactaggtgttgggggtcgaacctcagcgcccaagctaacgcgataagtaatccgcctggggagtacgtacgcaagtatgaaactcaaaggaattgacggggacccgcacaagcggtggagcatgtggtttaattcgacgcaacgcgaggaaccttaccagcgtttgacatcctaagaaattagcagagatgcttttgtgccccttcgggggaacttagtgacaggtggtgcatggctgtcgtcagctcgtgtcgtgagatgttgggttaagtcccgcaacgagcgcaacccctttcgtatgttgccatcattaagttgggcactcatgcgatactgcctgcgatgagcaggaggaaggtggggatgacgtcaagtcatcatgccccttatacgctgggctacacacgtgctacaatgggtagtacagagagtcgcaaacctgcgagggggagctaatctcagaaaactattctcagttcggattgtactctgcaactcgagtacatgaagttggaatcgctagtaatcgcaaatcagctatgttgcggtgaatacgttctcgggtcttgtacacaccgcccgt

SEQ ID NO: 38 - DNA, 1324 bp, Intestinibacter sp.; identical to SEQ ID NO: 28
  source 1..1324: mol_type = unassigned DNA

SEQ ID NO: 39 - DNA, 1657 bp, Helicobacter sp.; identical to SEQ ID NO: 8
  source 1..1657: mol_type = unassigned DNA

SEQ ID NO: 40 - DNA, 253 bp, Anaerobiospirillum sp.; identical to SEQ ID NO: 11
  source 1..253: mol_type = unassigned DNA

SEQ ID NO: 41 - DNA, 1342 bp, Corynebacterium sp.; identical to SEQ ID NO: 29
  source 1..1342: mol_type = unassigned DNA

SEQ ID NO: 42 - DNA, 1357 bp, Collinsella sp.; identical to SEQ ID NO: 12
  misc_difference 766: note = a, c, t, g, unknown or other
  source 1..1357: mol_type = unassigned DNA

SEQ ID NO: 43 - DNA, 1488 bp, Sarcina sp.
  source 1..1488: mol_type = unassigned DNA
agagtttgatcctggctcaggacgaacgctggcggcgtgcctaatacatgcaagtcgagcggagttactttgagagcttgctttcaaagtaacttagcggcggacgggtgagtaacacgtaggcaacctgccccttagactgggataactaccggaaacggtagctaataccggataatttcttttttctcctgaaggaagaatgaaagacggagcaatctgtcactgagggatgggcctgcggcgcattagctagttggtggggtaacggcccaccaaggcgacgatgcgtagccgacctgagagggtgatcggccacattggaactgagatacggtccagactcctacgggaggcagcagtggggaatattgcacaatgggggaaaccctgatgcagcaacgccgcgtgagtgatgaaggtcttcggattgtaaagctctgtctttagggacgataatgacggtacctaaggaggaagccacggctaactacgtgccagcagccgcggtaatacgtaggtggcaagcgttatccggatttactgggcgtaaagggagcgtaggcggatatttaagtgggatgtgaaatacccgagcttaacttgggagctgcattccaaactggatatctagagtgcaggagaggagaatggaattcctagtgtagcggtgaaatgcgtagagattaggaagaacaccagtggcgaaggcgattctctggactgtaactgacgctgaggctcgaaagcgtggggagcaaacaggattagataccctggtagtccacgccgtaaacgatgaataccaggtgtaggggccccaagcctctgtgccgccgctaacgcattaagtattccgcctggggagtacggtcgcaagattaaaactcaaaggaattgacggggacccgcacaagcagcggagcatgtggtttaattcgaagcaacgcgaagaaccttacctagacttgacatgtcctgaattaccagtaatgtgggaagttccttcgggaacaggaacacaggtggtgcatggttgtcgtcagctcgtgtcgtgagatgttgggttaagtcccgcaacgagcgcaacccttattgttagttggtaccattaagttgaccactctagcgagactgcccgggttaaccgggaggaaggtggggatgacgtcaaatcatcatgccccttatgtctagggctacacacgtgctacaatggcaagtacaaagagaagcaatactgtgaagtggagcaaaactcaaaaacttgtctcagttcggattgtaggctgaaactcgcctacatgaagctggagttgctagtaatcgcgaatcagaatgtcgcggtgaatacgttcccgggtcttgtacacaccgcccgtcacaccatgagagttggcaatacccgaagtccgtaagctaaccgtaaggaggcagcggccgaaggtagggtcagcgattggggtgaagtcgtaacaaggtaaccaa

SEQ ID NO: 44 - DNA, 1454 bp, Clostridium sp.; identical to SEQ ID NO: 13
  misc_difference 635: note = a, c, t, g, unknown or other
  misc_difference 704: note = a, c, t, g, unknown or other
  source 1..1454: mol_type = unassigned DNA

SEQ ID NO: 45 - DNA, 253 bp, Clostridium sp.; identical to SEQ ID NO: 14
  source 1..253: mol_type = unassigned DNA

SEQ ID NO: 46 - DNA, 1392 bp, Clostridium sp.
  misc_difference 811: note = a, c, t, g, unknown or other
  misc_difference 831..832: note = a, c, t, g, unknown or other
  misc_difference 836: note = a, c, t, g, unknown or other
  misc_difference 999: note = a, c, t, g, unknown or other
  misc_difference 1013: note = a, c, t, g, unknown or other
  misc_difference 1047: note = a, c, t, g, unknown or other
  misc_difference 1051: note = a, c, t, g, unknown or other
  misc_difference 1057: note = a, c, t, g, unknown or other
  misc_difference 1069: note = a, c, t, g, unknown or other
  misc_difference 1101: note = a, c, t, g, unknown or other
  misc_difference 1103: note = a, c, t, g, unknown or other
  misc_difference 1116: note = a, c, t, g, unknown or other
  misc_difference 1168: note = a, c, t, g, unknown or other
  misc_difference 1185: note = a, c, t, g, unknown or other
  misc_difference 1188: note = a, c, t, g, unknown or other
  misc_difference 1202: note = a, c, t, g, unknown or other
  misc_difference 1207: note = a, c, t, g, unknown or other
  misc_difference 1209..1210: note = a, c, t, g, unknown or other
  misc_difference 1221: note = a, c, t, g, unknown or other
  misc_difference 1230: note = a, c, t, g, unknown or other
  misc_difference 1284: note = a, c, t, g, unknown or other
  misc_difference 1287: note = a, c, t, g, unknown or other
  misc_difference 1296: note = a, c, t, g, unknown or other
  misc_difference 1391: note = a, c, t, g, unknown or other
  source 1..1392: mol_type = unassigned DNA
gggaatctccaggatctgattagcggcggacgggtgagtacacgtgggtaacctgcctcatagagtggaatagccttccgaaaggaagattaataccgcataacgttgaaagatggcatcatcattcaaccaaaggagcaatccgctatgagatggacccgcggcgcattagctagttggtggggtaacggcctaccaaggcgacgatgcgtagccgacctgagagggtgatcggccacattgggactgagacacggcccagactcctacgggaggcagcagtggggaatattgcacaatgggggaaaccctgatgcagcaacgccgcgtgagtgatgaaggttttcggatcgtaaagctctgtctttggggaagataatgacggtacccaaggaggaagccacggctaactacgtgccagcagccgcggtaatacgtaggtggcgagcgttatccggatttactgggcgtaaagggagcgtaggcggatgattaagtgggatgtgaaatacccgggctcaacttgggtgctgcattccaaactggttatctagagtgcaggagaggagagtggaattcctagtgtagcggtgaaatgcgtagagattaggaagaacaccagtggcgaaggcgactctctggactgtaactgacgctgaggctcgaaagcgtggggagcaaacaggattagataccctggtagtccacgccgtaaacgatgaatactaggtgtgggggtttcaacacctccgtgccgccgctaacgcattaagtattccgcctggggagtacggtcgcaagattaaaactnaaaggaattgacggggatcnncacnagtagcggagcatgtggtttaattcgaagcaacgcgaagaaccttacctacacttgacatcccttgcattactcttaatcgaggaaatctcttcggggacaaggtgacaggtggtgcatggttgtcgtcagctcgtgtcgtgagatgttgggttaagtcccgnaacgaggggaaccnttgtcgttagttactaccattaagttgaggactntagngagacngctgggttaacnaggaggaaggtggggatgactcaatctctggncnttatgtgtagggntacacacgtgctacaatggctggtacagagagatgcataccgggaggtggantcaatttaaaaacagtntcnttcggattgtaggntgaantnncctactgaagntggagttantagtaatcgcgaatcagaatgtcgcggtgaatacgttcccgggtcttgtacacnccncccgtcacnccatgagagttggcaatacccgaagtccgtgagctaaccgcaaggaggcagcggccgaaggtagggtcagcgattggggtgaagtcgtaacaggna

SEQ ID NO: 47 - DNA, 1362 bp, Faecalitalea sp.
  source 1..1362: mol_type = unassigned DNA
gatgaacgctggcggcatgcctaatacatgcaagtcgaacggagcgaatatggaagcttgcttccgtaagagctcagtggcgaacgggtgagtaacacgtaggtaacctgcccatgtgcccgggataactgctggaaacggtagctaaaaccggataggtgaataggaggcatctcttattcattaaaggacctgtaagggtgcgaacatggatggacctgcggcgcattagctggttggagtggtaacggcacaccaaggcgacgatgcgtagccgacctgagagggcgaacggccacattgggactgagacacggcccaaactcctacgggaggcagcagtagggaattttcgtcaatggggggaaccctgaacgagcaatgccgcgtgagtgaagaaggtcttcggatcgtaaagctctgttgtaagtgaagaacggtcagtagaggaaatgatactgaagtgacggtagcttaccagaaagccacggctaactacgtgccagcagccgcggtaatacgtaggtggcgagcgttatccggaatcattgggcgtaaagggtgcgcaggtggtacattaagtccgaagtaaaaggcagcagctcaactgctgttggctttggaaactggtgaactggagtgcaggagagggcgatggaattccatgtgtagcggtaaaatgcgtagatatatggaggaacaccagtggcgaaggcggtcgcctggcctgcaactgacactgaggcacgaaagcgtggggagcaaataggattagataccctagtagtccacgccgtaaacgatgagaactaagtgttggggagactcagtgctgcagttaacgcaataagttctccgcctggggagtatgcacgcaagtgtgaaactcaaaggaattgacgggggcccgcacaagcggtggagtatgtggtttaattcgaagcaacgcgaagaaccttaccaggccttgacatggatgtaaatgttctagagatagaaagatagctatacatcacacaggtggtgcatggttgtcgtcagctcgtgtcgtgagatgttgggttaagtcccgcaacgagcgcaacccttatcgcatgttaccagtattgagttagggactcatgcgagactgccggtgacaaaccggaggaaggtggggatgacgtcaaatcatcatgccccttatggcctgggctacacacgtactacaatggcggctacaaagagaagcgaacctgcgagggggagcggaactcataaaggccgtctcagttcggattggagtctgcaactcgactccatgaagtcggaatcgctagtaatcgcagatcagcatgctgcggtgaatacgttctcgggcct

SEQ ID NO: 48 - DNA, 1399 bp, Enterococcus durans; identical to SEQ ID NO: 31
  source 1..1399: mol_type = unassigned DNA

SEQ ID NO: 49 - DNA, 1414 bp, Streptococcus sp.
  source 1..1414: mol_type = unassigned DNA
agagtttgatcctggctcaggactaacgctggcggcgtgcctaatacatgcaagtagaacgctgaagactttagcttgctaaagttggaagagttgcgaacgggtgagtaacgcgtaggtaacctgcctactagcgggggataactattggaaacgatagctaataccgcataacagcatttaacccatgttagatgcttgaaaggagcaattgcttcactagtagatggacctgcgttgtattagctagttggtgaggtaacggctcaccaaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagagaagaacgtgtgtgagagtggaaagttcacacagtgacggtaacttaccagaaagggacggctaactacgtgccagcagccgcggtaatacgtaggtcccgagcgttgtccggatttattgggcgtaaagcgagcgcaggcggtttaataagtctgaagttaaaggcagtggcttaaccattgttcgctttggaaactgttagacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgaaaggtgttaggccctttccggggcttagttgctgcacgctaactgcattatgacactccgccaggggagtacgaccgctaggttgaaactcaaaggagttgacgggggccagcacaaccggtggagcatgtggttgaattggaagcaacgcgaagagccttaccaggtcttgacatcccgacgctattcctagagataggaagtttcttcgggacattcggtggcaggtggtgcatggtagtcgtcagctcgtgtcgtgagatgttgggttaagtcccgcaacgagcgcaacccctattgttagttgccatacattaagttgggcactctagcgagactgccggtaataaaccggaggaaggtggggatgacgtcaaatcatcatgccccttatgacctgggctacacacgacgctacaatggttggtacaacgagtcgcgagtcggtgacggcaagcaaatctcttaaagccaatctcagttcggattgtaggctgcaactcgcctacatgaagtcggaatcgctagtaatcgcggatcagcacgccgcggtgaatacgttcccgggccttgcactcaccgcccgtca

SEQ ID NO: 50 - DNA, 1517 bp, Streptococcus sp.
  source 1..1517: mol_type = unassigned DNA
agagtttgatcatggctcaggacgaacgccggcggcgtgcctaatacatgcaagtagaacgctgaagactttagcttgctaaagttggaagagttgcgaacgggtgagtaacgcgtaggtaacctgcctactagcgggggataactattggaaacgatagctaataccgtataacagcatttaacacatgttagatgcttgaaaggagcaattgcttcactagtagatggacctgcgttgtattagctagttggtgaggtaacggctcaccaaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagagaagaacgtgtgtgagagtggaaagttcacacagtgacggtaacttaccagaaagggacggctaactacgtgccagcagccgcggtaatacgtaggtcccgagcgttgtccggatttattgggcgtaaagcgagcgcaggcggtttaataagtctgaagttaaaggcagtggcttaaccattgttcgctttggaaactgttagacttgagtgcagaaggggagagtggaattccatgtgtagcggtgaaatgcgtagatatatggarggaaacaccggtggcgaaagcggctctctggtctgtaactgacgctgaggctcgagaagcgtggggagcaaacaggattagataccctggtagtccacgccgtaagcgatgagtgctaggtgttaggccctttccggggcttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgctattcctagagataggaagtttcttcggaacatcggtgacaggtggtgcatggttgtcgtcagctcgtgtcgtgagatgttgggttaagtcccgcaacgagcgcaacccctattgttagttgccatcattaagttgggcactctagcgagactgccggtaataaaccggaggaaggtggggatgacgtcaaatcatcatgccccttatgacctgggctacacacgtgctacaatggcggtcaacagagggaagcaatactgtgaagtggagcaaacccctaaaagccgtcccagttcggattgcaggctgcaacccgcctgtatgaagttggaatcgctagtaatcgcggatcagcatgccgcggtgaatacgttcccgggccttgtacacaccgcccgtcacaccatgagagtcgggaacacccgaagtccgtagcctaactttcacgagggggcgcggccgaaggtgggttcgataattggggtgaagtcgtaacaaggtaaccgta

SEQ ID NO: 51 - DNA, 252 bp, Streptococcus sp.
  source 1..252: mol_type = unassigned DNA
tacgtaggtcccgagcgttgtccggatttattgggcgtaaagcgagcgcaggcggtttaataagtctgaagttaaaggcagtggcttaaccatttttcgctttggaaactgttagacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacagg

SEQ ID NO: 52 - DNA, 1529 bp, Streptococcus sp.
  source 1..1529: mol_type = unassigned DNA
agagtttgatcctggctcaggacgaacgctggcggcgtgcctaatacatgcaagtagaacgctgaagaaaggagcttgcttcttttggatgagttgcgaacgggtgagtaacgcgtaggtaacctgccttgtagcgggggataactattggaaacgatagctaataccgcataacagcttttgacacatgttagaagcttgaaagatgcaattgcatcactacgagatggacctgcgttgtattagctagtaggtagggtaacggcctacctaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagagaagaacgtgtgtgagagtggaaagttcacacagtgacggtaacttaccagaaagggacggctaactacgtgccagcagccgcggtgatacgtaggtcccgagcgttgtccggatttattgggcgtaaagcgagcgcaggcggtttaataagtctgaagttaaaggcagtggcttaaccattgttcgctttggaaactgttaaacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttaggccctttccggggcttagtgccgcagctaacgcattaagtattccgcctggggagtacggtcgcaagattaaaactcaaaggaattgacgggggcccgcacaagcagcggagcatgtggtttaattcgaagcaacgcgaagaaccttacctagacttgacatctcctgcattactcttaatcgaggaagtcccttcggggacaggatgacaggtggtgcatggttgtcgtcagctcgtgtcgtgagatgttgggttaagtcccgcaacgagcgcaacccttattgttagttgccatcattaagttgggcactctagcgagactgcccgggttaaccgggaggaaggtggggatgacgtcaaatcatcatgccccttatgtctagggctacacacgtgctacaatggtcggtacaataagacgcaagcccgcgagggggagcaaaactggaaaaccgatctcagttcggattgtaggctgaaactcgcctacatgaagctggagttgctagtaatcgcgaatcagcatgtcgcggtgaatacgttcccgggccttgtacacaccgcccgtcacaccatgagagttggcaatacccaaagtacgtgatctaacccgcaagggaggaagcgtcctaaggtagggtcagcgattggggtgaagtcgtaacaaggtagccgtaggagaacctgcggctg

SEQ ID NO: 53 - DNA, 1398 bp, Streptococcus sp.
  source 1..1398: mol_type = unassigned DNA
agagtttgctcttgggtcaggatgaacgctggcggcgtgcttaacacatgcaagtcgagcgagaaaagttcttcggagcttttctagcggcggacgggtgagtaacacgtgggcaacctgcctcatagaggggaatagccttccgaaaggaagattaataccgcataacattgttgaaaggcatcttttaacaatcaaaggagcaatccgctatgagatgggcccgcggcgcattagctagttggtgaggtaacggctcaccaaggcgacgatgcgtagccgacctgagagggtgatcggccacattggaactgagacacggtccagactcctacgggaggcagcagtggggaatattgcacaatgggggaaaccctgatgcagcgacgccgcgtgagtgaagaagtatttcggtatgtaaagctctgttgtaagagaagaacgtgtgtgagagtggaaagttcacacagtgacggtaacttaccagagagggacggctaactacgtgccagcagccgcggtaatacgtaggtcccgagcgttgtccggatttattgggcgtaaagcgagcgcaggcggtttaataagtctgaagttaaaggcagtggcttaaccattgttcgctttggaaactgttagacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttaggccctttccggggcttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgctattcctagagataggaagtttcttcggaacatcggtgacaggtggtgcatggttgtcgtcagctcgtgtcgtgagatgttgggttaagtcccgcaacgagcgcaacccctattgttagttgccatcattaagttgggcactctagcgagactgccggtaataaaccggaggaaggtggggatgacgtcaaatcatcatgccccttatgacctgggctacacacgtgctacaatggttggtacaacgagtcgcgagtcggtgacggcaagcaaatctcttaaagccaatctcagttcggattgtaggctgcaactcgcctacatgaagtcggaatcgctagtaatcgcggatcagcacgccgcggtgaatacgttcccgggccttgcactcaccgcccgtca

SEQ ID NO: 54 - DNA, 1393 bp, Streptococcus sp.; identical to SEQ ID NO: 32
  misc_difference 1381: note = a, c, t, g, unknown or other
  source 1..1393: mol_type = unassigned DNA

SEQ ID NO: 55 - DNA, 1456 bp, Streptococcus sp.
  source 1..1456: mol_type = unassigned DNA
ttcaggacgaacgctggcggcgtgcctaatacatgcaagtagaacgctgaagactttagcttgctaaagttggaagagttgcgaacgggtgagtaacgcgtaggtaacctgcctactagcgggggataactattggaaacgatagctaataccgcataacagcatttaacccatgttagatgcttgaaaggagcaattgcttcactagtagatggacctgcgttgtattagctagttggtgaggtaacggctcaccaaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagagaagaacgtgtgtgagagtggaaagttcacacagtgacggtaacttaccagaaagggacggctaactacgtgccagcagccgcggtaatacgtaggtcccgagcgttgtccggatttattgggcgtaaagcgagcgcaggcggtttaataagtctgaagttaaaggcagtggcttaaccattgttcgctttggaaactgttagacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttaggccctttccggggcttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttaggagccagccgcctaagg

SEQ ID NO: 56 - DNA, 1442 bp, Streptococcus sp.
  source 1..1442: mol_type = unassigned DNA
ctaatacatgcgaggagaacgctgaagactttcttttgctatagttgggagagttgctaacgggtgagtaacgcgtaggtgacctgcctactagcgggggataactattgcaaacgatagctaataccgcataacagcctttaacccatgttagatgcttgaaaggagcaattgcttcactagtagatggacctgcgttgtattagctagttggtgaggtaacggctcaccaaggcgacgatacatagccgacctgagagggtgatcggccacactgggactgagacacggcccatactcctacgggaggcaccagtagggaatcttcgggaatgggggcaaccctgaccgagcaacgccgcgtgagtgaagaaggttttcggatcgtaaagctctgttgtaagagaagaacgtgtgtgagagtggaaagttcacactgtgacggtaacttaccagaaagggacggctaactacgtgccagcagccgcggtaatacgtaggtcccgagcgttgtccggatttattgggcgtaaagcgagcgcaggcggtttaataagtctgaagttaaaggcagtggcttaaccattgttcgctttggaaactgttagacttgagtgcataaggggagagtggaattccatgtgtagcggtgaaatgcgtagatatatggaggaacaccggtggcgaaagcggctctctggtctgtaactgacgttgaggctcgaaagcgtggggagcaaacaggattagataccctggtagtccacgctgtaaacgatgagtggtaggtgttaggccctttctggggtttagtgccgcagattacgcattaagccattcgcctggggagtacgaccgcaaggttgaaacttaaaggaattgacgggggcccgcacaagcggtggagcatgtggtttaattagaagcaacgcgaagaaccttaccaggtcttgacatcccgatgctattcttagagataggaagtttcttcggaacatcggtgacaggtggtgcatggttgtcgtcagctcgtgtcgagagatgttgggttaagtccctcaacgagcgcaacccctatttttatttgccatcattaagttgggcaatctagcgagactgccggtaataaaccggaggaaggtggggatgacgtcaaatcatcatgctccttatgtcatggggtacacacgtggtacaatggttggtacaacgagtcgcgagttggtgaaggcaagcaaatctcttaaagccaatatcagttcggattgtaggctgcaaatagcctacatgtagtcggaattgttagtaatcggggatcagcactccgcggtgaatacgtttccgggccttgtacaccccgcccgtctacaccacgagagtttgtaacacccgaagtcggtgaggtaactcttttaggagccagccgcctaaggtgggataga

SEQ ID NO: 57 - DNA, 1382 bp, Erysipelatoclostridium sp.
  source 1..1382: mol_type = unassigned DNA
agagtttgattatggctcaggatgaacgctggcggcgtgcctaatacatgcaagtcgaacgcgagcagcaatgctcgagtggcgaacgggtgagtaatacataagtaacctgccctagacagggggataactgctggaaacggcagctaagaccgcataggtatggacactgcatggtgaccatattaaaagtgccaaggcactggtagaggatggacttatggcgcattagctggttggtgaggtaacggctcaccaaggcgacgatgcgtagccgacctgagagggtgaccggccacactgggactgagacacggcccagactcctacgggaggcagcagtagggaattttcggcaatggggggaaccctgaccgagcaacgccgcgtgaaggaagaaggaattcgttctgtaaacttctgttataaaggaagaacggcggatatagggaatgatatccgagtgacggtactttatgagaaagccacggctaactacgtgccagcagccgcggtaatacgtaggtggcgagcgttatccggaattattgggcgtaaagagggagcaggcggcggcagaggtctgtggtgaaagactgaagcttaacttcagtaagccatagaaaccgggctgctagagtgcaggagaggatcgtggaattccatgtgtagcggtgaaatgcgtagatatatggaggaacaccagtggcgaaggcgacggtctggcctgtaactgacgctcattcccgaaagcgtggggagcaaataggattagataccctagtagtccacgccgtaaacgatgagtactaagtgttgggagtcaaatttcagtgctgcagttaacgcaataagtactccgcctgagtagtacgttcgcaagaatgaaactcaaaggaattgacgggggcccgcacaagcggtggagcatgtggtttaattcgatgatacgcgaggaaccttaccagggcttaaatgtgactgacaggtccggaaacggacttttcttcggacagttacaggtgctgcatggttgtcgtcagctcgtgccgtgaggtgtcaggttaagtcctataacgagcgcaacccctgtcgctagttgccagcgagtaatgtcgggaactctagcgagactgccagtgcaaactgcgaggaaggtggggatgacgtcaaatcatcacggcccttacgccctgggctacacacgtgctacaatggccggtacagagagcagccaccccgcgagggggagcgaatctacaaaaccggtcacagttcggatcggagtctgcaactcgactccgtgaagctggaatcgctagtaatcggatatcagccatgatccggtgaatacgttcccgggccttgtacacacccccgtc

SEQ ID NO: 58 - DNA, 1418 bp, Megasphaera sp.; identical to SEQ ID NO: 33
  source 1..1418: mol_type = unassigned DNA

SEQ ID NO: 59 - DNA, 1383 bp, unidentified
  source 1..1383: mol_type = unassigned DNA; note = Lachnospiraceae sequence
agagtttgatcctggctcaggatgaacgctggcggcgtgcttaacacatgcaagtcgaacgggaaacttttcattgaagcttcggcagatttggtctgtttctagtggcggacgggtgagtaacgcgtgggtaacctgccttatacagggggataacaaccagaaatggttgctaataccgcataagcgcacaggaccgcatggtccggtgtgaaaaactccggtggtataagatggacccgcgttggattagctagttggcagggtaacggcctaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaaatacgtgccagcagccgcggtaatacgtatggttcaagcgttatccggatttactgggtgtaaagggtgagtaggcggttatgcaagtcatatgtgaaatgtcggggctcaactccggcctgcataagaaactgtataactagagtgcaggagaggcaagcggaattcctagtgtagcggtgaaatgcgtagatattaggaagaacaccggtggcgaaggcggcttgctggactgttactgacgctgagtcacgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagtatgtggtttaattcgaagcaacgcgaagaaccttaccaggccttgacatggatataaatgttctagagatagaaagatagctatatatcacacaggtggtgcatggttgtcgtcagctcgtgtcgtgagatgttgggttaagtcccgcaacgagcgcaacccttgtcttctgttaccagcattgagttggggactcaggagagactgccggtgacaaaccggaggaaggtggggatgacgtcaaatcatcatgccccttatggcctgggctacacacgtactacaatggcgcctacaaagagcagcgacaccgcgaggtgaagcgaatctcataaagggcgtctcagttcggattgaagtctgcaactcgacttcatgaagtcggaatcgctagtaatcgcagatcagcatgctgcggtgaatacgttcccgggtcttgtactcaccgcccgtca

SEQ ID NO: 60 - DNA, 1361 bp, Blautia sp.
  source 1..1361: mol_type = unassigned DNA
gatgaacgctggcggcgtgcttaacacatgcaagtcgagcgaagcacttaagtggatctcttcggattgaaacttatttgactgagcggcggacgggtgagtaacgcgtgggtaacctgcctcatacagggggataacagttagaaatggctgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgtttttctagagtgccggagaggtaagcggaattcctagtgtagcggtgaaatgcgtagatattaggaggaacaccggtggcgaaggcggcttactggacgaccactgacgctgaggctcgaaagcgtggggagcaaacaggattagataccctggtagtccacgccgtaaaccgatgaataataggtgtcggggaacaatagttctttggtgccgcagcaaaacgcattaagtattccacctggggagtacgttcgcaagaatgaaactcaaaggaattgacggggacccgcacaagcggtggagcatgtggtttaattcgatgcaacgcgaagaaccttacctgctcttgacatcccactgaccggacagtaatgtgtccttttcttctgaacagtggagacaggtggtgcatggttgtcgtcagctcgtgtcgtgagatgttgggttaagtcccgcaacgagcgcaaccctcgtctttagtagccagcagtccggctgggcactctagagagactgccagggataacctggaggaaggcggggaggacgtcaaatcatcatgccccttacgagcagggctacacacgtgctacaatggcgtaaacaaagggaagcgaccccgtgaaggtgagcaaatctcaaaaataacgtctcagttcggattgtagtctgcaactcgactacatgaagctggaatcgctagtaatcgcgaatcagaatgtcgcggtgaataaaaggccgggtcttgcaca

SEQ ID NO: 61 - DNA, 253 bp, Alloprevotella sp.
  source 1..253: mol_type = unassigned DNA
tacggaaggtccaggcgttatccggatttattgggtttaaagggagcgcaggcggactcttaagtcagttgtgaaatacggcggctcaaccgtcggactgcagttgatactgggagtcttgagtacacgcagagatactggaattcatggtgtagcggtgaaatgctcagatatcatgaggaactccgatcgcgaaggcaggtatctggagtgtaactgacgctgaggctcgaaagtgcgggtatcaaacagg

SEQ ID NO: 62 - DNA, 1394 bp, Parabacteroides sp.; identical to SEQ ID NO: 18
  source 1..1394: mol_type = unassigned DNA

SEQ ID NO: 63 - DNA, 1445 bp, Bacteroides sp.
  source 1..1445: mol_type = unassigned DNA
tacaggcttaacacatgcaagtcgaggggcagcatgattgaagcttgcttcaattgatggcgaccggcgcacgggtgagtaacacgtatccaaccttccgtacactcagggatagcctttcgaaagaaagattaatacctgatggtatcttaagcacacatgtaattaagattaaagatttatcggtgtacgatggggatgcgttccattaggtagtaggcggggtaacggcccacctagcctacgatggatgggggttctgagaggaaggtcccccacattggaactgagacacggtccaaactcctacgggaggcagcagtgaggaatattggtcaatggacgagagtctgaaccagccaagtagcgtgaaggatgaaggtcctacggattgtaaacttcttttataagggaataaaacctcccacgtgtgggagcttgtatgtaccttatgaataagcatcggctaactccgtgccagcagccgcggtaatacggaggatgcgagcgttatccggatttattgggtttaaagggagcgcagacgggtcgttaagtcagctgtgaaagtttggggctcaaccttaaaattgcagttgatactggcgtccttgagtgcggttgaggtgtgcggaattcgtggtgtagcggtgaaatgcttagatatcacgaagaactccgattgcgaaggcagcacactaagccgtaactgacgttcatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaatatattggaaacagtatagccgtaaggcaaatgtgaaggtgctgcatggttgtcgtcagctcgtgccgtgaggtgtcggcttaagtgccataacgagcgcaacccttatcttcagttactaacaggtcatgctgaggactctggagagactgccgtcgtaagatgtgaggaaggtggggatgacgtcaaatcagcacggcccttacgtccggggctacacacgtgttacaatggggggtacagaaggccgctacctggtgacaggatgctaatcccaaaagcctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggctaagtcata

SEQ ID NO: 64 - DNA, 1522 bp, unidentified
  source 1..1522: mol_type = unassigned DNA; note = Escherichia-Shigella sequence
agagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggcggagcatgtggattaattcgatgcaacgcgaagaaccttacctgggtttgacatgcacaggacgcgtctagagataggcgttcccttgtggcctgtgtgcaggtggtgcatggctgtcgtcagctcgtgtcgtgagatgttgggttaagtcccgcaacgagcgcaacccttgtctcatgttgccagcacgtaatggtggggactcgtgagagactgccggggtcaactcggaggaaggtggggatgacgtcaagtcatcatgccccttatgtccagggcttcacacatgctacaatggccggtacaaagggctgcgatgccgcgaggttaagcgaatccttaaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccagtggcctaaccctcgggagggagctgtcgaaggtgggatcggcgattgggacgaagtcgtaacaaggtaaccgtaggggaacctgcggttg

SEQ ID NO: 65 - DNA, 1472 bp, unidentified; identical to SEQ ID NO: 35
  source 1..1472: mol_type = unassigned DNA; note = Escherichia-Shigella sequence

SEQ ID NO: 66 - DNA, 1374 bp, Blautia sp.
  source 1..1374: mol_type = unassigned DNA
agagtttgatcatggctcaggatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatgggtaggggttctgagaggaaggtcccccacattggaactgagacacggcccaaactcatacgggaggcagcagtggggaatattgcacaatgggggaaaccctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcagtgcaagtctgaagtgaaagcccggggctcaaccccgggactgctttggaaactgtgcagctagagtgtcggagaggcaagcggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactactaggtgtcggggagcaaagctcttcggtgccgcagccaacgcaataagtagtccacctggggagtacgttcgcaagaatgaaactcaaaggaattgacggggacccgcacaagcggtggagcatgtggtttaattcgaagcaacgcgaagaaccttacctgctcttgacatccctctgaccgctctttaatcggagctttccttcgggacagaggagacaggtggtgcatggttgtcgtcagctcgtgtcgtgagatgttgggttaagtcccgcaacgagcgcaacccttatggtcagttactacgcaagaggactctggccagactgccgttgacaaaacggaggaaggtggggatgacgtcaaatcatcatgccctttatgacttgggctacacacgtactacaatggcgttaaacaaagagaagcgagaccgcgaggtggagcaaaactcggaaacaacgtcccagttcggactgcaggctgcaactcgcctgcacgaagtcggaattgctagtaatcgcagatcagcatgctgcggtgaatacgttcccgggccttgcactcaccgcccgt

SEQ ID NO: 67 - DNA, 1440 bp, Tyzzerella sp.; identical to SEQ ID NO: 36
  source 1..1440: mol_type = unassigned DNA

SEQ ID NO: 68 - DNA, 1370 bp, Ruminococcus gauvreauii
  source 1..1370: mol_type = unassigned DNA
atgaacgctggcggcgtgcctaacacatgcaagtcgaacgaagcgcttgaacggatatcttcggactgaagttcttgcgactgagtggcggacgggtgagtaacgcgtgggtaacctgcctcatacagggggataacagttagaaatgactgctaataccgcataagcgcacagcttcgcatggagcagtgtgaaaaactccggtggtatgagatggacccgcgtcagattagctagttggcagggtaacggcctaccaaggcgacgatctgtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagataatgacggtacctgactaagaagctccggctaaatacgtgccagcagccgcggtaatacgtatggagcaagcgttatccggatttactgggtgtaaagggagcgtagacggtttgacaagtctgatgtgaaattccagggcttaaccctggacctgcattggaaactgtcggactagagtgtcggagaggtgagtggaattcctagtgtagcggtgaaatgcgtagatattaggaggaacaccagtggcgaaggcggctcactggacgataactgacgttgaggctcgaaagcgtggggagcaaacaggattagataccctggtagtccacgccgtaaacgatgtgtactaggtgttggggagcaaagctcttcggtgccgtcgcaaacgcagtaagtacaccacctggggagtacgttcgcaagaatgaaactcaaaggaattgacggggacccgcacaagcggtggagcatgtggtttaattcgaagcaacgcgaagaaccttaccaaatcttgacatcggagtgaccgctctttaatcggagctttccttcgggacactccagacaggtggtgcatggttgtcgtcagctcgtgtcgtgagatgttgggttaagtcccgcaacgagcgcaacccttatccttagtagccagcaagtgaagttgggcactctagggagactgccagggataacctggaggaaggtggggatgacgtcaaatcatcatgccccttatgatttgggctacacacgtgctacaatggcgtaaacaaagggaagcgatcacgtgagtgtgagcaaatctcaaaaataacgtcccagttcggactgtagtctgcaacccgactacacgaagctggaatcgctagtaatcgcaggtcagcatactgcggtgaatacgttcccgggtcttgcacacaccgcccgtca

SEQ ID NO: 69 - DNA, 1366 bp, Fusobacterium sp.; identical to SEQ ID NO: 37
  source 1..1366: mol_type = unassigned DNA

SEQ ID NO: 70 - DNA, 1361 bp, Fusobacterium sp.
  source 1..1361: mol_type = unassigned DNA
agagtttgatcctggctcaggacgaacgctggcggcgtgcctaacacatgcaagtcgagcgattctcttcggagaagagcggcggacgggtgagtaacgcgtgggtaacctgccctgtacacacggataacataccgaaaggtatgctaatacgggataatatataagagtcgcatgacttttatatcaaagatttttcggtacaggatggacccgcgtctgattagcttgttggcggggtaacggcccaccaaggcgacgatcagtagccgacctgagagggtgatcggccacattggaactgagacacggtccaaactcctacgggaggcagcagtggggaatattgcacaatgggcgcaagcctgatgcagcaacgccgcgtgagcgatgaaggccttcgggtcgtaaagctctgtcctcaaggaagataatgacggtacttgaggaggaagccccggctaactacatgccagcagccgcggtaatacgtatgtcgcaagcgttatccggatttattgggcgtaaagcgcgtctaggtggtttggtaagtctgatgtgaaaatgcggggctcaactccgtattgcgttggaaactgccaaactagagtactggagaggtaggcggaactacaagtgtagaggtgaaattcgtagatatttgtaggaatgccgatggggaagccagcctactggacagatactgacgctaaagcgcgaaagcgtgggtagcaaacaggattagataccctggtagtccacgccgtaaacgatgattactaggtgttgggggtcgaacctcagcgcccaagctaacgcgataagtaatccgcctggggagtacgtacgcaagtatgaaactcaaaggagttgacggggacccgcacaagcggtggagcatgtggtttaattcgacgcaacgcgaggaaccttaccagcgtttgacatcctaggaatgagaaagagatttcttagtgctccttcgggagaacctagagacaggtggtgcatggctgtcgtcagctcgtgtcgtgagatgttgggttaagtcccgcaacgagcgcaacccctattgtatgttgccatcattaagttgggcactcatgcgatactgcctgcgatgagcaggaggaaggtggggatgacgtcaagtcatcatgccccttatacgctgggctacacacgtgctacaatgggcagtacagagagaagcaataccgcgaggtggagccaaacttaaaaaccagtctcagttcggattgtaggctgaaactcgcctacatgaagctggagttactagtaatcgcgaatcagaatgtcgcggtgaatacgtacccgggtcttgtacacaccgcccgtca

SEQ ID NO: 71 - DNA, 1360 bp, unidentified
  source 1..1360: mol_type = unassigned DNA; note = Lachnospiraceae sequence
gatgaacccttgcggcgtgcttaacacatgcaagtcgaacgggaaacattttattgaagcttcggcagatctagcttgtttctagtggcggacgggtgagtaacgcgtgggcaacctgcctcacactgggggataacagtcagaaatggctgctaataccgcataagcgcacagcatcgcatgatgcagtgtgaaaaactccggtggtgtgagatggacccgcgttggattagctagttggcagggcagcggcctaccaaggcgacgatccatagccggcctgagagggtgaacggccacattgggactgagacacggcccagactcccacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggtgttgcaagtctgatgtgaaaggcgggggctcaacccctggactgcattggaaactgtgatactcgagtgccggagaggtaagcggaattcctagtgtagcggtgaaatgcgtagatattagaaggaataccagtggcgaaggcggcttactggacggtaactgacgttgaggctcgaaagcgtggggagcaaacaagattaagaaacctctgggtagtccacgcccgtaaacgaaggaataaaggggtcgggagcagagcttttcggtgccgcagcaaacccaataagtattccaccttgagaggacgttcgcaagaatgaaactcaaaggaattgacgggggacccgcacaagcggtggagcatgtggtttaattcgaagcaacgcgaagaaccttaccaagtcttgacatccctctgaccgcaccttaaccggtgctttccttcgggacagaggagacaggtggtgcatggttgtcgtcagctcgtgtcgtgagatgttgggttaagtcccgcaacgagcgcaacccttatccttagtagccagcggtccggccgggcactctggggagactgccagggataacctggaggaaggtggggatgacgtcaaatcatcatgccccttatgatttgggctacacacgtgctacaatggcgtaaacaaagggaagcgatcacgtgagtgcgagcaaatctcaaaaataacgtcccagttcggactgtagtctgcaacccgactacacgaagctggaatcgctagtaatcgcaggtcagcatactgcggtgaatacgttcccgggtcttgtac

SEQ ID NO: 72 - DNA, 1332 bp, Blautia sp.; identical to SEQ ID NO: 26
  source 1..1332: mol_type = unassigned DNA

SEQ ID NO: 73 - DNA, 1311 bp, unidentified
  misc_difference 725: note = a, c, t, g, unknown or other
  misc_difference 727: note = a, c, t, g, unknown or other
  misc_difference 740: note = a, c, t, g, unknown or other
  misc_difference 747..748: note = a, c, t, g, unknown or other
  source 1..1311: mol_type = unassigned DNA; note = Peptostreptococcaceae sequence
gatgaacgctggcggcgtgcctaacacatgcaagtcgagcgatttacttcggtaaagagcggcggacgggtgagtaacgcgtgggtaacctgccctgtacacacggataacataccgaaaggtatgctaatacgagataatatgcttttatcgcatggtagaagtatcaaagcttttgcggtacaggatggacccgcgtctgattagctagttggtaaggtaacggcttaccaaggcaacgatcagtagccgacctgagagggtgatcggccacattggaactgagacacggtccaaactcctacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggaattactgggcgtaaagggtgcgtaggtggtttcttaagtcagaggtgaaaggctacggctcaaccgtagtaagcctttgaaactgggaaacttgagtgcaggagaggagagtggaattcctagtgtagcggtgaaatgcgtagatattaggaggaacaccagttgcgaaggcggctctctggactgtaactgacactgaggcacgaaagcgtgggagcaaacaagattagntnccctggtagtccncgccgtnnccgcccataaagagctgtcggaggttacccccttcggtggcgcaggtaacgcaataaagaattccgcctgggaaggaacgcttcgcaagagtgaaattaaaaggaatagacggggacccgctcaagtagtggagcatgtggtttaattcgaagcaacgcgaagaactttctctaagcttgacatccttttgaccgatgcctaatagcatcaatcccttctgggacagaagtgacaggtggtgcatggttgttgtcagctcgtgtcgtgagatgttgggttaagtcccgcaacgagcgcaacccttgcctttagttgccagcattaagttgggcactctatagggactgccagggataacctggaggaaggtggggatgacgtcaaatcatcatgccccttatgcttagggctacacacgtgctacaatgggtggtacagagggcagccaagtcgtgaggcggagctaatcccttaaagccattctcagttcggattgtaggctgaaactcgcctacatgaagctggagttactagtaatcgcagatcagaatgatgcggtgaa

SEQ ID NO: 74 - DNA, 1324 bp, Intestinibacter sp.; identical to SEQ ID NO: 28
  source 1..1324: mol_type = unassigned DNA

SEQ ID NO: 75 - DNA, 1288 bp, Peptoclostridium sp.
  source 1..1288: mol_type = unassigned DNA
gaaagcggcggacgggtgagtaacgcgtaggcaacctgccccatacagagggatagcatctggaaacggatattaatacctcataatacttagagatcacatggtaactaagtcaaagatttatcggtatgggatgggcctgcgtctgattagctagttggtggggtaacggctcaccaaggcgacgatcagtagccgacctgagagggtgatcggccacattggaactgagacacggtccaaactcctacgggaggcagcagtggggaatattgcacaatgggcgcaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggatttactgggcgtaaagggtgcgtaggcggtctttcaagtcaggagttaaaggctacggctcaaccgtagtaagctcctgatactgtctgacttgagtgcaggagaggaaagcggaattcccagtgtagcggtgaaatgcgtagatattgggaggaacaccagtagcgaaggcggctttctggactgtaactgacgctgaggcacgaaagcgtggggagcaaacaggattagataccctggtagtccacgctgtaaacgatgagtactaggtgtcggaggttacccccttcggtgccgcagctaacgcattaagtactccgcctggggagtacgcacgcaagtgtgaaactcaaaggaattgacggggacccgcacaagtagcggagcatgtggtttaattcgaagcaacgcgaagaaccttacctaggcttgacatccttctgaccgaggactaatctcctctttccctccggggacagaagtgacaggtggtgcatggttgtcgtcagctcgtgtcgtgagatgttgggttaagtcccgcaacgagcgcaacccttgtctttagttgccatcattaagttgggcactctagagagactgccagggataacctggaggaaggtggggatgacgtcaaatcatcatgccccttatgcctagggctacacacgtgctacaatgggtggtacagagggcagccaagccgtgaggtggagcaaatcccttaaagccattctcagttcggattgtaggctgaaactcgcctacatgaagctggagttactagtaatcgcagatcagaatgctgcggtgaatgcgttcccgggtcttgcacacaccgcccgtca

SEQ ID NO: 76 - DNA, 1391 bp, Sutterella sp.
  source 1..1391: mol_type = unassigned DNA
ctggctcaggatgaacgctagcgacaggcttaacacatgcaagtcgaggggcatcacgggaggtagcaataccttctggtggcgaccggcgcacgggtgagtaacacgtatgcaacctgccctgtacagagggacaagcggtggaaacgccgtctaatcccgcatgcactcttccgggggcatccccgggagagtaaaggagagatccggtacaggatggacatgcggcgcattagttagttggcggggtaacggcccaccaagacgacgatgcgtaggggttctgagaggaaggtcccccacattggaactgagacacggtccaaactcctacgggaggcagcagtgaggaatattggtcaatgggcggaagcctgaaccagccaagtcgcgtgagggaagacggccctacgggttgtaaacctcttttgtcggggagcaatgccgcctttgcgaaggcggagggagagtacccgaagaaaaagcaccggctaactacgtgccagcagccgcggtaatacgtagggtgcaagcgttaatcggaattactgggcgtaaagcgtgcgcaggcggttctgtaagacagatgtgaaatccccgggctcaacctgggaattgcatttgtgactgcaggactagagttcatcagaggggggtggaattccaagtgtagcagtgaaatgcgtagatatttggaagaacaccaatggcgaaggcagccccctgggatgcgactgacgctcatgcacgaaagcgtggggagcaaacaggattagataccctggtagtccacgccctaaacgatgtctactggttgttggggattaatatccttggtaacgaagctaacgcgtgaagtagaccgcctggggagtacggtcgcaagattaaaactcaaaggaattgacggggacccgcacaagcggtggatgatgtggattaattcgatgatacgcgaggaaccttacccgggctcaaacggcacagtgatacttttgaaaggaggtagctctacggagactgtgccgaggtgctgcatggttgtcgtcagctcgtgccgtgaggtgtcggcttaagtgccataacgagcgcaacccctattgtcagttgccagcaggtaaagctggggactctgacgagactgccggcgcaagctgagaggaaggcggggatgacgtcaaatcagcacggcccttacgtccggggcgacacacgtgttacaatggcaggcacagcgggaagccacccggcgacggggagcggaacccgaaagcctgtctcagttcggatcggagtctgcaactcgactccgtgaagctggattcgctagtaatcgcgcatcagccatggcgcggtgaatacgttcccgggccttgtacacaccgcccgta

SEQ ID NO: 77 - DNA, 1357 bp, Collinsella sp.; identical to SEQ ID NO: 12
  misc_difference 766: note = a, c, t, g, unknown or other
  source 1..1357: mol_type = unassigned DNA

SEQ ID NO: 78 - DNA, 1432 bp, Phascolarctobacterium sp.
  source 1..1432: mol_type = unassigned DNA
agagttgatctggctcagattgaacgctggcggcaggcttaatacatgcaagtcgaacggtaacagcaaaaaagcttgcttttttggctgacgagtggcggacgggtgagtaatacctaggaagctgcctaaacgagggggataacacctggaaacgggtgctaataccgcatgataccgcaaggtcaaaggttggtttaccaatcgcgtttagatgcgcctaggagggattagctagttggtggggtaacggctcaccaaggcgatgatcagtagccggtctgagaggatgaacggccacattgggactgagacacggcccagactcctacgggaggcagcagtggggaatcttccgcaatgggcgaaagcctgacggagcaatgccgcgtgagtgatgaagggattcgtcccgtaaagctctgttgtatatgacgaatgtgcagattgtgaataatgatttgtaatgacggtagtatacgaggaagccacggctaactacgtgccagcagccgcggtaatacgtaggtggcgagcgttgtccggaattattgggcgtaaagagcatgtaggcggttttttaagtctggagtgaaaatgcggggctcaaccccgtatggctctggatactggaagacttgagtgcaggagaggaaaggggaattcccagtgtagcggtgaaatgcgtagatattgggaggaacaccagtggcgaaggcgcctttctggactgtgtctgacgctgagatgcgaaagccagggtagcgaacgggattagataccccggtagtcctggccgtaaacgatgggtactaggtgtgggaggtatcgaccccttccgtgccggagttaacgcaataagtaccccgcctggggagtacgtccgcaaggatgaaactcaaaggaattgacgggggcccgcacaagcggtggagtatgtggtttaattcgacgcaacgcgaagaaccttaccaaggcttgacattgattgaaagacctagagataggtccctctcttcggagacaagaaaacaggtggtgcatggctgtcgtcagctcgtgtcgtgagatgttgggttaagtcccgcaacgagcgcaacccctatcctatgttaccagcgggtaatgccggggactcataggagactgccaaggacaacttggaggaaggcggggatgacgtcaagtcatcatgccccttatgtcttgggctacacacgtactacaatggtcggcaacagagggaagcaaagccgcgaggcagagcaaaccccagaaacccgatctcagttcggattgcaggctgcaactcgcctgcatgaagtcggaatcgctagtaatcgcaggtcagcatactgcggtgattactatcccgggcgttgtactcaccgcccgtcaggcggagttcgtacttcaaatgtgccacactggg

SEQ ID NO: 79 - DNA, 253 bp, Clostridium sp.
  source 1..253: mol_type = unassigned DNA
tacgtaggtggcaagcgttgtccggatttactgggcgtaaagggagcgtaggcggatttttaagtgggatgtgaaatacccgggctcaacctgggtgctgcattccaaactggaaatctagagtgcaggaggggaaagtggaattcctagtgtagcggtgaaatgcgtagagattaggaagaacaccagtggcgaaggcgactttctggactgtaactgacgctgaggctcgaaagcgtggggagcaaacagg

SEQ ID NO: 80 - DNA, 253 bp, Clostridium sp.; identical to SEQ ID NO: 14
  source 1..253: mol_type = unassigned DNA

SEQ ID NO: 81 - DNA, 1402 bp, Turicibacter sp.
  source 1..1402: mol_type = unassigned DNA
agagtttgatcctggctcaggatgaacgctagcgataggcctaacacatgcaagtcgaggggcagcacatgagtagcaatacgatggtggcgaccggcgcacgggtgagtaacacgtatgcaacctacctttaacaggggaataacccgttgaaaaacggactaatactccataacacaggggtcccgcatgggaatatttgttaaagatttatcggttgaagatgggcatgcgttccattagctagttggtagggtaaaggcctaccaaggcgacgatgcgtagccgacctgagagggtgaacggccacactgggactgagacacggcccacactcctacgggaggcagcagtagggaatcttcggcaatgggcgaaagcctgaccgagcaacgccgcgtgaatgatgaaggccttcgggttgtaaaattctgttataagggaagaacgactttagtaggaaatggctagagtgtgacggtaccttatgagaaagccacggctaactacgtgccagcagccgcggtaatacgtaggtggcgagcgttatccggaattattgggcgtaaagagcgcgcaggtggttgattaagtctgatgtgaaagcccacggcttaaccgtggagggtcattggaaactggtcgacttgagtgcagaagagggaagtggaattccatgtgtagcggtgaaatgcgtagagatatggaggaacaccagtggcgaaggcggcttcctggtctgtaactgacactgaggcgcgaaagcgtggggagcaaacaggattagataccctggtagtccacgccgtaaacgatgagtgctaagtgttgggggtcgaacctcagtgctgaagttaacgcattaagcactccgcctggggagtacggtcgcaagactgaaactcaaaggaattgacggggacccgcacaagcggtggagcatgtggtttaattcgaagcaacgcgaagaaccttaccaggtcttgacataccattgaccgttctagagataggattttcccttcggggacaatggatacaggtggtgcatggttgtcgtcagctcgtgtcgtgagatgttgggttaggtcccgcaacgagcgcaacccctgtcgttagttgccagcattcagttggggactctaacgagactgccagtgacaaactggaggaaggtggggatgacgtcaaatcatcatgccccttatgacctgggctacacacgtgctacaatggttggtacaaagagaagcgaagcggtgacgtggagcaaacctcataaagccaatctcagttcggattgtaggctgcaactcgcctacatgaagttggaatcgctagtaatcgcgaatcagaatgtcgcggtgaatacgttcccgggtcttgtacacaccgcccgtca

SEQ ID NO: 82 - DNA, 1409 bp, Streptococcus sp.
  source 1..1409: mol_type = unassigned DNA
tgcaagtagaacgctgaagactggtgcttgcaccggttggaagagttgcgaacgggtgagtaacgcgtaggtaacctgcctgatagcgggggataactattggaaacgatagctaataccgcataacagggaataacacatgttatttttttgaaaggggcaattgctccactatcagatggacctgcgttgtattagctagtaggtgaggtaacggctcacctaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagagaagaacgttgagtagagtggaaagttactcaagtgacggtatcttaccagaaagggacggctaactacgtgccagcagccgcggtaatacgtaggtcccgagcgttgtccggatttattgggcgtaaagcgagcgcaggcggtttaataagtctgaagttaaaggcagtggctcaaccattgttcgctttggaaactgttaaacttgagtgcagaaggggagagtggaattccatgtgtagcggtgaaatgcgtagatatatggaggaacaccggtggcgaaagcggctctctggtctgtaactgacgctgaggctcgaaagcgtgggtagcgaacaggattagataccctggtagtccacgccgtaaacgatgagtgctaggtgttgggtcctttccgggactcagtgccgacgctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgctatccctagagatagggagttacttcggtacatcggtgacaggtggtgcatggttgtcgtcagctcgtgtcgtgagatgttgggttaagtcccgcaacgagcgcaacccctattgttagttgccatcattcagttgggcactctagcgagactgccggtaataaaccggaggaaggtggggatgacgtcaaatcatcatgccccttatgacctgggctacacacgtgctacaatggttggtacaacgagttgcgagtcggtgacggcaagctaatctcttaaagccaatctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccaaagtcggtgaggtaaccttcggagcc

SEQ ID NO: 83 - DNA, 1517 bp, Streptococcus sp.; identical to SEQ ID NO: 50
  source 1..1517: mol_type = unassigned DNA

SEQ ID NO: 84 - DNA, 252 bp, Streptococcus sp.; identical to SEQ ID NO: 51
  source 1..252: mol_type = unassigned DNA

SEQ ID NO: 85 - DNA, 1422 bp, Streptococcus sp.
  source 1..1422: mol_type = unassigned DNA
agagtttgatcctggctcaggacgaacgctggcggcgtgcctaatacatgcaagtcgagcgagcggaactaacagatttacttcggtaatgacgttaggaaagcgagcggcggatgggtgagtaacacgtggggaacctgccccatagtctgggataccacttggaaacaggtgctaataccggataagaaagcagatcgcatgatcagcttttaaaaggcggcgtaagctgtcgctatgggatggccccgcggtgcattagctagttggtaaggtaaaggcttaccaaggcaatgatgcatagccgagttgagagactgatcggccacattgggactgagacacggcccaaactcctacgggaggcagcagtagggaatcttccacaatggacgcaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtgccagcagccgcggtaatacgtaggtggcaagcgttgtccggatttattgggcgtaaagcgagcgcaggcggtttaataagtctgaagttaaaggcagtggcttaaccattgttcgctttggaaactgttagacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttaggccctttccggggcttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgctattcctagagataggaagtttcttcggaacatcggtgacaggtggtgcatggttgtcgtcagctcgtgtcgtgagatgttgggttaagtcccgcaacgagcgcaacccctattgttagttgccatcattaagttgggcactctagcgagactgccggtaataaaccggaggaaggtggggatgacgtcaagtcatcatgccccttatgacctgggctacacacgtgctacaatgggcagtacaacgagaagcgagcctgcgaaggcaagcgaatctctgaaagctgttctcagttcggactgcagtctgcaactcgactgcacgaagctggaatcgctagtaatcgcggatcagcacgccgcggtgaatacgttcccgggccttgcacacaccgcccgtca

SEQ ID NO: 86 - DNA, 1536 bp, Streptococcus sp.
  source 1..1536: mol_type = unassigned DNA
agagtttgatcctggctcaggacgaacgctggcggcgtgcctaatacatgcaagtggaacgcacagttagtatgtagtttactacaacattacttgtgagtcgcgaacgggtgagtaacgcgtaggtaacctgccttgtagcgggggataactattggaaacgatagctaataccgcataacagttgataactcatgttattagcttgaaagatgcaacagcatcactacgagatggacctgcgttgtattagctagttggtgaggtaaaggctcaccaaggccacgatacatagccgacctgagagggtgatcggccacattgggactgagacacggcccaaactcctacgggaggcagcagtagggaatcttcggcaatgggggcaaccctgaccgagcaacgccgcgtgagtgaagaaggttttcggatcgtaaagctctgttgtaagagaagaacgttgatgagagtggaaaattcatcaagtgacggtatcttaccagaaagggacggctaactacgtgccagcagccgcggtaatacgtaggtcccgagcgttgtccggatttattgggcgtaaagcgagcgcaggcggtttcgtaagtctgaagttaaaggcagtggctcaaccattgttcgctttggaaactgcgagacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttaggccctttccggggcttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgcccgctctagagatagagttttacttttgtacatcggtgacaggtggtgcatggttgtcgtcagctcgtgtcgtgagatgttgggttaagtcccgcaacgagcgcaacccctattgttagttgccatcattgagttgggcactctagcgagactgccggtaataaaccggaggaaggtggggatgacgtcaaatcatcatgccccttatgacctgggctacacacgtgctacaatggctggtacaacgagtcgcaagccggtgacggcaagctaatctcttaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccttttaggagccagccgcctaaggtgggatagatgattggggtgaagtcgtaacaaggtagccgtatcggaaggtgcggctg

SEQ ID NO: 87 - DNA, 1393 bp, Streptococcus sp.; identical to SEQ ID NO: 32
  misc_difference 1381: note = a, c, t, g, unknown or other
  source 1..1393: mol_type = unassigned DNA

SEQ ID NO: 88 - DNA, 1510 bp, Streptococcus sp.
  source 1..1510: mol_type = unassigned DNA
agagtttgatcctggctcaggacgaacgctggcggcgtgcctaatacatgcaagtagaacgctgaagaaaggagcttgcttcttttggatgagttgcgaacgggtgagtaacgcgtaggtaacctgccttgtagcgggggataactattggaaacgatagctaataccgcataacagcttttgacacatgttagaagcttgaaagatgcaattgcatcactacgagatggacctgcgttgtattagctagtaggtagggtaacggcctacctaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagagaagaacgtgtgtgagagtggaaagttcacacagtgacggtaacttaccagaaagggacggctaactacgtgccagcagccgcggtaatacgtaggtcccgagcgttgtccggatttattgggcgtaaagcgagcgcaggcggtttaataagtctgaagttaaaggcagtggcttaaccattgttcgctttggaaactgttaaacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttaggccctttccggggcttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccagtgaccgctctagagatagagtttttcttcggaacactggtgacaggtggtgcatggttgtcgtcagctcgtgtcgtgagatgttgggttaagtcccgcaacgagcgcaacccttattgttagttgccatcattcagttgggcactctagcgagactgccggtaataaaccggaggaaggtggggatgacgtcaaatcatcatgccccttatgacctgggctacacacgtgctacaatggttggtacaacgagtcgcaagtcggtgacggcaagcaaatctcttaaagccaatctcagttcggattgtaggctgcaactcgcctacatgaagcaggaattgctagtaatggcaggtcagcatactgccgtgaatacgttcccgggccttgtacacaccgcccgtcacaccatgagagtctgtaacacccgaagtcggtagtctaactacggaggacgccgccgaaggtgggacagataattggggtgaagtcgtaacaaggtagccgta

SEQ ID NO: 89 - DNA, 1442 bp, Streptococcus sp.; identical to SEQ ID NO: 56
  source 1..1442: mol_type = unassigned DNA

SEQ ID NO: 90 - DNA, 1397 bp, Anaerostipes sp.
  source 1..1397: mol_type = unassigned DNA
ctacctgcagtcgacgaacaccttatttgattttcttcggaactgaagatttggtgattgagtggcggacgggtgagtaacgcgtgggtaacctgccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagaaagtgaacggccacattgggactgagacacggcccaaactcctacgggaggcagcagtggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtagaggcttcggtgccgcagccaacgcagtaagtattccacctggggagtacgttcgcaagaatgaaactcaaaggaattgacggggacccgcacaagtagcggagcatgtggtttaattcgaagcaacgcgaagaaccttacctaagcttgacatccttttgaccgatgcctaatcgcatctttcccttcggggacagaagtgacaggtggtgcatggttgtcgtcagctcgtgtcgtgagatgttgggttaagtcccgcaacgagcgcaacccttgcctttagttgccatcattaagttgggcactctagagggactgccagggataacctggaggaaggtggggatgacgtcaaatcatcatgccccttatgcttagggctacacacgtgctacaatgggtggtacagagggcagcgaagtcgtgaggccaagctaatcccttaaagccattctcagttcggattgtaggctgaaacccgcctacatgaagctggagttactagtaatcgcagatcagaatgctgcggtgaatgcgttcccgggtcttgtacacaccgcccgtcacaccatgggagttgggggcgcccgaagccggctagctactttggaagcgt

SEQ ID NO: 91 - DNA, 1435 bp, Lachnoclostridium sp.
  source 1..1435: mol_type = unassigned DNA
ggggggcttaacacatgcaagtcgaacgaagcgctttcgctttaatcttcggaggaaagaggaagtgactgagtggcggacgggtgagtaacgcgtgggtaacctgcctcatacagggggataacagttagaaatggctgctaataccgcataagcatacagcaccgcatggtgcagtgtgaaaaactccggtggtataagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgaaggaagaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagtgcaagtctgaagtgaaagcccggggctcaaccccgtgactgctttggaaactgtgcttctagagtgtcggagaggtaagcggaattcctagtgtagcggtgaaatgcgtagatattaggaggaacatcagtggcgaaggcggcttactgggcgataactgacgttgaggctcgaaagcgtggggagcaaacaggattagataccctggtagtccacgccgtaaacgatgaatactaggtgtcgggaagcacagcttttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatcccggtgaccggacagtaatgtgtccttttcttcggaacaccggtgacaggtggtgcatggttgtcgtcagctcgtgtcgtgagatgttgggttaagtcccgcaacgagcgcaacccttatccccagtagccagcggttcggccgggcactctgaggagactgccagggataacctggaggaaggtggggatgacgtcaaatcatcatgccccttatgacttgggctacacacgtgctacaatggcgtaaacaaagggaagcgagaccgtgaggtggagcaaatcccaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttggaaatgcccgaagtcagtgacccaaccgcaaggagggagctgccgaaggcaggttcgataactg

SEQ ID NO: 92 - DNA, 1493 bp, Roseburia sp.
  source 1..1493: mol_type = unassigned DNA
agagtttgatcctggctcaggatgaacgctggcggcgtgcttaacacatgcaagtcgaacgaagcactttaacttgattttttcggaatgattgttcttgtgactgagtggcggacgggtgagtaacgcgtgggtaacctgcctcatacagggggataacagttagaaatgactgctaataccgcataagcgcacggtatcgcatgatacagtgtgaaaaactccggtggtatgagatggacccgcgtctgattagctagttggcggggtaacggcccaccaaggcgacgatcagtagccgacctgagagggtgaccggccacattgggactgagacacggcccaaactcctacgggaggcagcagtggggaatattgcacaatgggcgaaagcctgatgcagcgacgccgcgtgaacgaaaaagtatttcggtatgtaaagttctatcagcagggaagataatgacggtacctgactaagaagcaccggctaaatacgtgccagcagccgcggtaatacgtatggtgcaagcgttatccggatttactgggtgtaaagggagcgcaggcggtacggcaagtctgatgtgaaagcccggggctcaaccccggtactgcattggaaactgtcgaactagagtgtcggaggggtaagcggaattcctagtgtagcggtgaaatgcgtagatattaggaggaacaccagtggcgaaggcggcttactggacgacaactgacgctgaggcgcgaaagcgtggggagcaaacaggattagataccctggtagtccacgctgtaaacgatgaatactaggtgtgggaggactgaccccttccgtgccgcagttaacacaataagtattccacctggggagtacggccgcaaggctgaaactcaaaggaattgacgggggcccgcacaagcagtggattatgtggtttaattcgacgcaacgcgaagaaccttaccaggacttgacatccaactaacgaagtagagatacattaggtgcccttcggggaaagttgagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggcggggacgacgtcaaatcatcatgccccttatgtcctgggctacacacgtaatacaatggccgtcaacaaagggaagcaaagccgcgaggtggagcaaatccccaaaaacggtctcagttcggattgcaggctgcaactcgcctgcatgaagctggaattgctagtaatcgtggatcagcatgccacggtgaatacgttcccgggccttgtacacaccgcccgtcacaccatgagagtcgggaacacccgaagtccgtagtctaaccgcaaggagggcgcggccgaaggtgggtccggtaattggggtgaagtcgtaacaaggtaaccgt

SEQ ID NO: 93 - DNA, 1289 bp, Fusobacterium sp.
  source 1..1289: mol_type = unassigned DNA
agtggcgaacgggtgagtaacgcgtgaggaacctgcctttcagtgggggacaacagttggaaacgactgctaataccgcatgatactttttggaggcatctctgaaaagtcaaagctttatgtgctgaaagatggtctcgcgtctgattagctagttggtgaggtaacggctcaccaaggcgacgatcagtagccggtctgagaggatgaacggccacattgggactgagatacggcccagactcctacgggaggcagcagtggggaatattgggcaatgggggaaaccctgacccagcaacgccgcgtgaaggaagaaggccttcgggttgtaaacttcttttaccagggacgaagaacgtgacggtacctggagaaaaagcaacggctaactacgtgccagcagccgcggtaatacgtaggttgcaagcgttatccggatttattgggcgtaaagcgcgtctaggcggtttggtaagtctgatgtgaaaatgcggggctcaactccgtattgcgttggaaactgctaaactagagtactggagaggtaggcggaactacaagtgtagaggtgaaattcgtagatatttgtaggaatgccgatggggaagccagcctactggacagatactgacgctaaagcgcgaaagcgtggggagcaaacaggattagataccctggtagtccacgctgtaaacgatgagtactaggtgtcggaggttacccccttcggtgccgcagctaacgcattaagtactccgcctggggagtacgcacgcaagtgtggaactcaaaggaattgacggggacccgcacaagtagcggagcatgtggtttaattcgaagcaacgcgaagaaccttacctaggcttgacatccttctgaccgaggactaatctcctctttccctccggggacagaagtgacaggtggtgcatggttgtcgtcagctcgtgtcgtgagatgttgggttaagtcccgcaacgagcgcaacccttgtctttagttgccatcatttagttgggcactctggagagactgccagggataacctggaggaaggtggggatgacgtcaaatcatcatgccccttatgcctagggctacacacgtgctacaatgggtggtacagagggcagctaagccgtgaggtggagcaaatcccttaaagccattctcagttcggattgtaggctgaaactcgcctacatgaagctggagttactagtaatcgcagatcagaatgctgcggtgaatgcgttcccgggtcttgtacacaccgcccgtca

SEQ ID NO: 94 - DNA, 1366 bp, Fusobacterium sp.; identical to SEQ ID NO: 37
  source 1..1366: mol_type = unassigned DNA

SEQ ID NO: 95 - DNA, 1361 bp, Fusobacterium sp.; identical to SEQ ID NO: 70
  source 1..1361: mol_type = unassigned DNA

SEQ ID NO: 96 - DNA, 1415 bp, Fusobacterium sp.
  source 1..1415: mol_type = unassigned DNA
aatgcttaacacatgcaagtctacttgatccttcgggtgatggtggcggacgggtgagtaacgcgtaaagaacttgccttgcagtctgggacaacgtctggaaacggacgctaataccggatattatgcgagagtcgcatggctctttcatgaaagctatatgcgctgcaggagagctttgcgtcccattagttagttggtgaggtaacggctcaccaagaccgcgatgggtagccggcctgagagggtgaacggccacaaggggactgagacacggcccttactcctacgggaggcagcagtggggaatattggacaatggaccaaaagtctgatccagcaattctgtgtgcacgatgacggtcttaggattgtaaagtgctttcaatcgggaaaaagaaagtgatggtaccgatagaagaagcgacggctaaatacgtgccagcagccgcggtaatacgtatgtcgcaagcgttatccggatttattgggcgtaaagcgcgtctaggcggtctggtaagtctgatgtggaaatgcggggctcaactccgtattgcgttggaaactgccagactagagtactggagaggtgggcggaactacaagtgtagaggtgaaattcgtagatatttgtaggaatgccgatagagaagtcagctcactggacagatactgacgctgaagcgcgaaagcatggggagcaaacaggattagataccctggtagtccatgccgtaaacgatgattactaagcgtcgggggtcgaacctcggcactcaagctaacgcgataagtaatccgcctggggagtacgtacgcaagtatgaaactcaaaggaattgacggggacccgcacaagtggtggagcatgtggtttaattggacgcaacgcgaggaactttaccagcgtgtgacatcctaggaatgagaaagagatttttcagtgctccttcgggagaacccagagacaggtggtgcatggctgtggtcagctcgtgtcgtgagatgttgggttaagtcccgcaacgagcgcaacccctattgtatgttgccatcattaagttgggcaatcatgcgatgctgcctgcgacgagcaggaggaaggtggggatgaggtcaagtcatcatgcccgttatatgctgggctacacacgtgctacaatgggcagtacagagagaagcaaatatgcgaggaggagcaaatgtcagaaagctgttcgtagttcggattgtactctgcaactggagtacatgaagttggaatcagtagtaatcgcaaatcagcaatgttgcggtgaatacgttctcgggtctggtacacaccgcccgtcacaccacgagagttgattgcacctgaagtagcaggcctaaccgtaaggaagggtggtccgagggtgtggttagcgattggggtg

SEQ ID NO: 97 - DNA, 1395 bp, unidentified
  source 1..1395: mol_type = unassigned DNA; note = Lachnospiraceae sequence
agagtttgatcctggctcaggatgaacgctggcggcgtgcttaacacatgcaagtcgagcgaagcgcttttacggatttcttcggattgaagtgattgtgactgagcggcggacgggtgagtaacgcgtgggtaacctgcctcatacagggggataacagttagaaatgactgctaataccgcataagcgcacagtaccgcatgggtacggtgtgaaaaactccggtggtatgagatggacccgcgtctgattaggtagttggtggggtaacggcctaccaagccaacgatcagtagccgacctgagagggcgaccggccacattgggactgagacacggcccaaactcctacgggaggcagcagtggggaatattgcacaatgggggaaaccctgatgcagcgacgccgcgtgaaggatgaagtatttcggtatgtaaacttctatcagcagggaagaaaataacggtacctgagtaagaagccccggctaactacgtgccagcagccgcggtaatacgtagggggcaagcgttatccggatttactgggtgtaaagggagcgtagacggaagtgcaagtctgatgtgaaaacccgaggctcaaccacgggactgcattggaaactgtgcttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgacttactagggtgtcgggcagcaaagctgttcggttgccgcagccatcgcaataagtagtccacctgggggagtacgttcgcaagaatgaaactcaaaggaattgacggggacccgcacaagcggtggagcatgtggtttaattcgaagcaacgcgaagaaccttacctgctcttgacatccctctgaccggcaagtaatgttgcctttccttcgggacagaggagacaggtggtgcatggttgtcgtcagctcgtgtcgtgagatgttgggttaagtcccgcaacgagcgcaacccctatcttcagtagccagcatttaaggtgggcactcaggagagactgccagggataacctggaggaaggtggggatgacgtcaaatcatcatgccccttatgagcagggctacacacgtgctacaatggcgtaaacaaagggaagcgaaagggtgacctggagcaaatctcagaaataacgtctcagttcggattgtagtctgcaactcgactacatgaagctggaatcgctagtaatcgcgaatcagcatgtcgcggtgaatacgttcccgggtcttgtactcaccgcccgtca

SEQ ID NO: 98 - DNA, 1388 bp, unidentified
  source 1..1388: mol_type = unassigned DNA; note = Peptostreptococcaceae sequence
agagtttgatcctggctcaggatgaacgctggcggcgtgcctaacacatgcaagttgagcgatttacttcggtaaagagcggcggacgggtgagtaacgcgtgggtaacctaccctgtacacacggataacataccgaaaggtatgctaatacgggataatatatttgagaggcatctcttgaatatcaaaggtgagccagtacaggatggacccgcgtctgattagctagttggtaaggtaacggcttaccaaggcgacgatcagtagccgacctgagagggtgatcggccacattggaactgagacacggtccaaactcctacgggaggcagcagtggggaatattgcacaatgggcgaaagcctgatgcagcaacgccgcgtgagtgatgaaggccttcgggtcgtaaaactctgtcctcaaggaagataatgacggtacttgaggaggaagccccggctaactacgtgccagcagccgcggtaatacgtagggggctagcgttatccggatttactgggcgtaaagggtgcgtaggcggtctttcaagtcaggagtgaaaggctacggctcaaccgtagtaagctcttgaaactgggagacttgagtgcaggagaggagagtggaattcctagtgtagcggtgaaatgcgtagatattaggaggaacaccagttgcgaaggcggctctctggactgtaactgacgctgaggcacgaaagcgtggggagcaaacaggattagataccctggtagtccacgctgtaaacgatgagtactaggtgtcgggggttacccccttcggtgccgcagctaacgcattaagtactccgcctgggaagtacgctcgcaagagtgaaactcaaaggaattgacggggacccgcacaagtagcggagcatgtggtttaattcgaagcaacgcgaagaaccttacctaagcttgacatcccaatgacatctccttaatcggagagttcccttcggggacattggtgacaggtggtgcatggttgtcgtcagctcgtgtcgtgagatgttgggttaagtcccgcaacgagcgcaacccttgtctttagttgccatcattaagttgggcactctagagagactgccagggataacctggaggaaggtggggatgacgtcaaatcatcatgccccttatgcttagggctacacacgtgctgattatgctaaggaaataggatttactggacaattcttaatagagcctaagccaaaagagcctactaaacatcaatatgattttgatactgctactgttttaggatttttaagaaagtataatctggataaatacttcaaagtgaatatagaagcaaaccatgcaacacttgcaggacatactttccaacatgaattaa

SEQ ID NO: 99 - DNA, 1457 bp, unidentified
  source 1..1457: mol_type = unassigned DNA; note = Lachnospiraceae sequence
cgctggcggcgtgcttaacacatgcaagtcgaacgaagcgatttggaggaagttttcggatgaaatctgaattgactgagtggcggacgggtgagtaacgcgtgggtaacctgcctcacacagggggacaacagttagaaatggctgctaataccgcataagcgcacagcttcgcatgaagcagtgtgaaaaactccggtggtgtgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcttggcaagtctgaagtgaaagcccggggctcaaccccgggactgctttggaaactgtcaggctagagtgctggagaggtaagtggaattcctagtgtagcggtgaaatgcatagatattaggaggaacaccagtggcgaaggcggcttactggacagtaactgacgttgaggctcgaaagcgtggggagcaaacaggattagataccctggtagtccacgccgtaaacgatgaatactaggtgttggggagcaaagctcttcggtgccgtcgcaaacgcaataagtattccacctgggaagtacgttcgcaagaatgaaactcaaaggaattgacggggacccgcacaagcggtggagcatgtggtttaattcgaagcaacgcgaagaaccttaccaagtcttgacatcccattgaaaagcccgtaacggggttccctcttcggagcaatggagacaggtggtgcatggttgtcgtcagctcgtgtcgtgagatgttgggttaagtcccgcaacgagcgcaacccttatcctaagtagccagcaggtagagctgggcactcttgggagactgccagggacaacctggaggaaggtggggatgacgtcaaatcatcatgccccttatgatttgggctacacacgtgctacaatggcgtaaacaaagggaagcgaagctgtgaagctaagcaaatctcaaaaataacgtctcagttcggattgtagtctgcaactcgactacatgaagctggaatcgctagtaatcgcggatcagaatgccgcggtgaacacgttcccgggtcttgtacacaccgcccgtcacaccatgggagtcagtaacgcccgaagccagtgacctaaccgcaaggaaggagctgtcgaaggcgggaccgataactggggtgaagtcgtaa

(19) United States
(12) Patent Application Publication
(10) Pub. No.: US 2026/0068909 A1
(43) Pub. Date: Mar. 12, 2026

(54) METHODS AND COMPOSITIONS FOR TREATING INTESTINAL DISORDER

(71) Applicants: Mars, Incorporated, McLean, VA (US); The Trustees of the University of Pennsylvania, Philadelphia, PA (US)

(72) Inventors: Sally Christine PEREA, Lewisburg, OH (US); Daniel P. BEITING, Philadelphia, PA (US)

(73) Assignees: Mars, Incorporated, McLean, VA (US); The Trustees of the University of Pennsylvania, Philadelphia, PA (US)

(21) Appl. No.: 19/386,209

(22) Filed: Nov. 11, 2025

Related U.S. Application Data
(62) Division of application No. 17/425,080, now Pat. No. 12,501,917.
(60) Provisional application No. 62/796,021, filed on Jan. 23, 2019.

(51) Int. Cl. A23K 10/18; A23K 50/40; A23L 33/135; A61K 31/575; A61K 35/742; A61P 1/00; C12Q 1/06

(52) U.S. Cl. CPC A23K 10/18; A23K 50/40; A23L 33/135; A61K 31/575; A61K 35/742; A61P 1/00; C12Q 1/06; G01N 2800/50; G01N 2800/52

(57) ABSTRACT

Provided are methods for determining susceptibility of an intestinal disorder in a companion animal. These methods include measuring a first amount of a first intestinal microorganism and/or a second amount of a second intestinal microorganism in the companion animal; comparing the first amount of the first intestinal microorganism with a first reference amount of the first intestinal microorganism, and/or comparing the second amount of the second intestinal microorganism with a second reference amount of the second intestinal microorganism; and determining that the companion animal is susceptible of an intestinal disorder, when the first amount of the intestinal microorganism is higher than the first reference amount of the first intestinal microorganism, and/or when the second amount of the second intestinal microorganism is lower than the second reference amount of the second intestinal microorganism.

### CROSS-REFERENCE TO RELATED APPLICATIONS

This application is a divisional of U.S. patent application Ser. No. 17/425,080, filed Jul. 22, 2021, which is a U.S. National Stage Patent Application under 35 U.S.C. § 371 of International Patent Application No. PCT/US2020/014823, filed Jan. 23, 2020, which claims priority to U.S. Provisional Application No. 62/796,021, filed Jan. 23, 2019. The contents of each priority application are incorporated herein by reference in their entirety, and to which priority is claimed.

### INCORPORATION BY REFERENCE OF AN ELECTRONIC SEQUENCE LISTING

The present application is being filed with a Sequence Listing in electronic format. The contents of the electronic Sequence Listing (144182007910SEQLIST.xml; Size: 246,481 bytes; and Date of Creation: Nov. 11, 2025) is herein incorporated by reference in its entirety.

### FIELD

The presently disclosed subject matter relates to method, compositions and food products for improving intestinal health, treating intestinal dysbiosis and/or treating an intestinal disorder in a subject, e.g., a human or a companion animal.

### BACKGROUND

Inflammatory bowel disease (IBD), including Crohn's disease and ulcerative colitis, is a multi-factorial and debilitating disease characterized by chronic immune-pathology, disruption of intestinal homeostasis and altered composition of the gut microbiome (dysbiosis). Several lines of evidence point to resident gut bacteria as important factors in the etiology of IBD. First, disease is often more severe in areas of the intestine with the highest microbial biomass, and antibiotics are frequently used as an adjunct therapy with immunosuppressants or monoclonal antibodies for managing IBD^{1,2}. Second, genome-wide associations studies have identified numerous susceptibility loci in genes responsible for recognizing or responding to bacteria 3. Finally, in some mouse models of colitis, disease can be transferred to naive hosts via fecal transplant⁴⁻⁶, suggesting a causal role for gut microbes in disease. Collectively, these findings have led to a ‘two-hit’ model for IBD in which both host genetics and microbial factors influence disease presentation, highlighting an opportunity to develop novel treatments for IBD that target the microbiome. Thus, there is a need for novel methods and compositions for treating IBD and other intestinal disorders that target gut microbiome and metabolites thereof.

### SUMMARY OF THE INVENTION

The presently disclosed subject matter provides a pharmaceutical composition, dietary supplement and functional food for medicament. In certain embodiments, the pharmaceutical composition, dietary supplement or functional food comprises an effective amount of a bacterium capable of producing a first bile acid for use as a medicament. In certain embodiments, the pharmaceutical composition, dietary supplement or functional food further comprises an effective amount of a second bile acid. In certain embodiments, the pharmaceutical composition, dietary supplement or functional food is for the treatment of an intestinal disorder in a subject in need thereof.

In certain embodiments, the bacterium comprises a bile acid-inducible operon (bai operon). In certain embodiments, the bile acid-inducible operon (bai operon) comprises a nucleotide sequence that is at least about 90% homologous or identical to SEQ ID NO: 1 or 3, or any functional fragment thereof. In certain embodiments, the bile acid-inducible operon (bai operon) comprises the nucleotide sequence set forth in SEQ ID NO: 1 or 3.

In certain embodiments, the bacterium comprises a 16s rRNA comprising a nucleotide sequence that is at least about 90% homologous or identical to SEQ ID NO: 2 or 4. In certain embodiments, the bacterium comprises a 16s rRNA comprising the nucleotide sequence set forth in SEQ ID NO: 2 or 4.

In certain embodiments, the bacterium is C. hiranonis, C. scindens or combination thereof. In certain embodiments, the bacterium is C. hiranonis.

In certain embodiments, the first bile acid and/or the second bile acid is selected from the group consisting of chenodeoxycholic acid, cholic acid, glycochenodeoxycholic acid, glycocholic acid, taurocholic acid, taurochenodeoxycholic acid, taurodeoxycholic acid, glycodeoxycholic acid, ursodeoxycholic acid, glycoursodeoxycholic acid, tauroursodeoxycholic acid, taurolithocholic acid, alpha-muricholic acid, deoxycholic acid, gamma-muricholic acid, glycolithocholic acid, taurolithocholic acid, lithocholic acid, omega-muricholic acid and any combination thereof.

In certain embodiments, the first bile acid and/or the second bile acid is a secondary bile acid. In certain embodiments, the secondary bile acid is selected from the group consisting of taurodeoxycholic acid, glycodeoxycholic acid, ursodeoxycholic acid, glycoursodeoxycholic acid, tauroursodeoxycholic acid, taurolithocholic acid, alpha-muricholic acid, deoxycholic acid, gamma-muricholic acid, glycolithocholic acid, taurolithocholic acid, lithocholic acid, omega-muricholic acid and any combination thereof. In certain embodiments, the secondary bile acid is deoxycholic acid and/or lithocholic acid.

In certain embodiments, the subject is a dog. In certain embodiments, the intestinal disorder is an acute enteropathy or a chronic enteropathy. In certain embodiments, the chronic enteropathy is selected from the group consisting of food responsive enteropathy, antibiotic responsive enteropathy, and idiopathic inflammatory bowel disease (IBD). In certain embodiments, the intestinal disorder is idiopathic inflammatory bowel disease (IBD).

In certain embodiments, the bacterium is transformed with a vector comprising a bile acid-inducible operon (bai operon). In certain embodiments, the bacterium is selected from the genus of Clostridium.

In certain embodiments, the amount of the bacterium is between about 10 thousand CFU and about 100 trillion CFU. In certain embodiments, the second bile acid is between about 10 mg/unit dose and about 500 mg/unit dose.

In certain embodiments, the first bile acid and the second bile acid are the same. In certain embodiments, the first bile acid and the second bile acid are different.

The presently disclosed subject matter provides C. hiranonis for use as a functional food or supplement to prevent onset of a GI condition or as a medicament. In certain embodiments, the C. hiranonis is for the treatment of an intestinal disorder in a subject in need thereof. The presently disclosed subject matter provides C. scindens functional food or supplement to prevent onset of a GI condition or for use as a medicament. In certain embodiments, the C. scindens is for the treatment of an intestinal disorder in a subject in need thereof.

The presently disclosed subject matter provides deoxycholic acid for the treatment of inflammatory bowel disease (IBD) in a subject in need thereof. The presently disclosed subject matter provides lithocholic acid for the treatment of inflammatory bowel disease (IBD) in a subject in need thereof.

The presently disclosed subject matter provides a dietary supplement or a food product comprising an effective amount of a bacterium capable of producing a first bile acid. In certain embodiments, the dietary supplement or a food product further comprises an effective amount of a second bile acid. In certain embodiments, the food product improves intestinal health in a subject. In certain embodiments, the amount of the bacterium is between about 10 thousand CFU and about 100 trillion CFU. In certain embodiments, the second bile acid is between about 100 mg/daily serving dose and about 1000 mg/daily serving dose.

In certain embodiments, the food product is a pet food product. In certain embodiments, the food product is a dog food product.

The presently disclosed subject matter provides a method of treating an intestinal disorder in a subject in need thereof, comprising administering to the subject an effective amount of a pharmaceutical composition, dietary supplement or functional food disclosed herein, an effective amount of a food product disclosed herein, or combination thereof. In certain embodiments, the method further comprises monitoring an intestinal microorganism in the subject. In certain embodiments, the intestinal microorganism is sampled from a fecal sample of the subject.

The presently disclosed subject matter provides a method for determining susceptibility of an intestinal disorder in a companion animal. In certain embodiments, the method comprises:
a) measuring a first amount of a first intestinal microorganism and/or a second amount of a second intestinal microorganism in the companion animal;
b) comparing the first amount of the first intestinal microorganism with a first reference amount of the first intestinal microorganism, and/or comparing the second amount of the second intestinal microorganism with a second reference amount of the second intestinal microorganism, wherein the reference amounts of the intestinal microorganisms are determined based on the amounts of the intestinal microorganisms in a plurality of healthy companion animals; and
c) determining that the companion animal is susceptible of an intestinal disorder, when the first amount of the intestinal microorganism is higher than the first reference amount of the first intestinal microorganism, and/or when the second amount of the second intestinal microorganism is lower than the second reference amount of the second intestinal microorganism.

In certain embodiments, the first intestinal microorganism comprises one or more bacterium comprising a 16S rRNA comprising a nucleotide sequence that is at least about 90% homologous or identical to the 16S rRNA nucleotide sequence of HQ802983.1.1440, GQ449092.1.1375, GQ448744.1.1393, KF842598.1.1394, HG798451.1.1400, New.ReferenceOTU52, HK555938.1.1357, FJ957494.1.1454, FN667392.1.1495, New.ReferenceOTU54, HQ760911.1.1437, GQ006324.1.1342, FJ950694.1.1472, FM865905.1.1392, FJ506371.1.1371, FJ957528.1.1445, JF712675.1.1540, New.ReferenceOTU82, AB009242.1.1451, HQ751549.1.1448, AB506370.1.1516, DQ057365.1.1393, FN667422.1.1495, AJ270486.1.1241, FN668375.4306350.4307737, GQ867426.1.1494, GX182404.8.1529, JF224013.1.1362, GQ448246.1.1389, KC245406.1.1465, FN667084.1.1493, EU470512.1.1400, EU768569.1.1352, AY239462.1.1500, KC504009.1.1465, FM179752.1.1686, New.ReferenceOTU114, HK557089.3.1395, JQ208181.1.1352, HQ803964.1.1435, AM276759.1.1484, JN387556.1.1324, GQ448486.1.1387, HK694029.9.1487, HQ754680.1.1441, FN563300.1.1447, FP929060.3837.5503, GQ448506.1.1374, Enterococcus durans, C. perfringens, or E. coli.

In certain embodiments, the first intestinal microorganism is selected from the group consisting of HQ802983.1.1440, GQ449092.1.1375, GQ448744.1.1393, KF842598.1.1394, HG798451.1.1400, New.ReferenceOTU52, HK555938.1.1357, FJ957494.1.1454, FN667392.1.1495, New.ReferenceOTU54, HQ760911.1.1437, GQ006324.1.1342, FJ950694.1.1472, FM865905.1.1392, FJ506371.1.1371, FJ957528.1.1445, JF712675.1.1540, New.ReferenceOTU82, AB009242.1.1451, HQ751549.1.1448, AB506370.1.1516, DQ057365.1.1393, FN667422.1.1495, AJ270486.1.1241, FN668375.4306350.4307737, GQ867426.1.1494, GX182404.8.1529, JF224013.1.1362, GQ448246.1.1389, JF807116.1.1260, KC245406.1.1465, FN667084.1.1493, EU470512.1.1400, EU768569.1.1352, AY239462.1.1500, KC504009.1.1465, FM179752.1.1686, New.ReferenceOTU114, HK557089.3.1395, JQ208181.1.1352, HQ803964.1.1435, AM276759.1.1484, JN387556.1.1324, GQ448486.1.1387, HK694029.9.1487, HQ754680.1.1441, FN563300.1.1447, FP929060.3837.5503, GQ448506.1.1374, Enterococcus durans, C. perfringens, E. coli and any combination thereof. In certain embodiments, the first intestinal microorganism is C. perfringens, E. coli and any combination thereof.

In certain embodiments, the second intestinal microorganism comprises one or more bacterium comprising a 16S rRNA comprising a nucleotide sequence that is at least about 90% homologous or identical to the 16S rRNA nucleotide sequence of EU774020.1.1361, HQ793763.1.1451, HQ792787.1.1438, New.ReferenceOTU109, HQ792778.1.1436, or DQ113765.1.1450.

In certain embodiments, the second intestinal microorganism is selected from the group consisting of EU774020.1.1361, HQ793763.1.1451, HQ792787.1.1438, New.ReferenceOTU109, HQ792778.1.1436, DQ113765.1.1450, and any combination thereof.

In certain embodiments, the method further comprises providing a customized recommendation of a treatment regimen, and/or further monitoring the intestinal microorganism, when the first amount of the first intestinal microorganism is lower than the first reference amount of the first intestinal microorganism, and/or when the second amount of the second intestinal microorganism is higher than the second reference amount of the second intestinal microorganism.

The presently disclosed subject matter provides a method for determining responsiveness of a companion animal having an intestinal disorder to a diet. In certain embodiments, the method comprises:
a) measuring a first amount of a first intestinal microorganism and/or a second amount of a second intestinal microorganism in the companion animal;
b) comparing the first amount of the first intestinal microorganism with a first reference amount of the first intestinal microorganism, and/or comparing the second amount of the second intestinal microorganism with a second reference amount of the second intestinal microorganism, wherein the reference amounts of the intestinal microorganisms are determined based on the amounts of the intestinal microorganisms in a plurality of healthy companion animals; and
c) determining that the companion animal is responsive to the diet, when the first amount of the intestinal microorganism is higher than the first reference amount of the first intestinal microorganism, and/or when the second amount of the second intestinal microorganism is lower than the second reference amount of the second intestinal microorganism, or determining that the companion animal is non-responsive to the diet, when the first amount of the intestinal microorganism is lower than the first reference amount of the first intestinal microorganism, and/or when the second amount of the second intestinal microorganism is higher than the second reference amount of the second intestinal microorganism.

In certain embodiments, the first intestinal microorganism comprises one or more bacterium comprising a 16S rRNA comprising a nucleotide sequence that is at least about 90% homologous or identical to the 16S rRNA nucleotide sequence of ReferenceOTU45, JRPJ01000002.1034290.1035971, KF842598.1.1394, JF920309.1.1340, FJ978526.1.1378, New.ReferenceOTU54, HQ793763.1.1451, DQ113765.1.1450, DQ797046.1.1403, ACBW01000012.3536.5054, JN387556.1.1324, New.ReferenceOTU52, or JQ208053.1.1336.

In certain embodiments, the first intestinal microorganism is selected from the group consisting of New.ReferenceOTU45, JRPJ01000002.1034290.1035971, KF842598.1.1394, JF920309.1.1340, FJ978526.1.1378, New.ReferenceOTU54, HQ793763.1.1451, DQ113765.1.1450, DQ797046.1.1403, ACBW01000012.3536.5054, JN387556.1.1324, New.ReferenceOTU52, JQ208053.1.1336, and any combination thereof.

In certain embodiments, the second intestinal microorganism comprises one or more bacterium comprising a 16S rRNA comprising a nucleotide sequence that is at least about 90% homologous or identical to the 16S rRNA nucleotide sequence of HK693629.1.1491, GQ493166.1.1359, GQ491426.1.1332, FJ957494.1.1454, GQ449092.1.1375, GQ448486.1.1387, AMCI01001631.34.1456, or HK555938.1.1357.

In certain embodiments, the second intestinal microorganism is selected from the group consisting of HK693629.1.1491, GQ493166.1.1359, GQ491426.1.1332, FJ957494.1.1454, GQ449092.1.1375, GQ448486.1.1387, AMCI01001631.34.1456, HK555938.1.1357, and any combination thereof.

In certain embodiments, the method further comprises administering the diet to the companion animal when companion animal is determined as responsive to the diet. In certain embodiments, the method further comprises administering the diet, a steroid and optionally an antibiotic to the companion animal when companion animal is determined as non-responsive to the diet.

In certain embodiments, the determination in step c) occurs before administering the diet or the diet, the steroid and optionally the antibiotic to the companion animal.

The presently disclosed subject matter provides a method for determining effectiveness of a diet for treating an intestinal disorder in a companion animal. In certain embodiments, the method comprises:
a) measuring a first amount of a first intestinal microorganism and/or a second amount of a second intestinal microorganism in the companion animal after administering a diet to a companion animal for treating an intestinal disorder;
b) comparing the first amount of the first intestinal microorganism with a first reference amount of the first intestinal microorganism, and/or comparing the second amount of the second intestinal microorganism with a second reference amount of the second intestinal microorganism, wherein the reference amounts of the intestinal microorganisms are determined based on the amounts of the intestinal microorganisms in a plurality of healthy companion animals; and
c) determining that the diet is effective for treating an intestinal disorder, when the first amount of the intestinal microorganism is higher than the first reference amount of the first intestinal microorganism, and/or when the second amount of the second intestinal microorganism is lower than the second reference amount of the second intestinal microorganism, or determining that the diet is ineffective for treating an intestinal disorder, when the first amount of the intestinal microorganism is lower than the first reference amount of the first intestinal microorganism, and/or when the second amount of the second intestinal microorganism is higher than the second reference amount of the second intestinal microorganism.

In certain embodiments, the first intestinal microorganism comprises one or more bacterium comprising a 16S rRNA comprising a nucleotide sequence that is at least about 90% homologous or identical to the 16S rRNA nucleotide sequence of HK557089.3.1395, or GQ448336.1.1418.

In certain embodiments, the first intestinal microorganism is selected from the group consisting of HK557089.3.1395, GQ448336.1.1418, and combination thereof.

In certain embodiments, the second intestinal microorganism comprises one or more bacterium comprising a 16S rRNA comprising a nucleotide sequence that is at least about 90% homologous or identical to the 16S rRNA nucleotide sequence of KF842598.1.1394, GQ006324.1.1342, HQ802983.1.1440, JN387556.1.1324, FJ950694.1.1472, HG798451.1.1400, New.ReferenceOTU52, or GQ448468.1.1366.

In certain embodiments, the second intestinal microorganism is selected from the group consisting of KF842598.1.1394, GQ006324.1.1342, HQ802983.1.1440, JN387556.1.1324, FJ950694.1.1472, HG798451.1.1400, New.ReferenceOTU52, GQ448468.1.1366, and any combination thereof.

In certain embodiments, the method further comprises administering the diet to the companion animal when companion animal is determined as responsive to the diet. In certain embodiments, the method further comprises administering the diet, a steroid and optionally an antibiotic to the companion animal when companion animal is determined as non-responsive to the diet.

In certain embodiments, the determination in step c) occurs before administering the diet or the diet, the steroid and optionally the antibiotic to the companion animal.

In certain embodiments, the reference amount of an intestinal microorganism derived from a mean amount of the intestinal microorganism in a plurality of healthy companion animals. In certain embodiments, the amount of the intestinal bacterium is measured from a fecal sample of the subject.

The presently disclosed subject matter provides a diet for increase a population of a bacterium capable of producing a bile acid in a companion animal. In certain embodiments, the diet comprises protein, fat, crude fiber, total dietary fiber, carbohydrate, calcium, phosphorus, sodium, chloride, potassium, magnesium, iron, copper, manganese, zinc, iodine, selenium, vitamin A, vitamin D3, vitamin E, vitamin C, thiamine (vitamin B1), riboflavin (vitamin B2), pantothenic acid, niacin, pyridoxine (vitamin B6), folic acid, biotin, cobalannin (vitamin B12), choline, arginine, lysine, methionine, cystine, taurine, linoleic acid, arachidonic acid, Omega-6 fatty acids, Omega-3 fatty acids, EPA, and/or DHA.

In certain embodiments, the subject is a dog. In certain embodiments, the diet is a Royal Canin Veterinary Diet. In certain embodiments, the diet is selected from the group consisting of Ultamino, Hydrolyzed Protein Adult HP Dry, Hydrolyzed Protein Wet, Hydrolyzed Protein Adult PS Dry, Hydrolyzed Protein Moderate Calorie Dry, Hydrolyzed Protein Small Dog Dry, Hydrolyzed protein Treats, and any combination thereof.

In certain embodiments, the bacterium comprises a bile acid-inducible operon (bai operon). In certain embodiments, the bacterium is C. hiranonis, C. scindens or combination thereof. In certain embodiments, the bacterium is C. hiranonis.

The presently disclosed subject matter provides a Royal Canin Veterinary Diet for the treatment of an intestinal disorder in a dog, wherein the dog comprises a first amount of a first intestinal microorganism and/or a second amount of a second intestinal microorganism, and wherein the first amount of the first intestinal microorganism is higher than a first reference amount of the first intestinal microorganism, and/or the second amount of the second intestinal microorganism is lower than a second reference amount of the second intestinal microorganism.

In certain embodiments, the first intestinal microorganism comprises one or more bacterium comprising a 16S rRNA comprising a nucleotide sequence that is at least about 90% homologous or identical to the 16S rRNA nucleotide sequence of ReferenceOTU45, JRPJ01000002.1034290.1035971, KF842598.1.1394, JF920309.1.1340, FJ978526.1.1378, New.ReferenceOTU54, HQ793763.1.1451, DQ113765.1.1450, DQ797046.1.1403, ACBW01000012.3536.5054, JN387556.1.1324, New.ReferenceOTU52, or JQ208053.1.1336.

In certain embodiments, the first intestinal microorganism is selected from the group consisting of New.ReferenceOTU45, JRPJ01000002.1034290.1035971, KF842598.1.1394, JF920309.1.1340, FJ978526.1.1378, New.ReferenceOTU54, HQ793763.1.1451, DQ113765.1.1450, DQ797046.1.1403, ACBW01000012.3536.5054, JN387556.1.1324, New.ReferenceOTU52, JQ208053.1.1336, and any combination thereof.

In certain embodiments, the second intestinal microorganism comprises one or more bacterium comprising a 16S rRNA comprising a nucleotide sequence that is at least about 90% homologous or identical to the 16S rRNA nucleotide sequence of HK693629.1.1491, GQ493166.1.1359, GQ491426.1.1332, FJ957494.1.1454, GQ449092.1.1375, GQ448486.1.1387, AMCI01001631.34.1456, or HK555938.1.1357.

In certain embodiments, the second intestinal microorganism is selected from the group consisting of HK693629.1.1491, GQ493166.1.1359, GQ491426.1.1332, FJ957494.1.1454, GQ449092.1.1375, GQ448486.1.1387, AMCI01001631.34.1456, HK555938.1.1357, and any combination thereof.

In certain embodiments, the Royal Canin Veterinary Diet is selected from the group consisting of Ultamino, Hydrolyzed Protein Adult HP Dry, Hydrolyzed Protein Wet, Hydrolyzed Protein Adult PS Dry, Hydrolyzed Protein Moderate Calorie Dry, Hydrolyzed Protein Small Dog Dry, Hydrolyzed protein Treats, and any combination thereof.

The presently disclosed subject matter provides a bile acid for the treatment of an intestinal disorder in a dog. In certain embodiments, the bile acid is selected from the group consisting of chenodeoxycholic acid, cholic acid, glycochenodeoxycholic acid, glycocholic acid, taurocholic acid, taurochenodeoxycholic acid, taurodeoxycholic acid, glycodeoxycholic acid, ursodeoxycholic acid, glycoursodeoxycholic acid, tauroursodeoxycholic acid, taurolithocholic acid, alpha-muricholic acid, deoxycholic acid, gamma-muricholic acid, glycolithocholic acid, taurolithocholic acid, lithocholic acid, omega-muricholic acid and any combination thereof. In certain embodiments, the bile acid is a secondary bile acid. In certain embodiments, the secondary bile acid is selected from the group consisting of taurodeoxycholic acid, glycodeoxycholic acid, ursodeoxycholic acid, glycoursodeoxycholic acid, tauroursodeoxycholic acid, taurolithocholic acid, alpha-muricholic acid, deoxycholic acid, gamma-muricholic acid, glycolithocholic acid, taurolithocholic acid, lithocholic acid, omega-muricholic acid and any combination thereof. In certain embodiments, the secondary bile acid is deoxycholic acid and/or lithocholic acid.

The presently disclosed subject matter provides a kit comprising a presently disclosed pharmaceutical composition, dietary supplement, functional food, food product, diet or bile acid. In certain embodiments, the kit further comprises written instructions for treating and/or preventing an intestinal disorder.

### BRIEF DESCRIPTION OF THE DRAWINGS

FIGS. 1A-1C depict that diet therapy induces rapid and durable remission in canine model of chronic enteritis. FIG. 1A is a schematic showing clinical study design for identifying diet responsive (DR) and non-diet responsive (NDR) dogs. Antibiotics (Abtx) and Prednisone (Pred) treatments are indicated. Abbreviated Canine Chronic Enteropathy Clinical Activity Index (CCECAI) scores were assessed at four different time points in DR (n=20) (FIG. 1B) and NDR (n=9) (FIG. 1C) animals. ns=not significant, **p<0.01, ****p<0.0001 using Wilcoxon rank sum test.

FIGS. 2A-2F depict identification of microbial community profiles associated with treatment outcome. FIG. 2A is a ternary plot of phylum level OTUs from top 5 most abundant phyla among healthy (right), DR (left) and NDR (top) animals. Bubble size represents the log 2 OTU abundance. Relative abundance of E. coli (FIG. 2B) and C. perfringens (FIG. 2D) in animals with active disease (day 0) and healthy dogs. Spearman correlation between log 10 abundance of E. coli (FIG. 2C) or Clostridium sp. (FIG. 2E) and CCECAI disease score. FIG. 2F depicts differentially abundant OTUs between DR and DNR animals at day 0. Y-axis value represents the log 2 fold change for DR versus NDR. Arrow marks the OTU corresponding to C. perfringens. * p<0.05, **p<0.01 using Wilcoxon rank sum test (or Wilcoxon signed-rank test if available). Spearman correlations in panel C and E are significant (p<0.05) with correlation coefficients of 0.2109 and 0.2324, respectively.

FIGS. 3A-3G depict that therapeutic diet ameliorates dysbiosis associated with chronic enteritis. FIG. 3A depicts Pielou's evenness index for DR animals at different time points in the study. FIG. 3B depicts the principal coordinate analysis (PCoA) based on unweighted Unifrac distance for DR. FIG. 3C depicts the phylogenetic distance (unweighted Unifrac) to healthy controls for DR animals. FIG. 3D depicts the stream plot showing phylum level dynamics of microbiota structure for DR animals throughout the study. FIG. 3E depicts the volcano plot showing differentially abundant OTUs enriched in either DR dogs with active disease (day 0, red points) or in remission after diet therapy (day 14, blue points). Selected taxa (e.g., Escherichia-Shigella spp., Clostridium spp.) are labeled. The relative abundance of E. coli (FIG. 3F) and C. perfringens (FIG. 3G) in DR animals throughout the study and compared to healthy controls. ns=not significant, *p<0.05, **p<0.01, ***p<0.0001 using Wilcoxon rank sum test (or Wilcoxon signed-rank test if paired data was available).

FIGS. 4A-4F depict diet-induced changes in the microbiome associated with remission. FIG. 4A depicts Pielou's evenness index in NDR animals, and their phylogenetic distance (unweighted Unifrac) to healthy dogs is shown in FIG. 4B. FIG. 4C depicts the stream plot showing phylum level dynamics of microbiota structure for NDR animals throughout the study. Diet therapy began at day 0, metronidazole administration at day 14, and prednisone at day 28 (see methods). FIG. 4D depicts the bubble plot showing differentially abundant genera (fold change>2 and P<0.05) between day 14 versus day 0 for DR (left) and NDR (right) animals. Bubble size indicates absolute log fold change between day 14 and day 0, and color reflects direction of change. FIGS. 4E and 4F depict the relative abundance of E. coli (FIG. 4E) and C. perfringens (FIG. 4F) in NDR animals throughout the study and compared to healthy controls. ns=not significant, *p<0.05, **p<0.01, ***p<0.0001 using Wilcoxon rank sum test (or Wilcoxon signed-rank test if paired data was available).

FIGS. 5A-5H depict that diet-induced remission is associated with metabolic reprogramming and increased levels of secondary bile acids. FIG. 5A depicts a PCA analysis of KEGG pathways based on the results of Tax4Fun analysis. FIG. 5B depicts the first principal component (Dim 1) from panel A, for all time points. FIG. 5C depicts a heatmap showing the shift of metabolic potentials from fat/lipid metabolism to carbohydrate/sugar metabolism as DR animals receive diet therapy. FIG. 5D depicts the relative abundance of the KEGG pathway for secondary bile acid biosynthesis, predicted based on 16S sequence data. FIGS. 5E-5H depict the levels of deoxycholic (FIG. 5E) and lithocholic acid (FIG. 5F) measured in the stool of DR animals and NDR animals (FIGS. 5G and 5H). ns=not significant, *p<0.05, **p<0.01, ***p<0.0001 using Wilcoxon rank sum test (or Wilcoxon signed-rank test if paired data was available).

FIGS. 6A-6J depict that C. hiranonis is a diet-responsive species with the ability to produce secondary bile acids that inhibit the expansion of potential pathogens in vitro and in vivo. FIG. 6A depicts the Spearman correlations between the abundance of bacteria genera and the levels of bile acids. Only genera that have significant (P<0.05) correlations with bile acids are shown. FIGS. 6B-6E depict the in vitro growth of canine clinical isolates of E. coli (FIGS. 6B and 6C) or C. perfringens (FIGS. 6D and 6E) in the presence of varying concentrations of lithocholic acid or deoxycholic acid (mean±s.d. shown). The in vitro inhibition tests were biologically repeated 2 times. Each point in the graphs represent one replicate well in the assay. FIG. 6F depicts the relative abundance of the OTU corresponding to C. hiranonis (FJ957494.1.1454) in 16S rRNA sequencing data for DR and NDR animals. FIG. 6G depicts the coverage of the bile acid operon (bai) from the C. hiranonis reference (ASM15605v1) with whole genome sequencing reads produced C. hiranonis (teal) and C. perfringens (red) canine clinical isolates. FIG. 6H is a schematic showing experimental design for mouse experiments. FIG. 6I depicts the length of colon at day 8. FIG. 6J depicts E. coli Nissle strain CFUs measured in colon contents at day 8 (mean±s.d. shown for n=5 mice). Experiments were repeated 3 times with similar results. Data shown are from a representative experiment. ns=not significant, *p<0.05, **p<0.01, ***p<0.0001 using Wilcoxon signed rank sum test for relative abundance comparisons or t test for the in vitro culture experiments.

FIGS. 7A-7E depict that the bile acid producer, C. scindens, is associated with diet-induced remission in human pediatric Crohn's disease. Analysis of public data²³ from human pediatric Crohn's disease patients treated with exclusive enteral nutrition (EEN). Relative abundance of reads (mapping ratio) aligning to C. scindens reference (FIG. 7A) or bai operon (FIG. 7B) from 20 patients at pretreatment and 1, 4 and 8 weeks following administration of EEN. Patients that responded to treatment and entered remission (n=10, red) and those that failed therapy (n=10, green) are shown. FIGS. 7C and 7D depict Spearman correlations between log 10-transformed fecal calprotectin levels (FCP) and relative abundance of C. scindens (FIG. 7C) (R=−0.3515 for ‘Responsive’, P=0.0328; R=−0.0267 for ‘Non.Responsive’, P=0.8770) or bai operon (FIG. 7D) (R=−0.3944 for ‘Responsive’, P=0.0157; R=0.0490 for ‘Non.Responsive’, P=0.7766). FIG. 7E is a schematic showing proposed model for diet-induced remission. ns=not significant, *p<0.05, **p<0.01, ***p<0.0001 using Wilcoxon rank sum test for relative abundance comparisons.

FIG. 8 depicts detailed clinical design for canine chronic enteritis study.

FIGS. 9A-9D depict community structures of microbiomes in the dogs with CE and in the healthy dogs. Faith's phylogenetic diversity (FIG. 9A) and Shannon index (FIG. 9B) were compared between the samples from the dogs with CE (day 0) and the samples from healthy dogs. FIG. 9C depicts the ratios of microbiota compositions at a phylum level. FIG. 9D depicts the Unifrac (unweighted) distances within the microbiomes of the dogs with CE or within those of the healthy dogs. ns=not significant, *p<0.05, **p<0.01, ***p<0.0001 using Wilcoxon rank sum test.

FIGS. 10A-10C depict that microbiota community structure changes induced by diet therapy in diet responsive dogs. FIG. 10A depicts Faith's phylogenetic diversity. FIG. 10B depicts Shannon index diversity. FIG. 10C depicts principal coordinate Analysis (PCoA) based on Weighted Unifrac distance of the microbiomes. ns=not significant, *p<0.05, **p<0.01, ***p<0.0001 using Wilcoxon rank sum test.

FIG. 11 depicts that dynamics of microbiome changes at a phylum level for diet responsive dogs (DRs) and non-diet responsive dogs (NDRs).

FIG. 12 depicts principal component analysis based on the abundances of KO (KEGG Orthology) for the samples of day 0 and day 14.

FIG. 13 depicts concentrations of bile acids detected in the fecal samples of diet responsive dogs. NS=not significant, *p<0.05, **p<0.01, ***p<0.0001 using Wilcoxon signed-rank test.

FIG. 14 depicts relative abundance of C. hiranonis in diet responsive dogs calculated from metagenomic data.

### DETAILED DESCRIPTION OF THE INVENTION

To date, there remains a need for novel methods and compositions for treating IBD and other intestinal disorders that target gut microbiome and metabolites thereof. The present application relates to method, compositions and food products for improving intestinal health, treating intestinal dysbiosis and/or treating an intestinal disorder in a subject, e.g., a human or a companion animal, which is based, at least in part, on the discovery that intestinal microorganisms that produce bile acids can promote intestinal health and/or is associated with remission from an intestinal disorder after treatment, and that changes of intestinal microorganism population are associated to intestinal health status.

For clarity and not by way of limitation, the detailed description of the presently disclosed subject matter is divided into the following subsections:
1. Definitions;
2. Intestinal bacteria and health assessment tools relating to the same;
3. Pharmaceutical composition;
4. Food products;
5. Treatment methods; and
6 Kits.

### 1. Definitions

The terms used in this specification generally have their ordinary meanings in the art, within the context of the present disclosure and in the specific context where each term is used. Certain terms are discussed below, or elsewhere in the specification, to provide additional guidance to the practitioner in describing the methods and compositions of the present disclosure and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, or up to 10%, or up to 5%, or up to 1% of a given value. Alternatively, particularly with respect to biological systems or processes, the term can mean within an order of magnitude, e.g., within 5-fold or within 2-fold, of a value.

The term “effective treatment” or “effective amount” of a substance means the treatment or the amount of a substance that is sufficient to effect beneficial or desired results, including clinical results, and, as such, an “effective treatment” or an “effective amount” depends upon the context in which it is being applied. In the context of administering a composition to improving immunity, digestive function and/or decreasing inflammation, an effective amount of a composition described herein is an amount sufficient to improving immunity, digestive function and/or decreasing inflammation, as well as decrease the symptoms and/or reduce the likelihood of a digestive disorder and/or inflammation. An effective treatment described herein is a treatment sufficient to improving immunity, digestive function and/or decreasing inflammation, as well as decrease the symptoms and/or reduce the likelihood of a digestive disorder and/or inflammation. The decrease can be a 1%, 5%, 10%, 20%, 30%, 40%, 50%, 60%, 70%, 80%, 90%, 95%, 98% or 99% decrease in severity of symptoms of a digestive disorder or inflammation, or the likelihood of a digestive disorder or inflammation. An effective amount can be administered in one or more administrations. A likelihood of an effective treatment described herein is a probability of a treatment being effective, i.e., sufficient to treat or ameliorate a digestive disorder and/or inflammation, as well as decrease the symptoms.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more symptoms, diminishment of extent of a disorder, stabilized (i.e., not worsening) state of a disorder, prevention of a disorder, delay or slowing of the progression of a disorder, and/or amelioration or palliation of a state of a disorder. The decrease can be a 1%, 5%, 10%, 20%, 30%, 40%, 50%, 60%, 70%, 80%, 90%, 95%, 98% or 99% decrease in severity of complications or symptoms. “Treatment” can also mean prolonging survival as compared to expected survival if not receiving treatment.

As used herein, and as well-understood in the art, a “probiotic” is a preparation or composition comprising microorganisms that can provide health benefits when consumed. The microorganisms include, but are not limited to bacteria, fungi, yeasts and archaea. In certain embodiments, the probiotic can modify the microbiome in the GI system to enhance the balance of the microbiome in GI system, e.g., by acting as an inoculum for an increased population of beneficial microbes, and/or by antagonizing growth of deleterious microbes. In certain embodiments, the probiotic is an animal probiotic, e.g., a feline probiotic or a canine probiotic.

As used herein, and as well-understood in the art, a “prebiotic” is a substance or a composition that can induce the growth or activity of one or more beneficial microorganism (e.g., one or more probiotics, e.g., bacteria, fungi, yeasts and archaea). In certain embodiments, the prebiotic can modify the microbiome in the GI system to enhance the balance of the microbiome in GI system. In certain embodiments, the prebiotic is indigestible to an animal. In certain embodiments, the prebiotic can induce the growth or activity of one or more animal probiotics, e.g., a feline probiotic or a canine probiotic.

The term “pet food” or “pet food composition” or “pet food product” or “final pet food product” means a product or composition that is intended for consumption by a companion animal, such as a cat, a dog, a guinea pig, a rabbit, a bird or a horse. For example, but not by way of limitation, the companion animal can be a “domestic” dog, e.g., Canis lupus familiaris. In certain embodiments, the companion animal can be a “domestic” cat such as Felis domesticus. A “pet food” or “pet food composition” or “pet food product” or “final pet food product” includes any food, feed, snack, food supplement, liquid, beverage, treat, toy (chewable and/or consumable toys), meal substitute or meal replacement.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

“Pharmaceutical composition” and “pharmaceutical formulation,” as used herein, refer to a composition which is in such form as to permit the biological activity of an active ingredient contained therein to be effective, and which contains no additional components which are unacceptably toxic to a patient to which the formulation would be administered.

“Pharmaceutically acceptable,” as used herein, e.g., with respect to a “pharmaceutically acceptable excipient,” refers to the property of being nontoxic to a subject. A pharmaceutically acceptable ingredient in a pharmaceutical formulation can be an ingredient other than an active ingredient which is nontoxic. A pharmaceutically acceptable excipient can include a buffer, carrier, stabilizer, and/or preservative.

As used herein, the term “pharmaceutically acceptable salt” refers to any salt of a compound provided herein which retains its biological properties and which is not toxic or otherwise undesirable for pharmaceutical use. Such salts can be derived from a variety of organic and inorganic counter-ions well known in the art. Pharmaceutically acceptable salts further include, by way of example only and without limitation, sodium, potassium, calcium, magnesium, ammonium, tetraalkylammonium and the like, and when the compound contains a basic functionality, salts of non-toxic organic or inorganic acids.

### 2. Intestinal Microorganisms and Health Assessment Tools Relating to the Same

The presently disclosed subject matter provides intestinal microorganisms and combinations thereof, which is based, at least in part, on the discovery that intestinal microorganisms that produce bile acids can promote intestinal health and/or is associated with remission from an intestinal disorder after treatment, and that changes of intestinal microorganism population are associated to intestinal health status.

### Intestinal Microorganism Capable of Producing a Bile Acid

In certain embodiments, the intestinal microorganism is for use as a medicament. In certain embodiments, the intestinal microorganism is for the treatment of an intestinal disorder in a subject in need thereof.

In certain embodiments, the intestinal microorganism is a bacterium capable of producing a bile acid. In certain embodiments, the bile acid is chenodeoxycholic acid, cholic acid, glycochenodeoxycholic acid, glycocholic acid, taurocholic acid, taurochenodeoxycholic acid, taurodeoxycholic acid, glycodeoxycholic acid, ursodeoxycholic acid, glycoursodeoxycholic acid, tauroursodeoxycholic acid, taurolithocholic acid, alpha-muricholic acid, deoxycholic acid, gamma-muricholic acid, glycolithocholic acid, taurolithocholic acid, lithocholic acid, omega-muricholic acid or any combination thereof.

In certain embodiments, the bile acid is a primary bile acid. In certain embodiments, the primary bile acid is chenodeoxycholic acid, cholic acid, glycochenodeoxycholic acid, glycocholic acid, taurocholic acid, taurochenodeoxycholic acid or any combination thereof.

In certain embodiments, the bile acid is a secondary bile acid. In certain embodiments, the secondary bile acid is taurodeoxycholic acid, glycodeoxycholic acid, ursodeoxycholic acid, glycoursodeoxycholic acid, tauroursodeoxycholic acid, taurolithocholic acid, alpha-muricholic acid, deoxycholic acid, gamma-muricholic acid, glycolithocholic acid, taurolithocholic acid, lithocholic acid, omega-muricholic acid or any combination thereof. In certain embodiments, the secondary bile acid is deoxycholic acid and/or lithocholic acid.

In certain embodiments, the bacterium comprises a bile acid-inducible operon (bai operon). In certain embodiments, the bacterium comprises an enzyme having 7-dehydroxylation activity. In certain embodiments, the bai operon comprises a nucleotide sequence that is at least about 80% (e.g., at least about 85%, at least about 90%, or at least about 95%) homologous or identical to SEQ ID NO: 1 or 3, or any functional fragment thereof. In certain embodiments, the bai operon comprises the nucleotide sequence set forth in SEQ ID NO: 1 or 3. SEQ ID NO: 1 represents an exemplary sequence of C. hiranonis bile acid-inducible operon. SEQ ID NO: 3 represents an exemplary sequence of C. scindens bile acid-inducible operon.

| [SEQ ID NO: 1] | |
|---|---|
| 1 | gcaaattgat tttgattggt atttctttca ttcaaaatat ctcctttcct ttatttagct |
| 61 | gtattaaaat ttataaaaaa ttttcattgt taataaaaaa atattctttg ttagtattat |
| 121 | agcataattt ataaaaataa tgataatgtt ttaatattga aataataaat atgtaaaaag |
| 181 | gttggaaatt tatttaaaaa tgaccagaga taaaaagctc aggtcatttt ttttattatt |
| 241 | acaagtaatt tgaaaaaaat atatgaaatg aatggagaaa atataactga gatacatttg |
| 301 | ataatgaaaa aaacatttat cgaaattgta aatagactca ttgttataat taataaatat |
| 361 | ttattatggc atagttgtta aaattatacc ctaaagaaac gtttcctcaa aaagtgggtt |
| 421 | ataaaataaa tgttttttga cgaaagatgt gattttattt gtaccccttt tgtataaaga |
| 481 | ttaaacagta tttttgtata aatatattgt atacagtata gagaatgtcg atgtaaaaaa |
| 541 | gtatataaaa gtaaataata atcaaaaaaa ctagttttaa ttattaaaaa tgataaaaaa |
| 601 | tattaataaa ataaagagtc aaaaatactt gttagttaaa tcacagattt tgtctaagta |
| 661 | tagattaggt tttgtatttg aaaaggtcat ctatagtgtt gtaagaaagc gagttattag |
| 721 | cacatattgt atctcaaaaa aatgttaaga taatatcaag atagggcgat aaagaaaaaa |
| 781 | gcaaattgaa aaagaaaaaa gtaactataa gtttttacaa taaatcaaaa gagaattgat |
| 841 | tttaaaagag ggaggcaaaa taccgatatg aatgatgtga aatgtaaata ttttaataaa |
| 901 | tttaatacag gaatgtcaga ttttgttact ccaggaaaac agttagaata tgtagcaaaa |
| 961 | tgcaagccag atgaaaaagc tatcatatat atagataaag aagacaatgt gagagatatc |
| 1021 | acttggaagg aacttcacat agcttcaaat aaactagctt ggcatttaat gaaaaaggga |
| 1081 | tttggaaaag gtcaggtagc aatggtatct ttcccaaatg gtatagaaca tatattagca |
| 1141 | acattagctg tttggaaaac aggaggttgc tacatgccag tttcttgtaa gataacagat |
| 1201 | acagagcttg gtgatatatg cagaataata aaaccaacag tttcttttac agataaagaa |
| 1261 | atgccttgta gaacagaaag tataaaaata ggatcagtat tcgatgtttg taaagacgaa |
| 1321 | tcagaagaaa tgccagaaga tatagctgca aatccaaata tgatttctcc atctggagga |
| 1381 | acaacaggag agcctaagtt cataaaacag aatgtggcaa gtggcttatc tgatgaaatt |
| 1441 | ataaaaagct ggtttgaaat gtcaggtatg gaatttgaac aaagacaatt attagtagga |
| 1501 | ccacttttcc atggtgctcc tcatacagca gcatttaatg gattatttgt aggaaataca |
| 1561 | ttgataatac ctagaaattt aagacctgaa agtatagtta gatatataaa agaatacaaa |
| 1621 | atagaattta tacagatgat cccaacatta atgaatagaa taataaaatt agctgatgtt |
| 1681 | gataaagaag attttaaatc aataaaagca ctacaccata ctggtggata ttgttctcca |
| 1741 | tatttaaaag aaaagtggat cgatataata ggagctgaaa aagttcacga aatgtactct |
| 1801 | atgacagagg caatcggtat cacttgtata agaggagatg aatggcttaa acactatgga |
| 1861 | agcgtaggac ttccactagg aggaagcaga atatcaataa gagatgaaga aggaaatgaa |
| 1921 | ttaggaccac atgaggttgg agaaattcat atgacttcac caagtgcttg ttgcatgaca |
| 1981 | gaatacataa accataaacc acttgaaact aaagatggtg gatttagaag tgttggtgat |
| 2041 | ttcggttatg tagatgaaga tggatacctt tacttctcag atagaagaag cgacatgctt |
| 2101 | gttataggtg gagaaaacgt atttgcgact gaagttgaac cagtactacc agcttatgaa |
| 2161 | aaagtagttg atgctgtggt agttggaata cctgatgaag agtggggaag aagattacac |
| 2221 | gcaatagtac agaagaaaga agaagtttca gcagaagaat taatcgagta cttaggaaaa |
| 2281 | cacttattac catataaagt tccaaagagc tttacatttg ttccttgcat accaagaggt |
| 2341 | gacaatggaa aggtaaacag agataagatg ctaaaaggct taatagaaaa aaatctagtt |
| 2401 | aataaagttt gctaggatat aaattcagtt aactatctgc accaagtgca gtggaaaata |
| 2461 | aatcaaaatt aataaaataa attaataagg taaatttagg aggtctaaaa tgagttacga |
| 2521 | cgcacttttt tcaccattta aaatcagagg attagaactt aaaaacagaa tagttctacc |
| 2581 | aggtatgaat acaaaaatgg caaaaaataa acatgattta agcgatgata tgatagctta |
| 2641 | ccatgttgca agagcaaaag caggttgtgc attaaatata tttgaatgtg ttgcgctatg |
| 2701 | tccagcacct catgcatata tgtacatggg attatacaat gacaatcatg tagctcagtt |
| 2761 | aaaaaaatta acagatgctg ttcacgaagt tggcggtaaa atggctgttc agttatggca |
| 2821 | tggtggtttc agcccacaga tgttctttga taaaacaaat acattagaaa caccagatac |
| 2881 | tataacagtt gaacgtattc atgaaatagt taaagagttt ggagaaggtg caagaagagc |
| 2941 | tgttgaagct ggattcgatg cagttgaatt ccatgcagca cacagttact tacctcacga |
| 3001 | attcctaagt ccaggaatga acaaaagaac tgacgaatat ggtggaaact tcgaaaatcg |
| 3061 | ttgcagattc tgcttcgaag tagttgaagc tatacgtgca aatataccag aagatatgcc |
| 3121 | attcttcatg agagttgact gcatagatga gttaatggat gaagtaatga cagaagaaga |
| 3181 | aatagtagaa ttcataaata gatgtgctga tctaggagta gacgtagctg acttatcaag |
| 3241 | aggtaatgct cagtcattcg caacagttta cgaagttcct cctttcaact tacagcacgg |
| 3301 | tttcaatata gaaaacatat acaacatcaa aaaacagata aaaataccag taatgggtgt |
| 3361 | tggacgtata aacacaggag aaatggctaa ccaggtaata gcagatggaa aatttgactt |
| 3421 | agttggtata ggtcgtgctc agttagcaga tcaggattgg gttgctaaag ttagagaagg |
| 3481 | taaagaagat ttaatacgtc attgtatagg atgtgaccag ggatgctacg atgcagttat |
| 3541 | aaaccctcag atgactcata taacttgtac aagaaaccct cacttatgct tagaatacaa |
| 3601 | aggtatgcca aaaactgatg aacctaaaaa agttatgata atcggtggtg gtatggctgg |
| 3661 | tatattagca gctgaagtac ttaaaaaacg tggacatgaa ccagttatat tcgaagcttc |
| 3721 | tgatcactta gcaggacagt tcgtattagc aggtaaagct ccaatgaaag aagactgggc |
| 3781 | agctgcagct aaatgggaag ctgaagaagt agctcgttta ggaatagaag ttagatacaa |
| 3841 | tacaaaagtt actccagaat taatagaaga attcgctcca gaccacgttg ttatagctat |
| 3901 | aggatctgat tacgtagctc cagctatacc aggtatagat agtgacaaag tttacactca |
| 3961 | gtatcaggta ttaaaaggtg aagtagaacc aaaaggacat gtagcagtag ttggttgtgg |
| 4021 | attagttggt acagaagttg ctcagtactt agcagctaga ggagctcagg taacagctat |
| 4081 | agaaagaaaa ggtgttggta caggtctaag catgcttaga agaatgttca tgaacccaga |
| 4141 | attcaaatac tacaaaataa acaaaatgtc tggaactaac atagttggta tagaaccagg |
| 4201 | aaaacttcac tacataatga ctaacaagaa aactcaggaa gttactgaag gtgtgttaga |
| 4261 | atgtgatgca gcagtaatct gtacaggtat aactgctaga ccaagtgaag atttacagga |
| 4321 | aaaatgtaaa gaattaggtg ttccattcaa cgtaataggt gacgcagctg gtgctagaga |
| 4381 | tgctagaata gctactcagg aaggttacga agtaggtatg agtatataat ttaaaaatta |
| 4441 | tataattata taaattaaaa gttattaaat tacaagaaag aggcgaataa aatgacttta |
| 4501 | gaagcaagaa tagaagcatt agaaaaagaa atacagagat taaacgatat agaagctata |
| 4561 | aaacagttaa aagctaaata tttccgttgc ctagatggaa aattatggga tgaattagaa |
| 4621 | actactcttt ctcctaacat agaaacttct tactctgatg gaaaattagt attccacagc |
| 4681 | ccaaaagaag taactgaata tttagcagca gcaatgccta aagaagaaat aagtatgcac |
| 4741 | atgggacata ctccagaaat aactatagac agcgaaaata ctgctacagg aagatggtac |
| 4801 | ttagaagata acctaatatt cacagacgga aaatacaaaa acgttggaat aaacggtgga |
| 4861 | gcattctaca cagataaata tgaaaaaata gacggacagt ggtacataaa agaaactgga |
| 4921 | tatgttcgta tatttgaaga acatttcatg agagatccaa aaatacatat aactagcaac |
| 4981 | atgcataaag aaaaataata actgattgct aataaacaag atataaacag ggggctggta |
| 5041 | aacagccagc cctctgaaaa ataaactaaa aaactataat cttttaaaat cttaattaaa |
| 5101 | gtagaaggag ataagacaat gaacttagta caggacaaaa tagttataat aacaggtgga |
| 5161 | acaagtggta taggtctttg cgcagcaaaa atattcatgg ataacggtgc aacagtttct |
| 5221 | atattcggaa aaactcagga agaagtagat gctgctaaag cagaattaaa agaaactcac |
| 5281 | ccagataaag aagtattagg atttgctcca gatttaacta atagagatga agttatggct |
| 5341 | gcagttggtg cagtagctga aaaatacgga agattagacg ttatgataaa caatgctggt |
| 5401 | gttactagct caaacgtatt ctcaagagtt agcccagaag aattcacata tttaatggat |
| 5461 | ataaacgtta caggtgtatt ccatggtgct tgggctgctt accactgcct gaaaggtgaa |
| 5521 | aagaagatta taataaatac tgcttcagta acaggaatac acggatcatt atcaggagtt |
| 5581 | ggatacccaa caagtaaatc agctgttgta ggattcactc aggctcttgg tagagaaata |
| 5641 | atacgtaaaa acataagagt tgttggtgtt gcaccaggtg ttgttaacac tccaatggtt |
| 5701 | ggtaatatac cagatgaaat attagatgga tacctaagct cattcccaat gaagagaatg |
| 5761 | ttagaaccag aagaaatagc taacacttac ttattcttag cttctgactt agctagtggt |
| 5821 | ataacagcta caactgtaag cgttgacggt gcttatagac catcataaga tttactttaa |
| 5881 | tttaaaactg taattagata gataatacga cgattaatat aaaaaatgtt ctttaaaaga |
| 5941 | aaaggagaaa taaaatggct ggattaaaag attttcctaa atttggtgca ctttctggat |
| 6001 | taaaaatatt agatagtgga tctaacatag ctggacctct aggtggtgga cttttagcag |
| 6061 | aatgtggtgc tacagttata cacttcgaag gacctaaaaa acctgacaac cagagaggtt |
| 6121 | ggtatggata ccctcagaac cacagaaacc agttatcaat ggttgctgat ataaaatctg |
| 6181 | aagaaggtag aaaaatattc ttagacttaa taaaatgggc tgacatatgg gttgaatcat |
| 6241 | caaaaggtgg acagtacgac agactaagtc tttctgatga agttatatgg tcagtaaacc |
| 6301 | ctaaaatagc tatagttcac gtttctggat acggacaggt tggagatcca tcatacgtaa |
| 6361 | caaaagcttc ttatgatgct gttggacagg cattcagtgg atacatgtca ttaaatggtg |
| 6421 | ttaatgaagc attaaaaata aatccttacc taagtgactt cgtatgtgtt cttactactt |
| 6481 | gctgggcaat gttagcatgc tacgtaagta ctcagttaac tggaaaagga gaatctgtag |
| 6541 | acgttgctca gtacgaagca ttagctcgta taatggacgg acgtatgata cagtacgcta |
| 6601 | ctgatggtgt aagtgttcca aaaactggta acaaagatgc tcaggcagct ctattcagct |
| 6661 | tctatacttg taaagatgga agaactatat tcataggtat gactggtgct gaagtatgta |
| 6721 | agagaggatt ccctgtaata gggcttccag ttcctggtac aggtgaccct gacttcccag |
| 6781 | aaggattcac aggatggatg ataaatactc cagttggaca gagaatggaa aaagctatgg |
| 6841 | aagcattcgt tgctgaaaga actatgccag aagttgaaaa agctatgata gatgctcaga |
| 6901 | taccatgcca gagagtttat gatcttgaag actgcttaaa cgaccctcac tggaatgctc |
| 6961 | gtggaactat aatggaatgg gatgacccaa tgatgggaca cataaaaggt cttggattaa |
| 7021 | taaacaaatt caaaaacaac ccttctgaaa tatggagagg tgctccatta ttcggtatgg |
| 7081 | acaacagaga cataattaga gaccttggat attctgagga ggaagttaac gatttatacg |
| 7141 | ctaaaggtat tgtaaacgaa ttcgaccttg aaacaactat aaaacgttac aaacttgatc |
| 7201 | aggttatacc tcacatggct aaaaaagata aataagaaac gtattaaata ataaaatata |
| 7261 | aatgtcgagc ctgccagaat gagaattttg acaggcttga tattataacg aaatgttata |
| 7321 | aaaaaaacaa aataaaaatt gcttaaattt tatacaagga gaattgaaat gacagcaaca |
| 7381 | aacgcaaact ataaaaaagg ctttatccca tttgctatag cagcgttact agtaggtctt |
| 7441 | ataggtggtt tcacagccgt tctagcacct gcattcgtag cagatatggg tcttaacgat |
| 7501 | aacaatacta catggatagc actagcgctt gcaatgtcta cagctgcatg tgctccaata |
| 7561 | cttggtaaat taggtgacgt acttggacgt cgtaaaactt tattattagg aatcatagta |
| 7621 | ttcacaatag gtaacgtatt aacagcaata gcatcttcat taatattcat gctaggtgca |
| 7681 | agatttatag ttggggttgg tacagcggct atagctccag ttataatggc ttacatagtt |
| 7741 | acagaatatc caccagaaga aactggtaag ggattcgctc tttatatgtt aatatcaagt |
| 7801 | gctgcagttg ttgttggtcc aacttgtggt ggattaataa tgcaggcatt tggatggaga |
| 7861 | atgatgatgt gggtttgtgt tgccctttgt gtagtaacat tcttcatatg ttcagtaatg |
| 7921 | attaagaaaa cagactttga aaagaaaagt cttgataact tcgataaaaa aggtgcagta |
| 7981 | tgcgtactaa tattcttcag tttagtatta tgtataccat catttggaca gaatataggt |
| 8041 | tggacatcag cgccattcct aggtgttaca gcagtagctt tagtaacatt attcttatta |
| 8101 | ataaaagctg aaagcagtgc agaaaaccca atattaagtg gtaaatttat gaaacgtaaa |
| 8161 | gaattcatat taccagtatt aatattattc cttactcagg gattaatgca ggctaacatg |
| 8221 | actaacgtaa tattattcgt tagagctact cagccagaaa atacaataat atcaagtttc |
| 8281 | gcaatatcaa tcctttacat aggtatgtct ttaggttcag tattcatagg acctatggca |
| 8341 | gataaaaaag aaccaaaaac tgtacttaca ggatcacttc tattcactgg tataggttgt |
| 8401 | gcaatgatgt acttcttcac agaaactgca ccattcgcaa tgttagctgg atctctagga |
| 8461 | atgttaggta taggacttgg aggaaatgct acaatactaa tgaaagtttc attatctgga |
| 8521 | ttatctcagg cagaagctgg atcaggaaca ggaacatacg gattattcag agatatatca |
| 8581 | gctccatttg gtgttgcggt attcgtacca ctatttgcaa acacagttac aacaagaatg |
| 8641 | gctggagtaa tggctaacgg aactgcagaa gctgctgcta aatcattagc atctgtttct |
| 8701 | tctatacata cattagcatt agttgaagta tgctgtgtaa tattagcaat agttgcagtt |
| 8761 | agaatgctac caaaaataca caataaataa tttaaaaata ataacagagt tgaaaaaaca |
| 8821 | ctcaattaaa agaggggcct tgagcccctt ttttagtgta aaaatgacaa aatactatca |
| 8881 | atttatataa atgataatta aactcgtcaa ccaaagaaat attcacaaag tagataataa |
| 8941 | tagatattca aaaagtgata tattattagg caaaaagtgc aagaaattag cgagtattcg |
| 9001 | acaacttttt gtccaatggt agaaaagaat atttgttatc ataaatatag acaaagggct |
| 9061 | ttgaccaaaa ctaaggaaaa agtttgcata atataaaaaa taaaataaaa taaaaaaata |
| 9121 | aaaataaaat aaaagcgaaa ggaaaaaaca acatcatgga tatgaaaaat tctaaactat |
| 9181 | tctcaccttt aacaatagga tcattaacat taaacaacag agttggtatg gcaccaatga |
| 9241 | gtatggacta cgaagctgct gacggaacag ttccaaaaag attagcagat atatttgttc |
| 9301 | gtagagctga aggtggaaca ggatatgtaa caatagacgc ggtaacaata gatagtaaat |
| 9361 | ataaatatat gggtaataca actgctttag attctgatga tttagttact cagttcaaag |
| 9421 | aatttgcaac aagagttaga gaagcaggaa gcacattaat acctcaggtt atacatccag |
| 9481 | gaccagaatc aatatgtgga tacagacaca tagcaccact tggaccatca gttaatacaa |
| 9541 | atgctaactg ccacgtgagc cgtgctataa gtgtagatga aatacatgaa ataataaaac |
| 9601 | agtttggaca ggctgctaga agagttgaag aagcaggatg cggtggtata ggattacact |
| 9661 | gtgcacatgc ttacatgcta ccaggttcat tcttatctcc attaagaaac aaaagaatgg |
| 9721 | atgaatacgg cggatgtcta gataacagag caagattcgt aatagaaatg atagaagaag |
| 9781 | ttcgtagaaa tgtaagtcct gatttcccaa taatgcttag aatatctggg gatgaaagaa |
| 9841 | tgataggagg aaactcttta gaagatatgt tatacttagc tccaaaattt gttgaagctg |
| 9901 | gtgtaaatat gtttgaagtt tctggaggta ctcagtacga aggattagaa cacataatac |
| 9961 | caagtcagaa caaaagcata ggtgtaaacg tacacgaagc atctgaaatc aaaaaagttg |
| 10021 | tagatgttcc agtttacgct gttggtaaaa taaatgacat aagatacgct gctgaaatag |
| 10081 | ttgaaagagg actagttgat ggggtatcaa taggtagacc attattagca gatccagact |
| 10141 | tatgtaataa agcaaaagaa aacttatttg atgaaataac tccatgtgca agctgtggag |
| 10201 | gaagctgtat aagccgtact gcagatagac ctcagtgtcg ttgccatata aacccaagag |
| 10261 | ttggattcga atatgattat ccagaagttc cagctgaaaa atctaaaaaa gttctagttg |
| 10321 | taggtgctgg acctggtggt atgatggcag cagttacagc agctgaaaga ggacatgatg |
| 10381 | taacactttg ggaagctgac actcagatag gtggacagat aaacttagca gtagtagctc |
| 10441 | caggtaaaca ggaaatgact aaatggttat ctcacttaaa ctacagagct aaaaaagctg |
| 10501 | gagttaaaat ggtattagga aaagaagcta cagtagaaaa cataaaagaa tttgctccag |
| 10561 | aagcagttat agttgcaaca ggtgctagac cattagttcc accaataaaa ggaactcagg |
| 10621 | actacccagt tcttacagct catgacttct taagaggaaa attcgttata ccaaaaggaa |
| 10681 | aagtttgtgt actaggtgga ggagctgttg cttgtgaaac tgcagaaaca gtattagaaa |
| 10741 | acgctagacc aaacgcattc actagaggat ttgatgctag tatcggtgat gtagatgtta |
| 10801 | cattagtaga aatgttacca cagttattaa caggagtatg tgctccaaat agaactccat |
| 10861 | taataagaaa acttaaaaac aaaggtgttc atataaatgt aaatactaaa atattagaag |
| 10921 | taactgacca cgacgttaaa gttcagagag ctgacggtgc agaagaatgg ttaaaaggat |
| 10981 | tcgactacat actattcgga cttggttcta gaaactacga tccaatatct gaacagataa |
| 11041 | aagaattcgt tccagaagta cacgttgttg gggatgctaa gagagctaga caggcaagct |
| 11101 | ttgcaatgtg ggaagctttc gaagcagcat acagcttata a |
| [SEQ ID NO: 3] | |
| 1 | aaaagatatt aagcattaag aaaatgcaca aaaaatcagc gtgtgagagg gagggcaagg |
| 61 | agttgaagcg tgactttttt aacaagttta atttggggac atcgaacttt gtcacgccgg |
| 121 | gaaaacagtt ggaatacgtt tcggaatgca agccagattc tactgcggtc atttgcttag |
| 181 | ataaagaaca gaactgttcc gttattactt ggcatcagct gcacgtctat tccagccagc |
| 241 | tggcatggta ccttatagaa aatgagattg gcccggggtc gatcgtactt acaatgtttc |
| 301 | cgaacagcat cgagcacatt attgcggtat ttgcaatctg gaaggcgggc gcctgctata |
| 361 | tgcccatgtc ctataaggcg gcggaatccg agatcaggga ggcctgcgat accatccacc |
| 421 | cgaatgcggc ttttgcggaa tgcaagattc caggattaaa attctgcctt agcgcagacg |
| 481 | agatatatga ggcgatggaa ggaagatcca aggagatgcc ttcggaccgt ctggccaatc |
| 541 | cgaacatgat atccttatca ggcggaacca gcggaaagat gaagttcatc cgtcagaacc |
| 601 | ttccatgcgg gctggacgat gagacgatca gaagctggtc tttgatgtct ggaatgggat |
| 661 | ttgagcagcg ccagctgctg gtaggcccgc tgtttcatgg cgcgcctcac tccgcggcgt |
| 721 | ttaatggact gttcatgggc aacaccctgg tactgaccag gaacctttgc ccgggaaata |
| 781 | tcctgaacat gattaagaaa tataagattg aatttataca gatggtgccg accctgatga |
| 841 | accggcttgc caaactggag ggagtcggaa aagaagactt tgcatccctg aaggcgctgt |
| 901 | gccatacagg gggcgtctgt tctccctggc ttaagcagat ctggatcgac ctgctggggc |
| 961 | ctgaaaagat ctatgagatg tattccatga cggaatgcat cggccttacc tgcatccggg |
| 1021 | gagacgagtg ggtgaagcat ccgggaagca tcggacggcc agtgggcgat agcaaggtgt |
| 1081 | ctatccggga tgagaatggc aaggaagttg cgccttttga gattggcgag atctatatga |
| 1141 | cagcgccggc ctcctatctg gttaccgagt acatcaattg ggaaccgctg gaagtgaaag |
| 1201 | agggaggctt ccgaagcgta ggggatatcg gctacgtgga tgagcagggc tatctgtact |
| 1261 | tttctgaccg gcgcagcgac atgctggtat caggcggaga aaacgtgttc gccaccgaag |
| 1321 | tcgagacggc gcttttgaga tataaggata tcctggacgc tgtagtggta gggataccgg |
| 1381 | atgaagatct ggggcgaagg ctccatgcgg tcattgagac agggaaagag ataccggcag |
| 1441 | aggaactgaa aacattcctg agaaagtatc tgactccata taagatacca aagacgttcg |
| 1501 | agttcgtaag gagcatacga aggggagaca atggaaaggc cgacaggaag cggatcctgg |
| 1561 | aagattgtat tgcccgcggg ggatgattct ataaatgcaa agaaaacaaa ttatataaag |
| 1621 | gaggagtaac aaaatgagtt acgaagcact tttttcacca ttcaaggtca gaggactgga |
| 1681 | acttaaaaac cgtatcgtcc tgcctggaat gaacaccaag atggcaaaga acaagcacga |
| 1741 | cataggcgag gatatgatag cctaccatgt tgccagggca aaagcgggat gcgcgttaaa |
| 1801 | tatatttgaa tgcgtagcat tatgtccggc gcctcacgct tatatgtata tggggcttta |
| 1861 | tacggaccat catgtagaac agcttaagaa attgacggat gcagtccatg aagcaggcgg |
| 1921 | caagatgggc atccagctgt ggcatggagg attcagcccg cagatgttct ttgacgagac |
| 1981 | caacaccctg gaaactccgg acactcttac ggtagagagg attcatgaga tcgtagaaga |
| 2041 | attcggacgc ggcgcaagga tggctgttca ggctggattt gacgcagtag aattccatgc |
| 2101 | ggctcacagt tatctgcctc acgagttctt aagccctgga atgaacaaac gtacggatga |
| 2161 | gtacggcgga agttttgaga accgctgcag attctgttat gaagtcgttc aggcaatccg |
| 2221 | ttccaatatc ccggatgaca tgccattctt tatgcgtgca gactgcatcg acgaattaat |
| 2281 | ggaacagacc atgacagagg aagagatcgt tacatttatc aataagtgcg cagaacttgg |
| 2341 | cgtggatgtg gcagaccttt cccgtggaaa cgcgacttca ttcgcaaccg tatatgaagt |
| 2401 | tccgccattc aacctggctc atggcttcaa catagagaat atttacaaca tcaaaaagca |
| 2461 | gatcaatatc ccggttatgg gagttggccg tatcaataca ggagagatgg caaacaaggt |
| 2521 | cattgaagaa ggcaagtttg acctggtagg catcggacgc gcccagcttg cagatccaaa |
| 2581 | ctggatcacc aaagtaagag aaggcaaaga agacctgatc cgccactgta tcggatgtga |
| 2641 | ccagggatgc tatgacgcag tcatcaatcc aaagatgaag catatcacct gcacccacaa |
| 2701 | tccaggattg tgcttagagt atcagggaat gccaaagaca gacgctccta agaaagtcat |
| 2761 | gatcgtagga ggcggaatgg caggcatgat cgctgcggaa gtattaaaga ccagaggcca |
| 2821 | taacccggta atcttcgagg catccgacaa gcttgcagga cagttcaggc tggcaggcgt |
| 2881 | agcgccgatg aagcaggatt gggcagatgt tgcagaatgg gaagcaaaag aagtagagcg |
| 2941 | ccttggaatc gaagtacgtc tgaataccga agtgactgca gagaccatca aggaattcaa |
| 3001 | tccggataat gtcatcatcg cagtaggctc tacctatgcg ctgcctgaga ttccgggaat |
| 3061 | cgacagccca agcgtatact cccagtatca ggtactgaaa ggggaagtaa atccgacagg |
| 3121 | ccgtgtagcc gttatcggat gcggactggt tggtacggaa gtcgcagaac ttctggcatc |
| 3181 | cagaggcgca caggtaatcg cgatcgagag gaagggcgta ggtaccggcc ttagcatgct |
| 3241 | tcgcagaatg ttcatgaacc cggaattcaa atattacaag atcgccaaga tgtccggaac |
| 3301 | aaatgtcacc gctttagagc agggcaaggt tcactacatc atgacagaca agaagaccaa |
| 3361 | agaagtgacg cagggagtcc tggaatgcga cgctaccgtt atctgtacag gaattaccgc |
| 3421 | acgtccaagc gatgggctta aggcaagatg cgaagaactt ggaatcccgg ttgaggtgat |
| 3481 | cggagacgct gctggcgcaa gagactgcac gatcgcgaca cgcgaaggct atgacgcagg |
| 3541 | aatggcaatc tagaaaatca gaacttatca atcttacata tagaaaggat gatacatatg |
| 3601 | acattagaag agagagttga agcattagaa aaagaattgc aggagatgaa ggatattgag |
| 3661 | gcaatcaagg aactgaaagg aaagtatttc cgctgcctgg acggaaagat gtgggatgag |
| 3721 | ctggagacca ccctgtcacc aaatatcgta acctcttatt ccaacgggaa actggtattc |
| 3781 | catagcccga aggaagttac cgattactta aagagctcga tgccaaaaga agagatcagc |
| 3841 | atgcatatgg gccacacgcc ggagatcacc attgacagcg agactacggc tacgggcaga |
| 3901 | tggtatctgg aagatagact gatctttacg gacggtaagt acaaagacgt aggaatcaat |
| 3961 | ggcggcgcgt tctatacaga caaatatgag aagatagacg gccagtggta catccttgaa |
| 4021 | accggctatg tacgaatcta tgaagaacat ttcatgcgtg atccaaagat ccatatcacg |
| 4081 | atgaacatgc acaaataaga atattgtaaa agaaaggcag gagtaagagt atgaatctcg |
| 4141 | tacaagacaa agttacgatc atcacaggcg gcacaagagg tattggattc gccgctgcca |
| 4201 | aaatatttat cgacaatggc gcaaaagtat ccatcttcgg agagacgcag gaagaagtag |
| 4261 | atacagcgct tgcacagtta aaagaacttt atccggaaga agaggttctg ggattcgcgc |
| 4321 | cggatcttac atccagagac gcagttatgg cagcggtagg ccaggtagca cagaaatatg |
| 4381 | gcagactgga tgtcatgatc aacaatgcag gaattaccag caacaacgta ttctccagag |
| 4441 | tgtctgaaga agagttcaag catattatgg acatcaacgt aacaggcgta ttcaacggcg |
| 4501 | catggtgcgc ataccagtgc atgaaggatg ccaaaaaggg cgttatcatc aacacggcat |
| 4561 | ccgttacagg catcttcgga tcactctcag gcgtaggata tccggccagc aaggcaagcg |
| 4621 | tgatcggact cacccatgga cttggaagag agatcatccg caagaatatc cgtgtagtag |
| 4681 | gagtggctcc tggagttgtg aacacggata tgaccaatgg caatcctccg gagatcatgg |
| 4741 | aaggatatct gaaggcgctt ccgatgaaga gaatgcttga gccggaagag atcgctaatg |
| 4801 | tatacctgtt cctggcatct gacttggcaa gcggcattac ggctactacg gtcagcgtag |
| 4861 | acggggctta cagaccataa ttttaatttt tactaagtag aatatgtgat atagaaaagg |
| 4921 | agatataaaa acatggctgg aataaaagat tttccaaaat tcggagctct tgcagggctt |
| 4981 | aagatacttg acagcggatc taacatcgcc ggacctttag gcggaggcct tctggcagaa |
| 5041 | tgcggagcaa cggtcatcca ttttgaagga ccaaagaaac ctgataacca gagaggatgg |
| 5101 | tacggctatc cacagaatca ccgtaatcag ctgtctatgg tagcagacat caaatctgaa |
| 5161 | gaaggaagaa agatcttcct tgatctgatc aaatgggcag atatctgggt agagtcatcc |
| 5221 | aaaggcggac agtatgacag gctgggactt tccgatgaag tcatctggga agtaaatcct |
| 5281 | aagattgcca tcgtgcacgt atccggatat ggacagacag gagacccgtc ttacgttaca |
| 5341 | cgtgcatcct atgacgcagt aggccaggca ttcagcggct atatgtcact gaacggaaca |
| 5401 | acggaagcgc tgaagatcaa tccttatctg agcgatttcg tatgcggact taccacatgc |
| 5461 | tgggctatgc ttgcctgcta tgtaagcacc attcttaccg gaaaaggcga atctgttgac |
| 5521 | gttgcacagt acgaagcgct ggcacgtatc atggacggac gtatgatcca gtacgctaca |
| 5581 | gacggcgtga agatgccaag aaccggcaat aaggatgcgc aggctgccct gttcagcttc |
| 5641 | tacacctgta aagacggacg tacgatcttt atcggaatga ctggcgcgga agtatgtaag |
| 5701 | agaggcttcc cgatcatcgg acttccggta cctggaaccg gagacccgga cttcccggaa |
| 5761 | ggcttcacag gctggatgat ctatactcct gtaggacaga gaatggaaaa ggctatggag |
| 5821 | aagtatgtat ctgagcatac gatggaagaa gtagaggctg agatgcaggc acaccagatt |
| 5881 | ccatgccaga gagtatacga gctggaagac tgcctgaacg atcctcactg gaaagcacgt |
| 5941 | ggaactatta cggagtggga tgacccgatg atgggacata tcacaggcct tggactgatc |
| 6001 | aacaagttca agagaaatcc ttccgaaatc tggagaggcg ctccgctgtt cggtatggat |
| 6061 | aaccgcgata tcctgaaaga cctgggatat gacgatgcaa agatcgatga actctatgag |
| 6121 | cagggcatcg tcaatgaatt cgaccttgac actactatca aacgctatag actggatgaa |
| 6181 | gtaattccac atatgagaaa gaaagaggag taagagtatg agcaccgtag ccaatccaaa |
| 6241 | ttataagaaa ggttttgtcc cctttgcaat tgcagcactc ctggtgagcc tgatcggcgg |
| 6301 | ttttaccgcc gttctcggcc cggccttcgt ggcggaccag gggattgact ataataatac |
| 6361 | cacatggatt tccctggcgc tggcgatgtc ttccgccgca tgcgctccaa tccttggaaa |
| 6421 | actgggagac gtgctaggac gcaggacgac gctgcttctg ggtattgtga tctttgcggc |
| 6481 | cggcaatgtg ctgacagccg tagccacgtc cctgatattc atgctggcag cccgttttat |
| 6541 | cgtaggtatc ggaacagcag cgatctcacc gatcgttatg gcctatatcg taaccgagta |
| 6601 | tccgcaggag gagacaggaa aggcctttgg cctgtatatg ctgatctcca gcggcgccgt |
| 6661 | cgtggtagga cctacctgtg gcggcctgat catgaatgcg gctggctgga gagtcatgat |
| 6721 | gtgggtatgc gtcgctctgt gcgtcgttgt attcctgatc tgcacattct ccatcaagaa |
| 6781 | gactgcattt gagaagaaga gcatggcagg atttgacaag ccgggcgcag ccctggtagt |
| 6841 | cgtattcttc agtttgttcc tgtgcatccc atccttcgga cagaatatcg gatggtcttc |
| 6901 | cacagcattt atcgcagcag cggcagtagc gctggtagca cttttcatcc tggtaatggt |
| 6961 | agaaaagaaa gcgaagagtc cgatcatgaa cggcaagttt atggcacgca aggaattcgt |
| 7021 | gcttccagta ttgatcctgt tccttacaca gggacttatg atggcaaata tgaccaatgt |
| 7081 | catcgtgttc gtgcgctata cgcagccgga caatgtcatt atatcaagtt ttgcgatctc |
| 7141 | catcatgtac ataggaatgt ccttaggctc cgttatcatt ggacctgttg cagataagaa |
| 7201 | agagccaaag acggttctga cattctctct ggtactgaca gccatcggct gtgcgctgat |
| 7261 | gtatctgttc aaggcagatt cctccgtcgc tatctttgcg gcatccttgg gaatccttgg |
| 7321 | atttggcctt ggaggaaatg caaccatctt catgaaggta gcgctttccg gcctgtccag |
| 7381 | cgaagtagct ggctctggta ctggaaccta tggcctgttc agagatatct cggcaccatt |
| 7441 | cggcgtggca gtgttcgtgc ctatgtttgc caacggcgta acagcgaata ttgcgaaata |
| 7501 | cgcgtcaggc ggcatggaag aaggcgccgc tacggtaaaa gcagccatct catccatcca |
| 7561 | gacgctgaca ctggttgaac ttggatgtat cgttgtggga atcatccttg tgagaatgct |
| 7621 | gccaagaatc tatcagaaga aagaggcata aataagttaa gaaaagaggt aattataaat |
| 7681 | ggatatgaaa cattccagat tattttcgcc gcttcagatc ggatccctga cactgtctaa |
| 7741 | ccgtgtcggc atggctccca tgagcatgga ctatgaagca gcagacggaa ctgtgcccaa |
| 7801 | gaggctggcg gacgtatttg tccgccgcgc cgagggaggc acaggctacg tcatgatcga |
| 7861 | cgcggtgacg atagacagca agtatcctta tatgggaaat acaacggccc ttgaccgtga |
| 7921 | tgaactggtt ccccagttta aggaatttgc tgacagagta aaagaagcag gcagcacgct |
| 7981 | ggtgccgcag atcattcatc cgggtccgga atccgtatgc ggctaccggc atatcgctcc |
| 8041 | gcttggacct tctgccaaca ccaatgcaaa ctgccacgtg agcagatcga tcagcataga |
| 8101 | tgagatccat gacatcatta agcagttcgg ccaggcggca cgccgcgccg aagaagcagg |
| 8161 | atgcggggca atctccctgc actgcgcgca tgcgtatatg ctgccaggat ccttcctgtc |
| 8221 | accgcttcgc aacaagcgca tggatgaata tggcggaagc cttgacaacc gtgcccgttt |
| 8281 | cgtgatcgag atgattgagg aggcccgcag gaatgtgagt cctgatttcc cgatcttcct |
| 8341 | tcgtatctcc ggagacgaga gaatggtagg aggcaacagc cttgaagata tgctctacct |
| 8401 | ggcaccgaag ttcgaggctg ccggcgtaag catgctggaa gtatccggcg gaacccagta |
| 8461 | tgaaggcctg gaacatatca ttccttgcca gaataagagc aggggcgtca atgtatatga |
| 8521 | agcttctgag atcaagaaag tagtgggcat cccggtatac gcagtaggaa agatcaacga |
| 8581 | tatacgctat gcggcagaga tcgtagaacg cggcctggta gacggcgtgg ctatgggacg |
| 8641 | tccgcttctg gcagatccgg acctttgcaa gaaggcagtg gaaggccagt ttgacgagat |
| 8701 | cactccatgc gcaagctgcg gcggaagctg catcagccgt tctgaggcag cgcctgagtg |
| 8761 | ccattgccat attaatccaa ggcttggccg ggagtatgaa ttcccggatg tgcctgccga |
| 8821 | gaagtccaag aaggtactgg ttatcggcgc aggccctgga ggaatgatgg ctgccgtgac |
| 8881 | agctgcggaa cgcggccatg atgttacggt atgggaggct gacgacaaga tcggcggcca |
| 8941 | gctgaacctg gcagtagtgg ctcctggcaa gcaggagatg acccagtgga tggtacatct |
| 9001 | gaactatcgc gcgaagaaag caggcgtgaa gtttgaattc aataaagaag cgacggcaga |
| 9061 | agatgtcaag gcgctggcgc cggaagcagt gatcgttgct acaggcgcga agccgctggt |
| 9121 | tcctccgatt aaaggaacac aggattatcc ggtgcttact gcccatgatt tccttcgcgg |
| 9181 | caagttcgtg attccgaagg gacgcgtctg cgtgctggga ggaggcgcgg ttgcctgcga |
| 9241 | gactgccgag acagccctgg agaatgcacg tccgaattct tataccagag gatacgatgc |
| 9301 | aagcatcgga gatatcgatg tcacgcttgt ggagatgctt ccgcagctcc ttaccggcgt |
| 9361 | atgcgcgccg aaccgcgagc ctttgatccg caagttaaag agcaagggcg tacacatcaa |
| 9421 | cgtcaatacc aagatcatgg aagtaacaga ccatgaagta aaggttcaga gacaggatgg |
| 9481 | aacgcaggaa tggctggaag gatttgacta tgtcctcttt ggccttggtt ccagaaatta |
| 9541 | cgatccgctt tcagagaccc tcaaggaatt cgttccggaa gtacatgtca tcggcgatgc |
| 9601 | cgtaagggcg cgccaggcaa gctacgcaat gtgggaagga tttgagaagg catacagcct |
| 9661 | gtaaaagcgg tttgagtaaa aggaggctta agaaatggca gtgaaggcaa tctcaggctg |
| 9721 | cgacaaggat caggaactga tca |

In certain embodiments, the bacterium is transformed with a vector comprising a bile acid-inducible operon (bai operon). In certain embodiments, the bacterium stably expresses an enzyme having 7-dehydroxylation activity. In certain embodiments, the enzyme is a bile-acid 7-dehydroxylase. In certain embodiments, the enzyme is selected from the group consisting of a bile-acid 7-dehydroxylase, bile-acid 7-alpha-dehydroxylase, 7-alpha-dehydratase, bile acid CoA ligase, 3 alpha-HSDH, CoA transferase, 3-dehydro-4-7-alpha-oxidoreductase, 3-dehydro-4-7-beta-oxidoreductase, CA/CDCA transporter, 7-beta-dehydratase and AraC/XyIS. In certain embodiments, the enzyme comprises an amino acid sequence that is at least about 80% (e.g., at least about 85%, at least about 90%, or at least about 95%) homologous or identical to the amino acid of a bile-acid 7-dehydroxylase, bile-acid 7-alpha-dehydroxylase, 7-alpha-dehydratase, bile acid CoA ligase, 3 alpha-HSDH, CoA transferase, 3-dehydro-4-7-alpha-oxidoreductase, 3-dehydro-4-7-beta-oxidoreductase, CA/CDCA transporter, 7-beta-dehydratase__ or AraC/XyIS.

In certain embodiments, the bacterium is selected from the group consisting of Ruminococcus, Alloprevotella, Allisonella, Anaerostipes, Anaerobiospirillum, Bacteroides, Blautia, Clostridium sensu stricto 1, Collinsella, Coprococcus 1, Corynebacterium 1, Campylobacter, Enterococcus, Erysipelatoclostridium, Escherichia-Shigella, Faecalitalea, Fusobacterium, Clostridium, Helicobacter, Intestinibacter, Lachnoclostridium, Lactobacillus, Megasphaera, Methanobrevibacter, Parabacteroides, Porphyromonas, Phascolarctobacterium, Peptoclostridium, Prevotellaceae UCG-001, Pseudocitrobacter, Ruminiclostridium 9, Sarcina, Streptococcus, Succinivibrio, Treponema 2, Turicibacter, Tyzzerella, Tyzzerella 4 and any combination thereof. In certain embodiments, the bacterium is selected from the genus of Clostridium.

In certain embodiments, the intestinal microorganism comprises a 16s rRNA comprising a nucleotide sequence that is at least about 80% (e.g., at least about 85%, at least about 90%, or at least about 95%) homologous or identical to SEQ ID NO: 2. In certain embodiments, the intestinal microorganism comprises a 16s rRNA comprising the nucleotide sequence set forth in SEQ ID NO: 2 or 4. SEQ ID NO: 2 represents an exemplary sequence of 16S rRNA gene in C. hiranonis. SEQ ID NO: 4 represents an exemplary sequence of 16S rRNA gene in C. scindens.

| [SEQ ID NO: 2] | |
|---|---|
| 1 | acatgcaagt cgagcgattc tcttcggaga agagcggcgg acgggtgagt aacgcgtggg |
| 61 | taacctgccc tgtacacacg gataacatac cgaaaggtat gctaatacgg gataatatat |
| 121 | aagagtcgca tgacttttat atcaaagatt tttcggtaca ggatggaccc gcgtctgatt |
| 181 | agcttgttgg cggggtaacg gcccaccaag gcgacgatca gtagccgacc tgagagggtg |
| 241 | atcggccaca ttggaactga gacacggtcc aaactcctac gggaggcagc agtggggaat |
| 301 | attgcacaat gggcgcaagc ctgatgcagc aacgccgcgt gagcgatgaa ggccttcggg |
| 361 | tcgtaaagct ctgtcctcaa ggaagataat gacggtactt gaggaggaag ccccggctaa |
| 421 | ctacgtgcca gcagccgcgg taatacgtag ggggctagcg ttatccggat ttactgggcg |
| 481 | taaagggtgc gtaggcggtc tttcaagtca ggagttaaag gctacggctc aaccgtagta |
| 541 | agctcctgat actgtctgac ttgagtgcag gagaggaaag cggaattccc agtgtagcgg |
| 601 | tgaaatgcgt agatattggg aggaacacca gtagcgaagg cggctttctg gactgtaact |
| 661 | gacgctgagg cacgaaagcg tggggagcaa acaggattag ataccctggt agtccacgct |
| 721 | gtaaacgatg agtactagtt gtcggaggtt accccttcgg tgccgcagct aacgcattaa |
| 781 | gtactccgcc tggggagtac gcacgcaagt gtgaaactca aaggaattga cggggacccg |
| 841 | cacaagtagc ggagcatgtg gtttaattcg aagcaacgcg aagaacctta cctaggcttg |
| 901 | acatccttct gaccgaggac taatctcctc tttccctccg gggacagaag tgacaggtgg |
| 961 | tgcatggttg tcgtcagctc gtgtcgtgag atgttgggtt aagtcccgca acgagcgcaa |
| 1021 | cccttgtctt tagttgccat cattaagttg ggcactctag agagactgcc agggataacc |
| 1081 | tggaggaagg tggggatgac gtcaaatcat catgcccctt atgcctaggg ctacacacgt |
| 1141 | gctacaatgg gtggtacaga gggcagccaa gccgtgaggt ggagcaaatc ccttaaagcc |
| 1201 | attctcagtt cggattgtag gctgaaactc gcctacatga agctggagtt actagtaatc |
| 1261 | gcagatcaga atgctgcggt gaatgcgttc ccgggtcttg tacacaccgc ccgtcacacc |
| 1321 | atgggagttg gagacacccg aagccgacta tctaaccttt tgggagaagt cgtccccctc |
| 1381 | gaatcaatac ccc |
| [SEQ ID NO: 4] | |
| 1 | gagagtttga tcctggctca ggatgaacgc tggcggcgtg cctaacacat gcaagtcgaa |
| 61 | cgaagcgctt ccgctagatt ttcttcggag atgaaggcgg ctgcgactga gtggcggacg |
| 121 | ggtgagtaac gcgtgggcaa cctgccttgc actgggggat aacagccaga aatggctgct |
| 181 | aataccgcat aagaccgaag cgccgcatgg cgcagcggcc aaagccccgg cggtgcaaga |
| 241 | tgggcccgcg tctgattagg tagttggcgg ggtaacggcc caccaagccg acgatcagta |
| 301 | gccgacctga gagggtgacc ggccacattg ggactgagac acggcccaga ctcctacggg |
| 361 | aggcagcagt ggggaatatt gcacaatggg ggaaaccctg atgcagcgac gccgcgtgaa |
| 421 | ggatgaagta tttcggtatg taaacttcta tcagcaggga agaagatgac ggtacctgac |
| 481 | taagaagccc cggctaacta cgtgccagca gccgcggtaa tacgtagggg gcaagcgtta |
| 541 | tccggattta ctgggtgtaa agggagcgta gacggcgatg caagccagat gtgaaagccc |
| 601 | ggggctcaac cccgggactg catttggaac tgcgtggctg gagtgtcgga gaggcaggcg |
| 661 | gaattcctag tgtagcggtg aaatgcgtag atattaggag gaacaccagt ggcgaaggcg |
| 721 | gcctgctgga cgatgactga cgttgaggct cgaaagcgtg gggagcaaac aggattagat |
| 781 | accctggtag tccacgccgt aaacgatgac tactaggtgt cgggtggcaa ggccattcgg |
| 841 | tgccgcagca aacgcaataa gtagtccacc tggggagtac gttcgcaaga atgaaactca |
| 901 | aaggaattga cggggacccg cacaagcggt ggagcatgtg gtttaattcg aagcaacgcg |
| 961 | aagaacctta cctgatcttg acatcccgat gccaaagcgc gtaacgcgct ctttcttcgg |
| 1021 | aacatcggtg acaggtggtg catggttgtc gtcagctcgt gtcgtgaggt gttgggttaa |
| 1081 | gtcccgcaac gagcgcaacc cctatcttca gtagccagca tttcggatgg gcactctgga |
| 1141 | gagactgcca gggacaacct ggaggaaggt ggggatgacg tcaaatcatc atgcccctta |
| 1201 | tgaccagggc tacacacgtg ctacaatggc gtaaacaaag ggaggcgaac ccgcgagggt |
| 1261 | gggcaaatcc caaaaataac gtctcagttc ggattgtagt ctgcaactcg actacatgaa |
| 1321 | gctggaatcg ctagtaatcg cgaatcagaa tgtcgcggtg aatacgttcc cgggtcttgt |
| 1381 | acacaccgcc cgtcacacca tgggagtcag taacgcccga agccggtgac ccaacccgca |
| 1441 | agggagggag ccgtcgaagg tgggaccgat aactggggtg aagtcgtaac aaggtagccg |
| 1501 | tatcggaagg tgcggctgga tcacctcctt c |

In certain embodiments, the intestinal microorganism comprises C. hiranonis, C. scindens or combination thereof. In certain embodiments, the intestinal microorganism comprises C. hiranonis. In certain embodiments, the intestinal microorganism comprises C. scindens.

By “percentage of identity” between two nucleic acid or amino acid sequences in the sense of the present disclosure, it is intended to indicate a percentage of nucleotides or of identical amino acid residues between the two sequences to be compared, obtained after the best alignment (optimum alignment), this percentage being purely statistical and the differences between the two sequences being distributed randomly and over their entire length. The comparisons of sequences between two nucleic acid or amino acid sequences are traditionally carried out by comparing these sequences after having aligned them in an optimum manner, said comparison being able to be carried out by segment or by “comparison window”. The optimum alignment of the sequences for the comparison can be carried out, in addition to manually, by means of the local homology algorithm of Smith and Waterman (1981) [Ad. App. Math. 2:482], by means of the local homology algorithm of Neddleman and Wunsch (1970) [J. Mol. Biol. 48: 443], by means of the similarity search method of Pearson and Lipman (1988) [Proc. Natl. Acad. Sci. USA 85:2444), by means of computer software using these algorithms (GAP, BESTFIT, FASTA and TFASTA in the Wisconsin Genetics Software Package, Genetics Computer Group, 575 Science Dr., Madison, WI, or else by BLAST N or BLAST P comparison software).

The percentage of identity between two nucleic acid or amino acid sequences is determined by comparing these two sequences aligned in an optimum manner and in which the nucleic acid or amino acid sequence to be compared can comprise additions or deletions with respect to the reference sequence for an optimum alignment between these two sequences. The percentage of identity is calculated by determining the number of identical positions for which the nucleotide or the amino acid residue is identical between the two sequences, by dividing this number of identical positions by the total number of positions in the comparison window and by multiplying the result obtained by 100 in order to obtain the percentage of identity between these two sequences.

For example, it is possible to use the BLAST program, “BLAST 2 sequences” (Tatusova et al., “Blast 2 sequences—a new tool for comparing protein and nucleotide sequences”, FEMS Microbiol Lett. 174:247-250) available on the site www.ncbi.nlm.nih.gov, the parameters used being those given by default (in particular for the parameters “open gap penalty”: 5, and “extension gap penalty”: 2; the matrix chosen being, for example, the matrix “BLOSUM 62” proposed by the program), the percentage of identity between the two sequences to be compared being calculated directly by the program. It is also possible to use other programs such as “ALIGN” or “Megalign” (DNASTAR) software.

By amino acid sequence having at least about 80%, e.g., at least about 85%, at least about 90%, at least about 95% and at least about 98% identity with a reference amino acid sequence, those having, with respect to the reference sequence, certain modifications, in particular a deletion, addition or substitution of at least one amino acid, a truncation or an elongation are preferred. In the case of a substitution of one or more consecutive or nonconsecutive amino acid(s), the substitutions are preferred in which the substituted amino acids are replaced by “equivalent” amino acids. The expression “equivalent amino acids” is aimed here at indicating any amino acid capable of being substituted with one of the amino acids of the base structure without, however, essentially modifying the biological activities of the corresponding antibodies and such as will be defined later, especially in the examples. These equivalent amino acids can be determined either by relying on their structural homology with the amino acids which they replace, or on results of comparative trials of biological activity between the different antibodies capable of being carried out.

By way of non-limiting example, Table 1 represents the possibilities of substitution capable of being carried out without resulting in a profound modification of the biological activity of the corresponding modified antibody, the reverse substitutions being naturally envisageable under the same conditions.

TABLE 1

| Original | |
|---|---|
| residue | Substitution(s) |
| Ala (A) | Val, Gly, Pro |
| Arg (R) | Lys, His |
| Asn (N) | Gln |
| Asp (D) | Glu |
| Cys (C) | Ser |
| Gln (Q) | Asn |
| Glu (G) | Asp |
| Gly (G) | Ala |
| His (H) | Arg |
| Ile (I) | Leu |
| Leu (L) | Ile, Val, Met |
| Lys (K) | Arg |
| Met (M) | Leu |
| Phe (F) | Tyr |
| Pro (P) | Ala |
| Ser (S) | Thr, Cys |
| Thr (T) | Ser |
| Trp (W) | Tyr |
| Tyr (Y) | Phe, Trp |
| Val (V) | Leu, Ala |

### Intestinal Microorganism Indicating Intestinal Health

In certain embodiments, the intestinal microorganism can be used to indicate intestinal health in a subject. In certain embodiments, the intestinal microorganism is associated with an intestinal disorder. In certain embodiments, the intestinal microorganism is associated with a heathy intestinal status. In certain embodiments, the intestinal microorganism more abundant in a healthy subject compared to a subject having an intestinal disorder. In certain embodiments, the intestinal microorganism less abundant in a healthy subject compared to a subject having an intestinal disorder.

In certain embodiments, the intestinal microorganism comprises one or more bacteria and/or archaea of one or more phylum selected from the group consisting of Actinobacteria, Bacteroidetes, Euryarchaeota, Firmicutes, Fusobacteria, Proteobacteria and Spirochaetae.

In certain embodiments, the intestinal microorganism comprises one or more bacteria and/or archaea of one or more class selected from the group consisting of Actinobacteria, Bacilli, Bacteroidia, Clostridia, Coriobacteria, Erysipelotrichia, Fusobacteria, Gammaproteobacteria, Methanobacteria, and Spirochaetes.

In certain embodiments, the intestinal microorganism comprises one or more bacteria and/or archaea of one or more order selected from the group consisting of Bacteriodales, Clostridiales, Coriobacteriales, Corynebacteriales, Enterobacteriales, Erysipelotrichales, Fusobacteriales, Lactobacillaes, Methanobacteriales and Spirochaetales.

In certain embodiments, the intestinal microorganism comprises one or more bacteria and/or archaea of one or more family selected from the group consisting of Bacteroidaceae, Clostridiaceae 1, Coriobacteriaceae, Corynebacteriaceae, Enterobacteriaceae, Erysipelotrichaceae, Fusobacteriaceae, Lachnospiraceae, Methanobacteriaceae, Peptostreptococcaceae, Porphyromonadaceae, Prevotellaceae, Ruminococcaceae, Spirochaetaceae, and Streptococcaceae.

In certain embodiments, the intestinal microorganism comprises one or more bacteria and/or archaea of one or more genus selected from the group consisting of Ruminococcus, Alloprevotella, Allisonella, Anaerostipes, Anaerobiospirillum, Bacteroides, Blautia, Clostridium sensu stricto 1, Collinsella, Coprococcus 1, Corynebacterium 1, Campylobacter, Enterococcus, Erysipelatoclostridium, Escherichia-Shigella, Faecalitalea, Fusobacterium, Helicobacter, Intestinibacter, Lachnoclostridium, Lactobacillus, Megasphaera, Methanobrevibacter, Parabacteroides, Porphyromonas, Phascolarctobacterium, Peptoclostridium, Prevotellaceae UCG-001, Pseudocitrobacter, Ruminiclostridium 9, Sarcina, Streptococcus, Succinivibrio, Treponema 2, Turicibacter, Tyzzerella, and Tyzzerella 4.

In certain embodiments, the intestinal microorganism comprises one or more bacteria and/or archaea of one or more species selected from the group consisting of Enterococcus durans, E. coli and C. perfringens. In certain embodiments, the intestinal microorganism comprises E. coli and C. perfringens.

In certain embodiments, the intestinal microorganism is selected from the group consisting of C. hiranonis, C. scindens, Veillonellaceae, Streptococcaceae, Bacteroides, Fusobacterium, Collinsella, Sarcina, Clostridium sensu stricto 1, Faecalitalea, Streptococcus, Erysipelatoclostridium, Megasphaera, Blautia, Alloprevotella, Peptoclostridium, and any combination thereof. In certain embodiments, the intestinal microorganism is C. hiranonis, C. scindens or combination thereof.

In certain embodiments, the intestinal microorganism comprises one or more bacterium comprising a 16S rRNA comprising a nucleotide sequence that is at least about 80% (e.g., at least about 85%, at least about 90%, or at least about 95%, at least about 96%, at least about 97%, at least about 98%, at least about 99%, at least about 99.5%, or at least about 99.9%) homologous or identical to any sequence in Table 11.

In certain embodiments, the intestinal microorganism comprises one or more bacterium comprising a 16S rRNA comprising a nucleotide sequence that is at least about 80% (e.g., at least about 85%, at least about 90%, or at least about 95%, at least about 96%, at least about 97%, at least about 98%, at least about 99%, at least about 99.5%, or at least about 99.9%) homologous or identical to the 16S rRNA nucleotide sequence of HQ802983.1.1440, FJ950694.1.1472, HG798451.1.1400, New.ReferenceOTU52, New.ReferenceOTU82, GQ449092.1.1375, FJ506371.1.1371, GQ448744.1.1393, FJ957494.1.1454, HQ760911.1.1437, GQ006324.1.1342, GQ448246.1.1389, KC245406.1.1465, New.ReferenceOTU54, HQ751549.1.1448, JF712675.1.1540, JQ208181.1.1352, GX182404.8.1529, FP929060.3837.5503, FN667392.1.1495, FN667422.1.1495, HK557089.3.1395, HQ803964.1.1435, AM276759.1.1484, HK555938.1.1357, KF842598.1.1394, HQ792778.1.1436, FM865905.1.1392, FN563300.1.1447, HQ754680.1.1441, GQ867426.1.1494, EU470512.1.1400, AY239462.1.1500, New.ReferenceOTU114, FN668375.4306350.4307737, AB009242.1.1451, HQ792787.1.1438, AB506370.1.1516, DQ057365.1.1393, FN667084.1.1493, DQ113765.1.1450, HK694029.9.1487, AJ270486.1.1241, EU768569.1.1352, FM179752.1.1686, FJ957528.1.1445, KC504009.1.1465, GQ448506.1.1374, JF224013.1.1362, EU774020.1.1361, GQ448486.1.1387, HQ793763.1.1451, JN387556.1.1324, or New.ReferenceOTU109 in Table 11.

In certain embodiments, the intestinal microorganism comprises one or more bacteria and/or archaea of one or more Operational Taxonomic Units (OTUs) selected from the group consisting of HQ802983.1.1440, FJ950694.1.1472, HG798451.1.1400, New.ReferenceOTU52, New.ReferenceOTU82, GQ449092.1.1375, FJ506371.1.1371, GQ448744.1.1393, FJ957494.1.1454, HQ760911.1.1437, GQ006324.1.1342, GQ448246.1.1389, KC245406.1.1465, New.ReferenceOTU54, HQ751549.1.1448, JF712675.1.1540, JQ208181.1.1352, GX182404.8.1529, FP929060.3837.5503, FN667392.1.1495, FN667422.1.1495, HK557089.3.1395, HQ803964.1.1435, AM276759.1.1484, HK555938.1.1357, KF842598.1.1394, HQ792778.1.1436, FM865905.1.1392, FN563300.1.1447, HQ754680.1.1441, GQ867426.1.1494, EU470512.1.1400, AY239462.1.1500, New.ReferenceOTU114, FN668375.4306350.4307737, AB009242.1.1451, HQ792787.1.1438, AB506370.1.1516, DQ057365.1.1393, FN667084.1.1493, DQ113765.1.1450, HK694029.9.1487, AJ270486.1.1241, EU768569.1.1352, FM179752.1.1686, JF807116.1.1260, FJ957528.1.1445, KC504009.1.1465, GQ448506.1.1374, JF224013.1.1362, EU774020.1.1361, GQ448486.1.1387, HQ793763.1.1451, JN387556.1.1324, and New.ReferenceOTU109.

In certain embodiments, the intestinal microorganism comprises one or more bacterium comprising a 16S rRNA comprising a nucleotide sequence that is at least about 80% (e.g., at least about 85%, at least about 90%, or at least about 95%, at least about 96%, at least about 97%, at least about 98%, at least about 99%, at least about 99.5%, or at least about 99.9%) homologous or identical to the 16S rRNA nucleotide sequence of JRPJ01000002.1034290.1035971, JF920309.1.1340, FJ978526.1.1378, New.ReferenceOTU45, HK555938.1.1357, FJ957494.1.1454, New.ReferenceOTU52, DQ797046.1.1403, GQ449092.1.1375, AMCI01001631.34.1456, KF842598.1.1394, HQ793763.1.1451, DQ113765.1.1450, ACBW01000012.3536.5054, HK693629.1.1491, JQ208053.1.1336, GQ493166.1.1359, GQ448486.1.1387, GQ491426.1.1332, New.ReferenceOTU54, or JN387556.1.1324 in Table 11.

In certain embodiments, the intestinal microorganism comprises one or more bacteria and/or archaea of one or more Operational Taxonomic Units (OTUs) selected from the group consisting of JRPJ01000002.1034290.1035971, JF920309.1.1340, FJ978526.1.1378, New.ReferenceOTU45, HK555938.1.1357, FJ957494.1.1454, New.ReferenceOTU52, DQ797046.1.1403, GQ449092.1.1375, AMCI01001631.34.1456, KF842598.1.1394, HQ793763.1.1451, DQ113765.1.1450, ACBW01000012.3536.5054, HK693629.1.1491, JQ208053.1.1336, GQ493166.1.1359, GQ448486.1.1387, GQ491426.1.1332, New.ReferenceOTU54, and JN387556.1.1324.

In certain embodiments, the intestinal microorganism comprises one or more bacterium comprising a 16S rRNA comprising a nucleotide sequence that is at least about 80% (e.g., at least about 85%, at least about 90%, or at least about 95%, at least about 96%, at least about 97%, at least about 98%, at least about 99%, at least about 99.5%, or at least about 99.9%) homologous or identical to the 16S rRNA nucleotide sequence of GQ006324.1.1342, New.ReferenceOTU52, HG798451.1.1400, HK557089.3.1395, GQ448336.1.1418, KF842598.1.1394, FJ950694.1.1472, HQ802983.1.1440, GQ448468.1.1366, or JN387556.1.1324 in Table 11.

In certain embodiments, the intestinal microorganism comprises one or more bacteria and/or archaea of one or more Operational Taxonomic Units (OTUs) selected from the group consisting of GQ006324.1.1342, New.ReferenceOTU52, HG798451.1.1400, HK557089.3.1395, GQ448336.1.1418, KF842598.1.1394, FJ950694.1.1472, HQ802983.1.1440, GQ448468.1.1366, and JN387556.1.1324.

In certain embodiments, the intestinal microorganism comprises one or more bacterium comprising a 16S rRNA comprising a nucleotide sequence that is at least about 80% (e.g., at least about 85%, at least about 90%, or at least about 95%, at least about 96%, at least about 97%, at least about 98%, at least about 99%, at least about 99.5%, or at least about 99.9%) homologous or identical to the 16S rRNA nucleotide sequence of JRPJ01000002.1034290.1035971, New.ReferenceOTU45, GQ006324.1.1342, HK555938.1.1357, FJ957551.1.1489, FJ957494.1.1454, New.ReferenceOTU52, FM865905.1.1392, GQ016239.1.1362, HG798451.1.1400, EU461791.1.1414, GU303759.1.1517, New.ReferenceOTU114, AB506154.1.1541, EU774370.1.1398, HK557089.3.1395, HQ807346.1.1456, HQ748204.1.1442, GU179917.1.1382, GQ448336.1.1418, DQ804865.1.1390, GQ491757.1.1361, New.ReferenceOTU56, KF842598.1.1394, HQ802052.1.1445, GX182404.8.1529, FJ950694.1.1472, GQ448506.1.1374, HQ802983.1.1440, DQ793824.1.1370, GQ448468.1.1366, EU774020.1.1361, GQ491183.1.1360, GQ491426.1.1332, GQ493039.1.1311, JN387556.1.1324, and EU775983.1.1288 in Table 11.

In certain embodiments, the intestinal microorganism comprises one or more bacteria and/or archaea of one or more Operational Taxonomic Units (OTUs) selected from the group consisting of JRPJ01000002.1034290.1035971, New.ReferenceOTU45, GQ006324.1.1342, HK555938.1.1357, FJ957551.1.1489, FJ957494.1.1454, New.ReferenceOTU52, FM865905.1.1392, GQ016239.1.1362, HG798451.1.1400, EU461791.1.1414, GU303759.1.1517, New.ReferenceOTU114, AB506154.1.1541, EU774370.1.1398, HK557089.3.1395, HQ807346.1.1456, HQ748204.1.1442, GU179917.1.1382, GQ448336.1.1418, DQ804865.1.1390, GQ491757.1.1361, New.ReferenceOTU56, KF842598.1.1394, HQ802052.1.1445, GX182404.8.1529, FJ950694.1.1472, GQ448506.1.1374, HQ802983.1.1440, DQ793824.1.1370, GQ448468.1.1366, EU774020.1.1361, GQ491183.1.1360, GQ491426.1.1332, GQ493039.1.1311, JN387556.1.1324, and EU775983.1.1288.

In certain embodiments, the intestinal microorganism comprises one or more bacterium comprising a 16S rRNA comprising a nucleotide sequence that is at least about 80% (e.g., at least about 85%, at least about 90%, or at least about 95%, at least about 96%, at least about 97%, at least about 98%, at least about 99%, at least about 99.5%, or at least about 99.9%) homologous or identical to the 16S rRNA nucleotide sequence of GQ449137.1.1391, HK555938.1.1357, GQ358246.1.1466, New.ReferenceOTU82, New.ReferenceOTU52, GQ138615.1.1402, JN681884.1.1409, GU303759.1.1517, New.ReferenceOTU114, EU774881.1.1422, AB469559.1.1551, HK557089.3.1395, EU358719.1.1513, HQ748204.1.1442, GQ338727.1.1397, HQ803964.1.1435, FJ951866.1.1493, EU772870.1.1289, GQ448468.1.1366, EU774020.1.1361, HQ782658.1.1415, DQ794633.1.1395, FN668375.4306350.4307737, or GQ867445.1.1457 in Table 11.

In certain embodiments, the intestinal microorganism comprises one or more bacteria and/or archaea of one or more Operational Taxonomic Units (OTUs) selected from the group consisting of GQ449137.1.1391, HK555938.1.1357, GQ358246.1.1466, New.ReferenceOTU82, New.ReferenceOTU52, GQ138615.1.1402, JN681884.1.1409, GU303759.1.1517, New.ReferenceOTUI14, EU774881.1.1422, AB469559.1.1551, HK557089.3.1395, EU358719.1.1513, HQ748204.1.1442, GQ338727.1.1397, HQ803964.1.1435, FJ951866.1.1493, EU772870.1.1289, GQ448468.1.1366, EU774020.1.1361, HQ782658.1.1415, DQ794633.1.1395, FN668375.4306350.4307737, and GQ867445.1.1457.

### Health Assessment Tools

The presently disclosed subject matter further provides a health assessment tool relating to the microorganisms disclosed herein. In certain embodiments, the health assessment tool is for monitoring intestinal health status or dysbiosis. In certain embodiments, the health assessment tool comprises one or more probe for detecting an amount of one or more microorganisms disclosed herein. In certain embodiments, the health assessment tool comprises a microarray of one or more probe for detecting an amount of one or more microorganism disclosed herein. In certain embodiments, the probe comprises a nucleic acid probe for detecting a signature gene of a microorganism disclosed herein. In certain embodiments, the probe detects a 16S rRNA sequence of a microorganism disclosed herein. In certain embodiments, the probe comprises an antibody. In certain embodiments, the antibody binds to a surface protein/antigen of a microorganism disclosed herein.

In certain embodiments, the amount of the microorganism is measured from a fecal sample of the subject. In certain embodiments, the health assessment tool monitoring intestinal health status or dysbiosis by comparing the amount of the one or more microorganism with a reference amount of the one or more microorganism.

In certain embodiments, the health assessment tool comprises probes for detecting at least about 1, at least about 2, at least about 3, at least about 4, at least about 5, at least about 6, at least about 7, at least about 8, at least about 9, at least about 10, at least about 12, at least about 14, at least about 26 or more microorganisms disclosed herein. In certain embodiments, the health assessment tool comprises probes for detecting about 1, about 2, about 3, about 4, about 5, about 6, about 7, about 8, about 9, about 10, about 12, about 14, or about 26 microorganisms disclosed herein. In certain embodiments, the health assessment tool comprises probes for detecting between about 1 to about 500, between about 1 to about 100, between about 1 to about 26, between about 5 to about 100, between about 5 to about 26, between about 10 to about 26, between about 15 to about 50, or between about 50 to about 100 microorganisms disclosed herein.

In certain embodiments, the one or more microorganism comprises a bacterium comprising a 16S rRNA comprising a nucleotide sequence that is at least about 80% (e.g., at least about 85%, at least about 90%, or at least about 95%, at least about 96%, at least about 97%, at least about 98%, at least about 99%, at least about 99.5%, or at least about 99.9%) homologous or identical to any sequence in Table 11.

### 3. Pharmaceutical Composition

The presently disclosed subject matter provides a pharmaceutical composition for use as a medicament. In certain embodiments, the pharmaceutical composition comprises an effective amount of a bacterium capable of producing a first bile acid. In certain embodiments, the pharmaceutical composition further comprises an effective amount of a second bile acid. In certain embodiments, the bacterium is any bacterium disclosed in the above section. In certain embodiments, the first bile acid and/or the second bile acid is any bile acid disclosed in the above section or a pharmaceutically acceptable salt thereof. In certain embodiments, the first bile acid and the second bile acid are the same. In certain embodiments, the first bile acid and the second bile acid are different.

In certain embodiments, the bacterium comprised in the pharmaceutical composition is between about 1 thousand CFU and about 100 trillion CFU. In certain embodiments, the bacterium is between about 1 thousand CFU and about 1 trillion CFU, between about 1 million CFU and about 1 trillion CFU, between about 100 million CFU and about 100 billion CFU, between about 1 billion CFU and about 1 trillion CFU, between about 1 billion CFU and about 100 billion CFU, between about 100 million CFU and about 100 billion CFU, between about 1 billion CFU and about 50 billion CFU, between about 100 million CFU and about 50 billion CFU, or between about 1 billion CFU and about 10 billion CFU. In certain embodiments, the bacterium comprised in the pharmaceutical composition is at least about 1 thousand CFU, at least about 1 million CFU, at least about 10 million CFU, at least about 100 million CFU, at least about 1 billion CFU, at least about 10 billion CFU, at least about 100 billion CFU or more.

In certain embodiments, the second bile acid comprised in the pharmaceutical composition is between about 1 pg/unit dose and about 1 g/unit dose. In certain embodiments, the second bile acid comprised in the pharmaceutical composition is between about 10 pg/unit dose and about 1 g/unit dose, between about 10 pg/unit dose and about 500 mg/unit dose, between about 100 pg/unit dose and about 500 mg/unit dose, between about 1 mg/unit dose and about 500 mg/unit dose, between about 10 mg/unit dose and about 500 mg/unit dose, between about 100 mg/unit dose and about 500 mg/unit dose, between about 10 mg/unit dose and about 100 mg/unit dose, between about 50 mg/unit dose and about 300 mg/unit dose. In certain embodiments, the second bile acid comprised in the pharmaceutical composition is at least about 1 pg/unit dose, at least about 10 pg/unit dose, at least about 100 pg/unit dose, at least about 1 mg/unit dose, at least about 10 mg/unit dose, at least about 100 mg/unit dose, at least about 1 g/unit dose or more

The presently disclosed subject matter provides a bile acid for the treatment of an intestinal disorder in a dog. In certain embodiments, the bile acid is selected from the group consisting of chenodeoxycholic acid, cholic acid, glycochenodeoxycholic acid, glycocholic acid, taurocholic acid, taurochenodeoxycholic acid, taurodeoxycholic acid, glycodeoxycholic acid, ursodeoxycholic acid, glycoursodeoxycholic acid, tauroursodeoxycholic acid, taurolithocholic acid, alpha-muricholic acid, deoxycholic acid, gamma-muricholic acid, glycolithocholic acid, taurolithocholic acid, lithocholic acid, omega-muricholic acid and any combination thereof. In certain embodiments, the bile acid is a secondary bile acid. In certain embodiments, the secondary bile acid is selected from the group consisting of taurodeoxycholic acid, glycodeoxycholic acid, ursodeoxycholic acid, glycoursodeoxycholic acid, tauroursodeoxycholic acid, taurolithocholic acid, alpha-muricholic acid, deoxycholic acid, gamma-muricholic acid, glycolithocholic acid, taurolithocholic acid, lithocholic acid, omega-muricholic acid and any combination thereof. In certain embodiments, the secondary bile acid is deoxycholic acid and/or lithocholic acid.

In certain embodiments, the pharmaceutical composition is for the treatment of an intestinal disorder in a subject in need thereof. In certain embodiments, the intestinal disorder is selected from the ground consisting of irritable bowel syndrome, constipation, gastritis, colitis, inflammatory bowel disease (IBD), gastrointestinal ulcers, haemorrhagic gastroenteritis, diarrhea, Crohn's disease, ulcerative colitis, enteritis, antibiotic associated diarrhea, acute or chronic enteropathy, necrotizing enterocoloitis, and any combination thereof.

In certain embodiments, the subject is a dog. In certain embodiments, the intestinal disorder is an acute enteropathy or a chronic enteropathy. In certain embodiments, the intestinal disorder is a chronic enteropathy selected from the group consisting of food responsive enteropathy, antibiotic responsive enterophaty, and idiophathic inflammatory bowel disease (IBD).

In certain non-limiting embodiments, the subject is a mammal. In certain embodiments, the subject is a human. In certain embodiments, the subject is a companion animal is a feline (e.g., a domestic cat) or a canine (e.g., a domestic dog).

The exact dose and frequency of administration depends on the particular condition being treated, the age, weight and general physical condition of the particular patient as well as other medication the individual can be taking, as is well known to those skilled in the art. Generally, the daily dose of a pharmaceutical composition disclosed herein can be in the range of between about 0.01 mg to about 1000 mg/day. In certain embodiments, the pharmaceutical composition can be about 0.05 mg to about 1000 mg/day, about 0.1 mg to about 1000 mg/day, about 1 mg to about 500 mg/day, about 0.01 mg to about 500 mg/day, about 0.05 mg to about 200 mg/day, about 1 mg to about 500 mg/day, about 1 mg to about 200 mg/day, about 5 mg to about 500 mg/day, about 50 mg to about 200 mg/day, about 100 mg to about 200 mg/day, about 100 mg to about 1000 mg/day, about 20 mg to about 50 mg/day, or about 20 mg to about 100 mg/day.

In certain embodiments, the pharmaceutical composition disclosed herein can be administered from about 10 times per day to about once per day, from about 5 times per day to about once per day, or from about thrice per day to about once per day. In certain embodiments, the pharmaceutical composition disclosed herein can be administered once per day. In certain embodiments, the pharmaceutical composition disclosed herein can be administered once per two days, once per three days, once per four days, once per five days, once per six days, once a week, once per two weeks, once per three weeks, or once per month.

The pharmaceutical composition disclosed herein can be administered in a variety of forms. In certain embodiments, the pharmaceutical composition disclosed herein can be administered orally, parenterally, rectally. In certain embodiments, orally administered pharmaceutical composition in solid dosage forms can be administered as capsules, dragees, granules, pills, powders, and tablets. In certain embodiments, the pharmaceutical composition can be administered in liquid form as elixirs, emulsions, microemulsions, solutions, suspensions, and syrups. In certain embodiments, parenterally administered pharmaceutical composition can be administered as aqueous or oleaginous solutions or aqueous or oleaginous suspensions, which suspensions comprise crystalline, amorphous, or otherwise insoluble forms of the pharmaceutical composition. In certain embodiments, rectally administered pharmaceutical composition can be administered as creams, gels, lotions, ointments, and pastes.

Depending upon the form of administration, the pharmaceutical composition disclosed herein can be formulated or administered with or without a pharmaceutically acceptable excipient. In certain embodiments, the excipients include encapsulating materials or formulation additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, solution aid, and any combination thereof. In certain embodiments, the pharmaceutical composition disclosed herein is administered without a solubilization aid.

In certain embodiments, the pharmaceutical composition can separately be provided or packaged as kits.

### 4. Food Products

The presently disclosed subject matter provides a food product for improving intestinal health. In certain embodiments, the food product comprises an effective amount of a bacterium capable of producing a first bile acid. In certain embodiments, the food product further comprises an effective amount of a second bile acid. In certain embodiments, the bacterium is any bacterium disclosed in the above section. In certain embodiments, the first bile acid and/or the second bile acid is any bile acid disclosed in the above section or an edible salt thereof. In certain embodiments, the first bile acid and the second bile acid are the same. In certain embodiments, the first bile acid and the second bile acid are different.

In certain embodiments, the food product is a dietary supplement. In certain embodiments, the food product is a human food product. In certain embodiments, the food product is a pet food product, e.g., a cat food product or a dog food product. In certain embodiments, the food product is a dog food product. In certain embodiments, the food product is a pet dietary supplement.

In certain embodiments, the bacterium comprised in the pharmaceutical composition is between about 10 thousand CFU and about 100 trillion CFU. In certain embodiments, the bacterium is between about 1 thousand CFU and about 1 trillion CFU, between about 1 million CFU and about 1 trillion CFU, between about 100 million CFU and about 100 billion CFU, between about 1 billion CFU and about 1 trillion CFU, between about 1 billion CFU and about 100 billion CFU, between about 100 million CFU and about 100 billion CFU, between about 1 billion CFU and about 50 billion CFU, between about 100 million CFU and about 50 billion CFU, or between about 1 billion CFU and about 10 billion CFU. In certain embodiments, the bacterium comprised in the pharmaceutical composition is at least about 1 thousand CFU, at least about 1 million CFU, at least about 10 million CFU, at least about 100 million CFU, at least about 1 billion CFU, at least about 10 billion CFU, at least about 100 billion CFU or more.

In certain embodiments, the second bile acid comprised in the pharmaceutical composition is between about 1 μg/daily serving dose and about 1 g/daily serving dose. In certain embodiments, the second bile acid comprised in the pharmaceutical composition is between about 10 μg/daily serving dose and about 1 g/daily serving dose, between about 10 μg/daily serving dose and about 500 mg/daily serving dose, between about 100 μg/daily serving dose and about 500 mg/daily serving dose, between about 1 mg/daily serving dose and about 500 mg/daily serving dose, between about 10 mg/daily serving dose and about 500 mg/daily serving dose, between about 100 mg/daily serving dose and about 500 mg/daily serving dose, between about 10 mg/daily serving dose and about 100 mg/daily serving dose, between about 50 mg/daily serving dose and about 300 mg/daily serving dose. In certain embodiments, the second bile acid comprised in the pharmaceutical composition is at least about 1 μg/daily serving dose, at least about 10 μg/daily serving dose, at least about 100 μg/daily serving dose, at least about 1 mg/daily serving dose, at least about 10 mg/daily serving dose, at least about 100 mg/daily serving dose, at least about 1 g/daily serving dose or more.

In certain embodiments, a formulation of the presently disclosed subject matter can further comprise an additional active agent. Non-limiting examples of additional active agents that can be present within a formulation of the presently disclosed subject matter include a nutritional agent (e.g., amino acids, peptides, proteins, fatty acids, carbohydrates, sugars, nucleic acids, nucleotides, vitamins, minerals, etc.), a prebiotic, a probiotic, an antioxidant, and/or an agent that improves animal health.

In certain embodiments, the food product comprises one or more probiotic. In certain embodiments, the probiotic is a human probiotic. In certain embodiments, the probiotic is an animal probiotic. In certain embodiments, the animal probiotic is a feline probiotic. In certain embodiments, the animal probiotic is a canine probiotic. In certain embodiments, the probiotic is bifidobacterium, lactic acid bacterium and/or enterococcus. In certain embodiments, the probiotic is selected from the group consisting of any organism from lactic acid bacteria and more specifically from the following bacterial genera; Lactococcus spp., Pediococcus spp., Bifidobacterium spp. (e.g., B. longum B. bifidum, B. pseudolongum, B. animalis), Lactobacillus spp. (e.g. L. bulgaricus, L. acidophilus, L. brevis, L casei, L. rhamnosus, L. plantarum, L. reuteri, L. fermentum, Enterococcus spp. (e.g. E. faecium), Prevotella spp., Fusobacteria spp, Alloprevotella spp, and any combination thereof. In certain embodiments, the probiotic is administered to a companion animal in an amount of from about 1 colony forming unit (CFU) to about 100 billion CFUs per day for the maintenance of GI microflora. In certain embodiments, the probiotic is administered to a companion animal in an amount of from about 1 colony forming unit (CFU) to about 20 billion CFUs per day for the maintenance of GI microflora. In certain embodiments, the probiotic is administered to a companion animal in an amount of from about 1 billion CFUs to about 20 billion CFUs per day for the maintenance of GI microflora. In certain embodiments, the probiotic is administered to a companion animal in amounts of from about 0.01 billion to about 100 billion live bacteria per day. In certain embodiments, the probiotic is administered to a companion animal in amounts of from about 0.1 billion to about 10 billion live bacteria per day.

In certain embodiments, an additional prebiotic can be included, such as fructooligosaccharides (FOS), xylooligosaccharides (XOS), galactooligosaccharides (GOS), glucans, galactans, arabinogalactan, inulin and/or mannooligosaccharides. In certain embodiments, the additional prebiotic is administered in amounts sufficient to positively stimulate the GI microflora and/or cause one or more probiotics to proliferate.

In certain embodiments, the companion animal food product can further contain additives known in the art. In certain embodiments, such additives are present in amounts that do not impair the purpose and effect provided by the presently disclosed subject matter. Examples of contemplated additives include, but are not limited to, substances that are functionally beneficial to improving health, substances with a stabilizing effect, organoleptic substances, processing aids, substances that enhance palatability, coloring substances, and substances that provide nutritional benefits. In certain embodiments, the stabilizing substances include, but are not limited to, substances that tend to increase the shelf life of the product. In certain embodiments, such substances include, but are not limited to, preservatives, synergists and sequestrants, packaging gases, stabilizers, emulsifiers, thickeners, gelling agents, and humectants. In certain embodiments, the emulsifiers and/or thickening agents include, for example, gelatin, cellulose ethers, starch, starch esters, starch ethers, and modified starches.

In certain embodiments, the additives for coloring, palatability, and nutritional purposes include, for example, colorants; iron oxide, sodium chloride, potassium citrate, potassium chloride, and other edible salts; vitamins; minerals; and flavoring. The amount of such additives in a product typically is up to about 5% (dry basis of the product).

In certain embodiments, the companion animal food product is a dietary supplement. In certain embodiments, the dietary supplements include, for example, a feed used with another feed to improve the nutritive balance or performance of the total. In certain embodiments, the supplements include compositions that are fed undiluted as a supplement to other feeds, offered free choice with other parts of an animal's ration that are separately available, or diluted and mixed with an animal's regular feed to produce a complete feed. The AAFCO, for example, provides a discussion relating to supplements in the American Feed Control Officials, Incorp. Official Publication, p. 220 (2003). Supplements can be in various forms including, for example, powders, liquids, syrups, pills, tablets, encapsulated compositions, etc.

In certain embodiments, the companion animal food product is a treat. In certain embodiments, treats include, for example, compositions that are given to an animal to entice the animal to eat during a non-meal time. In certain embodiments, the companion animal food product is a treat for canines include, for example, dog bones. Treats can be nutritional, wherein the product comprises one or more nutrients, and can, for example, have a composition as described above for food. Non-nutritional treats encompass any other treats that are non-toxic.

In certain embodiments, a bacterium and/or a bile acid of the presently disclosed subject matter can be incorporated into the composition during the processing of the formulation, such as during and/or after mixing of other components of the product. Distribution of these components into the product can be accomplished by conventional means.

In certain embodiments, companion animal food products of the presently disclosed subject matter can be prepared in a canned or wet form using conventional companion animal food processes. In certain embodiments, ground animal (e.g., mammal, poultry, and/or fish) proteinaceous tissues are mixed with the other ingredients, such as milk fish oils, cereal grains, other nutritionally balancing ingredients, special purpose additives (e.g., vitamin and mineral mixtures, inorganic salts, cellulose and beet pulp, bulking agents, and the like); and water that sufficient for processing is also added. These ingredients are mixed in a vessel suitable for heating while blending the components. Heating of the mixture can be effected using any suitable manner, such as, for example, by direct steam injection or by using a vessel fitted with a heat exchanger. Following the addition of the last ingredient, the mixture is heated to a temperature range of from about 500° F. to about 2120° F. Temperatures outside this range are acceptable but can be commercially impractical without use of other processing aids. When heated to the appropriate temperature, the material will typically be in the form of a thick liquid. The thick liquid is filled into cans. A lid is applied, and the container is hermetically sealed. The sealed can is then placed into conventional equipment designed to sterilize the contents. This is usually accomplished by heating to temperatures of greater than about 2300° F. for an appropriate time, which is dependent on, for example, the temperature used and the composition.

In certain embodiments, companion animal food products of the presently disclosed subject matter can be prepared in a dry form using conventional processes. In certain embodiments, dry ingredients, including, for example, animal protein sources, plant protein sources, grains, etc., are ground and mixed together. In certain embodiments, moist or liquid ingredients, including fats, oils, animal protein sources, water, etc., are then added to and mixed with the dry mix. In certain embodiments, the mixture is then processed into kibbles or similar dry pieces. In certain embodiments, the companion animal food product is kibble. In certain embodiments, kibble is formed using an extrusion process in which the mixture of dry and wet ingredients is subjected to mechanical work at a high pressure and temperature and forced through small openings and cut off into kibble by a rotating knife. In certain embodiments, the wet kibble is then dried and optionally coated with one or more topical coatings which can include, for example, flavors, fats, oils, powders, and the like. In certain embodiments, kibble can also be made from the dough using a baking process, rather than extrusion, wherein the dough is placed into a mold before dry-heat processing.

In certain embodiments, treats of the presently disclosed subject matter can be prepared by, for example, an extrusion or baking process similar to those described above for dry food.

The presently disclosed subject matter provides a diet for increase a population of a bacterium capable of producing a bile acid in a companion animal. In certain embodiments, the diet comprises protein, fat, crude fiber, total dietary fiber, carbohydrate, calcium, phosphorus, sodium, chloride, potassium, magnesium, iron, copper, manganese, zinc, iodine, selenium, vitamin A, vitamin D3, vitamin E, vitamin C, thiamine (vitamin B1), riboflavin (vitamin B2), pantothenic acid, niacin, pyridoxine (vitamin B6), folic acid, biotin, cobalannin (vitamin B12), choline, arginine, lysine, methionine, cystine, taurine, linoleic acid, arachidonic acid, Omega-6 fatty acids, Omega-3 fatty acids, EPA, and/or DHA.

In certain embodiments, the subject is a dog. In certain embodiments, the diet is a Royal Canin Veterinary Diet. In certain embodiments, the diet is selected from the group consisting of Ultamino, Hydrolyzed Protein Adult HP Dry, Hydrolyzed Protein Wet, Hydrolyzed Protein Adult PS Dry, Hydrolyzed Protein Moderate Calorie Dry, Hydrolyzed Protein Small Dog Dry, Hydrolyzed protein Treats, and any combination thereof.

In certain embodiments, the bacterium comprises a bile acid-inducible operon (bai operon). In certain embodiments, the bacterium is C. hiranonis, C. scindens or combination thereof. In certain embodiments, the bacterium is C. hiranonis.

The presently disclosed subject matter provides a Royal Canin Veterinary Diet for the treatment of an intestinal disorder in a dog, wherein the dog comprises a first amount of a first intestinal microorganism and/or a second amount of a second intestinal microorganism, and wherein the first amount of the first intestinal microorganism is higher than a first reference amount of the first intestinal microorganism, and/or the second amount of the second intestinal microorganism is lower than a second reference amount of the second intestinal microorganism.

In certain embodiments, the first intestinal microorganism is selected from the group consisting of New.ReferenceOTU45, JRPJ01000002.1034290.1035971, KF842598.1.1394, JF920309.1.1340, FJ978526.1.1378, New.ReferenceOTU54, HQ793763.1.1451, DQ113765.1.1450, DQ797046.1.1403, ACBW01000012.3536.5054, JN387556.1.1324, New.ReferenceOTU52, JQ208053.1.1336, and any combination thereof. In certain embodiments, the second intestinal microorganism is selected from the group consisting of HK693629.1.1491, GQ493166.1.1359, GQ491426.1.1332, FJ957494.1.1454, GQ449092.1.1375, GQ448486.1.1387, AMCI01001631.34.1456, HK555938.1.1357, and any combination thereof.

### 5. Treatment Methods

In certain non-limiting embodiments, the presently disclosed subject matter provides for a method for improving intestinal health and/or treating an intestinal disorder of a subject in need thereof. In certain embodiments, the method can improve immunity, digestive function and/or decrease inflammation of a companion animal.

In certain non-limiting embodiments, the presently disclosed subject matter provides for a method for determining susceptibility of an intestinal disorder in a companion animal. In certain embodiments, the method comprises:
a) measuring a first amount of a first intestinal microorganism and/or a second amount of a second intestinal microorganism in the companion animal;
b) comparing the first amount of the first intestinal microorganism with a first reference amount of the first intestinal microorganism, and/or comparing the second amount of the second intestinal microorganism with a second reference amount of the second intestinal microorganism, wherein the reference amounts of the intestinal microorganisms are determined based on the amounts of the intestinal microorganisms in a plurality of healthy companion animals; and
c) determining that the companion animal is susceptible of an intestinal disorder, when the first amount of the intestinal microorganism is higher than the first reference amount of the first intestinal microorganism, and/or when the second amount of the second intestinal microorganism is lower than the second reference amount of the second intestinal microorganism.

In certain embodiments, the first intestinal microorganism comprises one or more bacterium comprising a 16S rRNA comprising a nucleotide sequence that is at least about 90% homologous or identical to the 16S rRNA nucleotide sequence of HQ802983.1.1440, GQ449092.1.1375, GQ448744.1.1393, KF842598.1.1394, HG798451.1.1400, New.ReferenceOTU52, HK555938.1.1357, FJ957494.1.1454, FN667392.1.1495, New.ReferenceOTU54, HQ760911.1.1437, GQ006324.1.1342, FJ950694.1.1472, FM865905.1.1392, FJ506371.1.1371, FJ957528.1.1445, JF712675.1.1540, New.ReferenceOTU82, AB009242.1.1451, HQ751549.1.1448, AB506370.1.1516, DQ057365.1.1393, FN667422.1.1495, AJ270486.1.1241, FN668375.4306350.4307737, GQ867426.1.1494, GX182404.8.1529, JF224013.1.1362, GQ448246.1.1389, JF807116.1.1260, KC245406.1.1465, FN667084.1.1493, EU470512.1.1400, EU768569.1.1352, AY239462.1.1500, KC504009.1.1465, FM179752.1.1686, New.ReferenceOTU114, HK557089.3.1395, JQ208181.1.1352, HQ803964.1.1435, AM276759.1.1484, JN387556.1.1324, GQ448486.1.1387, HK694029.9.1487, HQ754680.1.1441, FN563300.1.1447, FP929060.3837.5503, GQ448506.1.1374, Enterococcus durans, C. perfringens, or E. coli.

In certain embodiments, the first intestinal microorganism is selected from the group consisting of HQ802983.1.1440, GQ449092.1.1375, GQ448744.1.1393, KF842598.1.1394, HG798451.1.1400, New.ReferenceOTU52, HK555938.1.1357, FJ957494.1.1454, FN667392.1.1495, New.ReferenceOTU54, HQ760911.1.1437, GQ006324.1.1342, FJ950694.1.1472, FM865905.1.1392, FJ506371.1.1371, FJ957528.1.1445, JF712675.1.1540, New.ReferenceOTU82, AB009242.1.1451, HQ751549.1.1448, AB506370.1.1516, DQ057365.1.1393, FN667422.1.1495, AJ270486.1.1241, FN668375.4306350.4307737, GQ867426.1.1494, GX182404.8.1529, JF224013.1.1362, GQ448246.1.1389, JF807116.1.1260, KC245406.1.1465, FN667084.1.1493, EU470512.1.1400, EU768569.1.1352, AY239462.1.1500, KC504009.1.1465, FM179752.1.1686, New.ReferenceOTU114, HK557089.3.1395, JQ208181.1.1352, HQ803964.1.1435, AM276759.1.1484, JN387556.1.1324, GQ448486.1.1387, HK694029.9.1487, HQ754680.1.1441, FN563300.1.1447, FP929060.3837.5503, GQ448506.1.1374, Enterococcus durans, C. perfringens, E. coli and any combination thereof.

In certain embodiments, the first intestinal microorganism is C. perfringens, E. coli and any combination thereof.

In certain embodiments, the second intestinal microorganism comprises one or more bacterium comprising a 16S rRNA comprising a nucleotide sequence that is at least about 90% homologous or identical to the 16S rRNA nucleotide sequence of EU774020.1.1361, HQ793763.1.1451, HQ792787.1.1438, New.ReferenceOTU109, HQ792778.1.1436, or DQ113765.1.1450.

In certain embodiments, the second intestinal microorganism is selected from the group consisting of EU774020.1.1361, HQ793763.1.1451, HQ792787.1.1438, New.ReferenceOTU109, HQ792778.1.1436, DQ113765.1.1450, and any combination thereof.

In certain embodiments, the method further comprises providing a customized recommendation of a treatment regimen, and/or further monitoring the intestinal microorganism, when the first amount of the first intestinal microorganism is lower than the first reference amount of the first intestinal microorganism, and/or when the second amount of the second intestinal microorganism is higher than the second reference amount of the second intestinal microorganism.

In certain non-limiting embodiments, the presently disclosed subject matter provides for a method for determining responsiveness of a companion animal having an intestinal disorder to a diet. In certain embodiments, the method comprises:
a) measuring a first amount of a first intestinal microorganism and/or a second amount of a second intestinal microorganism in the companion animal;
b) comparing the first amount of the first intestinal microorganism with a first reference amount of the first intestinal microorganism, and/or comparing the second amount of the second intestinal microorganism with a second reference amount of the second intestinal microorganism, wherein the reference amounts of the intestinal microorganisms are determined based on the amounts of the intestinal microorganisms in a plurality of healthy companion animals; and
c) determining that the companion animal is responsive to the diet, when the first amount of the intestinal microorganism is higher than the first reference amount of the first intestinal microorganism, and/or when the second amount of the second intestinal microorganism is lower than the second reference amount of the second intestinal microorganism, or determining that the companion animal is non-responsive to the diet, when the first amount of the intestinal microorganism is lower than the first reference amount of the first intestinal microorganism, and/or when the second amount of the second intestinal microorganism is higher than the second reference amount of the second intestinal microorganism.

In certain embodiments, the first intestinal microorganism comprises one or more bacterium comprising a 16S rRNA comprising a nucleotide sequence that is at least about 90% homologous or identical to the 16S rRNA nucleotide sequence of ReferenceOTU45, JRPJ01000002.1034290.1035971, KF842598.1.1394, JF920309.1.1340, FJ978526.1.1378, New.ReferenceOTU54, HQ793763.1.1451, DQ113765.1.1450, DQ797046.1.1403, ACBW01000012.3536.5054, JN387556.1.1324, New.ReferenceOTU52, JQ208053.1.1336, and any combination thereof. In certain embodiments, the second intestinal microorganism is selected from the group consisting of HK693629.1.1491, GQ493166.1.1359, GQ491426.1.1332, FJ957494.1.1454, GQ449092.1.1375, GQ448486.1.1387, AMCI01001631.34.1456, or HK555938.1.1357.

In certain embodiments, the first intestinal microorganism is selected from the group consisting of New.ReferenceOTU45, JRPJ01000002.1034290.1035971, KF842598.1.1394, JF920309.1.1340, FJ978526.1.1378, New.ReferenceOTU54, HQ793763.1.1451, DQ113765.1.1450, DQ797046.1.1403, ACBW01000012.3536.5054, JN387556.1.1324, New.ReferenceOTU52, JQ208053.1.1336, and any combination thereof.

In certain embodiments, the second intestinal microorganism comprises one or more bacterium comprising a 16S rRNA comprising a nucleotide sequence that is at least about 90% homologous or identical to the 16S rRNA nucleotide sequence of HK693629.1.1491, GQ493166.1.1359, GQ491426.1.1332, FJ957494.1.1454, GQ449092.1.1375, GQ448486.1.1387, AMCI01001631.34.1456, or HK555938.1.1357.

In certain embodiments, the second intestinal microorganism is selected from the group consisting of HK693629.1.1491, GQ493166.1.1359, GQ491426.1.1332, FJ957494.1.1454, GQ449092.1.1375, GQ448486.1.1387, AMCI01001631.34.1456, HK555938.1.1357, and any combination thereof.

In certain embodiments, the method further comprises administering the diet to the companion animal when companion animal is determined as responsive to the diet. In certain embodiments, the method further comprises administering the diet, a steroid and optionally an antibiotic to the companion animal when companion animal is determined as non-responsive to the diet.

In certain embodiments, the determination in step c) occurs before administering the diet or the diet, the steroid and optionally the antibiotic to the companion animal.

In certain non-limiting embodiments, the presently disclosed subject matter provides for a method for determining effectiveness of a diet for treating an intestinal disorder in a companion animal. In certain embodiments, the method comprises:
a) measuring a first amount of a first intestinal microorganism and/or a second amount of a second intestinal microorganism in the companion animal before or after administering a diet to a companion animal for treating an intestinal disorder;
b) comparing the first amount of the first intestinal microorganism with a first reference amount of the first intestinal microorganism, and/or comparing the second amount of the second intestinal microorganism with a second reference amount of the second intestinal microorganism, wherein the reference amounts of the intestinal microorganisms are determined based on the amounts of the intestinal microorganisms in a plurality of healthy companion animals; and
c) determining that the diet is effective for treating an intestinal disorder, when the first amount of the intestinal microorganism is higher than the first reference amount of the first intestinal microorganism, and/or when the second amount of the second intestinal microorganism is lower than the second reference amount of the second intestinal microorganism, or determining that the diet is ineffective for treating an intestinal disorder, when the first amount of the intestinal microorganism is lower than the first reference amount of the first intestinal microorganism, and/or when the second amount of the second intestinal microorganism is higher than the second reference amount of the second intestinal microorganism.

In certain embodiments, the first intestinal microorganism comprises one or more bacterium comprising a 16S rRNA comprising a nucleotide sequence that is at least about 90% homologous or identical to the 16S rRNA nucleotide sequence of HK557089.3.1395, or GQ448336.1.1418. In certain embodiments, the first intestinal microorganism is selected from the group consisting of HK557089.3.1395, GQ448336.1.1418, and combination thereof.

In certain embodiments, the second intestinal microorganism comprises one or more bacterium comprising a 16S rRNA comprising a nucleotide sequence that is at least about 90% homologous or identical to the 16S rRNA nucleotide sequence of KF842598.1.1394, GQ006324.1.1342, HQ802983.1.1440, JN387556.1.1324, FJ950694.1.1472, HG798451.1.1400, New.ReferenceOTU52, or GQ448468.1.1366.

In certain embodiments, the second intestinal microorganism is selected from the group consisting of KF842598.1.1394, GQ006324.1.1342, HQ802983.1.1440, JN387556.1.1324, FJ950694.1.1472, HG798451.1.1400, New.ReferenceOTU52, GQ448468.1.1366, and any combination thereof.

In certain embodiments, the method further comprises administering the diet to the companion animal when companion animal is determined as responsive to the diet. In certain embodiments, the method further comprises administering the diet, a steroid and optionally an antibiotic to the companion animal when companion animal is determined as non-responsive to the diet.

In certain embodiments, the determination in step c) occurs before administering the diet or the diet, the steroid and optionally the antibiotic to the companion animal.

In certain embodiments, the reference amount of an intestinal microorganism derived from a mean amount of the intestinal microorganism in a plurality of healthy companion animals. In certain embodiments, the amount of the intestinal bacterium is measured from a fecal sample of the subject.

In certain embodiments, the method comprises administering to the subject an effective amount of a presently disclosed pharmaceutical composition, an effective amount of a presently disclosed food product, or any combination thereof. In certain embodiments, the method further comprises monitoring an intestinal microorganism in the subject. In certain embodiments, the intestinal microorganism is sampled from a fecal sample of the subject.

In certain embodiments, the intestinal microorganism is selected from the group consisting of Ruminococcus, Alloprevotella, Allisonella, Anaerostipes, Anaerobiospirillum, Bacteroides, Blautia, Clostridium sensu stricto 1, Collinsella, Coprococcus 1, Corynebacterium 1, Campylobacter, Enterococcus, Erysipelatoclostridium, Escherichia-Shigella, Faecalitalea, Fusobacterium, Helicobacter, Intestinibacter, Lachnoclostridium, Lactobacillus, Megasphaera, Methanobrevibacter, Parabacteroides, Porphyromonas, Phascolarctobacterium, Peptoclostridium, Prevotellaceae UCG-001, Pseudocitrobacter, Ruminiclostridium 9, Sarcina, Streptococcus, Succinivibrio, Treponema 2, Turicibacter, Tyzzerella, Tyzzerella 4 and any combination thereof.

In certain embodiments, the intestinal microorganism is selected from the group consisting of Escherichia-Shigella, Clostridium sensu stricto 1, Enterococcus, Fusobacterium and any combination thereof. In certain embodiments, the intestinal microorganism is E. coli, C. perfringens or combination thereof.

In certain embodiments, an amount of the intestinal bacterium is decreased after administration of the pharmaceutical composition. In certain embodiments, an amount of the intestinal bacterium is decreased within about 14 days after administration of the pharmaceutical composition. In certain embodiments, an amount of the intestinal bacterium is decreased within about 21 days, within about 14 days, within about 12 days, within about 10 days, within about 7 days, within about 6 days, within about 5 days, within about 4 days, within about 3 days, within about 2 days, or within about 1 day after administration of the pharmaceutical composition. In certain embodiments, an amount of the intestinal bacterium is decreased within about 1 day to about 21 days, within about 1 days to about 14 days, within about 3 days to about 14 days, within about 5 days to about 14 days, within about 7 days to about 14 days, within about 10 days to about 14 days, or within about 7 days to about 21 days after administration of the pharmaceutical composition.

In certain embodiments, the intestinal microorganism is selected from the group consisting of C. hiranonis, C. scindens, Veillonellaceae, Streptococcaceae, Bacteroides, Fusobacterium, Collinsella, Sarcina, Clostridium sensu stricto 1, Faecalitalea, Streptococcus, Erysipelatoclostridium, Megasphaera, Blautia, Alloprevotella, Peptoclostridium, and any combination thereof. In certain embodiments, the intestinal microorganism is C. hiranonis, C. scindens or combination thereof.

In certain embodiments, an amount of the intestinal microorganism is increased after administration of the pharmaceutical composition and/or the food product. In certain embodiments, the amount of the intestinal microorganism is increased within about 14 days after administration of the pharmaceutical composition and/or the food product. In certain embodiments, an amount of the intestinal bacterium is increased within about 21 days, within about 14 days, within about 12 days, within about 10 days, within about 7 days, within about 6 days, within about 5 days, within about 4 days, within about 3 days, within about 2 days, or within about 1 day after administration of the pharmaceutical composition. In certain embodiments, an amount of the intestinal bacterium is increased within about 1 day to about 21 days, within about 1 days to about 14 days, within about 3 days to about 14 days, within about 5 days to about 14 days, within about 7 days to about 14 days, within about 10 days to about 14 days, or within about 7 days to about 21 days after administration of the pharmaceutical composition.

In certain embodiments, the method comprises:
a) measuring a first amount of one or more intestinal microorganism in the subject;
b) administering a treatment regimen to the subject for treating the intestinal disorder;
c) measuring a second amount of the intestinal microorganism in the subject after step b); and
d) continuing administering the treatment regimen, when the second amount of the intestinal microorganism is reduced compared to the first amount of the intestinal microorganism.

In certain embodiments, the second amount of the intestinal microorganism is measured between about 7 days and about 14 days after step b). In certain embodiments, an amount of the intestinal microorganism is decreased within about 21 days, within about 14 days, within about 12 days, within about 10 days, within about 7 days, within about 6 days, within about 5 days, within about 4 days, within about 3 days, within about 2 days, or within about 1 day after step b). In certain embodiments, an amount of the intestinal bacterium is decreased within about 1 day to about 21 days, within about 1 days to about 14 days, within about 3 days to about 14 days, within about 5 days to about 14 days, within about 7 days to about 14 days, within about 10 days to about 14 days, or within about 7 days to about 21 days after step b).

In certain embodiments, the intestinal microorganism is measured from a fecal sample of the subject.

In certain embodiments, the method comprises:
a) measuring the first amount of one or more intestinal microorganism in the subject;
b) comparing the first amount of the intestinal microorganism with a reference amount of the intestinal microorganism, wherein the reference amount of the intestinal microorganism is determined based on the amount of the intestinal microorganism in a plurality of healthy subjects;
c) providing a customized recommendation of a treatment regimen, and/or further monitoring the intestinal microorganism, when the first amount of the intestinal microorganism is above the reference amount of the intestinal microorganism.

In certain embodiments, the method further comprises measuring a second amount of the intestinal microorganism in the subject after step c), and continuing the treatment regimen when the second amount of the intestinal microorganism is decreased compared to the first amount of the intestinal microorganism and is above the reference amount of the intestinal microorganism.

In certain embodiments, the second amount of the intestinal bacterium is measured between about 7 days and about 14 days after step c). In certain embodiments, an amount of the intestinal microorganism is decreased within about 21 days, within about 14 days, within about 12 days, within about 10 days, within about 7 days, within about 6 days, within about 5 days, within about 4 days, within about 3 days, within about 2 days, or within about 1 day after step b). In certain embodiments, an amount of the intestinal microorganism is decreased within about 1 day to about 21 days, within about 1 days to about 14 days, within about 3 days to about 14 days, within about 5 days to about 14 days, within about 7 days to about 14 days, within about 10 days to about 14 days, or within about 7 days to about 21 days after step c).

In certain embodiments, the intestinal microorganism is measured from a fecal sample of the subject.

In certain embodiments, the intestinal microorganism is selected from the group consisting of Ruminococcus, Alloprevotella, Allisonella, Anaerostipes, Anaerobiospirillum, Bacteroides, Blautia, Clostridium sensu stricto 1, Collinsella, Coprococcus 1, Corynebacterium 1, Campylobacter, Enterococcus, Erysipelatoclostridium, Escherichia-Shigella, Faecalitalea, Fusobacterium, Helicobacter, Intestinibacter, Lachnoclostridium, Lactobacillus, Megasphaera, Methanobrevibacter, Parabacteroides, Porphyromonas, Phascolarctobacterium, Peptoclostridium, Prevotellaceae UCG-001, Pseudocitrobacter, Ruminiclostridium 9, Sarcina, Streptococcus, Succinivibrio, Treponema 2, Turicibacter, Tyzzerella, Tyzzerella 4 and any combination thereof. In certain embodiments, the intestinal microorganism is selected from the group consisting of Escherichia-Shigella, Clostridium sensu stricto 1, Enterococcus, Fusobacterium and any combination thereof. In certain embodiments, the intestinal microorganism is E. coli, C. perfringens or combination thereof.

In certain embodiments, the treatment regimen comprises administering an effective amount of a presently disclosed pharmaceutical composition, an effective amount of a presently disclosed food product, or any combination thereof.

In certain non-limiting embodiments, the subject is a mammal. In certain embodiments, the subject is a human. In certain embodiments, the subject is a companion animal is a feline (e.g., a domestic cat) or a canine (e.g., a domestic dog). In certain non-limiting embodiments, the companion animal is at risk of an intestinal disorder and/or inflammation. In certain non-limiting embodiments, the companion animal is not known to be at risk of an intestinal disorder and/or inflammation. In certain non-limiting embodiments, the companion animal has an intestinal disorder and/or inflammation. In certain non-limiting embodiments, the companion animal is not known to have an intestinal disorder and/or inflammation. In certain non-limiting embodiments, the companion animal is under a treatment for a digestive disorder and/or inflammation. In certain non-limiting embodiments, the treatment is a dietary therapy. In certain embodiments, the companion animal is a dog. In certain embodiments, the intestinal disorder is an acute enteropathy or a chronic enteropathy. In certain embodiments, the intestinal disorder is a chronic enteropathy selected from the group consisting of food responsive enteropathy, antibiotic responsive enterophaty, and idiophathic inflammatory bowel disease (IBD).

In certain embodiments, the pharmaceutical composition and/or the food product can be administered to a subject from 20 times per day to once per day, from 10 times per day to once per day, or from 5 times per day to once per day. In certain embodiments, the pharmaceutical composition and/or the food product can be administered to a subject once per day, twice per day, thrice per day, 4 times per day, 5 times per day, 6 times per day, 7 times per day, 8 times per day, 9 times per day, 10 or more times per day. In certain embodiments, the pharmaceutical composition and/or the food product can be administered to a subject once per two days, once per three days, once per four days, once per five days, once per six days, once a week, once per two weeks, once per three weeks, or once per month. In certain embodiments, the food product can be administered to an animal in a constant manner, e.g., where the animal grazes on a constantly available supply of the subject food product.

In certain embodiments, the dosage of the pharmaceutical composition is between about 1 mg/kg body weight per day and about 5000 mg/kg body weight per day. In certain embodiments, the dosage of the pharmaceutical composition is between about 5 mg/kg body weight per day and about 1000 mg/kg body weight per day, between about 10 mg/kg body weight per day and about 500 mg/kg body weight per day, between about 10 mg/kg body weight per day and about 250 mg/kg body weight per day, between about 10 mg/kg body weight per day and about 200 mg/kg body weight per day, between about 20 mg/kg body weight per day and about 100 mg/kg body weight per day, between about 20 mg/kg body weight per day and about 50 mg/kg body weight per day or any intermediate range thereof. In certain embodiments, the dosage of the pharmaceutical composition is at least about 1 mg/kg body weight per day, at least about 5 mg/kg body weight per day, at least about 10 mg/kg body weight per day, at least about 20 mg/kg body weight per day, at least about 50 mg/kg body weight per day, at least about 100 mg/kg body weight per day, at least about 200 mg/kg body weight per day or more. In certain embodiments, the dosage of the pharmaceutical composition is no more than about 5 mg/kg body weight per day, no more than about 10 mg/kg body weight per day, no more than about 20 mg/kg body weight per day, no more than about 50 mg/kg body weight per day, no more than about 100 mg/kg body weight per day, no more than about 200 mg/kg body weight per day, no more than about 500 mg/kg body weight per day or more.

In certain embodiments, the amount of the pharmaceutical composition and/or the food product decreases over the course of feeding a companion animal. In certain embodiments, the concentration of the pharmaceutical composition and/or the food product increases over the course of feeding a companion animal. In certain embodiments, the concentration of the pharmaceutical composition and/or the food product is modified based on the age of the companion animal.

### 6. Kits

The presently disclosed subject matter provides kits for treating and/or preventing an intestinal disorder in a subject. In certain embodiments, the kit comprises an effective amount of the presently disclosed pharmaceutical composition, dietary supplement, functional food, food product, diet or any combination thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, the pharmaceutical composition, dietary supplement, functional food, food product, and/or diet are provided together with instructions for administering the same to a subject having or at risk of developing an intestinal disorder. The instructions generally include information about the use of the pharmaceutical composition, dietary supplement, functional food, food product, diet for the treatment and/or prevention of an intestinal disorder. In certain embodiments, the instructions include at least one of the following: description of the therapeutic agent; dosage schedule and administration for treatment or prevention of an intestinal disorder or symptoms thereof, precautions; warnings; indications; counter-indications; over-dosage information; adverse reactions; animal pharmacology; clinical studies; and/or references. The instructions can be printed directly on the container (when present), or as a label applied to the container, or as a separate sheet, pamphlet, card, or folder supplied in or with the container.

Advantageously, the kit can be packaged in per use groupings such that, for example, a daily prescription of each component can be identified in order to enhance patient compliance. Sets of the pharmaceutical composition can be identified in a variety of ways. For example, in certain embodiments, a set of the pharmaceutical composition, dietary supplement, functional food, food product, diet can be identified on the package containing the same. In certain embodiments, external instructions can be provided with a set or sets of the pharmaceutical composition, dietary supplement, functional food, food product, diet that, for example, identify a grouping and instruct a patient/animal owner appropriate times to take the pharmaceutical composition, dietary supplement, functional food, food product, diet of the kit.

### EXAMPLES

The presently disclosed subject matter will be better understood by reference to the following Example, which is provided as exemplary of the present disclosure, and not by way of limitation.

### Example 1

### Introduction

Although a wide range of environmental factors have been shown to influence the microbiome, diet is regarded as one of the most potent modulators of the composition and function of the gut-resident microbial community in healthy humans and other mammals^{7,8} and can act as both a risk factor and a treatment modality for IBD^{9,10} Epidemiologic data and studies in mice have shown that diets high in fat and/or low in fiber, as well as dietary additives such as emulsifiers, are either risk factors for IBD, or in some cases can directly compromise intestinal barrier function leading to disease¹¹⁻¹³ Diet can be also leveraged to treat IBD, with perhaps the clearest example of this being the use of exclusive enteral nutrition (EEN) as first-line therapy for pediatric Crohn's disease¹⁴. High remission rates (≥60%) are observed following EEN and, compared to corticosteroids, EEN achieves better patient growth along with a reduction in biomarkers of disease, such as fecal calprotectin and C-reactive protein¹⁵⁻¹⁸ Interestingly, EEN has a marked effect on the microbiome, but the precise nature of this effect has been complicated to discern, with some studies reporting reduced microbiome diversity following EEN therapy¹⁹⁻²¹, while others point to relatively unchanged^{22,23} or increased diversity²⁴.

The mechanisms by which diet impacts the gut microbiome to ameliorate IBD symptoms are unclear and are complicated to dissect from human subject research were diet is challenging to control, necessitating either retrospective studies in conjunction with extensive food intake surveys²⁵, controlled feeding studies²⁶, or focusing on populations with different subsistence practices²⁷⁻²⁹. In contrast, mouse models of colitis have yielded important insights into the pathophysiology of intestinal inflammation, but these often involve chemical or genetic perturbation, rather than spontaneous disease development. Moreover, the ubiquitous use of autoclaved food and acidified water for mouse husbandry, together with the tendency for cage effects to dominate in mouse microbiome studies, raises concerns about clinical relevance of diet-microbiome studies in these models of colitis. As companion animals, dogs share the same environment as humans, and spontaneously develop a chronic enteritis that clinically resembles human IBD, including similar gastrointestinal pathology, responsiveness to similar treatments^{30,31}, involvement of some of the same susceptibility loci³⁰⁻³², and shared disease-associated microbial taxa³³⁻³⁵ Intriguingly, after treatment with dietary therapy, over 50% of dogs with chronic enteritis enter a long-lasting state of remission³⁶, making the use of prescription diets the first-line treatment for IBD in companion animal medicine. A recent metagenomic study produced a catalog of over one million taxonomically and functionally annotated microbial genes from the canine gut and showed that compared to other mammals, such as the mouse and pig the microbial environment in dogs most closely resembles that of humans³⁷. Furthermore, the canine microbiome was markedly altered by diet change in a manner that resembles what has been reported in humans³⁷. Together, these data argue that dogs are an ideal animal model in which to study diet-microbiome interactions in the context of intestinal disease.

Despite the fact that the gut microbiome has been implicated in IBD pathogenesis, and that diet profoundly alters the microbiome and can be used to manage symptoms of IBD, there is limited insight into the mechanisms by which this occurs. In this study, treatment-naive dogs were examined with chronic enteritis and changes in their fecal microbial community structure and metabolites in response to treatment were monitored. By comparing changes over time in diet-responsive dogs, versus animals that failed diet therapy and required subsequent combination therapy, it was shown that diet induces rapid remission by shaping the community structure and re-programming the metabolic function of the microbiome. Notably, it was demonstrated that secondary bile acids, likely produced by clostridia, are involved in the diet induced alterations of microbiota community by inhibiting the growth of potential pathogens. These findings provide a general mechanism by which diet can modulate microbial communities to reduce GI disease.

### Methods

### Diagnosis and Treatment of Canine Chronic Enteritis (CCE)

Client-owned animals presenting with clinical signs of CCE were screened at the Ryan Veterinary Hospital of the University of Pennsylvania. All animal work was carried out in accordance within the guidelines of the University of Pennsylvania IACUC (Protocol 805283), and signed owner consent was obtained before enrollment. Dogs were screened if they had any one of the following clinical signs for ≥3 weeks' duration: vomiting, diarrhea or weight loss despite adequate caloric intake. Dogs were excluded from screening if they had been treated with a hydrolyzed protein diet, antibiotics, corticosteroids or probiotics within the previous two weeks. At the time of screening, the following were performed on each animal: complete physical examination, routine fecal screening (including zinc sulfate flotation for parasite identification, gram stain and culture for Salmonella spp. and Campylobacter spp.), complete blood count, serum biochemical profile, serum measurement of canine trypsin-like immunoreactivity, cobalamin and folate, urinalysis, abdominal ultrasound examination, and disease severity scoring using the Canine Chronic Enteropathy Clinical Activity Index (CCECAI)³⁶. If these initial screening tests failed to identify a cause for the clinical signs, upper and/or lower gastrointestinal endoscopy with mucosal biopsies was performed. Biopsies were fixed in formalin, embedded in paraffin, and sections were stained with hematoxylin and eosin, and slides were examined by a board-certified veterinary pathologist. Dogs were enrolled if histopathology revealed intestinal inflammation with no identifiable underlying cause (such as infectious agents). Dogs were excluded if another histopathologic diagnosis was identified.

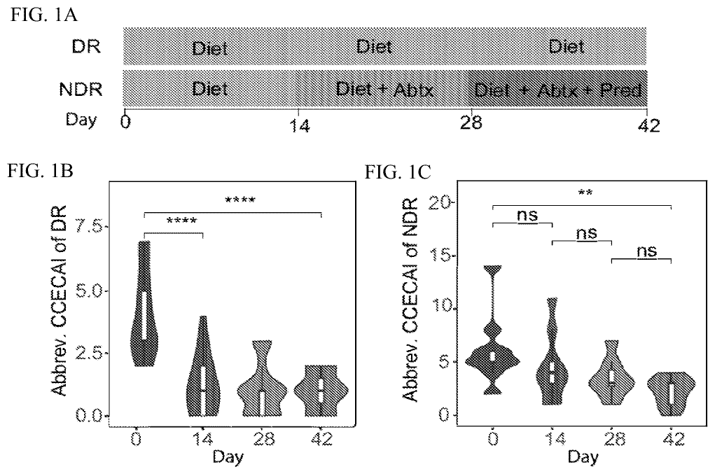

Three 14-day treatment tiers were included in the trial (FIG. 1A), and dogs were evaluated for a therapeutic response at the conclusion of each tier using CCECAI. Remission was determined using an abbreviated CCECAI that included scores to the first five indices (attitude/activity, appetite, vomiting, stool consistency, and stool frequency), and was defined as an abbreviated CCECAI score ≤2, with no score >1 for any of the five indices. Animals were first administered a therapeutic hydrolyzed protein diet (Royal Canin HP). Dogs that entered remission following this treatment were designated as diet-response (DR) and were maintained on therapeutic diet for the reminder of the trial. Animals that did not respond to therapeutic diet (NDR) subsequently began a two-week course of metronidazole (10 mg/kg PO q 12 hours) while being maintained on the therapeutic diet. Dogs that entered remission following antibiotic treatment were maintained on the combination of antibiotics and therapeutic diet for the reminder of the trial. Animals that still failed to show a favorable response remained on diet and metronidazole but received prednisone (1 mg/kg PO q 12 hours) (Tier 3) for the final 14 days of the trial. Dogs that presented with hypoalbuminemia (protein-losing enteropathy) at the initial screening were presumed to have more severe disease and poorer prognoses and thus were immediately administered all three interventions and were not included in the analyses. All dogs in which serum cobalamin was low at screening were supplemented with cyanocobalamin (50 mcg/kg SQ q 7 days) for the duration of the study. At the conclusion of the study, all animals returned to the clinic for the primary endpoint, which included a full re-evaluation of dogs including complete physical examination, complete blood count, serum chemistry, serum measurement of cobalamin and folate (if low at screening visit), urinalysis, CCECAI scoring and final fecal collections.

16S rRNA Gene Sequencing and Data Analysis

Genomic DNA was extracted from stool using the PowerSoil DNA Isolation Kit (MO BIO Laboratories, Carlsbad, CA) following the manufacturer's recommendations. A mock community pool containing purified genomic DNA from 12 known bacterial isolates was amplified and sequenced as a quality control. Additional controls included extraction of blank-processed samples (in which the DNA extraction process was followed without addition of input material), and water only, to determine background microbial signal. A dual-index amplicon sequencing method was employed for PCR amplification of the V4 region of the 16S rRNA gene⁶¹. Pico-green based Amplicons were sequenced on a MiSeq platform (Illumina, San Diego, CA) using 250 base pair paired-end chemistry. Reads were filtered to remove sequences with average Phred quality score ≤20 using Quantitative Insights into Microbial Ecology (QIIME)⁶² with filtering options (—q 20−p 0.75−r 3). Homopolymers >10 bp in length and sequences <248 bp and >255 bp were removed using Mothur⁶³. Chimeric sequences were identified and removed by usearch61⁶⁴ against the representative 16S sequences of SILVA128 (97_otus_16S.fasta)^{65,66} Quality-controlled sequences were then clustered against the SILVA128 database (SILVA_128_QIIME_release) using the open-reference OTU picking as implemented in QIIME with default parameters. The OTU table was rarefied to 10600 sequences per sample. In order to get a taxonomic assignment at species level for the OTUs from Clostridium sensu stricto 1, the corresponding representative sequences in SILVA database were used to search against NCBI ‘nr’ database. Species were temporarily assigned by the best hits (P<1e−5) and further confirmation were done by comparing the relative abundances of these species determined by metagenomic shotgun sequencing method and by 16S sequencing method. The OTU ‘New.ReferenceOTU52’ represents C. perfringens, which is the most dominant OTU in some dogs, and the OTU ‘FJ957494.1.1454’ is corresponding to C. hiranonis.

Analysis of OTU tables was carried out using the R statistical environment⁶⁷, the bioconductor suite of software⁶⁸, and the Phyloseq2 package⁶⁹. Singletons and OTUs with ambiguous annotations were removed from the OTU table. Alpha diversity (Shannon diversity index and Faith's Phylogenetic Diversity) and Beta diversity (weighted and unweighted UniFrac) were calculated using Phyloseq2. Pielou's evenness index was calculated according to the literature⁷⁰. Functional potential of microbial communities (KEGG pathways and KEGG Orthologs) was predicted by Tax4Fun⁷¹ with default parameters against SILVA123 database. Wilcoxon sum rank test was used for comparisons of KEGG pathways at different timepoints (FDR <0.05). Principal component analysis for KEGG pathways and orthologs was performed by the R package factoextra. For differential abundance analysis and association analysis, filtering was carried out to remove taxa with a max abundance <0.1% across all samples and present in <10% of all samples. The resulting 381 species accounted for an average 96.23% of the total microbial composition. DESeq2⁷² implemented in Phyloseq2 (test=“Wald”, fitType=“parametric”) was used for differential abundance analysis on different taxonomy levels (Fold change>2 and P value <0.05) using un-rarefied reads. The Spearman correlation was computed between the abundance of each microbial composition (Log-transformed) and the values of different factors (i.e., CCECAI for each dog, time points, concentration of each metabolite). To avoid taking log of the zero value, 1 read was added to the abundance for each composition before calculating the Spearman correlation. All p values in the above analysis were adjusted by the FDR (Benjamini-Hochberg) method for multiple comparisons except where noted.

### Metagenomic Sequencing and Data Analysis

Sequencing libraries were prepared using Illumina Nextera XT with 1 ng of canine stool collected at days 0, 14 and 42 from 19 out of the 20 diet-responsive dogs in the study. Sizing and quantification of libraries was carried out using a Tapestation 4200 (Agilent) and Qubit 3 (Thermo Fisher), respectively. Equimolar amounts of each library were pooled and sequenced on an Illumina NextSeq 500 instrument to produce 150 bp, paired-end sequences. Sequencing adapters and low quality reads were trimmed and filtered by Trimmomatic (v0.36) (leading:3 trailing:3 slidingwindow:4:15 minlen:36). High quality reads were mapped to the canine reference genome (CanFam3.1), using Bowtie2 v2.3.4.1 (-very-sensitive), and aligned reads were removed using SamTools 73. After host filtering, each sample was sequenced to a depth of >10 million paired-end reads (median depth=35.8 million). Taxonomic annotation for each sample was generated using Metaphlan2⁴⁶. The identified Clostridium spp. and Eubacterium spp. were further searched for the existence of genes involved in secondary bile acid production (bai operon) using tBlastn against the reference genomes of these species in GeneBank with the protein sequence of genes in 7α-dehydroxylation pathway (baiG, baiB, baiA, baiF, baiCD and baiE) (p-value ≤1e−5).

Metagenomic data from pediatric Crohn's disease patients before and after exclusive enteral nutrition (EEN) have been described previously²³ and were downloaded from European Nucleotide Archive (ENA) (SRP057027). The same filtering steps and settings for the metagenomic data analysis above in this study were used for these datasets. After filtering out human reads, taxonomic annotation for each sample using Metaphlan2 showed the presence of Clostridium. Among them, Clostridium scindens has been well known for the secondary BA producing ability. Paired reads with PCR duplicates removed by samtools 73 were aligned to the C. scindens reference genome (ASM15450v1, strain ATCC 35704) as well as strain VE202-05 (ASM47184v1) using bwa-mem (v0.7.17-r1188) 74 with default settings to estimate the abundances of bacteria among different samples (proportion of mapped reads in total reads). Wilcoxon sum rank test was used to test for significant differences in read mapping, and Spearman correlation was used to compare number of reads mapped with log-transformed fecal calprotectin (FCP) levels²³.

### Anaerobic Culture and Identification of Bacterial Isolates by Whole Genome Sequencing

Rectal swabs freshly collected from dogs with active disease (day 0) and/or in remission at the end of the study (day 42) were transferred to an anaerobic chamber (97.5% nitrogen, 2.5% hydrogen; Coy Labs, Grass Lake, MI) within one hour of collection. The tip of the swabs was homogenized in 1 mL of pre-reduced PBS with 1% cysteine (PBSc). Serial dilutions made in PBSc (down to 10⁻⁵) were plated on brain-heart infusion (BHI), yeast casitone fatty acid with carbohydrate (YCFAC)⁷⁵, gut microbiota medium (GMM)⁷⁶, and De Man, Rogosa and Sharpe (MRS)⁷⁷ agars (Anaerobe Systems, Morgan Hill, CA). After incubation at 37° C. for 1-3 days, single colonies were picked from plates and grown overnight in BHI, YCFAC, GMM, or MRS broth (Anaerobe Systems, Morgan Hill, CA). Overnight cultures were saved as glycerol stocks (25% glycerol) and frozen neat for DNA extraction. DNA was purified from bacterial isolates using the High Pure PCR template kit (Roche) and used for PCR with primers specific for the bacterial 16S rRNA gene, including 27F (5′-AGAGTTTGATCMTGGCTCAG-3′ (SEQ ID NO: 5)), 515F (5′-GTGCCAGCMGCCGCGGTAA-3′ (SEQ ID NO: 6)), and 1492R (5′-CGGTTACCTTGTTACGACTT-3′ (SEQ ID NO: 7)). PCR products were purified using QiaQuick PCR Purification kit (Qiagen), Sanger sequenced, and sequences were assembled using Geneious software v11.1.5 (Biomatters Inc.). The longest high quality stretch of assembled sequence (at least 800 bp) was used for BLAST to find closest the match in Genbank. In addition, for selected C. hiranonis, C. perfringens and E. coli isolates, 1 ng of DNA was used to construct sequencing libraries using Illumina Nextera XT. Libraries were sized and quantified as described above for metagenomic sequencing. For each sample, at least ˜10 million, 150 bp single-end reads were generated using an Illumina NextSeq 500 instrument. Quality control steps were the same as the metagenomic analysis above. High quality reads were mapped to the genome of C. hiranonis (ASM15605v1) using Stampy⁷⁸ (-substitutionrate=0.1), which allows mapping of reads that are highly divergent from the reference genome. PCR duplicates were removed by Samtools. Coverage of genomic regions representing the bai operon were calculated for each isolate to show the existence of genes in 7α-dehydroxylation pathway.

### Metabolomics and in Vitro Bacterial Growth Inhibition Assays

Bile acids were quantified in stool using a Waters Acquity uPLC System with a QDa single quadrupole mass detector and an autosampler (192 sample capacity) as described previously 79. Briefly, fecal samples were suspended in methanol (5 μL/mg stool), vortexed for 1 minute, and centrifuged at 13,000 g for 5 minutes. The supernatant was transferred to a new vial and analyzed on an Acquity uPLC with a Cortecs UPLC C-18+ 1.6 mm 2.1×50 mm column. All chemicals and reagents were mass spectrometry grade. Canine isolates of C. perfringens (n=3) and E. coli were revived from glycerol stocks in Modified Reinforced Clostridial Broth (MRCB, Fisher Scientific) or Luria broth (LB, Fisher Scientific), respectively and grown overnight in the anaerobic chamber at 37° C. Lithocholic and deoxycholic acids (Sigma) were dissolved in 100% ethanol (30 mg/mL). Growth inhibition by deoxycholic acid was determined by microbroth dilution and assessed by OD 630 after overnight growth. Due to low solubility (<1 mg/L), inhibition by lithocholic acid was assessed by counting colonies on agar plates with LCA (0, 0.1, 0.25, 0.5, 0.75, or 1 mg/mL and LB plates for E. coli, and 0, 0.01, 0.025, 0.05, 0.075, or 0.01 mg/mL and Columbia blood agar supplemented with 5% defibrinated sheep's blood for C. perfringens that were incubated anaerobically, at 37° C. for 24 (E. coli) or 48 (C. perfringens) hours.

### Mouse Experiments

Female C57BL/6 (7 weeks old) (Jackson Laboratory) were orally pre-colonized with a kanamycin-resistant E. coli strain (Nissle 1917) (1×10⁹ CFU/mouse) 4 days prior to the to the start of dextran sulfate sodium (DSS) treatment. Animals were randomly assigned to groups (cages) at baseline and drinking water was replaced with either filter-sterilized water (mock-treatment), or a filter-sterilized solution of 2.5% (w/v) DSS (relative molecular mass ˜40,000; Sigma-Aldrich) in water. The mice treated with mock or DSS were orally gavaged C. hiranonis (1×10⁸ CFU/mouse, in anaerobic PBS) or PBS (control) from days 0 to 4. C. hiranonis was grown overnight in MRCB, anaerobically, at 37° C. Culture density was assessed via optical density (630 nm) and the required volume of culture was spun at 10,000 g for 15 min. Bacterial pellets were resuspended in PBS to obtain a dose of 1×10⁸ CFU/mouse. All procedures were performed in accordance with the guidelines of the University of Pennsylvania Institutional Animal Care and Use Committee. The mice were then euthanized at day eight and colon contents and tissues were collected. Colon contents were weighted and cultured on LB agar plates with kanamycin (100 μg/mL) for 16 hours. Stool samples from baseline and colon contents from day eight were collected and stored at −80° C. for the detection of bile acid levels. Colons were fixed in formalin and sections stained with haematoxylin and eosin (H&E). Pathology was blindly evaluated by an board-certified veterinary pathologist (C.B.) according to standard criteria for DSS colitis.

### Data Availability

Raw 16S rRNA gene sequences for canine stool samples have been deposited in the Sequence Read Archive (SRA 80; accession number pending). Processed OTU tables and metadata can be accessed through MicrobiomeDB⁵⁶. Metagenomic and whole genome sequence data are also available on SRA (accession numbers pending).

### Results

### Dietary Therapy Induces Rapid and Durable Remission

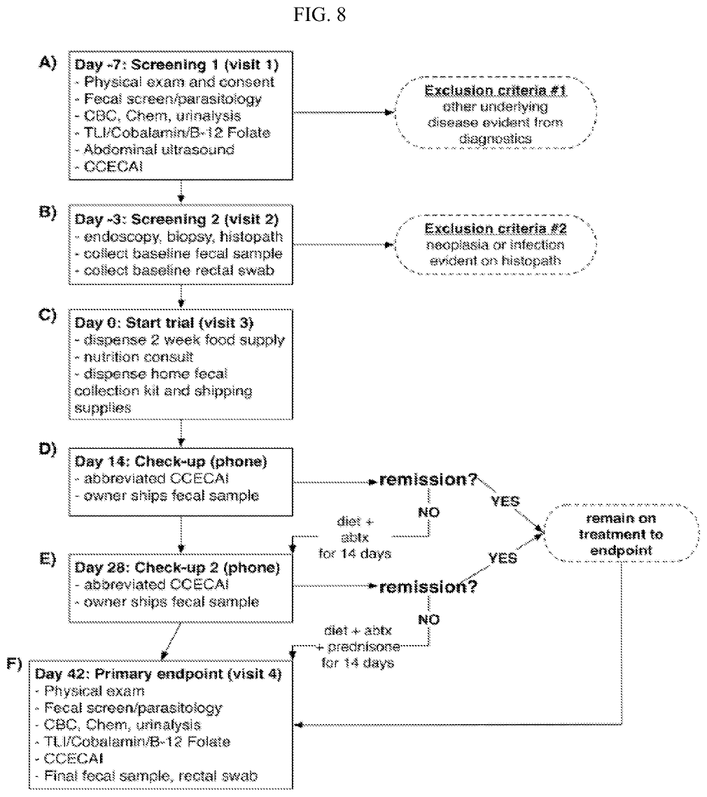

To investigate the impact of a therapeutic diet on disease and the microbiome, treatment-naive dogs (n=29) with chronic enteritis (CE) were enrolled in a study to evaluate the impact of diet on disease and the microbiome. Dogs with active disease were switched from their current diet to a commercially-available therapeutic hydrolyzed protein diet (FIG. 1A). Impact of treatment on disease was monitored using the Canine Chronic Enteropathy Clinical Activity Index (CCECAI; hereafter referred to as ‘disease score’), which is positively correlated with poor clinical outcome³⁶. After two weeks on therapeutic diet, 69% (20/29) of animals entered remission, marked by a reduction in the mean disease score from 4.1 (95% CI=4.8-3.3) to 1.3 (95% CI=1.8-0.7). These diet-responsive (DR) animals were maintained on diet for the remainder of the study with no additional interventions (FIG. 1). At the conclusion of the study (day 42), DR animals had an mean disease score of 0.9 (95% CI=1.3-0.6), constituting an >4-fold reduction in disease severity compared to day 0 (FIG. 1). In contrast, 31% (9/29) of animals failed to show a significant reduction in disease score after two weeks on therapeutic diet (FIG. 1C). These non-diet-responsive (NDR) animals presented with more severe disease scores (mean score=6.1; 95% CI=7.4-4.7) than DR animals (P<0.05 at day 0) and did not show a significant reduction after 2 week diet therapy (FIG. 1C). NDR animals were maintained on diet therapy for the reminder of the study, but also received combination therapy that included antibiotics (at day 14) and prednisone (at day 28) (FIG. 1A and FIG. 8, see methods), but showed only incremental improvement in disease scores (FIG. 1C). These data highlight a rapid clinical response to hydrolyzed diet in the majority of dogs with chronic enteritis.

Identification of Microbial Community Profiles Associated with Treatment Outcome

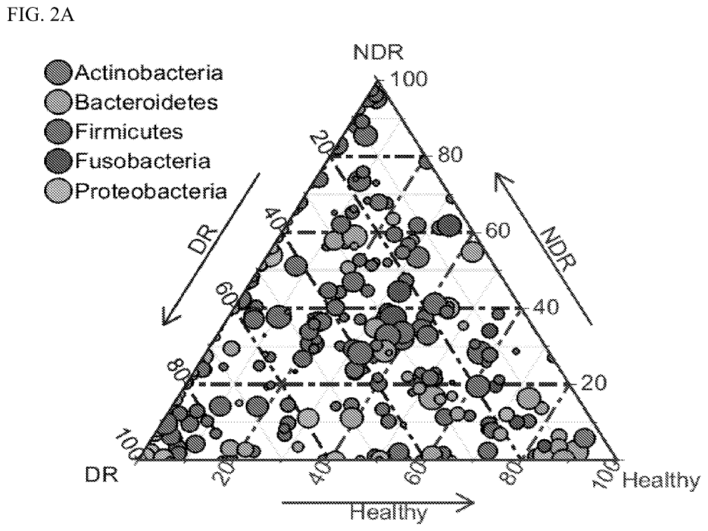

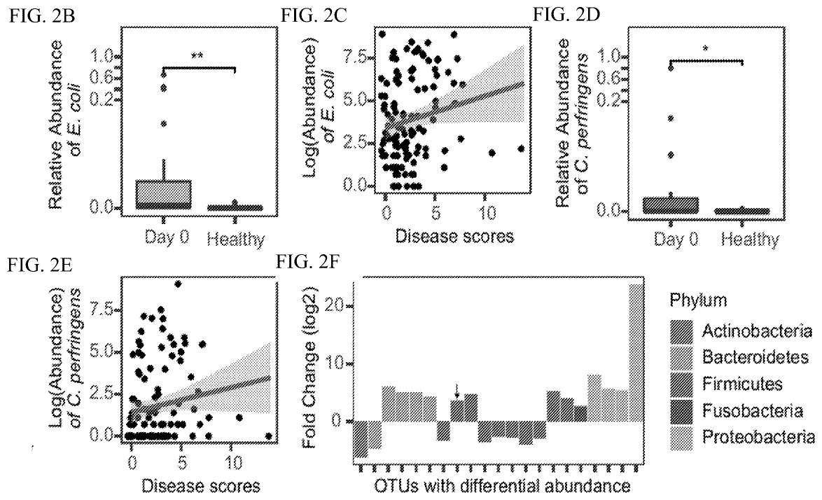

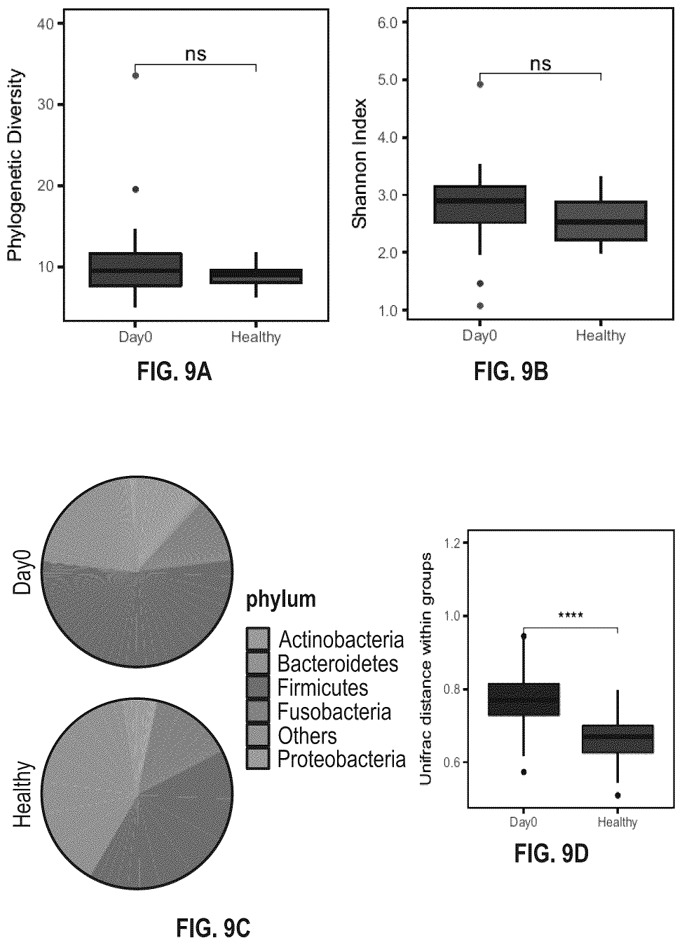

To determine whether treatment with hydrolyzed diet alone is sufficient to alter the microbial community in the gut, 16S rRNA gene profiling was carried out on fecal samples collected from DR, NDR and healthy control animals (n=11). Consistent with previous reports³⁸, it was found that the diversity of the canine fecal microbiome was not significantly altered in dogs with CE, compared to healthy controls (FIGS. 9A-B), and that the communities in both groups were predominantly comprised of Firmicutes, Bacteroidetes, Proteobacteria, Actinobacteria, and Fusobacteria (FIG. 9C). However, compared to healthy dogs, animals with CE showed greater between-individual distance in microbial community structure by unweighted Unifrac (FIG. 9D). Using a ternary plot visualization, an enrichment of Operational Taxonomic Units (OTUs) was observed from Firmicutes and Proteobacteria in animals with active disease, while Bacteroidetes were enriched in healthy animals (FIG. 2A). Interestingly, a subset of proteobacterial OTUs was highly enriched in DR animals compared to both NDR and healthy controls (FIG. 2A), tan points in lower left corner).

These differences prompted us to carry out a formal differential abundance analysis, identifying 55 OTUs that distinguish healthy animals from those with disease (Table 2). For example, Escherichia coli, which is commonly associated with intestinal diseases, was over-represented in animals with CE (FIG. 2B), showing a significant, albeit weak, positive correlation with disease score (R=0.2109, P=0.02626) (FIG. 2C). OTUs from Clostridium sensu stricto 1 were also enriched in CE, including Clostridium perfringens (FIG. 2D), which was also positively correlated with disease scores (FIG. 2E) (R=0.2324, P=0.01412). These bacteria have been implicated in large bowel diarrhea/colitis in dogs³⁹. Taken together with previously published work⁴⁰, these data demonstrate that dysbiosis during CE is marked by the presence of pathobionts. Next, whether the microbiome in DR and NDR animals differed prior to the start of treatment (day 0) was investigated. Although no differences were observed between the two groups in community diversity, evenness or distance from healthy controls (unweighted or weighted Unifrac), 21 OTUs were identified that were differentially abundant between DR and NDR animals, 13 of which were enriched in animals that ended up responding to diet treatment (FIG. 2F and Table 3). Interestingly, Proteobacteria and C. perfringens were found to be more abundant in DR animals (FIG. 2F). Collectively, these results highlight distinct microbial signatures during disease that are associated with different clinical outcomes following diet therapy.

Therapeutic Diet Ameliorates Dysbiosis Associated with Chronic Enteritis

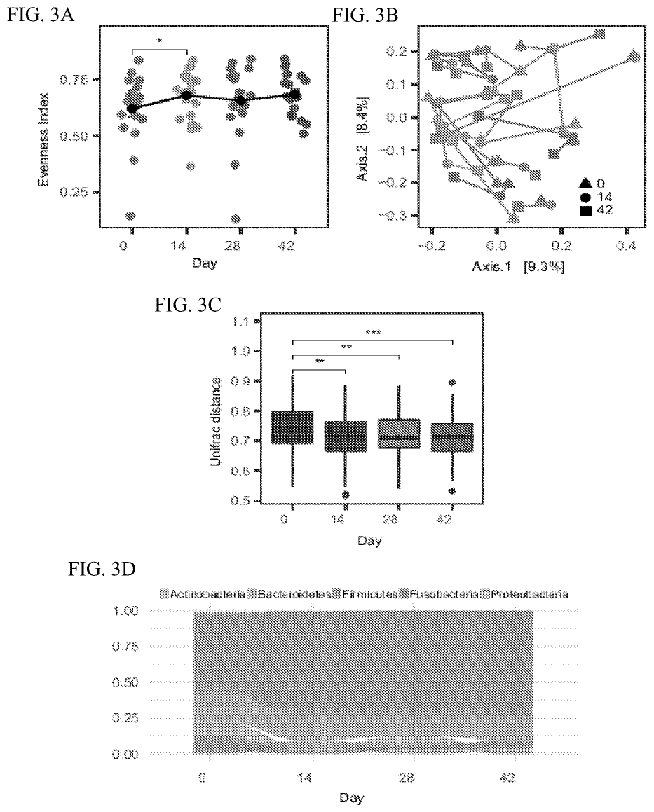

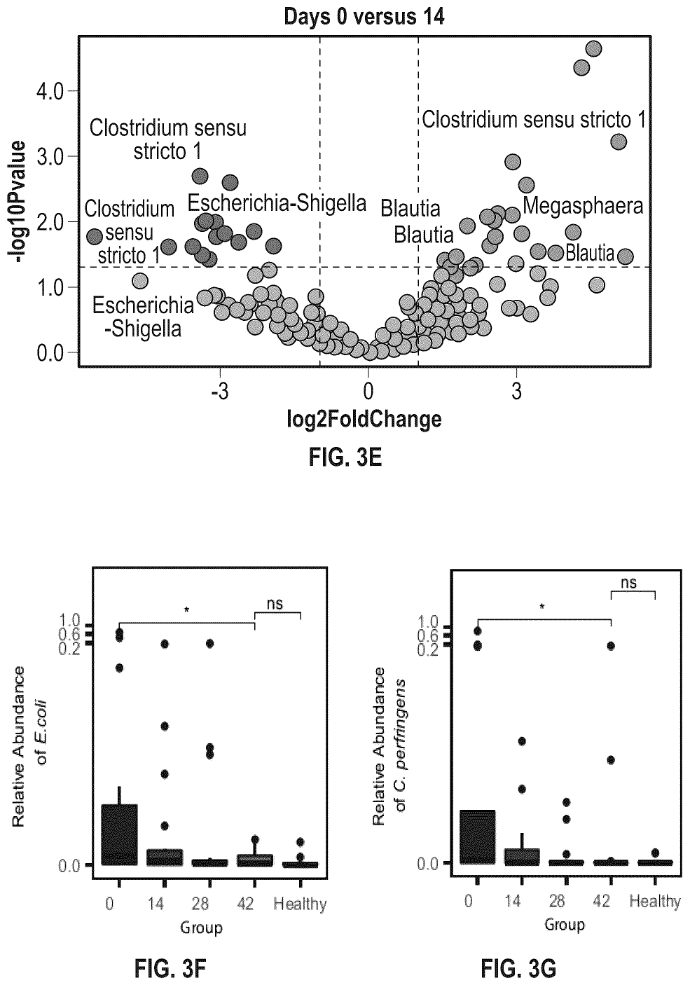

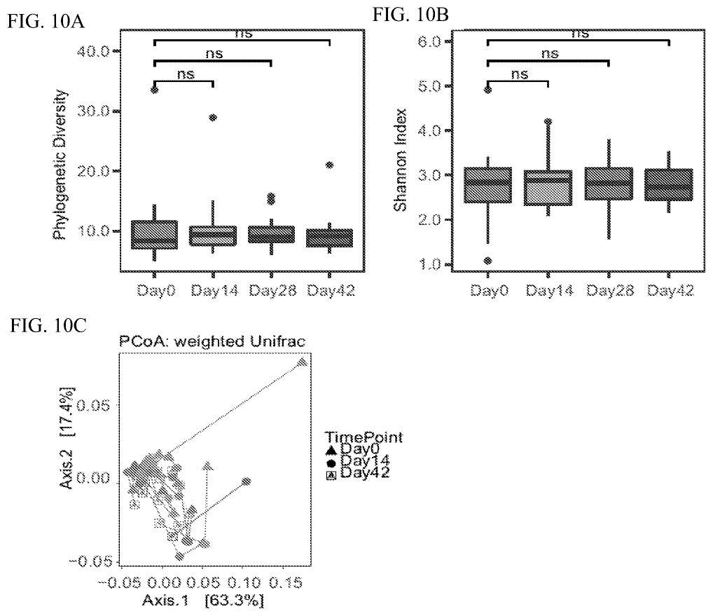

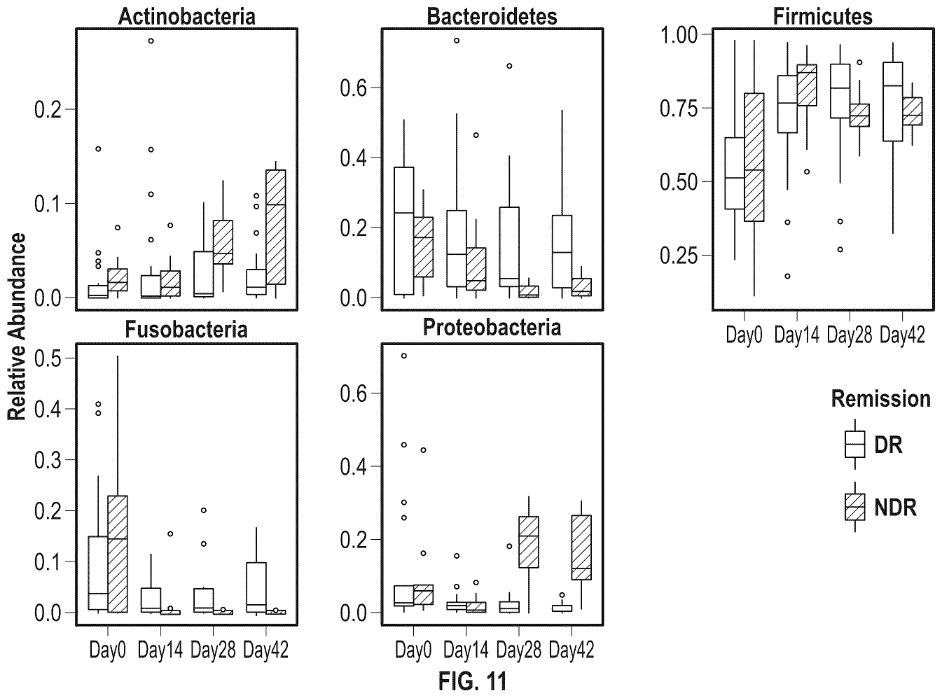

To assess whether diet-induced remission is accompanied by alterations in dysbiosis, the microbial community structures were compared before and after administration of therapeutic diet in DR animals. No significant change was observed in phylogenetic distance or shannon diversity (FIG. 10A-B) but did see a marked increase in community evenness following diet administration (FIG. 3A) when focusing on the top 40 most abundant OTUs among the samples, which account for 83% of the total reads. Principal coordinate analysis based on unweighted (FIG. 3B) or weighted (FIG. 10C) UniFrac showed a clear separation between dogs, even at day 0, before diet therapy was administered, highlighting heterogeneity in dysbiosis associated with clinical disease. Despite this baseline difference between animals, community structure underwent a marked shift away from disease-state by 14 and 42 days after diet therapy (FIG. 3B). Comparing unweighted Unifrac distances between DR and healthy animals at each time point, it was observed that diet-induced remission was marked by decreased phylogenetic distance relative to healthy controls, a trend that continued through day 42, when the phylogenetic similarity to day 0 was lowest and similarity to healthy dogs was highest (FIG. 3C).

Given that therapeutic diet shifted the community structure of the microbiome in DR animals, it was reasoned that composition of the fecal microbiome would be rapidly altered by dietary intervention. Administration of therapeutic diet was broadly characterized by an increase of Firmicutes and a decrease of Proteobacteria (FIG. 3D). Fourteen days after beginning diet therapy, ten genera were differentially abundant compared to pre-treatment (day 0) in DR animals (Table 4). Potential pathogenic genera associated with IBD were found under-represented after diet treatment. For example, Escherichia-Shigella, Clostridium sensu stricto 1, Enterococcus and Fusobacterium had a higher relative abundance at Day 0 and were significantly reduced after 14 days on therapeutic diet. When evaluated at species level, 36 OTUs were significantly differential abundant between samples collected at day 0 compared to day 14 (FIG. 3E) (Table 5). E. coli was typically enriched in the animals at day 0 in this study (FIG. 3E), and its relative abundance declined dramatically after two-weeks on therapeutic diet, eventually reaching levels nearly undetectable by day 42 that were also indistinguishable from levels observed in healthy dogs (FIG. 3F). C. perfringens also showed significant lower prevalence in the samples at day 14 and in healthy dogs, compared to day 0 samples (FIG. 3G). In turn, several increased OTUs were from the genera that have been suggested as beneficial commensals in human studies, such as Blautia⁴¹ (Table 5). Taken together, these results point to ameliorated dysbiosis with a reduction of pathobionts and increase of beneficial commensal taxa as a hallmark of diet therapy.

### Remission-Specific Changes in the Microbiome Following Diet Therapy

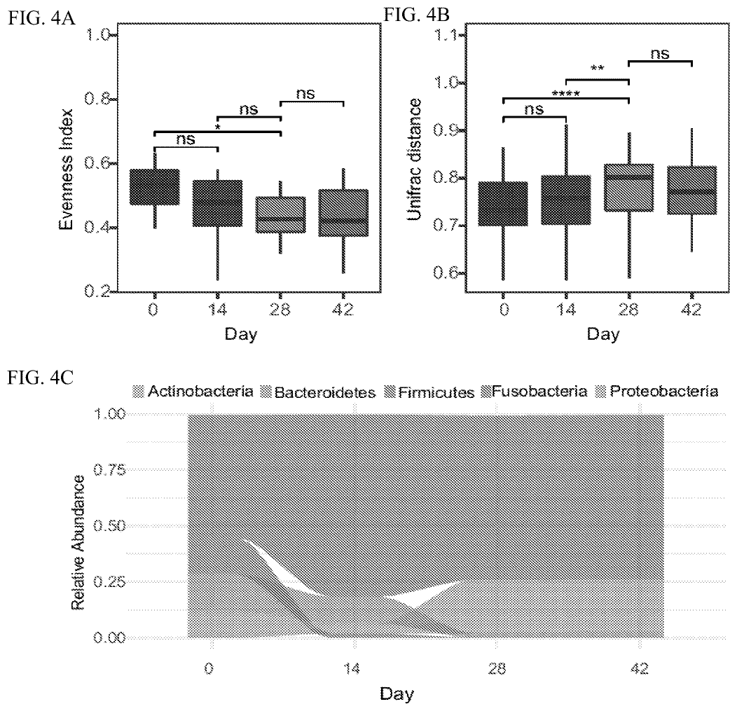

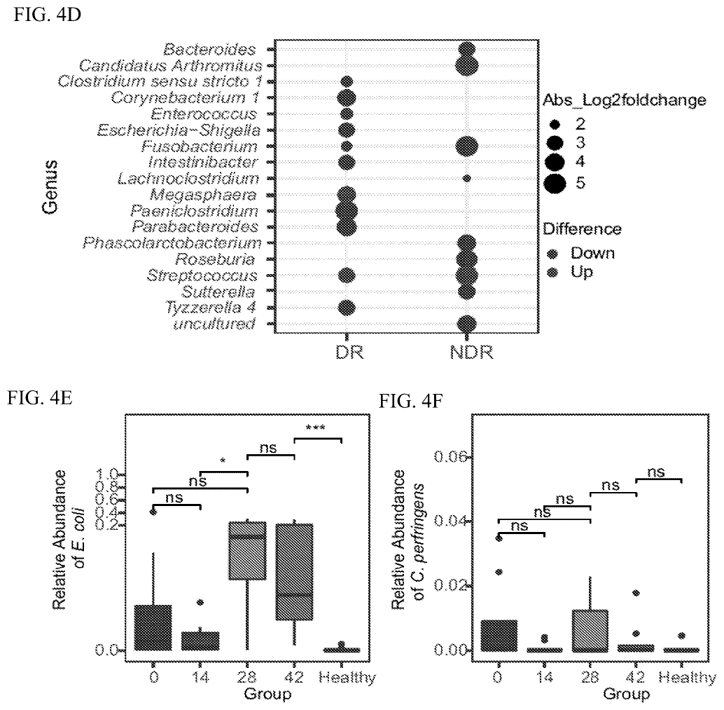

It was hypothesized that the changes observed following diet therapy in DR animals are associated with remission, rather than merely a response to diet that is independent of clinical outcome. To test this hypothesis, the impact of therapeutic diet on dogs that entered remission following diet therapy alone (DR), was compared with changes observed in non-diet-responsive (NDR) animals that failed to enter remission after diet therapy, and which require additional therapies after day 14. Whereas diet therapy in DR animals was associated with increased community evenness (FIG. 3A) and a decreased phylogenetic distance from health dogs (FIG. 3C), the same treatment in NDR dogs did not significantly affect the microbial community evenness or Unifrac distance to healthy dogs (FIGS. 4A and 4B). Just as it was observed in DR animals (FIG. 3D), diet also altered the gut microbiota compositions in NDR animals (FIG. 4C). Differential abundance analysis comparing NDR animals at day 0 versus day 14, when they received only therapeutic diet, identified 24 OTUs (Table 6). However, this shift was distinct from that observed in DR animals (FIG. 4D and FIG. 11). For example, diet therapy was associated with a decrease in the relative abundance of Fusobacterium and Phascolarctobacterium in NDR animals at day 14, compared to day 0, while these taxa were either unchanged or more modestly altered by diet therapy in DR animals. Conversely, Escherichia-Shigella, Enterococcus and some of Clostridium sensu stricto 1 are only reduced in animals that enter remission after diet treatment (FIG. 4D). The disease associated bacteria E. coli and C. perfringens were not significantly changed in NDR animals after diet therapy (FIGS. 4E and 4F). After 14 days on therapeutic diet, NDR dogs were maintained on diet, but were also administered metronidazole, an antibiotic that largely targets anaerobes. Interestingly, antibiotic treatment exacerbated dysbiosis, resulting in a precipitous decline in community evenness (FIG. 4A), increased distance from healthy controls (FIG. 4B), and increased relative abundance of potential pathogens (FIGS. 4E and 4F).

Diet-Induced Remission is Associated with Metabolic Reprogramming and Increased Levels of Secondary Bile Acids.

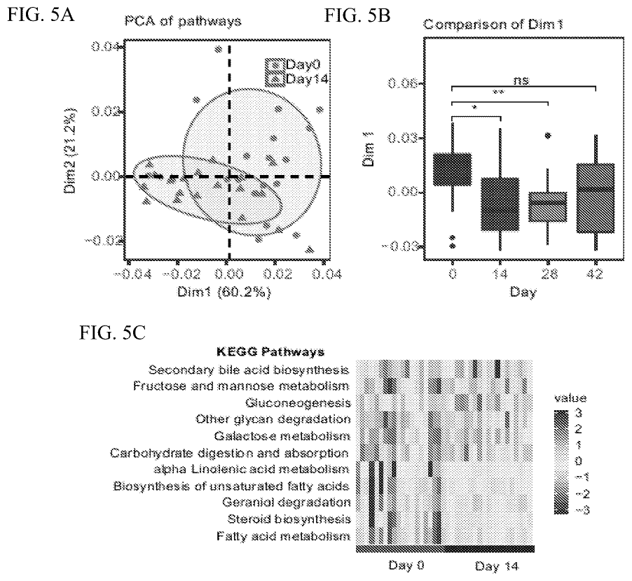

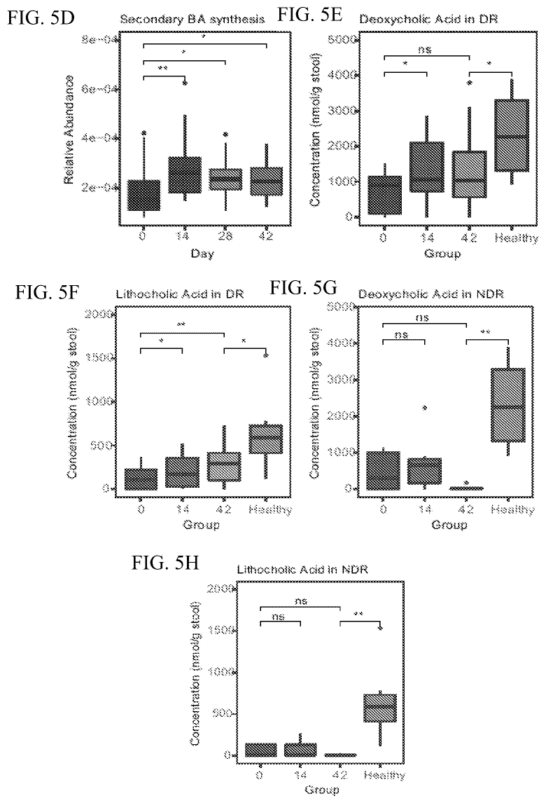

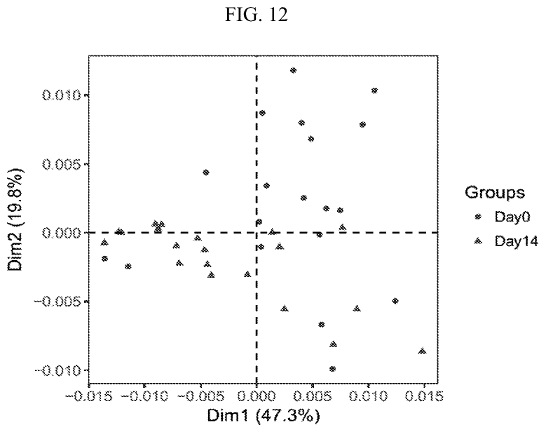

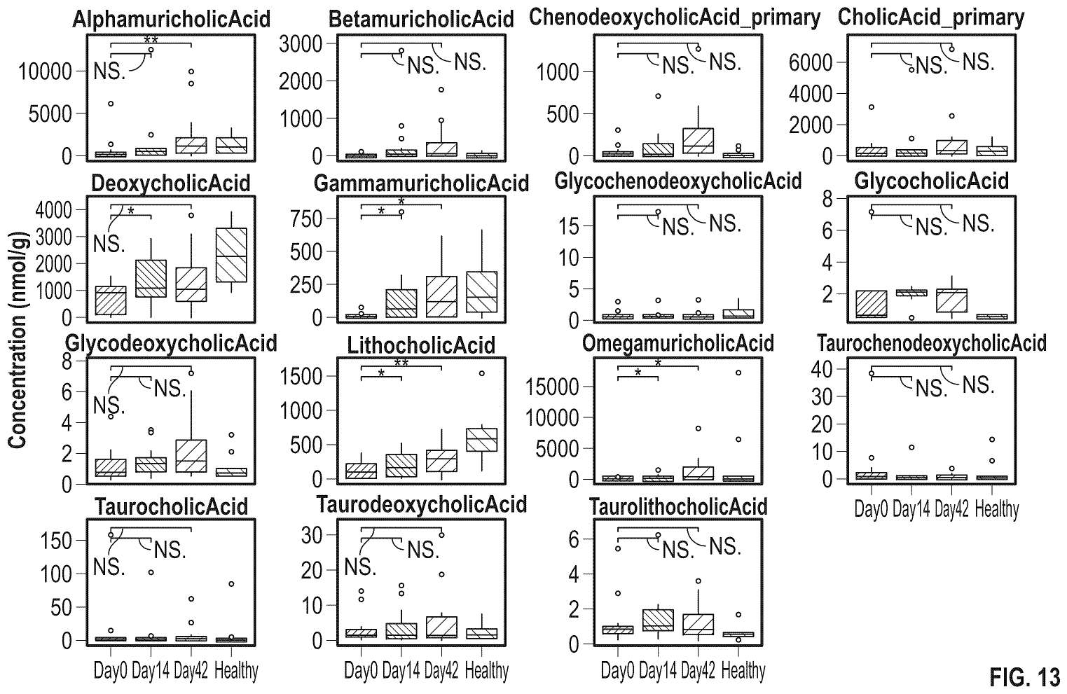

To determine if diet induced changes in microbial community structure would translate to altered microbial metabolism the 16S rRNA gene sequencing data were used to assess the relative abundance of predicted KEGG pathways. Principal component analysis of metabolic pathway abundance data showed a separation between samples from animals with active disease (day 0) and those in remission (day 14) (FIG. 12, FIGS. 5A and 5B). Differential abundance analysis identified 36 pathways were increased in relative abundance as a result of diet treatment in DR animals (Table 7), including several involved in carbohydrate metabolism and secondary bile acid synthesis (FIGS. 5C and 5D). In contrast, 50 pathways were reduced, including fatty acid and steroid biosynthesis (FIG. 5C). This shift in metabolic potential away from lipid biosynthesis toward carbohydrate and bile acid synthesis as animals entered remission prompted us to quantify bile acids in stool. Using targeted metabolomics, levels of 15 bile acids in stool of healthy dogs were measured, compared with stool collected at days 0, 14 and 42 in the study (FIG. 13, Table 10). Consistent with the 16S data, the secondary bile acids deoxycholic acid (FIG. 5E) and lithocholic acid (FIG. 5F) were high in healthy controls but low in animals with active disease (day 0) and were significantly increased after the diet treatment in DR animals at day 14 and 42 (FIGS. 5E and 5F, and Table 8). Notably, levels of these secondary bile acids were not elevated by diet treatment in NDR animals (FIGS. 5G and 5H), suggesting that this metabolic shift is linked to diet-induced remission.

Lithocholic and Deoxycholic Acid Inhibit the Growth of E. coli and C. perfringens, In Vitro.

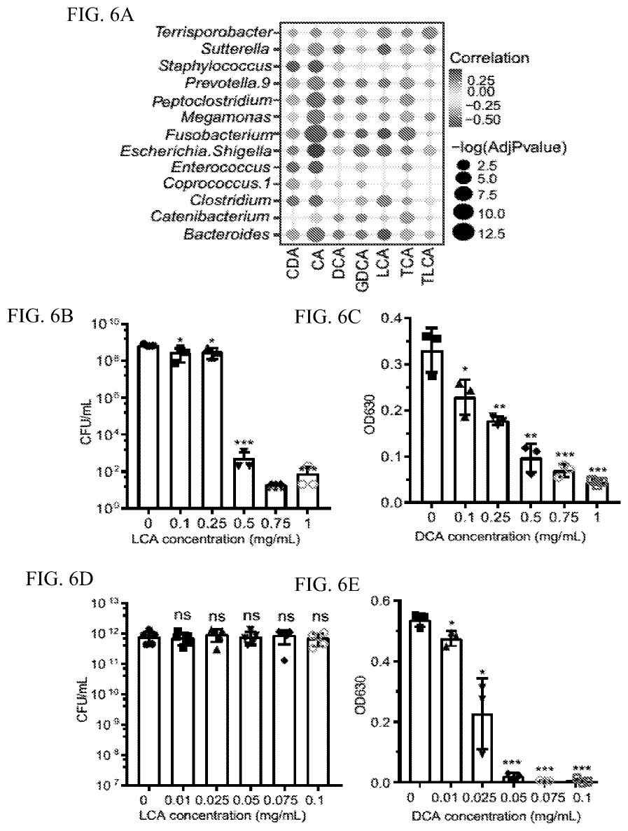

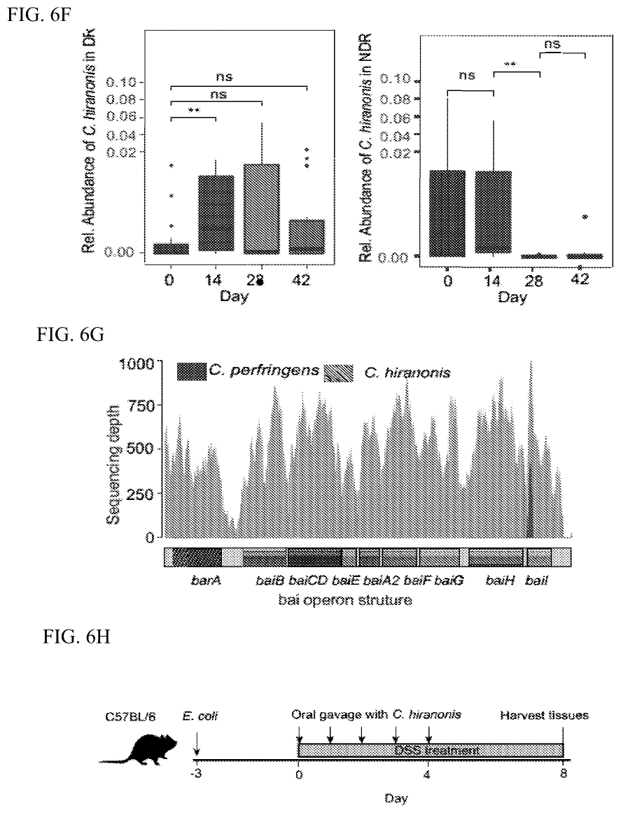

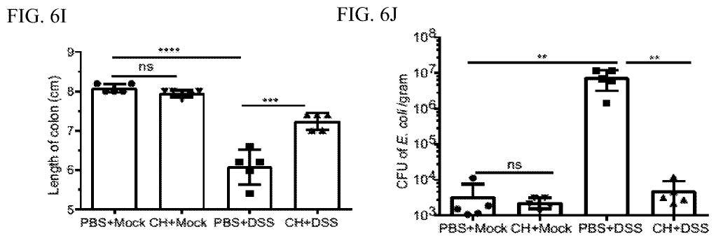

Diet-induced remodeling of the microbiome could be due, at least in part, to the inhibitory role of secondary bile acids on harmful bacteria. Correlation analysis of the metabolomics and microbiome data identified thirteen genera significantly associated with at least one bile acid (Spearman, R>0.04 or <−0.04) (FIG. 6A). The primary bile acid, cholic acid, was negatively correlated with 11 OTUs, consistent with the reported ability of this bile acid to negatively regulate bacterial growth⁴². It was also observed that the increase in secondary bile acids following diet treatment correlated with a reduction in relative abundance of certain bacteria (e.g., OTUs from Escherichia-Shigella, Clostridium and Fusobacterium) (Table 9). To directly test this hypothesis, lithocholic or deoxycholic acid were assessed for their ability to inhibit the in vitro growth of E. coli (n=1) or C. perfringens (n=3) isolates derived from the dogs with active disease, since these species or their genera were associated with disease in the animal model. Deoxycholic acid blocked the growth of both species at a concentration comparable to what was detected in the fecal samples (FIGS. 6C and 6E), while lithocholic acid blocked the growth of E. coli but not C. perfringens (FIGS. 6B and 6D, respectively). Collectively, these results show that the inhibitory activity of these bile acids varies for different bacteria and suggest that elevated secondary bile acids observed following diet therapy can contribute to the decrease of potentially harmful bacteria.

C. hiranonis is a Diet-Responsive Species with the Ability to Produce Secondary Bile Acids.

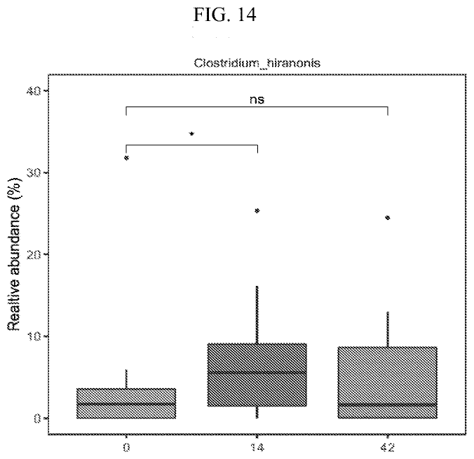

Next, the source of lithocholic and deoxycholic acids after diet treatment was identified. Production of these from primary bile acids requires the 7-dehydroxylation activity conferred by the bile acid-inducible (bai) operon—an activity unique to a limited number of anaerobes representing a small fraction of the microbiome, including some Clostridial and Eubacterial species⁴³. Given the finding that certain clostridial OTUs, as well as levels of lithocholic and deoxycholic acids, increase after diet-induced remission (FIG. 3G and FIGS. 5E-F, respectively), potential bile acid producers in DR animals were identified. Stool samples collected day 0, 14 and 42 after starting diet therapy were subjected to metagenomic sequencing. Taxonomic assignment of reads identified six Clostridium species (C. perfringens, C. hiranonis, C. nexile, C. colicanis, C. glycolicum and C. ramosum) and 2 Eubacterium species (Eubacterium biforme and E. dolichum) present in these samples at a relative abundance ≥0.01% in at least 10% samples. Of these species only C. hiranonis has been reported to have the bai operon 44, and this was confirmed by BLAST against the reference genomes of these species in GenBank. Moreover, the metagenomic data (FIG. 14) and 16S sequencing data showed that the relative abundance of C. hiranonis was significantly increased after diet treatment in DR animals (FIG. 6F, left) but not in NDR animals that failed diet therapy (FIG. 6F, right). Since the Clostridium genus exhibits a high level of genetic divergence, even at the species level, that canine C. hiranonis possesses the bai operon was confirmed. Anaerobic culture of rectal swabs collected during the study, followed by isolate picking and Sanger sequencing of full-length 16S rRNA gene, was used to assemble a canine culture collection from 7 dogs with chronic enteritis before and/or after treatment. In total, 49 Clostridium isolates belonging to 5 species were identified (C. baratii, C. perfringens, C. sartagoforme, C. hiranonis, and C. lactatifermentans). 82% (31/39) of the clostridial isolates from animals with active disease were C. perfringens, consistent with the reported involvement of this organism in canine 39 and human 45 gastrointestinal disease. Two C. hiranonis isolates were obtained from independent diet-responsive animals in remission at day 42. These C. hiranonis isolates and three C. perfringens isolates were selected for whole genome sequencing. Reads were aligned to the reference C. hiranoni, revealing an intact bai operon in canine C. hiranonis, but not C. perfringens (FIG. 6G). Taken together, these data point to C. hiranonis, a species originally isolated from human stool 44, as a likely bile acid producer associated with diet-induced remission in dogs.

C. hiranonis Inhibits Inflammation-Induced Expansion of E. coli in a Mouse Model of DSS Colitis

The ability of C. hiranonis to produce secondary bile acids, combined with the observation that lithocholic and deoxycholic acids were potent inhibitors of E. coli and C. perfringens growth, in vitro, prompted us to test whether C. hiranonis could restrict expansion of potential pathogens in vivo during inflammation. Mice were first colonized with drug-selectable E. coli (Nissle 1917 strain), inflammation was triggered by administration of dextran sodium sulfate (DSS) in the drinking water, and animals were either orally administered PBS daily (mock) or C. hiranonis (FIG. 6H). DSS treatment resulted in reduced colon length (FIG. 6I), and a dramatic bloom of the E. coli Nissle strain (FIG. 6J). In contrast, DSS-treated mice that received C. hiranonis daily showed a marked reduction of colonic shortening, and a near complete abrogation of E. coli expansion. Taken together with the finding that lithocholic and deoxycholic acids can inhibit growth of pathobionts, these data suggest that C. hiranonis or secondary bile acids produced by this species, mediate colonization resistance during enteritis.

C. scindens is Associated with Diet-Induced Remission in Pediatric Crohn's Disease

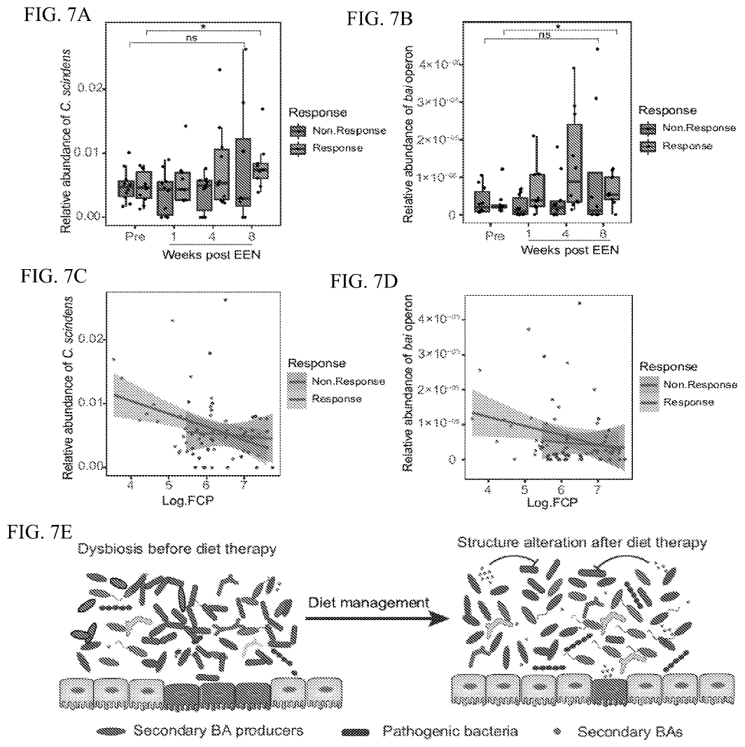

Given that high remission rates are observed in both dogs and humans following dietary therapy, it was hypothesized that a similar induction of bai operon-containing clostridia can occur in pediatric Crohn's disease patients being treated with exclusive enteral nutrition (EEN). To test this, publicly available data from a recent study that examined approximately 20 patients before and after treatment with EEN were analyzed²³, in which half responded to treatment while other half failed EEN therapy. Classifications of bacterial taxa present in each sample using standard metagenomic analysis methods⁴⁶ revealed the presence of C. scindens, which is recognized for having high 7-dehydroxylation activity 44,47. The relative abundance was further estimated using the proportion of total reads that map the reference genome of C. scindens. As shown in FIG. 7A, this bacterium increased significantly from pretreatment to 8 weeks post-EEN, as did the number of reads mapping to the bai operon (FIG. 7B). Remarkably, this increase was only observed in patients that entered remission following EEN (Responsive, n=10) but not those that failed therapy (Non.Responsive, n=10) (FIGS. 7A and 7B). In addition, the correlation analysis between the relative abundance of C. scindens and fecal calprotectin (FCP), a biomarker of disease activity for IBD²³ indicated a significantly negative correlation (FIG. 7C) in EEN ‘Responsive’ patients (R=−0.3515, P=0.03287), but not in EEN ‘Non.Responsive’ patients (R=−0.0267, P=0.877). Similarly, a significant negative correlation between bai operon and FCP was observed in diet-responsive (R=−0.3944, P=0.0157), but not non-responsive patients (R=0.0490, P=0.7766) (FIG. 7D). These results collectively point to bile acid producing clostridia as key features of diet-induced remission and potent inhibitors of pathobiont colonization in both animals and humans (FIG. 7E).

### Discussion

Using a diet-responsive animal model to study the role of the microbiome in chronic enteritis, remission-specific changes in microbiome composition and function were identified. All animals enrolled in the study had active disease, yet their baseline microbiome composition differed greatly (FIG. 3), perhaps reflecting variation in their environment, genetic background (breed), age and weight. This variation in composition supports the idea that enteric disease is driven not by a single dysbiotic state, but rather dysbiosis reflects a loss of community stability⁴⁸. Rather than dramatic changes in microbial community structure following diet therapy, a shift from lipid metabolism to carbohydrate and bile acid synthesis was observed. Although the metabolomics analysis was focused on bile acids, a broader picture of the metabolites produced before and after diet therapy, as well as the macro- and micro-nutrients present in the diets themselves, can improve the understanding of the mechanisms by which diet achieves remission.

One important open question is precisely how therapeutic diets such as EEN or prescription pet foods alter the microbiome and whether there are general principles that could be used to guide the development of better dietary therapies. Studies in pediatric Crohn's disease have reported higher remission rates with EEN compared to partial enteral nutrition (PEN), which includes some table food. These observations have led some to postulate that a highly monotonous diet that is reduced in complexity can constitute an essential part of nutritional therapies for IBD. Consistent with this notion, mice fed a monotonous diet exhibited lower microbial diversity and were more susceptible to DSS colitis than mice fed an alternating diet⁴⁹. However, the prevalence and treatment of chronic enteritis in veterinary medicine highlights that disease routinely develops even when diets are monotonous and that rapid and robust remission can be achieved with solid food. Hydrolyzed protein diets, such as the one used in the study, have been shown to be effective in the management of canine chronic enteropathies^{50,51} and have previously been shown to be more effective for long term management when compared to a highly digestible diet formulated with non-hydrolyzed protein sources^{50,51}. While it is uncertain what characteristic of these hydrolyzed formulas are driving the response, the low molecular weight of hydrolyzed proteins can reduce their ability to be recognized by the immune system while providing improved digestibility. In summary, the results suggest that the dog would be a useful model to dissect the beneficial and harmful roles of different diets, particularly since formulated diets have long been a standard of care for treating numerous diseases in companion animals.

Secondary bile acids and bile acid-producing clostridial species were identified as key features of diet-induced remission in humans and dogs. These findings complement recent studies examining the mechanisms by which fecal microbiota transplant (FMT) cure Clostridium difficile infection⁵². Buffy et al. identified Clostridium scindens as associated with resistance to C. difficile infection in both humans and mice, and they showed that transfer of C. scindens, or a consortium containing this organism, protected mice from C. difficile challenge. Moreover, inhibition of C. difficile growth in vitro by C. scindens was associated with secondary bile acid production. These data are consistent with microbiological studies showing that primary bile acids induce germination of C. difficile, while certain secondary bile acids can block vegetative growth 53 Although C. difficile was not observed in the animals, C. perfringens and E. coli were identified as major disease-associated taxa, and it was shown that physiologic levels of secondary bile acids potently block in vitro growth of these organisms. It is not known whether bile acids can restrict these organisms in vivo in the canine model but elucidating these mechanisms could have important health implications beyond veterinary medicine. Although C. difficile is the leading cause of nosocomial diarrhea in humans, C. perfringens and E. coli are both common human commensals and have been implicated in both diarrheal disease and colitis in humans and dogs. Moreover, the ability of C. perfringens to produce numerous toxins make it a leading cause of foodborne illness and soft tissue infections. Interestingly, when data from a cohort of pediatric Crohn's disease patients before and after diet therapy were examined, it was found that C. scindens was associated with diet-induced remission (FIG. 7), and a related study showed that sustained remission following EEN was characterized by low levels of proteobacteria, while patients that relapsed showed a marked increase in proteobacteria⁵⁴. The data, together with previous studies in dogs^{33,35} highlight the importance of leveraging animal models and advocate for the use of newly developed analytical methods⁵⁵ and database approaches^{56,57} for comparing across multiple microbiome studies to take a ‘One Health’ approach that could identify conserved themes in host-microbiome interactions.

C. difficile infections frequently arise after antibiotic treatment, a phenomenon attributed to the effect of antibiotics on secondary bile acid levels⁵⁸. Interestingly, it was also observed that antibiotics antagonized the diet-induced shifts in microbiome composition and function, promoting a more dysbiotic state coincident with dramatically reduced levels of lithocholic and deoxycholic acid (FIG. 4 and FIG. 5). Taken together, these data support a more general model for microbe-microbe interactions in the gut in which bile acid producing clostridia restrict the growth of a range of bile acid-sensitive pathobionts to limit disease and highlight that these processes are exquisitely sensitive to antimicrobials. The parallels between the findings and those reported for FMT and C. scindens would suggest that FMT might also be beneficial for treating enteritis. Clinical trials testing this hypothesis in IBD patients have shown moderate success, in marked contrast to C. difficile infections where FMT is curative for the vast majority of patients⁵⁹. This discrepancy can be related to different pathobionts contributing to IBD pathogenesis. Interestingly, colitis is a common side-effect observed in cancer patients undergoing immune checkpoint blockade, and a recent study demonstrated complete resolution of this colitis following FMT⁶⁰, raising the possibility that bile acid producers can be important in treating certain types of colitis.

TABLE 2
OTUs with differential abundances between samples of healthy dogs and dogs with
CCE at day 0 (Fold change > 2 and P-value < 0.05).

| | | log2 | | | | | | | | |
|---|---|---|---|---|---|---|---|---|---|---|
| | Base | Fold | | | | | | | | |
| OTU | Mean | Change | P value | Kingdom | Phylum | Class | Order | Family | Genus | Species |
| HQ802983.1.1440 | 134.78 | −9.14 | 3.28E−10 | Bacteria | Firmicutes | Clostridia | Clostridiales | Lachnospiraceae | Tyzzerella 4 | NA |
| FJ950694.1.1472 | 2052.29 | −5.56 | 6.45E−09 | Bacteria | Proteobacteria | Gammaproteobacteria | Enterobacteriales | Enterobacteriaceae | Escherichia-Shigella | Escherichia coli |
| HG798451.1.1400 | 30.74 | −6.70 | 1.14E−07 | Bacteria | Firmicutes | Bacilli | Lactobacillies | Enterococcaceae | Enterococcus | Enterococcus durans |
| New.ReferenceOTU52 | 676.55 | −6.51 | 8.89E−07 | Bacteria | Firmicutes | Clostridia | Clostridiales | Clostridiaceae 1 | Clostridium sensu stricto 1 | perfringes |
| New.ReferenceOTU82 | 35.49 | −4.87 | 6.27E−05 | Bacteria | Firmicutes | Clostridia | Clostridiales | Clostridiaceae 1 | Clostridium sensu stricto 1 | NA |
| GQ449092.1.1375 | 69.30 | −7.04 | 7.97E−05 | Bacteria | Firmicutes | Clostridia | Clostridiales | Lachnospiraceae | Tyzzerella | NA |
| FJ506371.1.1371 | 34.88 | −5.40 | 0.00012 | Bacteria | Firmicutes | Erysipelotrichia | Erysipelotrichales | Erysipelotrichaceae | Erysipelatoclostridium | NA |
| GQ448744.1.1393 | 81.06 | −6.86 | 0.00011 | Bacteria | Bacteroidetes | Bacteroidia | Bacteroidales | Bacteroidaceae | Bacteroides | Ambiguous_taxa |
| FJ957494.1.1454 | 19.18 | −6.33 | 0.00037 | Bacteria | Firmicutes | Clostridia | Clostridiales | Clostridiaceae 1 | Clostridium sensu stricto 1 | Ambiguous_taxa |
| HQ760911.1.1437 | 11.66 | −5.91 | 0.00045 | Bacteria | Firmicutes | Clostridia | Clostridiales | Lachnospiraceae | Anaerostipes | NA |
| GQ006324.1.1342 | 10.73 | −5.77 | 0.00084 | Bacteria | Actinobacteria | Actinobacteria | Corynebacteriales | Corynebacteriaceae | Corynebacterium 1 | uncultured bacterium |
| GQ448246.1.1389 | 313.30 | −3.87 | 0.00079 | Bacteria | Bacteroidetes | Bacteroidia | Bacteroidales | Bacteroidaceae | Bacteroides | Ambiguous_taxa |
| KC245406.1.1465 | 3.79 | −3.70 | 0.0007 | Bacteria | Firmicutes | Clostridia | Clostridiales | Lachnospiraceae | Lachnoclostridium | uncultured bacterium |
| New.ReferenceOTU54 | 39.95 | −5.91 | 0.0009 | Bacteria | Firmicutes | Clostridia | Clostridiales | Lachnospiraceae | Blautia | uncultured bacterium |
| HQ751549.1.1448 | 10.41 | −4.70 | 0.0012 | Bacteria | Firmicutes | Clostridia | Clostridiales | Lachnospiraceae | uncultured | NA |
| JF712675.1.1540 | 6.38 | −5.03 | 0.0012 | Bacteria | Proteobacteria | Gammaproteobacteria | Enterobacteriales | Enterobacteriaceae | Escherichia-Shigella | uncultured bacterium |
| JQ208181.1.1352 | 148.49 | −2.99 | 0.0012 | Bacteria | Firmicutes | Clostridia | Clostridiales | Lachnospiraceae | [Ruminococcus] | Ambiguous_taxa |
| | | | | | | | | | gauvreauii group | |
| GX182404.8.1529 | 3.29 | −4.08 | 0.0020 | Bacteria | Proteobacteria | Gammaproteobacteria | Enterobacteriales | Enterobacteriaceae | Escherichia-Shigella | NA |
| FP929060.3837.5503 | 375.58 | −1.82 | 0.0020 | Bacteria | Firmicutes | Clostridia | Clostridiales | Lachnospiraceae | NA | NA |
| FN667392.1.1495 | 12.19 | −5.97 | 0.0025 | Bacteria | Firmicutes | Bacilli | Lactobacillus | Lactobacilluse | Lactobacillus | uncultured bacterium |
| FN667422.1.1495 | 5.84 | −4.33 | 0.0034 | Bacteria | Firmicutes | Bacilli | Lactobacillies | Lactobacilliae | Lactobacillus | Ambiguous_taxa |
| HK557089.3.1395 | 1340.69 | −3.02 | 0.0046 | Bacteria | Firmicutes | Bacilli | Lactobacillies | Streptococcaceae | Streptococcus | uncultured bacterium |
| HQ803964.1.1435 | 335.70 | −2.90 | 0.0046 | Bacteria | Firmicutes | Clostridia | Clostridiales | Lachnospiraceae | Lachnoclostridium | uncultured bacterium |
| AM276759.1.1484 | 6.84 | −2.87 | 0.0045 | Bacteria | Firmicutes | Clostridia | Clostridiales | Lachnospiraceae | Blautia | uncultured bacterium |
| HK555938.1.1357 | 21.72 | −6.40 | 0.0054 | Bacteria | Actinobacteria | Coriobacteriia | Coriobacteriales | Coriobacteriaceae | Collinsella | uncultured bacterium |
| KF842598.1.1394 | 22.60 | −6.80 | 0.0054 | Bacteria | Bacteroidetes | Bacteroidia | Bacteroidales | Porphyromonadaceae | Parabacteroides | NA |
| HQ792778.1.1436 | 5.38 | 3.62 | 0.0058 | Bacteria | Bacteroidetes | Bacteroidia | Bacteroidales | Bacteroidaceae | Bacteroides | uncultured bacterium |
| FM865905.1.1392 | 8.52 | −5.45 | 0.0066 | Bacteria | Firmicutes | Clostridia | Clostridiales | Clostridiaceae 1 | Clostridium | NA |
| | | | | | | | | | sensu stricto 1 | |
| FN563300.1.1447 | 1147.14 | −1.92 | 0.0064 | Bacteria | Firmicutes | Clostridia | Clostridiales | Lachnospiraceae | Blautia | uncultured bacterium |
| HQ754680.1.1441 | 10.15 | −2.20 | 0.0065 | Bacteria | Firmicutes | Clostridia | Clostridiales | Lachnospiraceae | NA | NA |
| GQ867426.1.1494 | 3.36 | −4.08 | 0.0072 | Bacteria | Proteobacteria | Gammaproteobacteria | Enterobacteriales | Enterobacteriaceae | Escherichia-Shigella | uncultured bacterium |
| EU470512.1.1400 | 2.07 | −3.40 | 0.0079 | Bacteria | Proteobacteria | Gammaproteobacteria | Enterobacteriales | Enterobacteriaceae | Escherichia-Shigella | uncultured bacterium |
| AY239462.1.1500 | 2.71 | −3.20 | 0.0080 | Bacteria | Firmicutes | Clostridia | Clostridiales | Lachnospiraceae | [Ruminococcus] | Ambiguous_taxa |
| | | | | | | | | | gauvreauii group | |
| New.Reference OTU114 | 8.57 | −3.10 | 0.0091 | Bacteria | Firmicutes | Bacilli | Lactobacillies | Streptococcaceae | Streptococcus | uncultured bacterium |
| FN668375.4306350.4307737 | 9.14 | −4.09 | 0.0093 | Bacteria | Firmicutes | Clostridia | Clostridiales | Peptostreptococcaceae | NA | NA |
| AB009242.1.1451 | 8.33 | −4.81 | 0.0097 | Bacteria | Spirochaetae | Spirochactes | Spirochactales | Spirochaetaceae | Treponema 2 | NA |
| HQ792787.1.1438 | 1.61 | 3.45 | 0.0128 | Bacteria | Bacteroidetes | Bacteroidia | Bacteroidales | Bacteroidaceae | Bacteroides | uncultured bacterium |
| AB506370.1.1516 | 5.92 | −4.63 | 0.0194 | Bacteria | Bacteroidetes | Bacteroidia | Bacteroidales | Prevotellaceae | Prevotellaceae UCG-001 | Ambiguous_taxa |
| DQ057365.1.1393 | 5.11 | −4.42 | 0.0202 | Bacteria | Firmicutes | Clostridia | Clostridiales | Lachnospiraceae | Lachnoclostridium | Ambiguous_taxa |
| FN667084.1.1493 | 8.26 | −3.53 | 0.0216 | Bacteria | Firmicutes | Bacilli | Lactobacillus | Lactobacilloeae | Lactobacillus | uncultured bacterium |
| DQ113765.1.1450 | 1500.29 | 3.82 | 0.0230 | Bacteria | Bacteroidetes | Bacteroidia | Bacteroidales | Bacteroidaceae | Bacteroides | NA |
| HK694029.9.1487 | 6.57 | −2.66 | 0.0244 | Bacteria | Firmicutes | Erysipelotrichia | Erysipelotrichales | Erysipelotrichaceae | Faecalitalea | NA |
| AJ270486.1.1241 | 10.92 | −4.24 | 0.0290 | Bacteria | Firmicutes | Clostridia | Clostridiales | Lachnospiraceae | Coprococcus 1 | Ambiguous_taxa |
| EU768569.1.1352 | 5.69 | −3.37 | 0.0314 | Bacteria | Firmicutes | Clostridia | Clostridiales | Ruminococcaceae | Ruminiclostridium 9 | uncultured bacterium |
| FM179752.1.1686 | 1.66 | −3.11 | 0.0324 | Bacteria | Proteobacteria | Gammaproteobacteria | Enterobacteriales | Enterobacteriaceae | Pseudocitrobacter | NA |
| JF807116.1.1260 | 2.54 | −3.70 | 0.0351 | Bacteria | Euryarchaeota | Methanobacteria | Methanobacteriales | Methanobacteriaceae | Methanobrevibacter | uncultured archaeon |
| FJ957528.1.1445 | 14.75 | −5.09 | 0.0356 | Bacteria | Firmicutes | Clostridia | Clostridiales | Clostridiaceae 1 | Sarcina | uncultured bacterium |
| KC504009.1.1465 | 3.67 | −3.15 | 0.0350 | Bacteria | Proteobacteria | Gammaproteobacteria | Enterobacteriales | Enterobacteriaceae | Escherichia-Shigella | NA |
| GQ448506.1.1374 | 304.58 | −1.58 | 0.0335 | Bacteria | Firmicutes | Clostridia | Clostridiales | Lachnospiraceae | Blautia | uncultured bacterium |
| JF224013.1.1362 | 2.89 | −3.89 | 0.0390 | Bacteria | Bacteroidetes | Bacteroidia | Bacteroidales | Porphyromonadaceae | Porphyromonas | uncultured bacterium |
| EU774020.1.1361 | 12.85 | 2.51 | 0.0391 | Bacteria | Fusobacteria | Fusobacteriia | Fusobacteriales | Fusobacteriaceae | Fusobacterium | uncultured bacterium |
| GQ448486.1.1387 | 48.95 | −2.72 | 0.0384 | Bacteria | Firmicutes | Clostridia | Clostridiales | Lachnospiraceae | Blautia | uncultured bacterium |
| HQ793763.1.1451 | 13.41 | 3.32 | 0.0434 | Bacteria | Bacteroidetes | Bacteroidia | Bacteroidales | Bacteroidaceae | Bacteroides | NA |
| JN387556.1.1324 | 164.12 | −2.78 | 0.0459 | Bacteria | Firmicutes | Clostridia | Clostridiales | Peptostreptococcaceae | Intestinibacter | NA |
| New.Reference OTU109 | 34.47 | 3.54 | 0.0488 | Bacteria | Bacteroidetes | Bacteroidia | Bacteroidales | Bacteroidaceae | Bacteroides | NA |

TABLE 3
OTUs with differential abundances between day 0-samples of
diet responsive dogs and diet non-responsive dogs.

| | | log2 | | | |
|---|---|---|---|---|---|
| | Base | Fold | | | |
| OTU | Mean | Change | P value | Kingdom | Phylum |
| JRPJ01000002.1034290.1035971 | 111.72 | 8.05 | 0.000330185 | Bacteria | Proteobacteria |
| JF920309.1.1340 | 22.79 | 5.56 | 0.00409 | Bacteria | Proteobacteria |
| FJ978526.1.1378 | 8.93 | 5.36 | 0.04034 | Bacteria | Proteobacteria |
| New.ReferenceOTU45 | 47.39 | 23.74 | 3.66E−14 | Bacteria | Proteobacteria |
| HK555938.1.1357 | 19.48 | −6.16 | 0.04481 | Bacteria | Actinobacteria |
| FJ957494.1.1454 | 20.74 | −3.37 | 0.01429 | Bacteria | Firmicutes |
| New.ReferenceOTU52 | 1115.31 | 3.54 | 0.01242 | Bacteria | Firmicutes |
| DQ797046.1.1403 | 14.74 | 4.64 | 0.02361 | Bacteria | Firmicutes |
| GQ449092.1.1375 | 15.00 | −3.58 | 0.04561 | Bacteria | Firmicutes |
| AMCI01001631.34.1456 | 77.88 | −4.66 | 0.00349 | Bacteria | Bacteroidetes |
| KF842598.1.1394 | 6.52 | 5.98 | 0.02931 | Bacteria | Bacteroidetes |
| HQ793763.1.1451 | 5.57 | 5.09 | 0.01396 | Bacteria | Bacteroidetes |
| DQ113765.1.1450 | 201.87 | 5.02 | 0.00453 | Bacteria | Bacteroidetes |
| ACBW01000012.3536.5054 | 4.37 | 4.18 | 0.02909 | Bacteria | Bacteroidetes |
| HK693629.1.1491 | 23.00 | −2.59 | 0.01037 | Bacteria | Firmicutes |
| JQ208053.1.1336 | 14.50 | 2.58 | 0.04454 | Bacteria | Fusobacteria |
| GQ493166.1.1359 | 231.28 | −2.75 | 0.00391 | Bacteria | Firmicutes |
| GQ448486.1.1387 | 39.63 | −4.02 | 0.00023 | Bacteria | Firmicutes |
| GQ491426.1.1332 | 461.01 | −2.89 | 0.03894 | Bacteria | Firmicutes |
| New.ReferenceOTU54 | 18.10 | 5.21 | 0.00152 | Bacteria | Firmicutes |
| JN387556.1.1324 | 167.47 | 3.99 | 0.00791 | Bacteria | Firmicutes |
| OTU | Class | Order | Family | Genus | Species |
| JRPJ01000002.1034290.1035971 | Epsilon- | Campylo- | Helicobacteraceae | Helicobacter | Ambiguou_taxa |
| | proteobacteria | bacterales | | | |
| JF920309.1.1340 | Epsilon- | Campylo- | Campylobacteraceae | Campylo- | NA |
| | proteobacteria | bacterales | | bacter | |
| FJ978526.1.1378 | Gamma- | Aero- | Succinivibrionaceae | Succini- | uncultured |
| | proteobacteria | monadales | | vibrio | bacterium |
| New.ReferenceOTU45 | Gamma- | Aero- | Succinivibrionaceae | Anaerobio- | Ambiguou_taxa |
| | proteobacteria | monadales | | spirillum | |
| HK555938.1.1357 | Coriobacteriia | Corio- | Coriobacteriaceae | Collinsella | uncultured |
| | | bacteriales | | | bacterium |
| FJ957494.1.1454 | Clostridia | Clostridiales | Clostridiaceae 1 | Clostridium | Ambiguou_taxa |
| | | | | sensu stricto 1 | |
| New.ReferenceOTU52 | Clostridia | Clostridiales | Clostridiaceae 1 | Clostridium | NA |
| | | | | sensu stricto 1 | |
| DQ797046.1.1403 | Negativicutes | Seleno- | Veillonellaceae | Allisonella | uncultured |
| | | monadales | | | bacterium |
| GQ449092.1.1375 | Clostridia | Clostridiales | Lachnospiraceae | Tyzzerella | NA |
| AMCI01001631.34.1456 | Bacteroidia | Bacteroidales | Bacteroidaceae | Bacteroides | uncultured |
| | | | | | bacterium |
| KF842598.1.1394 | Bacteroidia | Bacteroidales | Porphyromonadaceae | Para- | NA |
| | | | | bacteroides | |
| HQ793763.1.1451 | Bacteroidia | Bacteroidales | Bacteroidaceae | Bacteroides | NA |
| DQ113765.1.1450 | Bacteroidia | Bacteroidales | Bacteroidaceae | Bacteroides | NA |
| ACBW01000012.3536.5054 | Bacteroidia | Bacteroidales | Bacteroidaceae | Bacteroides | uncultured |
| | | | | | bacterium |
| HK693629.1.1491 | Clostridia | Clostridiales | Lachnospiraceae | Blautia | NA |
| JQ208053.1.1336 | Fusobacteriia | Fusobacteriales | Fusobacteriaceae | Fusobacterium | NA |
| GQ493166.1.1359 | Clostridia | Clostridiales | Lachnospiraceae | NA | NA |
| GQ448486.1.1387 | Clostridia | Clostridiales | Lachnospiraceae | Blautia | uncultured |
| | | | | | bacterium |
| GQ491426.1.1332 | Clostridia | Clostridiales | Lachnospiraceae | Blautia | uncultured |
| | | | | | bacterium |
| New.ReferenceOTU54 | Clostridia | Clostridiales | Lachnospiraceae | Blautia | uncultured |
| | | | | | bacterium |
| JN387556.1.1324 | Clostridia | Clostridiales | Peptostreptococcaceae | Intestinibacter | NA |

TABLE 4
Genera with differential abundances between samples of dogs at day 14 and day 0
(day 14 versus day 0) for diet responsive dogs.

| OUT | Base | log2Fold- | P | | | | | | |
|---|---|---|---|---|---|---|---|---|---|
| IDs | Mean | Change | value | Kingdom | Phylum | Class | Order | Family | Genus |
| GQ006324.1.1342 | 15.64 | −3.74 | 0.00137 | Bacteria | Actino- | Actino- | Coryne- | Coryne- | Coryne- |
| | | | | | bacteria | bacteria | bacteriales | bacteriaceae | bacterium 1 |
| New.Ref- | 1991.15 | −2.20 | 0.01222 | Bacteria | Firmicutes | Clostridia | Clostridiales | Clostridiaceae 1 | Clostridium |
| erenceOTU52 | | | | | | | | | sensu stricto 1 |
| HG798451.1.1400 | 25.03 | −2.31 | 0.00911 | Bacteria | Firmicutes | Bacilli | Lacto- | Enterococcaceae | Enterococcus |
| | | | | | | | bacillales | | |
| HK557089.3.1395 | 6043.88 | 3.13 | 0.00124 | Bacteria | Firmicutes | Bacilli | Lacto- | Streptococcaceae | Streptococcus |
| | | | | | | | bacillales | | |
| GQ448336.1.1418 | 34.22 | 3.62 | 0.02080 | Bacteria | Firmicutes | Negativicutes | Seleno- | Veillonellaceae | Megasphaera |
| | | | | | | | monadales | | |
| KF842598.1.1394 | 4.25 | −4.15 | 0.02349 | Bacteria | Bacteroidetes | Bacteroidia | Bacteroidales | Porphyro- | Parabacteroides |
| | | | | | | | | monadaceae | |
| FJ950694.1.1472 | 1259.08 | −3.07 | 0.00109 | Bacteria | Proteo- | Gamma- | Entero- | Entero- | Escherichia- |
| | | | | | bacteria | proteobacteria | bacteriales | bacteriaceae | Shigella |
| HQ802983.1.1440 | 40.41 | −3.23 | 0.002803 | Bacteria | Firmicutes | Clostridia | Clostridiales | Lachnospiraceae | Tyzzerella 4 |
| GQ448468.1.1366 | 2058.39 | −2.10 | 0.01036 | Bacteria | Fusobacteria | Fusobacteriia | Fuso- | Fusobacteriaceae | Fusobacterium |
| | | | | | | | bacteriales | | |
| JN387556.1.1324 | 161.95 | −3.09 | 0.01172 | Bacteria | Firmicutes | Clostridia | Clostridiales | Peptostrepto- | Intestinibacter |
| | | | | | | | | coccaceae | |

TABLE 5
OTUs with differential abundances between samples day 14
and day 0 in diet responsive dogs (day 14 versus day 0).

| | | log2 | | | |
|---|---|---|---|---|---|
| OTU IDs + | Base | Fold | | | |
| A2:K39 | Mean | Change | P value | Kingdom | Phylum |
| JRPJ01000002.1034290.1035971 | 38.04 | −3.39 | 0.034 | Bacteria | Proteo- |
| | | | | | bacteria |
| New.ReferenceOTU45 | 32.86 | −5.37 | 0.017 | Bacteria | Proteo- |
| | | | | | bacteria |
| GQ006324.1.1342 | 10.99 | −3.41 | 0.002 | Bacteria | Actino- |
| | | | | | bacteria |
| HK555938.11357 | 13.49 | 5.19 | 0.034 | Bacteria | Actino- |
| | | | | | bacteria |
| FJ957551.1.1489 | 5.64 | 2.44 | 0.023 | Bacteria | Firmicutes |
| FJ957494.1.1454 | 22.52 | 2.91 | 0.001 | Bacteria | Firmicutes |
| New.ReferenceOTU52 | 899.64 | −2.89 | 0.015 | Bacteria | Firmicutes |
| FM865905.1.1392 | 21.51 | −3.54 | 0.024 | Bacteria | Firmicutes |
| GQ016239.1.1362 | 12.55 | 3.10 | 0.015 | Bacteria | Firmicutes |
| HG798451.11400 | 21.63 | −1.92 | 0.023 | Bacteria | Firmicutes |
| EU461791.1.1414 | 5.48 | 2.9 | 0.043 | Bacteria | Firmicutes |
| GU303759.1.1517 | 22.26 | 2.50 | 0.010 | Bacteria | Firmicutes |
| New.ReferenceOTU114 | 33.26 | 3.17 | 0.002 | Bacteria | Firmicutes |
| AB506154.11541 | 7.45 | 2.91 | 0.008 | Bacteria | Firmicutes |
| EU774370.1.1398 | 2.18 | 3.43 | 0.029 | Bacteria | Firmicutes |
| HK557089.3.1395 | 4123.56 | 2.62 | 0.007 | Bacteria | Firmicutes |
| HQ807346.1.1456 | 11.88 | 4.56 | 2.35E−05 | Bacteria | Firmicutes |
| HQ748204.1.1442 | 15.13 | 4.30 | 4.58E−05 | Bacteria | Firmicutes |
| GU179917.1.1382 | 29.72 | 2.15 | 0.044 | Bacteria | Firmicutes |
| GQ448336.1.1418 | 49.68 | 4.13 | 0.014 | Bacteria | Firmicutes |
| DQ804865.11390 | 32.02 | 3.80 | 0.030 | Bacteria | Firmicutes |
| GQ491757.1.1361 | 6.02 | 1.79 | 0.034 | Bacteria | Firmicutes |
| New.ReferenceOTU56 | 33.71 | 5.05 | 0.000604 | Bacteria | Bacteroidetes |
| KF842598.1.1394 | 4.29 | −4.06 | 0.024 | Bacteria | Bacteroidetes |
| HQ802052.1.1445 | 3.40 | −3.37 | 0.010 | Bacteria | Bacteroidetes |
| GX182404.81529 | 2.29 | −3.22 | 0.035 | Bacteria | Proteobacteria |
| FJ950694.1.1472 | 1165.27 | −2.81 | 0.002 | Bacteria | Proteobacteria |
| GQ448506.11374 | 489.25 | 2.00 | 0.011 | Bacteria | Firmicutes |
| HQ802983.1.1440 | 25.95 | −2.62 | 0.020 | Bacteria | Firmicutes |
| DQ793824.11370 | 11.84 | −3.29 | 0.009 | Bacteria | Firmicutes |
| GQ448468.1.1366 | 2756.51 | −2.30 | 0.013 | Bacteria | Fusobacteria |
| EU774020.1.1361 | 2.57 | −3.07 | 0.011 | Bacteria | Fusobacteria |
| GQ491183.11360 | 618.96 | 1.51 | 0.040 | Bacteria | Firmicutes |
| GQ491426.11332 | 506.28 | 2.55 | 0.017 | Bacteria | Firmicutes |
| GQ493039.11311 | 82.93 | −3.08 | 0.016 | Bacteria | Firmicutes |
| JN387556.1.1324 | 135.02 | −3.10 | 0.010 | Bacteria | Firmicutes |
| EU775983.1.1288 | 2.84 | 2.40 | 0.008 | Bacteria | Firmicutes |
| OTU + A2:K39 | Class | Order | Family | Genus | Species |
| JRPJ01000002.1034290.1035971 | Epsilon- | Campylo- | Helico- | Helicobacter | Ambiguous_taxa |
| | proteobacteria | bacterales | bacteraceae | | |
| New.ReferenceOTU45 | Gamma- | Aeromonadales | Succini- | Anaero- | Ambiguous_taxa |
| | proteobacteria | | vibrionaceae | biospirillum | |
| GQ006324.1.1342 | Actinobacteria | Coryne- | Coryne- | Coryne- | uncultured |
| | | bacteriales | bacteriaceae | bacterium 1 | bacterium |
| HK555938.11357 | Coriobacteriia | Coryne- | Corio- | Collinsella | uncultured |
| | | bacteriales | bacteriaceae | | bacterium |
| FJ957551.1.1489 | Clostridia | Clostridiales | Clostridiaceae | Sarcina | uncultured |
| | | | 1 | | bacterium |
| FJ957494.1.1454 | Clostridia | Clostridiales | Clostridiaceae | Clostridium | Ambiguous_taxa |
| | | | 1 | sensu stricto 1 | |
| New.ReferenceOTU52 | Clostridia | Clostridiales | Clostridiaceae | Clostridium | NA |
| | | | 1 | sensu stricto 1 | |
| FM865905.1.1392 | Clostridia | Clostridiales | Clostridiaceae | Clostridium | NA |
| | | | 1 | sensu stricto | |
| | | | | 1 | |
| GQ016239.1.1362 | Erysipelo- | Erysipelo- | Erysipelo- | Faecalitalea | Ambiguous_taxa |
| | trichia | trichales | trichaceae | | |
| HG798451.11400 | Bacilli | Lacto- | Enteroc- | Enterococcus | Enterococcus |
| | | bacillales | occaceae | | durans |
| | | Lacto- | | | |
| EU461791.1.1414 | Bacilli | bacillales | Strepto- | Streptococcus | uncultured |
| | | cillales | coccaceae | | bacterium |
| GU303759.1.1517 | Bacilli | Lacto- | Strepto- | Streptococcus | uncultured |
| | | bacillales | coccaceae | | bacterium |
| New.ReferenceOTU114 | Bacilli | Lacto- | Strepto- | Streptococcus | uncultured |
| | | bacillales | coccaceae | | bacterium |
| AB506154.11541 | Bacilli | Lacto- | Strepto- | Streptococcus | uncultured |
| | | bacillales | coccaceae | | bacterium |
| EU774370.1.1398 | Bacilli | Lacto- | Strepto- | Streptococcus | uncultured |
| | | bacillales | coccaceae | | bacterium |
| HK557089.3.1395 | Bacilli | Lacto- | Streptoc- | Streptococcus | uncultured |
| | | bacillales | occaceae | | bacterium |
| HQ807346.1.1456 | Bacilli | Lacto- | Streptoc- | Streptococcus | uncultured |
| | | bacillales | occaceae | | bacterium |
| HQ748204.1.1442 | Bacilli | Lacto- | Streptoc- | Streptococcus | uncultured |
| | | bacillales | occaceae | | bacterium |
| GU179917.1.1382 | Erysipelotrichia | Erysipelo- | Erysipel- | Erysipelato- | Ambiguous_taxa |
| | | trichales | otrichaceae | clostridium | |
| GQ448336.1.1418 | Negativicutes | Seleno- | Veillonel- | Megasphaera | uncultured |
| | | monadales | laceae | | bacterium |
| DQ804865.11390 | Clostridia | Clostridiales | Lachnos- | NA | NA |
| | | | piraceae | | |
| GQ491757.1.1361 | Clostridia | Clostridiales | Lachnos- | Blautia | uncultured |
| | | | piraceae | | bacterium |
| New.ReferenceOTU56 | Bacteroidia | Bacteroidales | Prevotel- | Alloprevotella | Ambiguous_taxa |
| | | | laceae | | |
| KF842598.1.1394 | Bacteroidia | Bacteroidales | Porphyro- | Parabacteroides | NA |
| | | | monadaceae | | |
| HQ802052.1.1445 | Bacteroidia | Bacteroidales | Bacteroidaceae | Bacteroides | Ambiguous_taxa |
| GX182404.81529 | Gamma | Entero- | Entero- | Escherichia- | NA |
| | proteobacteria | bacteriales | bacteriaceae | | |
| | | | | Shigella | |
| FJ950694.1.1472 | Gamma | Entero- | Entero- | Escherichia- | Escherichia |
| | proteobacteria | bacteriales | bacteriaceae | Shigella | coli |
| GQ448506.11374 | Clostridia | Clostridiales | Lachnos- | Blautia | uncultured |
| | | | piraceae | | bacterium |
| HQ802983.1.1440 | Clostridia | Clostridiales | Lachnos- | Tyzzerella 4 | NA |
| | | | piraceae | | |
| DQ793824.11370 | Clostridia | Clostridiales | Lachnos- | [Ruminococcus] | uncultured |
| | | | piraceae | gauvreauii | bacterium |
| | | | | group | |
| GQ448468.1.1366 | Fusobacteriia | Fusobacteriales | Fusobac- | Fusobacterium | uncultured |
| | | | teriaceae | | bacterium |
| EU774020.1.1361 | Fusobacteriia | Fusobacteriales | Fusobac- | Fusobacterium | uncultured |
| | | | teriaceae | | bacterium |
| GQ491183.11360 | Clostridia | Clostridiales | Lachnos- | NA | NA |
| | | | piraceae | | |
| GQ491426.11332 | Clostridia | Clostridiales | Lachnos- | Blautia | uncultured |
| | | | piraceae | | bacterium |
| GQ493039.11311 | Clostridia | Clostridiales | Peptostrepto- | NA | NA |
| | | | coccaceae | | |
| JN387556.1.1324 | Clostridia | Clostridiales | Peptostrepto- | Intestinibacter | NA |
| | | | coccaceae | | |
| EU775983.1.1288 | Clostridia | Clostridiales | Peptostrepto- | Peptoclostridium | uncultured |
| | | | coccaceae | | bacterium |

TABLE 6
OTUs with differential abundances between samples of day 0 and day 14 (day 0 versus
day 14) for die non-responsive dogs (Fold change > 2 and P-value < 0.05).

| | | log2 | | | | | | | | |
|---|---|---|---|---|---|---|---|---|---|---|
| | Base | Fold | | | | | | | | |
| OTU | Mean | Change | P value | Kingdom | Phylum | Class | Order | Family | Genus | Species |
| GQ449137.1.1391 | 461.15 | −3.51 | 0.0187 | Bacteria | Proteo- | Betaproteo- | Burk- | Alcalige | Sutterella | NA |
| | | | | | bacteria | bacteria | holderiales | naceae | | |
| HK555938.1.1357 | 30.11 | −6.18 | 0.0121 | Bacteria | Actino- | Corio- | Corio- | Corio- | Collinsella | uncultured |
| | | | | | bacteria | bacteriia | bacteriales | bacteriaceae | | bacterium |
| GQ358246.1.1466 | 302.41 | −3.70 | 0.0182 | Bacteria | Firmi- | Nega- | Seleno- | Acidamin | Phasco- | uncultured |
| | | | | | cutes | tivicutes | monadales | ococcaceae | larcto | Veillonel- |
| | | | | | | | | | bacterium | laceae |
| | | | | | | | | | | bacterium |
| New.ReferenceOTU82 | 61.40 | −3.34 | 0.0262 | Bacteria | Firmi- | Clostridia | Clos- | Clostridi- | Clostridium | NA |
| | | | | | cutes | | tridiales | aceae 1 | sensu | |
| | | | | | | | | | stricto 1 | |
| New.ReferenceOTU52 | 222.71 | −4.55 | 0.0022 | Bacteria | Firmi- | Clostridia | Clos- | Clostridi- | Clostridium | NA |
| | | | | | cutes | | tridiales | aceae 1 | sensu | |
| | | | | | | | | | stricto 1 | |
| GQ138615.1.1402 | 321.54 | −3.56 | 0.0059 | Bacteria | Firmi- | Erysipelo- | Erysipelo- | Erysipelo- | Turici- | uncultured |
| | | | | | cutes | trichia | trichales | trichaceae | bacter | bacterium |
| JN681884.1.1409 | 384.68 | 3.09 | 0.0084 | Bacteria | Firmi- | Bacilli | Lacto- | Strepto- | Strepto- | NA |
| | | | | | cutes | | bacillales | coccaceae | coccus | |
| GU303759.1.1517 | 48.18 | 2.96 | 0.0180 | Bacteria | Firmi- | Bacilli | Lacto- | Strepto- | Strepto- | uncultured |
| | | | | | cutes | | bacillales | coccaceae | coccus | bacterium |
| New.ReferenceOTU114 | 53.48 | 4.21 | 8.04E−05 | Bacteria | Firmi- | Bacilli | Lacto- | Strepto- | Strepto- | uncultured |
| | | | | | cutes | | bacillales | coccaceae | coccus | bacterium |
| EU774881.1.1422 | 3.84 | 3.39 | 0.0224 | Bacteria | Firmi- | Bacilli | Lacto- | Strepto- | Strepto- | uncultured |
| | | | | | cutes | | bacillales | coccaceae | coccus | bacterium |
| AB469559.1.1551 | 13.56 | 5.00 | 0.0016 | Bacteria | Firmi- | Bacilli | Lacto- | Strepto- | Strepto- | uncultured |
| | | | | | cutes | | bacillales | coccaceae | coccus | bacterium |
| HK557089.3.1395 | 9232.07 | 4.47 | 2.88E−05 | Bacteria | Firmi- | Bacilli | Lacto- | Strepto- | Strepto- | uncultured |
| | | | | | cutes | | bacillales | coccaceae | coccus | bacterium |
| EU358719.1.1513 | 12.02 | 2.71 | 0.0180 | Bacteria | Firmi- | Bacilli | Lacto- | Strepto- | Strepto- | uncultured |
| | | | | | cutes | | bacillales | coccaceae | coccus | bacterium |
| HQ748204.1.1442 | 17.52 | 2.85 | 0.0045 | Bacteria | Firmi- | Bacilli | Lacto- | Strepto- | Strepto- | uncultured |
| | | | | | cutes | | bacillales | coccaceae | coccus | bacterium |
| GQ338727.1.1397 | 9.95 | 6.38 | 0.0313 | Bacteria | Firmi- | Clostridia | Clos- | Lachnos- | Anaero- | uncultured |
| | | | | | cutes | | tridiales | piraceae | stipes | bacterium |
| HQ803964.1.1435 | 247.41 | −2.80 | 0.0294 | Bacteria | Firmi- | Clostridia | Clos- | Lachnos- | Lachno- | uncultured |
| | | | | | cutes | | tridiales | piraceae | clostridium | bacterium |
| FJ951866.1.1493 | 7.83 | −5.45 | 0.0177 | Bacteria | Firmi- | Clostridia | Clos- | Lachnos- | Roseburia | NA |
| | | | | | cutes | | tridiales | piraceae | | |
| EU772870.1.1289 | 34.63 | −4.27 | 0.0079 | Bacteria | Fuso- | Fuso- | Fuso- | Fusobac- | Fuso- | uncultured |
| | | | | | bacteria | bacteriia | bacteriales | teriaceae | bacterium | bacterium |
| GQ448468.1.1366 | 4335.90 | −4.03 | 0.0125 | Bacteria | Fuso- | Fuso- | Fuso- | Fusobac- | Fuso- | uncultured |
| | | | | | bacteria | bacteriia | bacteriales | teriaceae | bacterium | bacterium |
| EU774020.1.1361 | 7.55 | −4.76 | 0.0112 | Bacteria | Fuso- | Fuso- | Fuso- | Fusobac- | Fuso- | uncultured |
| | | | | | bacteria | bacteriia | bacteriales | teriaceae | bacterium | bacterium |
| HQ782658.1.1415 | 506.92 | −6.12 | 0.0001 | Bacteria | Fuso- | Fuso- | Fuso- | Fusobac- | Fuso- | Ambi- |
| | | | | | bacteria | bacteriia | bacteriales | teriaceae | bacterium | guous_taxa |
| DQ794633.1.1395 | 23.54 | −3.68 | 0.0209 | Bacteria | Firmi- | Clostridia | Clos- | Lachnos- | NA | NA |
| | | | | | cutes | | tridiales | piraceae | | |
| FN668375.4306350.4307737 | 12.13 | −5.24 | 0.0016 | Bacteria | Firmi- | Clostridia | Clos- | Peptostrepto- | NA | NA |
| | | | | | cutes | | tridiales | coccaceae | | |
| GQ867445.1.1457 | 24.68 | −2.23 | 0.0150 | Bacteria | Firmi- | Clostridia | Clos- | Lachnos- | NA | NA |
| | | | | | cutes | | tridiales | piraceae | | |

TABLE 7
Comparisons of KEGG pathways between different timepoints in the trial for diet
responsive dogs.

| KEGG pathways | Days0vs14_lfc | Days0vs14_pvalue | Days0vs14_fdr |
|---|---|---|---|
| ko00100; Steroid biosynthesis | −2.04 | 0.000209808 | 0.005895615 |
| ko00312; beta-Lactam resistance | 0.44 | 7.25E−05 | 0.005895615 |
| ko00524; Butirosin and neomycin biosynthesis | 0.31 | 0.000164032 | 0.005895615 |
| ko00630; Glyoxylate and dicarboxylate | −0.39 | 0.000125885 | 0.005895615 |
| metabolism | | | |
| ko00910; Nitrogen metabolism | −0.39 | 0.000209808 | 0.005895615 |
| ko03070; Bacterial secretion system | −0.46 | 0.000125885 | 0.005895615 |
| ko04144; Endocytosis | −1.76 | 0.000209808 | 0.005895615 |
| ko04912; GnRH signaling pathway | −1.76 | 0.000209808 | 0.005895615 |
| ko05210; Colorectal cancer | −1.63 | 0.000209808 | 0.005895615 |
| ko05416; Viral myocarditis | −1.63 | 0.000209808 | 0.005895615 |
| ko00640; Propanoate metabolism | −0.25 | 0.000267029 | 0.006821372 |
| ko00010; Glycolysis/Gluconeogenesis | 0.19 | 0.000419617 | 0.006936017 |
| ko00311; Penicillin and cephalosporin biosynthesis | 0.32 | 0.000419617 | 0.006936017 |
| ko00920; Sulfur metabolism | −0.38 | 0.000419617 | 0.006936017 |
| ko04721; Synaptic vesicle cycle | −1.15 | 0.000419617 | 0.006936017 |
| ko04962; Vasopressin-regulated water reabsorption | −1.15 | 0.000419617 | 0.006936017 |
| ko05150; Staphylococcus aureus infection | 0.86 | 0.000335693 | 0.006936017 |
| ko00020; Citrate cycle (TCA cycle) | −0.31 | 0.000644684 | 0.006967545 |
| ko00052; Galactose metabolism | 0.40 | 0.000644684 | 0.006967545 |
| ko00240; Pyrimidine metabolism | 0.12 | 0.000522614 | 0.006967545 |
| ko00410; beta-Alanine metabolism | −0.41 | 0.000644684 | 0.006967545 |
| ko00473; D-Alanine metabolism | 0.29 | 0.000644684 | 0.006967545 |
| ko00592; alpha-Linolenic acid metabolism | −1.38 | 0.000644684 | 0.006967545 |
| ko00633; Nitrotoluene degradation | −0.67 | 0.000644684 | 0.006967545 |
| ko03410; Base excision repair | 0.10 | 0.000522614 | 0.006967545 |
| ko04115; p53 signaling pathway | −1.57 | 0.000522614 | 0.006967545 |
| ko00550; Peptidoglycan biosynthesis | 0.18 | 0.000965118 | 0.008748331 |
| ko00909; Sesquiterpenoid and triterpenoid | −1.52 | 0.000965118 | 0.008748331 |
| biosynthesis | | | |
| ko04621; NOD-like receptor signaling pathway | 0.43 | 0.000965118 | 0.008748331 |
| ko04930; Type II diabetes mellitus | 0.17 | 0.000965118 | 0.008748331 |
| ko05168; Herpes simplex infection | −1.28 | 0.000965118 | 0.008748331 |
| ko00281; Geraniol degradation | −0.58 | 0.001411438 | 0.01166512 |
| ko00540; Lipopolysaccharide biosynthesis | −0.53 | 0.001411438 | 0.01166512 |
| ko04622; RIG-I-like receptor signaling pathway | 0.57 | 0.001411438 | 0.01166512 |
| ko00071; Fatty acid metabolism | −0.55 | 0.001693726 | 0.012863159 |
| ko00120; Primary bile acid biosynthesis | 0.51 | 0.001693726 | 0.012863159 |
| ko00121; Secondary bile acid biosynthesis | 0.51 | 0.001693726 | 0.012863159 |
| ko00430; Taurine and hypotaurine metabolism | −0.32 | 0.00202179 | 0.013856655 |
| ko00590; Arachidonic acid metabolism | −0.39 | 0.00202179 | 0.013856655 |
| ko05012; Parkinsons disease | −0.60 | 0.00202179 | 0.013856655 |
| ko05111; Vibrio cholerae pathogenic cycle | −0.58 | 0.00202179 | 0.013856655 |
| ko00051; Fructose and mannose metabolism | 0.18 | 0.002399445 | 0.014046748 |
| ko00310; Lysine degradation | −0.27 | 0.002399445 | 0.014046748 |
| ko00351; DDT degradation | −0.89 | 0.002399445 | 0.014046748 |
| ko00520; Amino sugar and nucleotide sugar | 0.14 | 0.002399445 | 0.014046748 |
| metabolism | | | |
| ko00561; Glycerolipid metabolism | 0.27 | 0.002399445 | 0.014046748 |
| ko01040; Biosynthesis of unsaturated fatty acids | −0.32 | 0.002399445 | 0.014046748 |
| ko04011; MAPK signaling pathway-yeast | 0.27 | 0.002399445 | 0.014046748 |
| ko00130; Ubiquinone and other terpenoid-quinone | −0.38 | 0.002838135 | 0.014241355 |
| biosynthesis | | | |
| ko00380; Tryptophan metabolism | −0.58 | 0.002838135 | 0.014241355 |
| ko00680; Methane metabolism | −0.15 | 0.002838135 | 0.014241355 |
| ko02060; Phosphotransferase system (PTS) | 0.52 | 0.002838135 | 0.014241355 |
| ko04626; Plant-pathogen interaction | −0.09 | 0.002838135 | 0.014241355 |
| ko04940; Type I diabetes mellitus | 0.09 | 0.002838135 | 0.014241355 |
| ko05110; Vibrio cholerae infection | −1.3 | 0.002838135 | 0.014241355 |
| ko05145; Toxoplasmosis | −1.25 | 0.002838135 | 0.014241355 |
| ko00300; Lysine biosynthesis | 0.07 | 0.003341675 | 0.015915434 |
| ko02040; Flagellar assembly | −0.63 | 0.003341675 | 0.015915434 |
| ko04973; Carbohydrate digestion and absorption | 0.46 | 0.003341675 | 0.015915434 |
| ko05340; Primary immunodeficiency | 0.29 | 0.003917694 | 0.018347867 |
| ko00511; Other glycan degradation | 0.45 | 0.004577637 | 0.020098686 |
| ko00791; Atrazine degradation | −0.24 | 0.004577637 | 0.020098686 |
| ko00983; Drug metabolism-other enzymes | 0.14 | 0.004577637 | 0.020098686 |
| ko03430; Mismatch repair | 0.13 | 0.004577637 | 0.020098686 |
| ko00230; Purine metabolism | 0.08 | 0.005329132 | 0.022689184 |
| ko04260; Cardiac muscle contraction | −0.76 | 0.005329132 | 0.022689184 |
| ko00620; Pyruvate metabolism | −0.06 | 0.00617981 | 0.025918306 |
| ko00190; Oxidative phosphorylation | −0.11 | 0.007144928 | 0.028277814 |
| ko00330; Arginine and proline metabolism | −0.13 | 0.007144928 | 0.028277814 |
| ko00943; Isoflavonoid biosynthesis | 0.55 | 0.007144928 | 0.028277814 |
| ko04614; Renin-angiotensin system | 0.52 | 0.007144928 | 0.028277814 |
| ko00062; Fatty acid elongation | 0.43 | 0.008232117 | 0.030437168 |
| ko02020; Two-component system | −0.19 | 0.008232117 | 0.030437168 |
| ko03008; Ribosome biogenesis in eukaryotes | 0.22 | 0.008232117 | 0.030437168 |
| ko04122; Sulfur relay system | −0.23 | 0.008232117 | 0.030437168 |
| ko04910; Insulin signaling pathway | 0.22 | 0.008232117 | 0.030437168 |
| ko00471; D-Glutamine and D-glutamate | 0.15 | 0.00945282 | 0.033623321 |
| metabolism | | | |
| ko00500; Starch and sucrose metabolism | 0.20 | 0.00945282 | 0.033623321 |
| ko00860; Porphyrin and chlorophyll metabolism | −0.24 | 0.00945282 | 0.033623321 |
| ko05132; Salmonella infection | −0.33 | 0.010826111 | 0.038026714 |
| ko00603; Glycosphingolipid biosynthesis-globo | 0.37 | 0.012359619 | 0.041844012 |
| series | | | |
| ko03030; DNA replication | 0.08 | 0.012359619 | 0.041844012 |
| ko05142; Chagas disease (American | −0.63 | 0.012359619 | 0.041844012 |
| trypanosomiasis) | | | |
| ko00720; Carbon fixation pathways in prokaryotes | −0.12 | 0.014068604 | 0.045968344 |
| ko01057; Biosynthesis of type II polyketide | −0.50 | 0.014068604 | 0.045968344 |
| products | | | |
| ko04146; Peroxisome | −0.21 | 0.014068604 | 0.045968344 |

TABLE 8
Comparisons of bile acids between samples at different timepoints for diet responsive
dogs.

| Bile acid | Days0vs14_fc | Days0vs14_pvalue | Days0vs42_fc | Days0vs42_pvalue |
|---|---|---|---|---|
| AlphamuricholicAcid | 1.74 | 0.192517572 | 2.91 | 0.006835938 |
| DeoxycholicAcid | 1.74 | 0.019058892 | 1.70 | 0.1015625 |
| GammamuricholicAcid | 16.18 | 0.024390241 | 18.33 | 0.014266187 |
| LithocholicAcid | 1.50 | 0.053710938 | 2.02 | 0.010826921 |
| OmegamuricholicAcid | 8.22 | 0.022494271 | 33.55 | 0.042315275 |

TABLE 9
Spearman correlations between abundance of OTUs and concentration of Bile acids in
diet responsive dogs.

| | | Spearman | | Adjusted |
|---|---|---|---|---|
| Taxa | Bile acid | correlation | Pvalue | Pvalue |
| Fusobacterium_uncultured.bacterium | Chenodeoxy- | −0.468045036 | 0.000533 | 0.022042405 |
| | cholic.Acid_primary | | | |
| Bacteroides_NA | Chenodeoxy- | −0.474431034 | 0.000436 | 0.022042405 |
| | cholic.Acid_primary | | | |
| Fusobacterium_uncultured.bacterium | Cholic.Acid_primary | −0.646842527 | 3.11E−08 | 3.86E−06 |
| Fusobacterium_Ambiguous_taxa | Cholic.Acid_primary | −0.60748964 | 3.36E−07 | 2.09E−05 |
| Bacteroides_NA | Cholic.Acid_primary | −0.592565032 | 7.64E−07 | 2.37E−05 |
| Peptoclostridium_uncultured.bacterium | Cholic.Acid_primary | −0.561738193 | 3.67E−06 | 9.11E−05 |
| Megamonas_uncultured.bacterium | Cholic.Acid_primary | −0.518655946 | 2.57E−05 | 0.000532 |
| Bacteroides_uncultured.bacterium | Cholic.Acid_primary | −0.499276241 | 5.69E−05 | 0.001007674 |
| Prevotella.9_uncultured.bacterium | Cholic.Acid_primary | −0.4938089 | 7.05E−05 | 0.001093381 |
| Escherichia.Shigella_Escherichia.coli | Cholic.Acid_primary | 0.487797189 | 8.90E−05 | 0.001226177 |
| Sutterella_NA | Cholic.Acid_primary | −0.456507609 | 0.000279 | 0.003458925 |
| Staphylococcus_Ambiguous_taxa | Cholic.Acid_primary | 0.418106228 | 0.000984 | 0.01108803 |
| Escherichia.Shigella_uncultured.bacterium | Cholic.Acid_primary | 0.407320492 | 0.001366 | 0.014111373 |
| Enterococcus_Enterococcus.durans | Cholic.Acid_primary | 0.394491205 | 0.00199 | 0.018979727 |
| Fusobacterium_uncultured.organism | Cholic.Acid_primary | −0.391629335 | 0.00216 | 0.019129515 |
| Faecalibacterium_uncultured.bacterium | Cholic.Acid_primary | −0.382982602 | 0.002755 | 0.022772609 |
| Clostridium.sensu.stricto.1_NA | Cholic.Acid_primary | 0.365158178 | 0.004459 | 0.030717827 |
| Phascolarctobacterium_uncultured. | Cholic.Acid_primary | −0.365156122 | 0.004459 | 0.030717827 |
| Veillonellaceae.bacterium | | | | |
| Erysipelatoclostridium_NA | Cholic.Acid_primary | 0.369363009 | 0.003989 | 0.030717827 |
| Lactobacillus_uncultured.bacterium | Cholic.Acid_primary | 0.362651494 | 0.004761 | 0.030741248 |
| Enterobacter_NA | Cholic.Acid_primary | 0.361092059 | 0.004958 | 0.030741248 |
| Fusobacterium_Ambiguous_taxa | Deoxycholic.Acid | 0.544270884 | 5.29E−05 | 0.00439602 |
| uncultured.bacterium_uncultured.bacterium | Deoxycholic.Acid | −0.536454836 | 7.09E−05 | 0.00439602 |
| Enterococcus_NA | Glycochenode- | 0.551370353 | 0.000275 | 0.034071581 |
| | oxycholic.Acid | | | |
| Fusobacterium_uncultured.bacterium | Glycocholic.Acid | −0.4919575 | 0.000383 | 0.047524989 |
| Escherichia.Shigella_Escherichia.coli | Glycochenode- | −0.500010873 | 0.00016 | 0.006632914 |
| | oxycholic.Acid | | | |
| Escherichia.Shigella_uncultured.bacterium | Glycochenode- | −0.503678278 | 0.000141 | 0.006632914 |
| | oxycholic.Acid | | | |
| Escherichia.Shigella_NA | Glycochenode- | −0.513698449 | 9.83E−05 | 0.006632914 |
| | oxycholic.Acid | | | |
| Bacteroides_NA | Lithocholic.Acid | 0.602216745 | 1.21E−05 | 0.001495011 |
| Escherichia.Shigella_Escherichia.coli | Lithocholic.Acid | −0.505136952 | 0.000402 | 0.01214716 |
| Clostridium.sensu.stricto.1_uncultured.bacterium | Lithocholic.Acid | −0.498644383 | 0.00049 | 0.01214716 |
| Alloprevotella_NA | Lithocholic.Acid | 0.525651352 | 0.000209 | 0.01214716 |
| Fusobacterium_uncultured.bacterium | Lithocholic.Acid | 0.465544021 | 0.00127 | 0.022503866 |
| Terrisporobacter_uncultured.bacterium | Lithocholic.Acid | −0.468902352 | 0.001158 | 0.022503866 |
| Fusobacterium_Ambiguous_taxa | Taurocholic.Acid | −0.597078223 | 9.46E−07 | 0.000117322 |
| Bacteroides_uncultured.bacterium | Taurocholic.Acid | −0.478414977 | 0.000167 | 0.006908181 |
| Fusobacterium_uncultured.bacterium | Taurocholic.Acid | −0.467425093 | 0.000247 | 0.007641602 |
| Peptoclostridium_uncultured.bacterium | Taurocholic.Acid | −0.447367036 | 0.000485 | 0.012022892 |
| Prevotella.9_uncultured.bacterium | Taurocholic.Acid | −0.432173393 | 0.000788 | 0.015017592 |
| Catenibacterium_uncultured.bacterium | Taurocholic.Acid | −0.429812058 | 0.000848 | 0.015017592 |
| Bacteroides_NA | Taurocholic.Acid | −0.419308054 | 0.001168 | 0.018102789 |
| Megamonas_uncultured.bacterium | Taurocholic.Acid | −0.395318261 | 0.002339 | 0.032219997 |
| Sutterella_NA | Taurocholic.Acid | −0.379322634 | 0.003614 | 0.044819403 |
| Parasutterella_uncultured.organism | Taurocholic.Acid | 0.372251442 | 0.004352 | 0.049062689 |
| Terrisporobacter_uncultured.bacterium | Taurolithocholic.Acid | −0.47287393 | 0.000104 | 0.012896126 |
| Escherichia.Shigella_NA | Taurolithocholic.Acid | −0.408085979 | 0.000993 | 0.046070117 |
| Prevotellaceae.UCG.003_uncultured.bacterium | Taurolithocholic.Acid | −0.404397284 | 0.001115 | 0.046070117 |

TABLE 10
Comparison of the amount of bile acids in the fecal samples of healthy dogs, diet
responsive dogs with CCE and diet non-responsive dogs with CCE.

| | | | Mean | | | Std. | | |
|---|---|---|---|---|---|---|---|---|
| Group | Timepoint | Bile Acid | (nmol/g) | CI lower | CI upper | Error | (mg/g) | # % |
| DR | 0 | Deoxycholic | 753.4492 | 385.5109 | 1121.3876 | 167.1698 | 0.295783059 | 0.029578 |
| DR | 14 | Deoxycholic | 1232.6501 | 787.5515 | 1677.7488 | 209.9616 | 0.483903915 | 0.04839 |
| DR | 42 | Deoxycholic | 1229.1665 | 663.6408 | 1794.6923 | 266.7693 | 0.482536351 | 0.048254 |
| NDR | 0 | Deoxycholic | 487.2588 | −186.5451 | 1161.0627 | 242.6858 | 0.191284162 | 0.019128 |
| NDR | 14 | Deoxycholic | 736.7662 | −124.4111 | 1597.9436 | 335.0126 | 0.289233781 | 0.028923 |
| NDR | 42 | Deoxycholic | 33.16975 | −57.53601 | 123.87551 | 32.66975 | 0.013021515 | 0.001302 |
| Healthy | | Deoxycholic | 2328.5853 | 1519.0883 | 3138.0823 | 357.8429 | 0.914137388 | 0.091414 |
| DR | 0 | Lithocholic | 132.0341 | 46.31136 | 217.75684 | 38.94744 | 0.049720424 | 0.004972 |
| DR | 14 | Lithocholic | 209.8332 | 120.17778 | 299.48862 | 42.29218 | 0.079017434 | 0.007902 |
| DR | 42 | Lithocholic | 266.74216 | 153.50886 | 379.97547 | 53.41432 | 0.100447789 | 0.010045 |
| NDR | 0 | Lithocholic | 56.86471 | −39.08019 | 152.80961 | 34.55674 | 0.021413692 | 0.002141 |
| NDR | 14 | Lithocholic | 74.4107 | −46.58682 | 195.40822 | 47.07009 | 0.028021031 | 0.002802 |
| NDR | 42 | Lithocholic | 1.718967 | −1.665428 | 5.103362 | 1.218967 | 0.000647316 | 6.47E−05 |
| Healthy | | Lithocholic | 601.7535 | 314.6058 | 888.9013 | 126.9354 | 0.22660388 | 0.02266 |

Note:
The values <1 nmol/g were under the limit of detection; so an appropriate value 0.5 was used for the calculation.

TABLE 11
16S rRNA Sequences of OTUs in Tables 2-5
OTUs in Table 2
JRPJ01000002.1034290.1035971
AGAGTTTGATCCTGGCTCAGAGTGAACGCTGGCGGCGTGCCTAATACATGCAAGTCGAACGATGAAACTTCTAGCT
TGCTAGAAGTGGATTAGTGGCGCACGGGTGAGTAATGCATAGGTAACATGCCCTTTAGTCTGGGATAGCCACTGGA
AACGGTGATTAATACTGGATACTCCCTACGGGGGAAAGGGGCTTTCAATAAAGAATTTCTCTTTTTAGTGTTTTGT
GTTGTTGGCACAAAATTCTAGTATTTGGAATGAGAAATTGGTGTTGTGAAGCAATTTGTGCGGAGATTAGACTTAG
TGTCTGTCGTGTCAGCAAATTGCGAACTCATCGATTTATCATCCAAAGACGAATTTTTTATTGAAAGCCTTCGCTA
AAGGATTGGCCTATGTCCTATCAGCTTGTTGGTGAGGTAATGGCTCACCAAGGCTATGACGGGTATCCGGCCTGAG
AGGGTGATCGGACACACTGGAACTGAGACACGGTCCAGACTCCTACGGGAGGCAGCAGTAGGGAATATTGCTCAAT
GGGGGAAACCCTGAAGCAGCAACGCCGCGTGGAGGATGAAGGTTTTAGGATTGTAAACTCCTTTTGTAAGAGAAGA
TTATGACGGTATCTTACGAATAAGCACCGGCTAACTCCGTGCCAGCAGCCGCGGTAATACGGAGGGTGCAAGCGTT
ACTCGGAATCACTGGGCGTAAAGAGCGCGTAGGCGGGTGGTCAAGTCAGATGTGAAATCCTGTAGCTTAACTACAG
AACTGCATTTGAAACTGACCATCTAGAGTATGGGAGAGGTAGGTGGAATTCTTGGTGTAGGGGTAAAATCCGTAGA
GATCAAGAGGAATACTCATTGCGAAGGCGACCTGCTGGAACATTACTGACGCTGATGCGCGAAAGCGTGGGGAGCA
AACAGGATTAGATACCCTGGTAGTCCACGCCCTAAACGATGAATGCTAGTTGTTGTGAGGCTTGTCCTTGCAGTAA
TGCAGCTAACGCATTAAGCATTCCGCCTGGGGAGTACGGTCGCAAGATTAAAACTCAAAGGAATAGACGGGGACCC
GCACAAGCGGTGGAGCATGTGGTTTAATTCGATGATACGCGAAGAACCTTACCTAGGCTTGACATTGATAGAATCT
ACTAGAGATAGTGGAGTGCCCTTTTAGGGAGCTTGAAAACAGGTGCTGCACGGCTGTCGTCAGCTCGTGTCGTGAG
ATGTTGGGTTAAGTCCCGCAACGAGCGCAACCCTCGTCCTTAGTTGCTAGCAGTTTGGCTGAGCACTCTAAGGAGA
CTGCCTTCGTAAGGAGGAGGAAGGTGAGGACGACGTCAAGTCATCATGGCCCTTACGCCTAGGGCTACACACGTGC
TACAATGGGGTGCACAAAGAGATGCAATAGTGTGAGCTGGAGCCAATCTCTAAAACATCTCTCAGTTCGGATTGTA
GTCTGCAACTCGACTACATGAAGCTGGAATCGCTAGTAATCGCAAATCAGCAATGTTGCGGTGAATACGTTCCCGG
GTCTTGTACTCACCGCCCGTCACACCATGGGAGTTGTATTTGCCTTAAGTCGGAATGCTAAATTGGCTACCGCCCA
CGGCAGATGCAGCGACTGGGGTGAAGTCGTAACAAGGTAACCGTAGGTGAACCTGCGGTTG (SEQ ID NO: 8)
JF920309.1.1340
AGTGAACGCTGGCGGCGTGCCTAATACATGCAAGTCGAACGATGAAGCTTCTAGCTTGCTAGAAGTGGATTAGTGG
CGCACGGGTGAGTAAGGTATAGTTAATCTGCCCTACACAAGAGGACAACACCTAGAAATGGGTGCTAATACTCTAT
ACTCCTGCTTAACACAAGTTGAGTAGGGAAAGTTTTTCGGTGTAGGATGAGACTATATAGTATCAGCTAGTTGGTA
AGGTAAAGGCTTACCAAGGCTATGACGCTTAAGAGGTCTGAGAGGATGATCTCTCACACTGGAACTGAGACACGGT
CCAGACTCCTACGGGAGGCAGCAGTAGGGAATATTGCGCAATGGGCGAAAGCCTGACGCAGCAACGCCGCGTGGAG
GATGACACTTTTAGGAGCGTAAACTCCTTTTCTTAGGGAAGAATTCTGACGGTACCTAAGGAATAAGCACCGGCTA
ACTCCGTGCCAGCAGCCGCGGTAATACGGAGGGTGCAAGCGTTACTCGGAATCACTGGGCGTAAAGGGCGCGTAGG
CGGATTATCAAGTCTCTTGTGAAATCTAATGGCTTAACCATTAAACTGCTTGGGAAACTGATAGTCTAGAGTGAGG
GAGAGGCAGATGGAATTGGTGGTGTAGGGGTAAAATCCGTAGATATCACCAAGAATACCCATTGCGAAGGCGATCT
GCTGGAACTCAACTGACGCTAAGGCGCGAAAGCGTGGGGAGCAAACAGGATTAGATACCCTGGTAGTCCACGCCCT
AAACGATGTATGCTAGTTGTTGGGCTGCTAGTCAGCTCAGTAATGCAGCTAACGCATTAAGCATACCGCCTGGGGA
GTACGGTCGCAAGATTAAAACTCAAAGGAATAGACGGGGACCCGCACAAGCGGTGGAGCATGTGGTTTAATTCGAA
GATACGCGAAGAACCTTACCTAGGCTTGATATCCAACAAAGCTTCTAGAGATAGAAGTGTGCTAGCTTGCTAGAAT
GTTGAGACAGGTGCTGCACGGCTGTCGTCAGCTCGTGTCGTGAGATGTTGGGTTAAGTCCCGCAACGAGCGCAACC
CACGTATTTAGTTGCTAACACTTCGGGTGAGCACTCTAAATAGACTGCCTTCGTAAGGAGGAGGAAGGTGTGGACG
ACGTCAAGTCATCATGGCCCTTATGCCTAGGGCGACACACGTGCTACAATGGCATATACAATGAGACGCAATACCG
CGAGGTGGAGCAAATCTATAAAATATGTCCCAGTTCGGATTGTTCTCTGCAACTCGAGAGCATGAAGCCGGAATCG
CTAGTAATCGCAAATCAGCCATGTTGCGGTGAATACGTTCCCGGGTCT (SEQ ID NO: 9)
FJ978526.1.1378
CATGCAAGTCGAACGGTAACATAGAGGAAGCTTGCTTTCTCTGATGACGAGTGGCGGACGGGTGAGTAAGGTCTGG
GAAACTGCCTGACAGAGGGGGACAACAACTGGAAACGGTTGCTAATACCGCATACACCCTGAGGGGGAAAGTCGAA
AGACGCTGTCAGATGTGCCCAGATGGGATTAGCTAGTAGGTGAGGTAAAGGCTCACCTAGGCGACGATCTCTAGCT
GGTCTGAGAGGATGATCAGCCACATTGGGACTGAGACACGGCCCAGACTCCTACGGGAGGCAGCAGTAGGGAATAT
TGCACAATGGGGGGAACCCTGATGCAGCCATGCCGCGTGTGTGAAGAAGGCCTTCGGGTTGTAAAGCACTTTCAGA
GGGGAGGAAAATGACGTTACCCTCAGAAGAAGCACCGGCTAACTCCGTGCCAGCAGCCGCGGTAATACGGAGGGTG
CAAGCGTTAATCGGAATAACTGGGCGTAAAGGGCATGCAGGCGGTTCTGCAAGTAGGGTGTGAAAGCCCGGGGCTC
AACCTCGGAATTGCACTCTAAACTGTGGGACTAGAGTATTGCAGGGGGAGACGGAATTCCAGGTGTAGCGGTGGAA
TGCGTAGAGATCTGGAAGAACACCAAAGGCGAAGGCAGTCTCCTGGGCAAATACTGACGCTCATATGCGAAAGCGT
GGGTAGCAAACAGGATTAGATACCCTGGTAGTCCACGCCGTAAACGATGTTGATTAGAAGCTTGCTTGTAAGAGTG
GGTTTCGCAGCTAACGCGATAAATCAACCGCCTGGGGAGTACGGCCGCAAGGTTAAAACTCAAATGAATTGACGGG
GGCCCGCACAAGCGGTGGAGCATGTGGTTTAATTCGACGCAACGCGATGAACCTTACCTGATCTTGACATCGCGAG
AATTACTTGTAATGAGTAAGTGCCTTCGGGAACTCGCAGACAGGTGCTGCATGGCTGTCGTCAGCTCGTGTCGTGA
GATGTTGGGTTAAGTCCCGCAACGAGCGCAACCCTTGTCCTTTGTTGCCAGCGGGTAGAGCCGGGAACTCAAAGGA
GACTGCCAGTGATAAACTGGAGGAAGGTAGGGATGACGTCAAGTCATCATGGCCCTTACGGTCAGGGCTACACACG
TGCTACAATGGGGCGTACAGAGGGAAACGAAACTGCGAGGTGGAGTGGAACCCAGAAAGCGTCCCTAAGTTCGGAT
TGGAGTCTGCAACTCGACTCCATGAAGTCGGAATCGCTAGTAATCGCAAATCAGAATGTTGCGGTGAATACGTTCC
CGGGCCTTGTACACACCGCCCGTCACACCATGGGAGTGGATTGCACCAGAAGTGGCCAGCCTAACTGCAAAGAGGG
CGGTACCACG (SEQ ID NO: 10)
New.ReferenceOTU45
TACGGAGGGTGCAAGCGTTAATCGGAATAACTGGGCGTAAAGGGCATGTAGGCGGAAAGGCAAGCAAGATGTGAAA
GACCTGGGCTCAACCTGGGTTGGTCATTTTGAACTACCTTTCTAGAGTATTGCAGAGGGAGATGGAATTTCAGGTG
TAGCGGTGGAATGCGTAGATATCTGAAAGAACACCAGAGGCGAAGGCGGTCTCCTGGGCAAATACTGACGCTGAGG
TGCGAAAGCGTGGGGAGCAAACAGG (SEQ ID NO: 11)
HK555938.1.1357
ACGGCACCCCTCTCCGGAGGGAAGCGAGTGGCGAACGGCTGAGTAACACGTGGAGAACCTGCCCCCTCCCCCGGGA
TAGCCGCCCGAAAGGACGGGTAATACCGGATACCCCCGGGCGCCGCATGGCGCCCGGGCTAAAGCCCCGACGGGAG
GGGATGGCTCCGCGGCCCATCAGGTAGACGGCGGGGTGACGGCCCACCGTGCCGACAACGGGTAGCCGGGTTGAGA
GACCGACCGGCCAGATTGGGACTGAGACACGGCCCAGACTCCTACGGGAGGCAGCAGTGGGGAATCTTGCGCAATG
GGGGGAACCCTGACGCAGCGACGCCGCGTGCGGGACGGAGGCCTTCGGGTCGTAAACCGCTTTCAGCAGGGAAGAG
TCAAGACTGTACCTGCAGAAGAAGCCCCGGCTAACTACGTGCCAGCAGCCGCGGTAATACGTAGGGGGCGAGCGTT
ATCCGGATTCATTGGGCGTAAAGCGCGCGTAGGCGGCCCGGCAGGCCGGGGGTCGAAGCGGGGGGCTCAACCCCCC
GAAGCCCCCGGAACCTCCGCGGCTTGGGTCCGGTAGGGGAGGGTGGAACACCCGGTGTAGCGGTGGAATGCGCAGA
TATCGGGTGGAACACCGGTGGCGAAGGCGGCCCTCTGGGCCGAGACCGACGCTGAGGCGCGAAAGCTGGGGGAGCG
AACAGGATTAGATACCCTGGTAGTCCCAGCCGTAAACGATGGACGCTGGGTGTGGGGGGACGATCCCCCCGTGCCG
CAGCCNACGCATTAAGCGTCCCGCCTGGGGAGTACGGCCGCAAGGCTAAAACTCAAAGGAATTGACGGGGGCCCGC
ACAAGCAGCGGAGCATGTGGCTTAATTCGAAGCAACGCGAAGAACCTTACGGCGCATCCCCCCGAGGCCCACGGGG
GGTCCGCCGCGTGGGTCAGAGGAGCGCATACGGGAGGTGCATGGTTGTCGTCAGCTCGTGTCGTGAGATGTTGGGT
TAAGTCCCGCAACGAGCGCAACCCCCGCCGCGTGTTGCCATCGGGTGATGCCGGGAACCCACGCGGGACCGCCGCC
GTCAAGGCGGAGGAGGGCGGGGACGACGTCAAGTCATCATGCCCCTTATGCCCTGGGCTGCACACGTGCTACAATG
GCCGGTACAGAGGGATGCCACCCCGCGAGGGGGAGCGGATCCCGGAAAGCCGGCCCCAGTTCGGATTGGGGGCTGC
AACCCGCCCCCATGAAGTCGGAGTTGCTAGTAATCGCGGATCAGCATGCCGCGGTGAATGCGTTCCCGGGCCTTGT
ACACACCGCCCGTCACACCACCCGAGTCGTCTGCACCCGAAGTCGCCGGCCCAACCGCAAGGGGG (SEQ ID NO:
12)
FJ957494.1.1454
TGAGTTTGATCATGGCTCAGGACGAACGCTGGCGGCGTGCCTAACACATGCAAGTCGAGCGATGAAATTTTCTTCG
GAAAATGGATTAGCGGCGGACGGGTGAGTAACACGTGGGTAACCTGCCCTATAGAGAGGGATAGCCTTCCGAAAGG
GAGATTAATACCTCATAATATCCTAGTATCGCATGATACATGGATTAAAGGAGCAATCCGCTATAGGATGGACCCG
CGGCGCATTAGCTAGTTGGTGAGGTAACGGCTCACCAAGGCGACGATGCGTAGCCGACCTGAGAGGGTGATCGGCC
ACATTGGGACTGAGACACGGCCCAGACTCCTACGGGAGGCAGCAGTGGGGAATATTGCACAATGGGGGAAACCCTG
ATGCAGCAACGCCGCGTGAGTGATGACGGTCTTCGGATTGTAAAGCTCTGTCTTTAGGGACGATAATGACGGTACC
TAAGGAGGAAGCCACGGCTAACTACGTGCCAGCAGCCGCGGTAATACGTAGGTGGCAAGCGTTGTCCGGATTTACT
GGGCGTAAAGGGAGCGTAGGCGGATCTTTAAGTGGGATGTGAAATACTCGGGCTCAACCTGGGGGCTGCATTCCAA
ACTGGGGATCTAGAGTACAGGAGGGGNGAGTGGAATTCCTAGTGTAGCGGTGAAATGCGTAGAGATTAGGAAGAAC
ACCAGTGGCGAAGGCGACTNTCTGGACTGTAACTGACGCTGAGGCTCGAAAGCGTGGGGAGCAAACAGGATTAGAT
ACCCTGGTAGTCCACGCCGTAAACGATGAATACTAGGTGTAGGGGGTGTCAACTCCCCCTGTGCCGCCGCTAACGC
ATTAAGTATTCCGCCTGGGGAGTACGGTCGCAAGATTAAAACTCAAAGGAATTGACGGGGGCCCGCACAAGTAGCG
GAGCATGTGGTTTAATTCGACGCAACGCGAAGAACCTTACCTAGACTTGACATCTTCTGCATTACCCTTAATCGGG
GAAGTTCCTTCGGGGACAGAATGACAGGTGGTGCATGGTTGTCGTCAGCTCGTGTCGTGAGATGTTGGGTTAAGTC
CCGCAACGAGCGCAACCCTTAAGCTTAGTTGCCATCATTAAGTTGGGCACTCTAAGTTGACTGCCGGTGACAAACC
GGAGGAAGGTGGGGATGACGTCAAATCATCATGCCCCTTATGTCTAGGGCTACACACGTGCTACAATGGCAAGTAC
AAAGAGAAGCAATACTGTGAAGTGGAGCAAAACTCAAAAACTTGTCTCAGTTCGGATTGTAGGCTGAAACTCGCCT
ACATGAAGCTGGAGTTGCTAGTAATCGCGAATCAGAATGTCGCGGTGAATACGTTCCCGGGTCTTGTACACACCGC
CCGTCACACCATGAGAGTTGGCAATACCCGAAGTCCGTAAGCTAACCGTAAGGAGGCAGCGGCCGAAGGTAGGGTC
AGCGATGGGG (SEQ ID NO: 13)
New.ReferenceOTU52
TACGTAGGTGGCGAGCGTTATCCGGATTTACTGGGCGTAAAGGGAGCGTAGGCGGATGATTAAGTGGGATGTGAAA
TACCCGGGCTCAACTTGGGTGCTGCATTCCAAACTGGTTATCTAGAGTGCAGGAGAGGAGAGTGGAATTCCTAGTG
TAGCGGTGAAATGCGTAGAGATTAGGAAGAACACCAGTGGCGAAGGCGACTCTCTGGACTGTAACTGACGCTGAGG
CTCGAAAGCGTGGGGAGCAAACAGG (SEQ ID NO: 14)
DQ797046.1.1403
AGAGTTTGATCCTGGCTCAGGACGAACGCTGGCGGCATGCTTAACACATGCAAGTCGAACGGACTGATTCCTTCGG
GATGAAAGTTAGTGGCGAACGGGTGAGTAATGTATGAGCAACCTGCCTCTGTCAACGGGATAACAGTTGGAAACGA
CTGCTAATACGGTATATGACCACGGCACCGCATGGTGCAGCGGTAAAAGATTTTATCGGACAGAGATGGGCTCATA
TCCCATTAGGTAGTTGGTGAGATAACAGCCCACCAAGCCGACGATCAGTAGCCGGTCTGAGAGGATGAACGGCCAC
ACTGGAACTGAGACACGGTCCAGACTCCTACGGGAGGCAGCAGTGGGGAATCTTCCGCAATGGACGAAAGTCTGAC
GGAGCAACGCCGCGTGAACGATGAAGGTCTTCGGATTGTAAAGTTCTGTGATCCGGGACGAAGGCATTGATTGAGA
ACATTGATTGATGTTGACGGTACCGGAAAAGCAAGCCACGGCTAACTACGTGCCAGCAGCCGCGGTAATACGTAGG
TGGCAAGCGTTGTCCGGAATTATTGGGCGTAAAGCGCGCGCAGGCGGCCGTGCAAGTCCATCTTAAAAGCGTGGGG
CTTAACCCCATGAGGGGATGGAAACTGCAGGGCTGGAGTGTCGGAGGGGAAAGTGGAATTCCTAGTGTAGCGGTGA
AATGCGTAGAGATTAGGAAGAACACCGGTGGCGAAGGCGACTTTCTAGACGACAACTGACGCTGAGGCGCGAAAGC
GTGGGGAGCAAACAGGATTAGATACCCTGGTAGTCCACGCCGTAAACGATGGATACTAGGTGTAGGAGGTATCGAC
CCCTTCTGTGCCGGAGTTAACGCAATAAGTATCCCGCCTGGGAAGTACGATCGCAAGATTAAAACTCAAAGGAATT
GACGGGGGCCCGCACAAGCGGTGGAGTATGTGGTTTAATTCGACGCAACGCGAAGAACCTTACCAAGCCTTGACAT
TGATCGCAATCCGCAGAAATGCGGAGTTCCTCTTCGGAGGACGAGAAAACAGGTGGTGCACGGCTGTCGTCAGCTC
GTGTCGTGAGATGTTGGGTTAAGTCCCGCAACGAGCGCAACCCCTATCTTCTGTTGCCAGCACGTAAAGGTGGGAA
CTCAGGAGAGACCGCCGCGGACAACGCGGAGGAAGGCGGGGATGACGTCAAGTCATCATGCCCCTTATGGCTTGGG
CTACACACGTACTACAATGGGTGCAAACAAAGAGAAGCGAAGTCGCGAGATGGAGCGGACCTCATAAACGCACTCC
CAGTTCAGATTGCAGGCTGCAACCCGCCTGCATGAAGTAGGAATCGCTAGTAATCGCGGGTCAGCATACCGCGGTG
AATACGTTCCCGGGCCTTGTACACACCGCCCGTCA (SEQ ID NO: 15)
GQ449092.1.1375
CTGGCTCAGGATGAACGCTGGCGGCGTGCTTAACACATGCAAGTCGAACGAAGAGGGTTAGAATGAGAGCTTCGGC
AGGATTTCTTTCCATCTTAGTGGCGGACGGGTGAGTAACGTGTGGGCAACCTGCCCTGTACTGGGGGATAATCATT
GGAAACGATGACTAATACCGCATGTGGTTCTCGGAAGGCATCTTCTGAGGAAGAAAGGATTTATTCGGTACAGGAT
GGGCCCGCATCTGATTAGCTAGTTGGTGAGATAACAGCCCACCAAGGCGACGATCAGTAGCCGACCTGAGAGGGTG
ATCGGCCACATTGGGACTGAGACACGGCCCAAACTCCTACGGGAGGCAGCAGTGGGGAATATTGCACAATGGGCGA
AAGCCTGATGCAGCAACGCCGCGTGAAGGATGAAGGGTTTCGGCTCGTAAACTTCTATCAATAGGGAAGAAACAAA
TGACGGTACCTAAATAAGAAGCCCCGGCTAACTACGTGCCAGCAGCCGCGGTAATACGTAGGGGGCAAGCGTTATC
CGGAATTACTGGGTGTAAAGGGAGCGTAGGCGGCATGGTAAGCCAGATGTGAAAGCCTTGGGCTTAACCCGAGGAT
TGCATTTGGAACTATCAAGCTAGAGTACAGGAGAGGAAAGCGGAATTCCTAGTGTAGCGGTGAAATGCGTAGATAT
TAGGAAGAACACCAGTGGCGAAGGCGGCTTTCTGGACTGAAACTGACGCTGAGGCTCGAAAGCGTGGGGAGCAAAC
AGGATTAGATACCCTGGTAGTCCACGCCGTAAACGATGAGTGCTAGGTGTCGGGGAGGAATCCTCGGTGCCGTAGC
TAACGCAATAAGCACTCCACCTGGGGAGTACGACCGCAAGGTTGAAACTCAAAGGAATTGACGGGGGCCCGCACAA
GCGGTGGAGCATGTGGTTTAATTCGAAGCAACGCGAAGAACCTTACCAAGGCTTGACATCCCGATGACCGTCCTAG
AGATAGGACTTCTCTTCGGAGCATCGGTGACAGGTGGTGCATGGTTGTCGTCAGCTCGTGTCGTGAGATGTTGGGT
TAAGTCCCGCAACGAGCGCAACCCTTGTCACTAGTTGCTACGAAAGGGCACTCTAGTGAGACTGCCGGTGACAAAC
CGGAGGAAGGTGGGGATGACGTCAAGTCCTCATGGCCCTTATGGGTAGGGCTTCACACGTCATACAATGGTCGGAA
CAGAGGGCAGCGAAGCCGTGAGGCGGAGCCAATCCCAGAAAACCGATCGTAGTCCGGATTGCAGTCTGCAACTCGA
CTGCATGAAGTCGGAATCGCTAGTAATCGCGGATCAGCATGCCGCGGTGAATACGTTCCCGGGTCTTGTACACACC
GCCCGTA (SEQ ID NO: 16)
AMCI01001631.34.1456
GGCGCACGGGTGAGTAACACGTATCCAACCTGCCGATAACTCGGGGATAGCCTTTCGAAAGAAAGATTAATACCCG
ATGGCATGTAAAGACCTCCTGGTCTTTACATTAAAGAATTTCGGTTATCGATGGGGATGCGTTCCATTAGATAGTA
GGCGGGGTAACGGCCCACCTAGTCCACGATGGATAGGGGTTCTGAGAGGAAGGTCCCCCACATTGGAACTGAGACA
CGGTCCAAACTCCTACGGGAGGCAGCAGTGAGGAATATTGGTCAATGGACGCGAGTCTGAACCAGCCAAGTAGCGT
GAAGGAAGACTGCCCTATGGGTTGTAAACTTCTTTTATACGGGAATAAAGTATTCCACGTGTGGGATTTTGTATGT
ACCGTATGAATAAGGATCGGCTAACTCCGTGCCAGCAGCCGCGGTAATACGGAGGATCCGAGCGTTATCCGGATTT
ATTGGGTTTAAAGGGAGCGTAGGTGGAAGATTAAGTCAGCCTGTGAAAGTTTGCGGCTTAACCGTAAAATTGCAGT
TGATACTGGTTTTCTTGAGTGCAGTAGAGGTGGGCGGAATTCGTGGTGTAGCGGTGAAATGCTTAGATATCACGAA
GAACTCCGATTGCGAAGGCAGCTCACTGGACTGTAACTGACACTGATGCTCGAAAGTGTGGGTATCAAACAGGATT
AGATACCCTGGTAGTCCACACAGTAAACGATGAATACTCGCTGTTTGCGATATACAGTAAGCGGCCAAGCGAAAGC
GTTAAGTATTCCACCTGGGGAGTACGCCGGCAACGGTGAAACTCAAAGGAATTGACGGGGGCCCGCACAAGCGGAG
GAACATGTGGTTTAATTCGATGATACGCGAGGAACCTTACCCGGGCTTAAATTACACCTGAATAGATTGGAAACAT
TTTAGCCGCAAGGCAGGTGTGAAGGTGCTGCATGGTTGTCGTCAGCTCGTGCCGTGAGGTGTCGGCTTAAGTGCCA
TAACGAGCGCAACCCTTATCTTCAGTTACTAACAGTTATAGCTGAGGACTCTGAAGAGACTGCCGTCGTAAGATGT
GAGGAAGGTGGGGATGACGTCAAATCAGCACGGCCCTTACGTCCGGGGCTACACACGTGTTACAATGGGGGGTACA
GAAGGCTGCTACCTGGCGACAGGATGCCAATCCTTAAATCCTCTCTCAGTTCGGACTGGAGTCTGCAACCCGACTC
CACGAAGCTGGATTCGCTAGTAATCGCGCATCAGCCATGGCGCGGTGAATACGTTCCCGGGCCTTGTACACACCGC
CCGTCAAGCCATGAAAGCCGGGGGTACCTGAAGTGCGTAACCGCAAGGAGCGTCCTAGGGTAAAACTGGTAATTGG
GGCTAAGTCGTAACAAGGTAGCCGTACCGGAAGGTGCGGCTG (SEQ ID NO: 17)
KF842598.1.1394
AGAGTTTGATCCTGGCTCAGGATGAACGCTAGCGACAGGCTTAACACATGCAAGTCGAGGGGCAGCATGATTTGTA
GCAATACAGATTGATGGCGACCGGCGCACGGGTGAGTAACGCGTATGCAACTTACCTATCAGAGGGGGATAGCCCG
GCGAAAGTCGGATTAATACCCCATAAAACAGGGGTCCCGCATGGGAATATTTGTTAAAGATTCATCGCTGATAGAT
AGGCATGCGTTCCATTAGGCAGTTGGCGGGGTAACGGCCCACCAAACCGACGATGGATAGGGGTTCTGAGAGGAAG
GTCCCCCACATTGGTACTGAGACACGGACCAAACTCCTACGGGAGGCAGCAGTGAGGAATATTGGTCAATGGCCGA
GAGGCTGAACCAGCCAAGTCGCGTGAAGGAAGAAGGATCTATGGTCTGTAAACTTCTTTTATAGGGGAATAAAGTG
GAGGACGTGTCCTTTTTTGTATGTACCCTATGAATAAGCATCGGCTAACTCCGTGCCAGCAGCCGCGGTAATACGG
AGGATGCGAGCGTTATCCGGATTTATTGGGTTTAAAGGGTGCGTAGGTGGTGATTTAAGTCAGCGGTGAAAGTTTG
TGGCTCAACCATAAAATTGCCGTTGAAACTGGGTTACTTGAGTGTGTTTGAGGTAGGCGGAATGCGTGGTGTAGCG
GTGAAATGCATAGATATCACGCAGAACTCCGATTGCGAAGGCAGCTTACTAAACCATAACTGACACTGAAGCACGA
AAGCGTGGGGATCAAACAGGATTAGATACCCTGGTAGTCCACGCAGTAAACGATGATTACTAGGAGTTTGCGATAC
AATGTAAGCTCTACAGCGAAAGCGTTAAGTAATCCACCTGGGGAGTACGCCGGCAACGGTGAAACTCAAAGGAATT
GACGGGGGCCCGCACAAGCGGAGGAACATGTGGTTTAATTCGATGATACGCGAGGAACCTTACCCGGGTTTGAACG
TAGTCTGACCGGAATGGAAACACTCCTTCTAGCAATAGCAGATTACAAGGTGCTGCATGGTTGCCTCAACTCCGGC
CCGGAAGGTCCGGCTTAATTGCCATAACAAGCGCACCCTTTTACCAAGGTTCAAACAGGTGAAGCTTGAAGACTCT
GTGGAACCTCCCCCCTAACCTGTGAGAAGAAGTGGGGATACACTCAATAAACCACGGCCCTTAATCCCGGGGGGAA
CACTGGTTACAATGGGTTGGGAAAGGGGGCTTCCTGGCGACAGGATGCTAATCTCCAAACCATGTCTCAGTTCGGA
TCGGAGTCTGCAACTCGACTCCGTGAAGCTGGATTCGCTAGTAATCGCGCATCAGCCATGGCGCGGTGAATACGTT
CCCGGGCCTTGTACACACCGCCCGTC (SEQ ID NO: 18)
HQ793763.1.1451
GATGAACGCTAGCTACAGGCTTAACACATGCAAGTCGAGGGGCAGCATGGTCTTAGCTTGCTAAGGCTGATGGCGA
CCGGCGCACGGGTGAGTAACACGTATCCAACCTGCCGTCTACTCTTGGCCAGCCTTCTGAAAGGAAGATTAATCCA
GGATGGGATCATGAGTTCACATGTCCGCATGATTAAAGGTATTTTCCGGTAGACGATGGGGATGCGTTCCATTAGA
TAGTAGGCGGGGTAACGGCCCACCTAGTCAACGATGGATAGGGGTTCTGAGAGGAAGGTCCCCCACATTGGAACTG
AGACACGGTCCAAACTCCTACGGGAGGCAGCAGTGAGGAATATTGGTCAATGGGCGCGAGCCTGAACCAGCCAAGT
AGCGTGAAGGATGACTGCCCTATGGGTTGTAAACTTCTTTTGTCCGGGAATAAAACCGCCTACGTGTAGGCGCTTG
TATGTACCGGTACGAATAAGCATCGGCTAACTCCGTGCCAGCAGCCGCGGTAATACGGAGGATGCGAGCGTTATCC
GGATTTATTGGGTTTAAAGGGAGCGCAGACGGGTTTTTAAGTCAGCTGTGAAAGTTTGGGGCTCAACCTTAAAATT
GCAGTTGATACTGGAGACCTTGAGTGCAGTTGAGGCAGGCGGAATTCGTGGTGTAGCGGTGAAATGCTTAGATATC
ACGAAGAACTCCGATTGCGAAGGTAGCTTGCTAAAGTGTAACTGACGTTCATGCTCGAAAGTGTGGGTATCAAACA
GGATTAGATACCCTGGTAGTCCACACGGTAAACGATGGATACTCGCTGTTGGCGATATACGGTCAGCGGCTTAGCG
AAAGCGTTAAGTATCCCACCTGGGGAGTACGCCGGCAACGGTGAAACTCAAAGGAATTGACGGGGGCCCGCACAAG
CGGAGGAACATGTGGTTTAATTCGATGATACGCGAGGAACCTTACCCGGGCTTAAATTGCACTGGACTATTCTGGA
AACAGGATATTCTTCGGACCAGTGTGAAGGTGCTGCATGGTTGTCGTCAGCTCGTGCCGTGAGGTGTCGGCTTAAG
TGCCATAACGAGCGCAACCCTTGCTGCCAGTTACTAACAGGTAATGCTGAGGACTCTGGCGGGACTGCCATCGTAA
GATGCGAGGAAGGTGGGGATGACGTCAAATCAGCACGGCCCTTACGTCCGGAGCTACACACGTGTTACAATGGTAG
GTACAGAGGGTAGCTACCCAGCGATGGGATGCGAATCTCGAAAGCCTATCTCAGTTCGGATTGGAGGCTGAAACCC
GCCTCCATGAAGTTGGATTCGCTAGTAATCGCGCATCAGCCATGGCGCGGTGAATACGTTCCCGGGCCTTGTACAC
ACCGCCCGTCAAGCCATGGGAGCCGGGGGTACCTGAAGTACGTAACCGCAAGGATCGTCCTAGGGTAAAACTGGTG
ACTGGGG (SEQ ID NO: 19)
DQ113765.1.1450
GATGAACGCTAGCTACAGGCTTAACACATGCAAGTCGAGGGGCAGCATGAAGTTTGCTTGCAAACTTTGATGGCGA
CCGGCGCACGGGTGAGTAACGCGTATCCAACCTCCCGCATACTCGGGGATAGCCTTCTGAAAGGAAGATTAATACC
CGATGGTATCTTAAGCGCACATGCAATTAAGATTAAAGAATTTCGGTATGCGATGGGGATGCGTTCCATTAGGTAG
TAGGCGGGGTAACGGCCCACCTAGCCATCGATGGATAGGGGTTCTGAGAGGAAGGTCCCCCACATTGGAACTGAGA
CACGGTCCAAACTCCTACGGGAGGCAGCAGTGAGGAATATTGGTCAATGGGCGCGAGCCTGAACCAGCCAAGTAGC
GTGAAGGATGACTGCCCTATGGGTTGTAAACTTCTTTTGTCCGGGAATAAAACCGCCTACGTGTAGGCGCTTGTAT
GTACCGGTACGAATAAGCATCGGCTAACTCCGTGCCAGCAGCCGCGGTAATACGGAGGATGCGAGCGTTATCCGGA
TTTATTGGGTTTAAAGGGAGCGCAGACGGGTTTTTAAGTCAGCTGTGAAAGTTTGGGGCTCAACCTTAAAATTGCA
GTTGATACTGGAGACCTTGAGTGCAGTTGAGGCAGGCGGAATTCGTGGTGTAGCGGTGAAATGCTTAGATATCACG
AAGAACTCCGATTGCGAAGGCAGCTTGCTAAAGTGTAACTGACGTTCATGCTCGAAAGTGTGGGTATCAAACAGGA
TTAGATACCCTGGTAGTCCACACGGTAAACGATGGATACTCGCTGTTGGCGATATACGGTCAGCGGCTTAGCGAAA
GCGTTAAGTATCCCACCTGGGGAGTACGCCGGCAACGGTGAAACTCAAAGGAATTGACGGGGGCCCGCACAAGCGG
AGGAACATGTGGTTTAATTCGATGATACGCGAGGAACCTTACCCGGGCTTAAATTGCACTGGACTTTCCCGGAAAC
GGGATTTTCTTCGGACCAGTGTGAAGGTGCTGCATGGTTGTCGTCAGCTCGTGCCGTGAGGTGTCGGCTTAAGTGC
CATAACGAGCGCAACCCTTGCTGCCAGTTACTAACAGGTAATGCTGAGGACTCTGGCGGGACTGCCATCGTAAGAT
GCGAGGAAGGTGGGGATGACGTCAAATCAGCACGGCCCTTACGTCCGGGGCTACACACGTGTTACAATGGGGGGTA
CAGAAGGCCGCTACCCGGCAACGGGATGCCAATCTCCAAAACCCCTCTCAGTTCGGACTGGAGTCTGCAACCCGAC
TCCACGAAGCTGGATTCGCTAGTAATCGCGCATCAGCCACGGCGCGGTGAATACGTTCCCGGGCCTTGTACACACC
GCCCGTCAAGCCATGAAAGCCGGGGGTACCTGAAGTGCGTAACCGCAAGGAGCGCCCTAGGGTAAAACTGGTAATT
GGGGCT (SEQ ID NO: 20)
ACBW01000012.3536.5054
AGAGTTTGATCCTGGCTCAGGATGAACGCTAGCTACAGGCTTAACACATGCAAGTCGAGGGGCAGCGGGATTGAAG
CTTGCTTCAATTGCCGGCGACCGGCGCACGGGTGAGTAACGCGTATCCAACCTTCCGCTTACTCGGGGATAGCCTT
TCGAAAGAAAGATTAATACCCGATGGTATCTTAAGCACGCATGAGATTAAGATTAAAGATTTATCGGTAAGCGATG
GGGATGCGTTCCATTAGGCAGTTGGCGGGGTAACGGCCCACCAAACCTACGATGGATAGGGGTTCTGAGAGGAAGG
TCCCCCACATTGGAACTGAGACACGGTCCAAACTCCTACGGGAGGCAGCAGTGAGGAATATTGGTCAATGGGCGAG
AGCCTGAACCAGCCAAGTAGCGTGAAGGATGACGGCCCTACGGGTTGTAAACTTCTTTTGTGCGGGAATAAAGGAA
CCTACGTGTAGGTTTTTGCATGTACCGTAACGAATAAGCATCGGCTAACTCCGTGCCAGCAGCCGCGGTAATACGG
AGGATGCGAGCGTTATCCGGATTTATTGGGTTTAAAGGGAGCGTAGACGGGTTTTTAAGTCAGCTGTGAAAGTTTG
GGGCTCAACCTTAAAATTGCAGTTGAAACTGGAGACCTTGAGTACGGTTGAGGCAGGCGGAATTCGTGGTGTAGCG
GTGAAATGCTTAGATATCACGAAGAACCCCGATTGCGAAGGCAGCCTGCTAAGCCGCCACTGACGTTGAGGCTCGA
AAGTGCGGGTATCAAACAGGATTAGATACCCTGGTAGTCCGCACGGTAAACGATGGATACTCGCTGTTGGCGATAG
ACAGTCAGCGGCCAAGCGAAAGCGTTAAGTATCCCACCTGGGGAGTACGCCGGCAACGGTGAAACTCAAAGGAATT
GACGGGGGCCCGCACAAGCGGAGGAACATGTGGTTTAATTCGATGATACGCGAGGAACCTTACCCGGGCTTGAACT
GCAGTGGAATTATCCGGAAACGGATAAGCGAGCAATCGCCGCTGTGGAGGTGCTGCATGGTTGTCGTCAGCTCGTG
CCGTGAGGTGTCGGCTTAAGTGCCATAACGAGCGCAACCCTTGCTGCCAGTTACTAACAGGTCATGCTGAGGACTC
TGGCAGGACTGCCATCGTAAGATGCGAGGAAGGTGGGGATGACGTCAAATCAGCACGGCCCTTACGTCCGGGGCTA
CACACGTGTTACAATGGGGAGTACAGAGGGCAGCTACCGGGCGACCGGATGCGAATCCCGAAAGCTCCTCTCAGTT
CGGACTGGAGTCTGCAACCCGACTCCACGAAGCTGGATTCGCTAGTAATCGCGCATCAGCCACGGCGCGGTGAATA
CGTTCCCGGGCCTTGTACACACCGCCCGTCAAGCCATGAAAGCCGGGGGTACCTGAAGTACGTAACCGCGAGGATC
GTCCTAGGGTAAAACCGGTAATTGGGGCTAAGTCGTAACAAGGTAGCCGTACCGGAAGGTGCGGCTG (SEQ ID
NO: 21)
HK693629.1.1491
AGAGTTTGATCCTGGCTCAGGATGAACGCTGGCGGCGTGCTTAACACATGCAAGTCGAACGAGAAACATTTTAATG
AAGCTTCGGCAGATTTAGTTTGTTTCTAGTGGCGGACGGGTGAGTAACGCGTGGGTAACCTGCCTCACACTGGGGG
ATAACAGTCAGAAATGACTGCTAATACCGCATAAGCGCACGGAACCGCATGGTTTTGTGTGAAAAACTCCGGTGGT
GTGAGATGGACCCGCGTTGGATTAGCCAGTTGGCAGGGTAACGGCCTACCAAAGCGACGATCCATAGCCGGCCTGA
GAGGGTGAACGGCCACATTGGGACTGAGACACGGCCCAGACTCCTACGGGAGGCAGCAGTGGGGAATATTGCACAA
TGGGGGAAACCCTGATGCAGCGACGCCGCGTGAAGGAAGAAGTATCTCGGTATGTAAACTTCTATCAGCAGGGAAG
ATAATGACGGTACCTGACTAAGAAGCCCCGGCTAACTACGTGCCAGCAGCCGCGGTAATACGTAGGGGGCAAGCGT
TATCCGGATTTACTGGGTGTAAAGGGAGCGTAGACGGAGCAGCAAGTCTGATGTGAAAGGCAGGGGCTCAACCCCT
GGACTGCATTGGAAACTGTTGATCTTGAGTACCGGAGGGGTAAGCGGAATTCCTAGTGTAGCGGTGAAATGCGTAG
ATATTAGGAGGAACACCAGTGGCGAAGGCGGCTTACTGGACGGTAACTGACGTTGAGGCTCGAAAGCGTGGGGAGC
AAACAGGATTAGATACCCTGGTAGTCCACGCCGTAAACGATGAATACTAGGTGTCGGGTGGCAGAGCCATTCGGTG
CCGCAGCAAACGCAGTAAGTATTCCACCTGGGGAGTACGTTCGCAAGAATGAAACTCAAAGGAATTGACGGGGACC
CGCACAAGCGGTGGAGCATGTGGTTTAATTCGAAGCAACGCGAAGAACCTTACCAAGTCTTGACATCCCTCTGACC
GGTCCTTAACCGGACCTTTCCTTCGGGACAGAGGAGACAGGTGGTGCATGGTTGTCGTCAGCTCGTGTCGTGAGAT
GTTGGGTTAAGTCCCGCAACGAGCGCAACCCCTATCCCCAGTAGCCAGCATTTAAGGTGGGCACTCTGAGGAGACT
GCCAGGGATAACCTGGAGGAAGGCGGGGATGACGTCAAATCATCATGCCCCTTATGATTTGGGCTACACACGTGCT
ACAATGGCGTAAACAAAGGGAAGCAGAGCGGTGACGCCGAGCAAATCCCAAAAATAACGTCCCAGTTCGGACTGCA
GTCTGCAACTCGACTGCACGAAGCTGGAATCGCTAGTAATCGCGGATCAGAATGCCGCGGTGAATACGTTCCCGGG
TCTTGTACACACCGCCCGTCACACCATGGGAGTCAGTAACGCCCGAAGTCAGTGACCTAACCGAAAGGGAGGAGCT
GCCGAAGGCGGGACGGATGACTGGGGTGAAGTCGTAACAAGGTAACC (SEQ ID NO: 22)
JQ208053.1.1336
GATGAACGCTGACAGAATGCTTAACACATGCAAGTCTACTTGAATTCACTTCGGTGATAGTAAGGTGGCGGACGGG
TGAGTAACACGTAAAGAACTTGCCTTACAGTCTGGGACAACTATTGGAAACGATAGCTAATACCGGATATTATGCG
AGAGTCGCATGACTCTTGTATGAAAGCTATATGCGCTGTAAGAGAGCTTTGCGTCCCATTAGCTAGTTGGTGAGGT
AACGGCTCACCAAGGCCACGATGGGTAGCCGGCCTGAGAGGGTGAACGGCCACAAGGGGACTGAGACACGGCCCTT
ACTCCTACGGGAGGCAGCAGTGGGGAATATTGGACAATGGACCAAAAGTCTGATCCAGCAATTCTGTGTGCACGAT
GACGGTCTTAGGATTGTAAAGTGCTTTCAATTGGGAAGAAAAAAATGACGGTACCAATAGAAGAAGCGACGGCTAA
ATACGTGCCAGCAGCCGCGGTAATACGTATGTCGCAAGCGTTATCCGGATTTATTGGGCGTAAAGCGCGTCTAGGT
GGTTTGGTAAGTCTGATGTGAAAATGCGGGGCTCAACTCCGTATTGCGTTGGAAACTGCCTAACTAGAGTATCGGA
GAGGTGGGCGGAACTACAAGTGTAGAGGTGAAATTCGTAGATATTTGTAGGAATGCCGATAGAGAAGTCAGCTCAC
TGGACGAATACTGACACTGAAGCGCGAAAGCATGGGGAGCAAACAGGATTAGATACCCTGGTAGTCCATGCTGTAA
ACGATGATTACTAAGCGTCGGGGGTCGAACCTCGGCACTCAAGCTAACGCGATAAGTAATCCGCCTGGGGAGTACG
TACGCAAGTATGAAACTCAAAGGAATTGACGGGGACCCGCACAAGTGGTGGAGCATGTGGTTTAATTCGACGCAAC
GCGAGGAACCTTACCAGCGTTTGACATCCTAGGAATGAGAAAGAGATTTCTTAGTGCTCCTTCGGGAGAACCTAGA
GACAGGTGGTGCATGGCTGTCGTCAGCTCGTGTCGTGAGATGTTGGGTTAAGTCCCGCAACGAGCGCAACCCCTAT
TGTATGTTGCCATCATTAAGTTGGGCACTCATGCGATACTGCCTGCGATGAGCAGGAGGAAGGTGGGGATGACGTC
AAGTCATCATGCCCCTTATACGCTGGGCTACACACGTGCTACAATGGGCAGTACAGAGAGAAGCAAATCTGCGAGG
AGGAGCAAATCTCACAAAGCTGTTCGTAGTTCGGATTGTACTCTGCAACTCGAGTACATGAAGTTGGAATCACTAG
TAATCGCAAATCAGCTATGTTGCGGTGAATACGTTCTCGGGTCT (SEQ ID NO: 23)
GQ493166.1.1359
GATGAACGCTGGCGGCGTGCTTAACACATGCAAGTCGAACGGGAAACATTTTAATGAAGCTTCGGCAGATTTAGCT
TGTTTCTAGTGGCGGACGGGTGAGTAACGCGTGGGTAACCTGCCTCACACTGGGGGATAACAGTCAGAAATGACTG
CTAATACCGCATAAGCGCACGGAACCGCATGGTTTTGTGTGAAAAACTCCGGTGGTGTGAGATGGACCCGCGTTGG
ATTAGCCAGTTGGCAGGGTAACGGCCTACCAAAGCGACGATCCATAGCCGGCCTGAGAGGGTGAACGGCCACATTG
GGACTGAGACACGGCCCAGACTCCTACGGGAGGCAGCAGTGGGGAATATTGCACAATGGGGGAAACCCTGATGCAG
CGACGCCGCGTGAAGGAAGAAGTATCTCGGTATGTAAACTTCTATCAGCAGGGAAGAAAATGACGGTACCTGACTA
AGAAGCCCCGGCTAACTACGTGCCAGCAGCCGCGGTAATACGTAGGGGGCAAGCGTTATCCGGATTTACTGGGTGT
AAAGGGAGCGTAGACGGAATGGCAAGTCTGATGTGAAAGGCAGGGGCTCAACCCCTGGACTGCATTGGAAACTGTC
AGTCTTGAGTACCGGAGGGGTAAGCGGAATTCCTAGTGTAGCGGTGAAATGCGTAGATATTAGGAGGAACACCAGT
GGCGAAGGCGGCTTACTGGACGGTAACTGACGTTGAGGCTCGAAAGCGTGGGGAGCAAACAGGATTAGATACCCTG
GTAGTCCACGCCGTAAACGATGAATACGAGGTGTCGGGTGGGCAAAGCCATTCGGTGCCGCAGCAAACGCAAAAAG
TAATCCCACCTGGGGGAGTACGTTCCCAAGAATGAAACTCAAAGGAAATAGCGGGGACCCGCACAAGCGGTGGAGC
ATGTGGTGTATTTGAAGCAACGCGAAGAACCTTACCAAGTCTTGACATCCCTCTGACCGGTCCTTAACCGGACCTC
TCCTTCGGGACAGGGGAGACAGGTGGTGCATGGTTGTCGTCAGCTCGTGTCGTGAGATGTTGGGTTAAGTCCCGCA
ACGAGCGCAACCCCTATCCTTAGTAGCCAGCATCTGAGGTGGGCACTCTGAGGAGACTGCCAGGGATAACCTGGAG
GAAGGCGGGGAGGACGTCAAATCATCATGCCCCCTATGATTTGGGCTACACACGTGCTACAATGGCGTAAACAAAG
GGAAGCAGAGCGGTGACGCCGAGCAAATCCCAAAAATAACGTCCCAGTTCGGACTGCAGTCTGCAACTCGACTGCA
CGAAGCTGGAATCGCTAGTAATCGCGGATCAGAATGCCGCGGTGAATAAAAGCCCGGGTCTTGCACT (SEQ ID
NO: 24)
GQ448486.1.1387
AGAGTTTGATCATGGCTCAGGATGAACGCTGGCGGCGTGCTTAACACATGCAAGTCGAACGGGAATTACTTTATTG
AAGCTTTGGTCGATTTAATTTAATTATAGTGGCGGACGGGTGAGTAACGCGTGGGTAACCTGCCTTATACAGGGGG
ATAACAGTCAGAAATGGCTGCTAATACCGCATAAGCGCACAGAGCTGCATGGCTCAGTGTGAAAAACTCCGGTGGT
ATAAGATGGACCCGCGTTGGATTAGTTGGTTGGTGGGGTAACGGCCCACCAAGGCGACGATCCATAGCCGGCCTGA
GAGGGTGAACGGCCACATTGGGACTGAGACACGGCCCAGACTCATACGGGAGGCAGCAGTGGGGAATATTGCACAA
TGGGGGAAACCCTGATGCAGCGACGCCGCGTGAAGGAAGAAGTATCTCGGTATGTAAACTTCTATCAGCAGGGAAG
ATAGTGACGGTACCTGACTAAGAAGCCCCGGCTAACTACGTGCCAGCAGCCGCGGTAATACGTAGGGGGCAAGCGT
TATCCGGATTTACTGGGTGTAAAGGGAGCGTAGACGGTGTGGCAAGTCTGATGTGAAAGGCATGGGCTCAACCTGT
GGACTGCATTGGAAACTGTCATACTTGAGTGCCGGAGGGGTAAGCGGAATTCCTAGTGTAGCGGTGAAATGCGTAG
ATATTAGGAGGAACACCAGTGGCGAAGGCGGCTTACTGGACGGTAACTGACGTTGAGGCTCGAAAGCGTGGGGAGC
AAACAGGATTAGATACCCTGGTAGTCCACGCCGTAAACGATGAATACTAGGTGTCGGGTGGCAAAGCCATTCGGTG
CCGTCGCAAACGCAGTAAGTATTCCACCTGGGGAGTACGTTCGCAAGAATGAAACTCAAAGGAATTGACGGGGACC
CGCACAAGCGGTGGAGCATGTGGTTTAATTCGAAGCAACGCGAAGAACCTTACCAAGTCTTGACATCCGCCTGACC
GATCCTTAACCGGATCTTTCCTTCGGGACAGGCGAGACAGGTGGTGCATGGTTGTCGTCAGCTCGTGTCGTGAGAT
GTTGGGTTAAGTCCCGCAACGAGCGCAACCCCTATCCTCAGTAGCCAGCATTAAGTTGGGCACTCATGCGATACTG
CCTGCGATGAGCAGGAGGAAGGTGGGGATGACGTCAAGTCATCATGCCCCTTATACGCTGGGCTACACACGTGCTA
CAATGGGTAGTACAGAGAGTCGCAAACCTGCGAGGGGGAGCTAATCTCAGAAAACTATTCTCAGTTCGGATTGTAC
TCTGCAACTCGAGTACATGAAGTTGGAATCGCTAGTAATCGCAAATCAGCTATGTTGCGGTGAATACGTTCTCGGG
TCTTGCACTCACCGCCCGT (SEQ ID NO: 25)
GQ491426.1.1332
GCTCAGGATGAACGCTGGCGGCGTGCTTAACACATGCAAGTCGAGCGAAGCACTTGCCATTGACTCTTCGGAAGAT
TTGGCATTTGACTGAGCGGCGGACGGGTGAGTAACGCGTGGGTAACCTGCCTCATACGGGGGAATAACAGTTAGAA
ATGGCTGCTAATGCCGCATAACCGCACAGGACCGCATGGACTGGTGTGAAAAACTGAGGTGGTATGAGATGGGCCC
GCGTCTGATTAGGTTAGTTGGCGGGGTAACGGCCCACCAAGCCGACGATCAGTAGCCGACCTGAGAGGGACCGGCC
ACATTGGGACTGAGACATGGCCCAGACTCCTACGGGAGGCAGCAGTGGGGAATATTGCACAATGGAGGAAACTCTG
ATGCAGCGACGCCGCATGAAGGAAGAAGTATCTCGGTATGTAAACTTCTATCAGCAGGGAAGAAAATGACGGTACC
TGACTAAGAAGCCCCGGCTAACTACGTGCCAGCAGCCGCGGTAATACGTAGGGGGCAAGCGTTATCCGGATTTACT
GGGTGTAAAGGGAGCGTAGACGGACGGGCAAGTCTGATGTGAAAGCCCGGGGCTTAACCCCGGGACTGCATTGGAA
ACTGTCCATCTTGAGTGCCGGAGAGGTAAGCGGAATTCCTAGTGTAGCGGTGAAATGCGTAGATATTAGGAGGAAC
ACCAGTGGCGAAGGCGGCTTACTGGACGGTAACTGACGTTGAGGCTCGAAAGCGTGGGGAGCAAACAGGATTAGAT
ACCCTGGTAGTCCACGCCGTAAACGATCAATAATGGGTGTCGGGTTGCAAAGCAATCCGGTGCCGCAGCAAACGCA
GTAAGTATTCCCCCTCGGGAGTACGTTCGCAAGAATGAAACTCAAAGGAAGGGACGGGGATCCGCACAAGCGGCGG
AGCATGTGGTTTAATTAGAAGCAACGCGAAGAACCTTACCAAGTCTTGACATCTGCCTGACCGTTCCTTAACCGGA
ACTATCTTTCGGGACAGGCAAGACAGGTGGTGCATGGTTGTCGTCAGCTCGTGTCGTGAGATGTTGGGTTAAGTCC
CGCAACGAGCGCAACCCCTGTCCTTAGTAGCCAGCAGTCCGGCTGGGCACTCTAGGGAGACTGCCGGGGGTAACCC
GGAGGAAGGCGGGGAGGAGGTCAAATCATCATGCCCCCCCTGATTTGGGCTACACACGTGGTACAATGGCGTAAAC
AAAGGGAAGCGGAGTGGTGACGCTGAGCAAATCTCAAAAATAACGTCCCACTTCGGACTGCAGTCTGCAACTCGAC
TGCACGAAGCTGGAATCGCTAGTAATCGCGAATCAGAATG (SEQ ID NO: 26)
New.ReferenceOTU54
TACGTAGGGGGCAAGCGTTATCCGGATTTACTGGGTGTAAAGGGAGCGTAGACGGCATGGCAAGTCTGATGTGAAA
GGCAGGGGCTCAACTCCTGGACTGCATTGGAAACTGCCAGGCTTGAGTGCCGGAGGGGTAAGCGGAATTCCTAGTG
TAGCGGTGAAATGCGTAGATATTAGGAGGAACACCAGTGGCGAAGGCGGCTTACTGGACGGTAACTGACGTTGAGG
CTCGAAAGCGTGGGGAGCAAACAGG (SEQ ID NO: 27)
JN387556.1.1324
CGTAAGTAACCTGCCCTGTACACACGGATAACATACCGAAAGGTATGCTAATACGGGATAATATATTTTGATCGCA
TGGTCGAGATATCAAAGCTCCGGCGGTACACCAGGGACCCCCGACAGAGGAGCTAGTTGGTAGTAATGTCACCAAG
GCGACGATCAGAAGCCGAACTGAGAGGGGGATCCGCACATGACTGAGACACGGTCAAACTCCTACGGGAGGCAGCA
GTGGGGAATATGCCAATGGGCGAAAGCTGATGCAGCACGCGCGTGAGCGATGAGGCTCGGGTCGTAAAGCTCGTCT
CAAGGAAGATAATGACGGTACTTGAGGAGGAAGCCCCGGCTAACTACGTGCCAGCAGCCGCGGTAATACGTAGGGG
GCTAGCGTTATCCGGAATTACTGGGCGTAAAGGGTGCGTAGGCGGTCTTTCAAGTCAGGAGTGAAAGGCTACGGCT
CAACCGTAGTAAGCTCTTGAAACTGTAAGACTTGAGTGCAGGAGAGGAGAGTGGAATTCCTAGTGTAGCGGTGAAA
TGCGTAGATATTAGGAGGAACACCAGTTGCGAAGGCGGCTCTCTGGACTGTAACTGACGCTGAGGCACGAAAGCGT
GGGGAGCAAACAGGATTAGATACCCTGGTAGTCCACGCCGTAAACGATGAGTACTAGCTGTCGGAGGTTACCCCCT
TCGGTGGCGCAGCTAACGCATTAAGTACTCCGCCTGGGAAGTACGCTCGCAAGAGTGAAACTCAAAGGAATTGACG
GGGACCCGCACAAGTAGCGGAGCATGTGGTTTAATTCGAAGCAACGCGAAGAACCTTACCTAAGCTTGACATCCCA
CTGACCCTTCCCTAATCGGAAGCTTCCCTTCGGGACAGTGGTGACAGGTGGTGCATGGTTGTCGTCAGCTCGTGTC
GTGAGATGTTGGGTTAAGTCCCGCAACGAGCGCAACCCTTGCCTTTAGTTGCCAGCATTAAGTTGGGCACTCTAGA
GGGACTGCCAGGGATAACCCGGAGGAGTGGGGATGACGTCAAATCATCATGCCCTTATGCTAGGCTACACACGTGC
TACAATGGGTGGTCAGAGGCCAGCCAGTCGTGAGGCCGAGCTATCCCATAAGCCATTCTCGTCCGGATTGTAGGCT
GAACTCGCCTACATGAGCTGGAATTACAAGTATGCGATCGATGCTGCGTGATGCGTCCGGGTCTTGTACACACCGC
CCGTCACACCATGGGAGTTGGGGGCGCCCGAAGCCGGATTGCTAACCTTTTGGAAGCGTCCGTCGAAGGTGAAACC
AATAACTGGGGTGAAGTCGTAACAAGGTAACC (SEQ ID NO: 28)
OTUs in Table 3
GQ006324.1.1342
GACGAACGCTGGCGGCGTGCTTAACACATGCAAGTCGAACGGAAAGGCCCCAGCTCGCTGGGGTACTCGAGTGGCG
AACGGGTGAGTAACACGTGGGTGATCTGCCTTGCACTCTGGGATAAGCTTGGGAAACTGGGTCTAATACCGGATAT
GAACTGCCTTTAGTGTGGTGGTTGGAAAGTTTTTTCGGTGCAAGATGAGCTCGCGGCCTATCAGCTTGTTGGTGGG
GTAATGGCCTACCAAGGCGTCGACGGGTAGCCGGCCTGAGAGGGTGTACGGCCACATTGGGACTGAGATACGGCCC
AGACTCCTACGGGAGGCAGCAGTGGGGAATATTGCACAATGGGCGGAAGCCTGATGCAGCGACGCCGCGTGGGGGA
TGACGGCCTTCGGGTTGTAAACTCCTTTCGACAGGGACGAAGCTTTTTGTGACGGTACCTGTATAAGAAGCACCGG
CTAACTACGTGCCAGCAGCCGCGGTAATACGTAGGGTGCGAGCGTTGTCCGGAATTACTGGGCGTAAAGAGCTCGT
AGGTGGTTTGTCGCGTCGTCTGTGAAATTCCGGGGCTTAACTCCGGGCGTGCAGGCGATACGGGCATAACTTGAGT
ACTGTAGGGGAGACTGGAATTCCTGGTGTAGCGGTGAAATGCGCAGATATCAGGAGGAACACCGGTGGCGAAGGCG
GGTCTCTGGGCAGTAACTGACGCTGAGGAGCGAAAGCATGGGGAGCGAACAGGATTAGATACCCTGGTAGTCCATG
CCGTAAACGGTGGGCGCTAGGTGTGGGTTTCCTTCCACGGGATCCGTGCCGTAGCTAACGCATTAAGCGCCCCGCC
TGGGGAGTACGGCCGCAAGGCTAAAACTCAAAGGAATTGACGGGGGCCCGCACAAGCGGCGGAGCATGTGGATTAA
TTCGATGCAACGCGAAGAACCTTACCTGGGCTTGACATACACTGGATCGGGCTAGAGATAGTCTTTCCCTTTGTGG
CTGGTGTACAGGTGGTGCATGGTTGTCGTCAGCTCGTGTCGTGAGATGTTGGGTTAAGTCCCGCAACGAGCGCAAC
CCTTGTCTTATGTTGCCAGCATTTGGTTGGGGACTCATGAGAGACTGCCGGGGTCAACTCGGAGGAAGGTGGGGAT
GACGTCAAATCATCATGCCCCTTATGTCCAGGGCTTCACACATGCTACAATGGTCGGTACAACGCGCAGCGACACT
GTGAGGTGGAGCGAATCGCTGAAAGCCGGCCTTAGTTCGGATTGGGGTCTGCAACTCGACCCCATGAAGTCGGAGT
CGCTAGTAATCGCAGATCAGCAATGCTGCGGTGAATACGTTCCCGGGCCT (SEQ ID NO: 29)
New.ReferenceOTU52
TACGTAGGTGGCGAGCGTTATCCGGATTTACTGGGCGTAAAGGGAGCGTAGGCGGATGATTAAGTGGGATGTGAAA
TACCCGGGCTCAACTTGGGTGCTGCATTCCAAACTGGTTATCTAGAGTGCAGGAGAGGAGAGTGGAATTCCTAGTG
TAGCGGTGAAATGCGTAGAGATTAGGAAGAACACCAGTGGCGAAGGCGACTCTCTGGACTGTAACTGACGCTGAGG
CTCGAAAGCGTGGGGAGCAAACAGG (SEQ ID NO: 30)
HG798451.1.1400
CTTGTGTCACCAACCATAGGGAGGGGGAAAACATGGAAACGGGGTTCATACCGCATAACTTTTTTAGCCCAATGCA
TAAGAAGAAAGGCCTTTCGGGTTTCGGTAAAGGAGGCCCCCGCGGCTCTTATAGTGTGTGTGGAAGTAACCGCTTC
CACAAGGCCCAGGTTTCATACCCGACTGGAGAGTGTGTTCGCCACACTGGGGAAAGGACCCCCGGCCCAGTCTCTC
TAGGGGAGGCAGCAGTAGGAATTTTCGGCAAAGGAAAAAATTTCTGACCGAACAACGCCGGTTGAATGAAGAAGTT
TTTCGGATCGAAAAACTCTGTTGTTAGAGAAGAACAAGGACGTTAGTAACTGAACGTCCCCTGACGGTATCTAACC
AGAAAGCCACGGCTAATTACGTGCCAGCAGCCGCGGTAATACGTAGGTGGCAAGCGTTGTCCGGATTTATTGGGCG
TAAAGCGAGCGCAGGCGGTTTCTTAAGTCTGATGTGAAAGCCCCCGGCTCAACCGGGGAGGGTCATTGGAAACTGG
GAGACTTGAGTGCAGAAGAGGAGAGTGGAATTCCATGTGTAGCGGTGAAATGCGTAGATATATGGAGGAACACCAG
TGGCGAAGGCGGCTCTCTGGTCTGTAACTGACGCTGAGGCTCGAAAGCGTGGGGAGCAAACAGGATTAGATACCCT
GGTAGTCCACGCCGTAAACGATGAGTGCTAAGTGTTGGAGGGTTTCCGCCCTTCAGTGCTGCAGCAAACGCATTAA
GCACTCCGCCTGGGGAGTACGACCGCAAGGTTGAAACTCAAAGGAATTGACGGGGGCCCGCACAAGCGGTGGAGCA
TGTGGTTTAATTCGAAGCAACGCGAAGAACCTTACCAGGTCTTGACATCCTTTGACCACTCTAGAGATAGAGCTTT
CCCTTCGGGGACAAAGTGACAGGTGGTGCATGGTTGTCGTCAGCTCGTGTCGTGAGATGTTGGGTTAAGTCCCGCA
ACGAGCGCAACCCTTATTGTTAGTTGCCATCATTTAGTTGGGCACTCTAGCGAGACTGCCGGTGACAAACCGGAGG
AAGGTGGGGATGACGTCAAATCATCATGCCCCTTATGACCTGGGCTACACACGTGCTACAATGGGAAGTACAACGA
GTCGCTAGACCGCGAGGTCATGCAAATCTCTTAAAGCTTCTCTCAGTTCGGATTGCAGGCTGCAACTCGCCTGCAT
GAAGCCGGAATCGCTAGTAATCGCGGATCAGCACGCCGCGGTGAATACGTTCCCGGGCCTTGTACACACCGCCCGT
CACACCACGAGAGTTTGTAACACCCGAAGTCGGTGAGGTAACCTTTTTGGAGCCAGCCGCCTAAGGTGGGATAGAT
GATTGGGGTGAAGTCGTAACCAACGTATGCC (SEQ ID NO: 31)
HK557089.3.1395
AGACTTTAGCTTGCTAAAGTTGGAAGAGTTGCGAACGGGTGAGTAACGCGTAGGTAACCTGCCTACTAGCGGGGGA
TAACTATTGGAAACGATAGCTAATACCGCATAACAGCATTTAACCCATGTTAGATGCTTGAAAGGAGCAATTGCTT
CACTAGTAGATGGACCTGCGTTGTATTAGCTAGTTGGTGAGGTAACGGCTCACCAAGGCGACGATACATAGCCGAC
CTGAGAGGGTGATCGGCCACACTGGGACTGAGACACGGCCCAGACTCCTACGGGAGGCAGCAGTAGGGAATCTTCG
GCAATGGGGGCAACCCTGACCGAGCAACGCCGCGTGAGTGAAGAAGGTTTTCGGATCGTAAAGCTCTGTTGTAAGA
GAAGAACGTGTGTGAGAGTGGAAAGTTCACACAGTGACGGTAACTTACCAGAAAGGGACGGCTAACTACGTGCCAG
CAGCCGCGGTAATACGTAGGTCCCGAGCGTTGTCCGGATTTATTGGGCGTAAAGCGAGCGCAGGCGGTTTAATAAG
TCTGAAGTTAAAGGCAGTGGCTTAACCATTGTTCGCTTTGGAAACTGTTAGACTTGAGTGCAGAAGGGGAGAGTGG
AATTCCATGTGTAGCGGTGAAATGCGTAGATATATGGAGGAACACCGGTGGCGAAAGCGGCTCTCTGGTCTGTAAC
TGACGCTGAGGCTCGAAAGCGTGGGGAGCAAACAGGATTAGATACCCTGGTAGTCCACGCCGTAAACGATGAGTGC
TAGGTGTTAGGCCCTTTCCGGGGCTTAGTGCCGCAGCTAACGCATTAAGCACTCCGCCTGGGGAGTACGACCGCAA
GGTTGAAACTCAAAGGAATTGACGGGGGCCCGCACAAGCGGTGGAGCATGTGGTTTAATTCGAAGCAACGCGAAGA
ACCTTACCAGGTCTTGACATCCCGATGCTATTCCTAGAGATAGGAAGTTTCTTCGGAACTGTGAGACTTGAGGGCA
GAAGGGTAGAGTGCACTTGTATGGGGAGCTGTGGAATGCGTTCCCGCAACGAGCGCAACCCCTATTGTTAGTTGCC
ATCATTAAGTTGGGCACTCTAGCGAGACTGCCGGTAATAAACCGGAGGAAGGTGGGGATGACGTCAAATCATCATG
CCCCTTATGACCTGGGCTACACACGTGCTACAATGGTTGGTACAACGAGTCGCGAGTCGGTGACGGCAAGCAAATC
TCTTAAAGCCAATCTCAGTTCGGATTGTAGGCTGCAACTCGCCTACATGAAGTCGGAATCGCTAGTAATCGCGGAT
CAGCACGCCGCGGTGAATACGTTCCCGGGCCTTGTACACACCGCCCGTCACACCACGAGAGTTTGTAACACCCGAA
GTCGGTGAGGTANCCTTTTAGGAGC (SEQ ID NO: 32)
GQ448336.1.1418
AGAGTTTGATCATGGCTCAGGACGAACGCTGGCGGCGTGCTTAACACATGCAAGTCGAACGAGAAGAGATGAGAAG
CTTGCTTCTTATCTCTTCGAGTGGCAAACGGGTGAGTAACGCGTAAGCAACCTGCCCTTCAGATGGGGACAACAGC
TGGAAACGGCTGCTAATACCGAATACGTTCTTTTTGTCGCATGGCAGAGGGAAGAAAGGGAGGCTCTTCGGAGCTT
TCGCTGAAGGAGGGGCTTGCGTCTGATTAGCTAGTTGGAGGGGTAACGGCCCACCAAGGCGACGATCAGTAGCCGG
TCTGAGAGGATGAACGGCCACATTGGGACTGAGACACGGCCCAGACTCCTACGGGAGGCAGCAGTGGGGAATCTTC
CGCAATGGACGAAAGTCTGACGGAGCAACGCCGCGTGAACGATGACGGCCTTCGGGTTGTAAAGTTCTGTTATACG
GGACGAATGGCGTAGCGGTCAATACCCGTTACGAGTGACGGTACCGTAAGAGAAAGCCACGGCTAACTACGTGCCA
GCAGCCGCGGTAATACGTAGGTGGCAAGCGTTGTCCGGAATTATTGGGCGTAAAGGGCGCGCAGGCGGCGTCGTAA
GTCGGTCTTAAAAGTGCGGGGCTTAACCCCGTGAGGGGACCGAAACTGCGATGCTAGAGTATCGGAGAGGAAAGCG
GAATTCCTAGTGTAGCGGTGAAATGCGTAGATATTAGGAGGAACACCAGTGGCGAAAGCGGCTTTCTGGACGACAA
CTGACGCTGAGGCGCGAAAGCCAGGGGAGCAAACGGGATTAGATACCCCGGTAGTCCTGGCCGTAAACGATGGATA
CTAGGTGTAGGAGGTATCGACCCCTTCTGTGCCGGAGTTAACGCAATAAGTATCCCGCCTGGGGAGTACGGCCGCA
AGGCTGAAACTCAAAGGAATTGACGGGGGCCCGCACAAGCGGTGGAACATGTGGTTTAATTCGATGATACGCGAGG
AACCTTACCCGGGCTTAAATTGCAGTGGAATGATGTGGAAACATGTCAGTGAGCAATCACCGCTGTGAAGGTGCTG
CATGGTTGTCGTCAGCTCGTGCCGTGAGGTGTCGGCTTAAGTGCCATAACGAGCGCAACCCTTATCTTCAGTTACT
AACAGGTCATGCTGAGGACTCTGGAGAGACTGCCGTCGTAAGATGTGAGGAAGGTGGGGATGACGTCAAATCAGCA
CGGCCCTTACGTCCGGGGCTACACACGTGTTACAATGGGGGGTACAGAGGGCCGCTACCACGCGAGTGGATGCCAA
TCCCAAAAACCTCTCTCAGTTCGGACTGGAGTCTGCAACCCGACTCCACGAAGCTGGATTCGCTAGTAATCGCGCA
TCAGCCACGGCGCGGTGAATACGTTCCCGGGCCTTGCACTCACCGCCCGT (SEQ ID NO: 33)
KF842598.1.1394
AGAGTTTGATCCTGGCTCAGGATGAACGCTAGCGACAGGCTTAACACATGCAAGTCGAGGGGCAGCATGATTTGTA
GCAATACAGATTGATGGCGACCGGCGCACGGGTGAGTAACGCGTATGCAACTTACCTATCAGAGGGGGATAGCCCG
GCGAAAGTCGGATTAATACCCCATAAAACAGGGGTCCCGCATGGGAATATTTGTTAAAGATTCATCGCTGATAGAT
AGGCATGCGTTCCATTAGGCAGTTGGCGGGGTAACGGCCCACCAAACCGACGATGGATAGGGGTTCTGAGAGGAAG
GTCCCCCACATTGGTACTGAGACACGGACCAAACTCCTACGGGAGGCAGCAGTGAGGAATATTGGTCAATGGCCGA
GAGGCTGAACCAGCCAAGTCGCGTGAAGGAAGAAGGATCTATGGTCTGTAAACTTCTTTTATAGGGGAATAAAGTG
GAGGACGTGTCCTTTTTTGTATGTACCCTATGAATAAGCATCGGCTAACTCCGTGCCAGCAGCCGCGGTAATACGG
AGGATGCGAGCGTTATCCGGATTTATTGGGTTTAAAGGGTGCGTAGGTGGTGATTTAAGTCAGCGGTGAAAGTTTG
TGGCTCAACCATAAAATTGCCGTTGAAACTGGGTTACTTGAGTGTGTTTGAGGTAGGCGGAATGCGTGGTGTAGCG
GTGAAATGCATAGATATCACGCAGAACTCCGATTGCGAAGGCAGCTTACTAAACCATAACTGACACTGAAGCACGA
AAGCGTGGGGATCAAACAGGATTAGATACCCTGGTAGTCCACGCAGTAAACGATGATTACTAGGAGTTTGCGATAC
AATGTAAGCTCTACAGCGAAAGCGTTAAGTAATCCACCTGGGGAGTACGCCGGCAACGGTGAAACTCAAAGGAATT
GACGGGGGCCCGCACAAGCGGAGGAACATGTGGTTTAATTCGATGATACGCGAGGAACCTTACCCGGGTTTGAACG
TAGTCTGACCGGAATGGAAACACTCCTTCTAGCAATAGCAGATTACAAGGTGCTGCATGGTTGCCTCAACTCCGGC
CCGGAAGGTCCGGCTTAATTGCCATAACAAGCGCACCCTTTTACCAAGGTTCAAACAGGTGAAGCTTGAAGACTCT
GTGGAACCTCCCCCCTAACCTGTGAGAAGAAGTGGGGATACACTCAATAAACCACGGCCCTTAATCCCGGGGGGAA
CACTGGTTACAATGGGTTGGGAAAGGGGGCTTCCTGGCGACAGGATGCTAATCTCCAAACCATGTCTCAGTTCGGA
TCGGAGTCTGCAACTCGACTCCGTGAAGCTGGATTCGCTAGTAATCGCGCATCAGCCATGGCGCGGTGAATACGTT
CCCGGGCCTTGTACACACCGCCCGTC (SEQ ID NO: 34)
FJ950694.1.1472
CGCCCTGATTGACGGCTATACACATGCAAGTCGAACGGTAACAGGAAACAGCTTGCTTCTTTGCTGACGAGTGGCG
GACGGGTGAGTAATGTCTGGGAAACTGCCTGATGGAGGGGGATAACTACTGGAAACGGTAGCTAATACCGCATAAC
GTCGCAAGACCAAAGAGGGGGACCTTCGGGCCTCTTGCCATCGGATGTGCCCAGATGGGATTAGCTAGTAGGTGGG
GTAACGGCTCCATCCCTAGGCGAGCCGAATCCTTAGCCTGGTCTGAGAGGAATGACCAGCCACACTGGGACTGAGA
ACACGGTCCAGACTCCTACGGGAGGCAGCAGTGGGGAATATTGCACAATGGGCGCAAGCCTGATGCAGCCATGCCG
CGTGTATGAAGAAGGCCTTCGGGTTGTAAAGTACTTTCAGCGGGGAGGAAGGGAGTAAAGTTAATACCCTTTGCTC
ATTGACGTTACCCGCAGAAGAAGCACCGGCTAACTCCGTGCCAGCAGCCGCGGTAATACGGAGGGTGCAAGCGTTA
ATCGGAATTACTGGGCGTAAAGCGCACGCAGGCGGTTTGTTAAGTCAGATGTGAAATCCCCGGGCTCAACCTGGGA
ACTGCATCTGATACTGGCAAGCTTGAGTCTCGTAGAGGGGGGTAGAATTCCAGGTGTAGCGGTGAAATGCGTAGAG
ATCTGGAGGAATACCGGTGGCGAAGGCGGCCCCCTGGACGAAGACTGACGCTCAGGTGCGAAAGCGTGGGGAGCAA
ACAGGATTAGATACCCTGGTAGTCCACGCCGTAAACGATGTCGACTTGGAGGTTGTGCCCTTGAGGCGTGGCTTCC
GGAGCTAACGCGTTAAGTCGACCGCCTGGGGAGTACGGCCGCAAGGTTAAAACTCAAATGAATTGACGGGGGCCCG
CACAAGCGGTGGAGCATGTGGTTTAATTCGATGCAACGCGAAGAACCTTACCTGGTCTTGACATCCACGGGAAGTT
TTCAGAGATGAGAATGTGCCTTCGGGAACCGTGAGACAGGTGCTGCATGGCTGTCGTCAGCTCGTGTTGTGAAATG
TTGGGTTAAGTCCCGCAACGAGCGCAACCCTTATCCTTTGTTGCCAGCGGTCCGGCCGGGAACTCAAAGGAGACTG
CCAGTGATAAACTGGAGGAAGGTGGGGATGACGTCCAGGTCATCATGGCCCTTACGAACCAGGGCTACACACGTGC
CTACAATGGACGCATCCAAAGAGAGAGCGAACCCTGCCCGCGAGAGCAAGCGGACCTCATAAAGTGCGTCGTAGTC
CGGATTGGAGTCTGCAACTCGACTCCATGAAGTCGGAATCGCTAGTAATCGTGGATCAGAATGCCACGGTGAATAC
GTTCCCGGGCCTTGTACACACCGCCCGTCACACCATGGGAGTGGGTTGCAAAAGAAGTAGGTAGCTTAACCTTCGG
GAGGGCGCTTACCACTTTGGATGCGAGG (SEQ ID NO: 35)
HQ802983.1.1440
TAAGATGAACGCTGGCGGCGTGCTTAACACATGCAAGTCCTATGAAGCGCTTAAACGGATTTCTTCGGATTGAAGT
TTTTGTGACTGAGTGGCGGACGGGTGAGTAACGCGTGGGTAACTTGCCTCATACAGGGGGATAACAGTTAGAAATG
ACTGCTAATACCGCATAAGCGCACAGTGCTGCATGGCACAGTGTGAAAAACTCCGGTGGTATGAGATGGACCCGCG
TCTGATTAGCTAGTTGGTGGGGTAACGGCCTACCAAGGCGACGATCAGTAGCCGGCCTGAGAGGGTGAACGGCCAC
ATTGGGACTGAGACACGGCCCAAACTCCTACGGGAGGCAGCAGTGGGGAATATTGCACAATGGGGGAAACCCTGAT
GCAGCGACGCCGCGTGAGCGAAGAAGTATTTCGGTATGTAAAGCTCTATCAGCAGGGAAGAAAATGACGGTACCTG
ACTAAGAAGCACCGGCTAAATACGTGCCAGCAGCCGCGGTAATACGTATGGTGCAAGCGTTATCCGGATTTACTGG
GTGTAAAGGGAGCGTAGACGGTTGTGTAAGTCTGATGTGAAAGCCCGGGGCTCAACCCCGGGACTGCATTGGAAAC
TATGTAACTAGAGTGTCGGAGAGGTAAGCGGAATTCCTAGTGTAGCGGTGAAATGCGTAGATATTAGGAGGAACAC
CAGTGGCGAAGGCGGCTTACTGGACGATCACTGACGTTGAGGCTCGAAAGCGTGGGGAGCAAACAGGATTAGATAC
CCTGGTAGTCCACGCCGTAAACGATGACTACTAGGTGTCGGGGCCCATAAGGGCTTCGGTGCCGCAGCAAACGCAA
TAAGTATTCCACCTGGGGAGTACGTTCGCAAGAATGAAACTCAAAGGAATTGACGGGGACCCGCACAAGCGGTGGA
GCATGTGGTTTAATTCGAAGCAACGCGAAGAACCTTACCTGGTCTTGACATCCCACTGACCGGACAGTAATGTGTC
CTTTCCTCCGGGACAGTGGAGACAGGTGGTGCATGGTTGTCGTCAGCTCGTGTCGTGAGATGTTGGGTTAAGTCCC
GCAACGAGCGCAACCCCTATCCTTAGTAGCCAGCAGTAAGATGGGCACTCTAGGGAGACTGCCAGGGATAACCTGG
AGGAAGGTGGGGATGACGTCAAATCATCATGCCCCTTATGACTTGGGCTACACACGTGCTACAATGGCGTAAACAA
AGTGAAGCGAAGTCGTGAGGCCAAGCAAATCACAAAAATAACGTCTCAGTTCGGATTGTAGTCTGCAACTCGACTA
CAAGAAGCTGGAATCGCTAGTAATCGCAGATCAGAATGCTGCGGTGAATACGTTCCCGGGTCTTGTACACACCGCC
CGTCACACCATGGGAGTCGAAAATGCCCGAAGTCGGTGACCTAACGAAAGAAGGAGCCGCCGAAGGCAGGTT
(SEQ ID NO: 36)
GQ448468.1.1366
AGAGTTTGATCCTGGCTCAGGATGAACGCTGACAGAATGCTTAACACATGCAAGTATACTTGATCCTTCGGGTGAT
GGTGGCGGACGGGTGAGTAACGCGTAAAGAACTTGCCCTGCAGTCTGGGACAACATTTGGAAACGAATGCTAATCC
CGCATAAGCCCACAGCTCGGCATCGAGCAGAGGGAAAAGGAGTGATCTGCTTTGAGATGGCCTCGCGTCCGATTAG
CTGGTTGGTGAGGTGACGGCCCATCAAGGCAACGATCGGTAGCCGGACTGAGAGGTTGAACGGCCACATTGGGATT
GAGACACGGCCCTTACTCCTACGGGAGGCAGCAGTGGGGAATATTGGACAATGGACCAAAAGTCTGATCCAGCAAT
TCTGTGTGCACGATGAAGTTTTTCGGAATGTAAAGTGCTTTCAGTTGGGACGAAGTAAGTGACGGTACCAACAGAA
GAAGCGACGGCTAAATACGTGCCAGCAGCCGCGGTAATACGTATGTCGCAAGCGTTATCCGGATTTATTGGGCGTA
AAGCGCGTCTAGGCGGTTTGGTAAGTCTGATGTGAAAATGCGGGGCTCAACTCCGTATTGCGTTGGAAACTGCCAA
ACTAGAGTACTGGAGAGGTGGGCGGAACTACAAGTGTAGAGGTGAAATTCGTAGATATTTGTAGGAATGCCGATGG
GGAAGCCAGCCCACTGGACAGATACTGACGCTAAAGCGCGAAAGCGTGGGTAGCAAACAGGATTAGATACCCTGGT
AGTCCACGCCGTAAACGATGATTACTAGGTGTTGGGGGTCGAACCTCAGCGCCCAAGCTAACGCGATAAGTAATCC
GCCTGGGGAGTACGTACGCAAGTATGAAACTCAAAGGAATTGACGGGGACCCGCACAAGCGGTGGAGCATGTGGTT
TAATTCGACGCAACGCGAGGAACCTTACCAGCGTTTGACATCCTAAGAAATTAGCAGAGATGCTTTTGTGCCCCTT
CGGGGGAACTTAGTGACAGGTGGTGCATGGCTGTCGTCAGCTCGTGTCGTGAGATGTTGGGTTAAGTCCCGCAACG
AGCGCAACCCCTTTCGTATGTTGCCATCATTAAGTTGGGCACTCATGCGATACTGCCTGCGATGAGCAGGAGGAAG
GTGGGGATGACGTCAAGTCATCATGCCCCTTATACGCTGGGCTACACACGTGCTACAATGGGTAGTACAGAGAGTC
GCAAACCTGCGAGGGGGAGCTAATCTCAGAAAACTATTCTCAGTTCGGATTGTACTCTGCAACTCGAGTACATGAA
GTTGGAATCGCTAGTAATCGCAAATCAGCTATGTTGCGGTGAATACGTTCTCGGGTCTTGTACACACCGCCCGT
(SEQ ID NO: 37)
JN387556.1.1324
CGTAAGTAACCTGCCCTGTACACACGGATAACATACCGAAAGGTATGCTAATACGGGATAATATATTTTGATCGCA
TGGTCGAGATATCAAAGCTCCGGCGGTACACCAGGGACCCCCGACAGAGGAGCTAGTTGGTAGTAATGTCACCAAG
GCGACGATCAGAAGCCGAACTGAGAGGGGGATCCGCACATGACTGAGACACGGTCAAACTCCTACGGGAGGCAGCA
GTGGGGAATATGCCAATGGGCGAAAGCTGATGCAGCACGCGCGTGAGCGATGAGGCTCGGGTCGTAAAGCTCGTCT
CAAGGAAGATAATGACGGTACTTGAGGAGGAAGCCCCGGCTAACTACGTGCCAGCAGCCGCGGTAATACGTAGGGG
GCTAGCGTTATCCGGAATTACTGGGCGTAAAGGGTGCGTAGGCGGTCTTTCAAGTCAGGAGTGAAAGGCTACGGCT
CAACCGTAGTAAGCTCTTGAAACTGTAAGACTTGAGTGCAGGAGAGGAGAGTGGAATTCCTAGTGTAGCGGTGAAA
TGCGTAGATATTAGGAGGAACACCAGTTGCGAAGGCGGCTCTCTGGACTGTAACTGACGCTGAGGCACGAAAGCGT
GGGGAGCAAACAGGATTAGATACCCTGGTAGTCCACGCCGTAAACGATGAGTACTAGCTGTCGGAGGTTACCCCCT
TCGGTGGCGCAGCTAACGCATTAAGTACTCCGCCTGGGAAGTACGCTCGCAAGAGTGAAACTCAAAGGAATTGACG
GGGACCCGCACAAGTAGCGGAGCATGTGGTTTAATTCGAAGCAACGCGAAGAACCTTACCTAAGCTTGACATCCCA
CTGACCCTTCCCTAATCGGAAGCTTCCCTTCGGGACAGTGGTGACAGGTGGTGCATGGTTGTCGTCAGCTCGTGTC
GTGAGATGTTGGGTTAAGTCCCGCAACGAGCGCAACCCTTGCCTTTAGTTGCCAGCATTAAGTTGGGCACTCTAGA
GGGACTGCCAGGGATAACCCGGAGGAGTGGGGATGACGTCAAATCATCATGCCCTTATGCTAGGCTACACACGTGC
TACAATGGGTGGTCAGAGGCCAGCCAGTCGTGAGGCCGAGCTATCCCATAAGCCATTCTCGTCCGGATTGTAGGCT
GAACTCGCCTACATGAGCTGGAATTACAAGTATGCGATCGATGCTGCGTGATGCGTCCGGGTCTTGTACACACCGC
CCGTCACACCATGGGAGTTGGGGGCGCCCGAAGCCGGATTGCTAACCTTTTGGAAGCGTCCGTCGAAGGTGAAACC
AATAACTGGGGTGAAGTCGTAACAAGGTAACC (SEQ ID NO: 38)
OTUs in Table 4
JRPJ01000002.1034290.1035971
AGAGTTTGATCCTGGCTCAGAGTGAACGCTGGCGGCGTGCCTAATACATGCAAGTCGAACGATGAAACTTCTAGCT
TGCTAGAAGTGGATTAGTGGCGCACGGGTGAGTAATGCATAGGTAACATGCCCTTTAGTCTGGGATAGCCACTGGA
AACGGTGATTAATACTGGATACTCCCTACGGGGGAAAGGGGCTTTCAATAAAGAATTTCTCTTTTTAGTGTTTTGT
GTTGTTGGCACAAAATTCTAGTATTTGGAATGAGAAATTGGTGTTGTGAAGCAATTTGTGCGGAGATTAGACTTAG
TGTCTGTCGTGTCAGCAAATTGCGAACTCATCGATTTATCATCCAAAGACGAATTTTTTATTGAAAGCCTTCGCTA
AAGGATTGGCCTATGTCCTATCAGCTTGTTGGTGAGGTAATGGCTCACCAAGGCTATGACGGGTATCCGGCCTGAG
AGGGTGATCGGACACACTGGAACTGAGACACGGTCCAGACTCCTACGGGAGGCAGCAGTAGGGAATATTGCTCAAT
GGGGGAAACCCTGAAGCAGCAACGCCGCGTGGAGGATGAAGGTTTTAGGATTGTAAACTCCTTTTGTAAGAGAAGA
TTATGACGGTATCTTACGAATAAGCACCGGCTAACTCCGTGCCAGCAGCCGCGGTAATACGGAGGGTGCAAGCGTT
ACTCGGAATCACTGGGCGTAAAGAGCGCGTAGGCGGGTGGTCAAGTCAGATGTGAAATCCTGTAGCTTAACTACAG
AACTGCATTTGAAACTGACCATCTAGAGTATGGGAGAGGTAGGTGGAATTCTTGGTGTAGGGGTAAAATCCGTAGA
GATCAAGAGGAATACTCATTGCGAAGGCGACCTGCTGGAACATTACTGACGCTGATGCGCGAAAGCGTGGGGAGCA
AACAGGATTAGATACCCTGGTAGTCCACGCCCTAAACGATGAATGCTAGTTGTTGTGAGGCTTGTCCTTGCAGTAA
TGCAGCTAACGCATTAAGCATTCCGCCTGGGGAGTACGGTCGCAAGATTAAAACTCAAAGGAATAGACGGGGACCC
GCACAAGCGGTGGAGCATGTGGTTTAATTCGATGATACGCGAAGAACCTTACCTAGGCTTGACATTGATAGAATCT
ACTAGAGATAGTGGAGTGCCCTTTTAGGGAGCTTGAAAACAGGTGCTGCACGGCTGTCGTCAGCTCGTGTCGTGAG
ATGTTGGGTTAAGTCCCGCAACGAGCGCAACCCTCGTCCTTAGTTGCTAGCAGTTTGGCTGAGCACTCTAAGGAGA
CTGCCTTCGTAAGGAGGAGGAAGGTGAGGACGACGTCAAGTCATCATGGCCCTTACGCCTAGGGCTACACACGTGC
TACAATGGGGTGCACAAAGAGATGCAATAGTGTGAGCTGGAGCCAATCTCTAAAACATCTCTCAGTTCGGATTGTA
GTCTGCAACTCGACTACATGAAGCTGGAATCGCTAGTAATCGCAAATCAGCAATGTTGCGGTGAATACGTTCCCGG
GTCTTGTACTCACCGCCCGTCACACCATGGGAGTTGTATTTGCCTTAAGTCGGAATGCTAAATTGGCTACCGCCCA
CGGCAGATGCAGCGACTGGGGTGAAGTCGTAACAAGGTAACCGTAGGTGAACCTGCGGTTG (SEQ ID NO: 39)
New.ReferenceOTU45
TACGGAGGGTGCAAGCGTTAATCGGAATAACTGGGCGTAAAGGGCATGTAGGCGGAAAGGCAAGCAAGATGTGAAA
GACCTGGGCTCAACCTGGGTTGGTCATTTTGAACTACCTTTCTAGAGTATTGCAGAGGGAGATGGAATTTCAGGTG
TAGCGGTGGAATGCGTAGATATCTGAAAGAACACCAGAGGCGAAGGCGGTCTCCTGGGCAAATACTGACGCTGAGG
TGCGAAAGCGTGGGGAGCAAACAGG (SEQ ID NO: 40)
GQ006324.1.1342
GACGAACGCTGGCGGCGTGCTTAACACATGCAAGTCGAACGGAAAGGCCCCAGCTCGCTGGGGTACTCGAGTGGCG
AACGGGTGAGTAACACGTGGGTGATCTGCCTTGCACTCTGGGATAAGCTTGGGAAACTGGGTCTAATACCGGATAT
GAACTGCCTTTAGTGTGGTGGTTGGAAAGTTTTTTCGGTGCAAGATGAGCTCGCGGCCTATCAGCTTGTTGGTGGG
GTAATGGCCTACCAAGGCGTCGACGGGTAGCCGGCCTGAGAGGGTGTACGGCCACATTGGGACTGAGATACGGCCC
AGACTCCTACGGGAGGCAGCAGTGGGGAATATTGCACAATGGGCGGAAGCCTGATGCAGCGACGCCGCGTGGGGGA
TGACGGCCTTCGGGTTGTAAACTCCTTTCGACAGGGACGAAGCTTTTTGTGACGGTACCTGTATAAGAAGCACCGG
CTAACTACGTGCCAGCAGCCGCGGTAATACGTAGGGTGCGAGCGTTGTCCGGAATTACTGGGCGTAAAGAGCTCGT
AGGTGGTTTGTCGCGTCGTCTGTGAAATTCCGGGGCTTAACTCCGGGCGTGCAGGCGATACGGGCATAACTTGAGT
ACTGTAGGGGAGACTGGAATTCCTGGTGTAGCGGTGAAATGCGCAGATATCAGGAGGAACACCGGTGGCGAAGGCG
GGTCTCTGGGCAGTAACTGACGCTGAGGAGCGAAAGCATGGGGAGCGAACAGGATTAGATACCCTGGTAGTCCATG
CCGTAAACGGTGGGCGCTAGGTGTGGGTTTCCTTCCACGGGATCCGTGCCGTAGCTAACGCATTAAGCGCCCCGCC
TGGGGAGTACGGCCGCAAGGCTAAAACTCAAAGGAATTGACGGGGGCCCGCACAAGCGGCGGAGCATGTGGATTAA
TTCGATGCAACGCGAAGAACCTTACCTGGGCTTGACATACACTGGATCGGGCTAGAGATAGTCTTTCCCTTTGTGG
CTGGTGTACAGGTGGTGCATGGTTGTCGTCAGCTCGTGTCGTGAGATGTTGGGTTAAGTCCCGCAACGAGCGCAAC
CCTTGTCTTATGTTGCCAGCATTTGGTTGGGGACTCATGAGAGACTGCCGGGGTCAACTCGGAGGAAGGTGGGGAT
GACGTCAAATCATCATGCCCCTTATGTCCAGGGCTTCACACATGCTACAATGGTCGGTACAACGCGCAGCGACACT
GTGAGGTGGAGCGAATCGCTGAAAGCCGGCCTTAGTTCGGATTGGGGTCTGCAACTCGACCCCATGAAGTCGGAGT
CGCTAGTAATCGCAGATCAGCAATGCTGCGGTGAATACGTTCCCGGGCCT (SEQ ID NO: 41)
HK555938.1.1357
ACGGCACCCCTCTCCGGAGGGAAGCGAGTGGCGAACGGCTGAGTAACACGTGGAGAACCTGCCCCCTCCCCCGGGA
TAGCCGCCCGAAAGGACGGGTAATACCGGATACCCCCGGGCGCCGCATGGCGCCCGGGCTAAAGCCCCGACGGGAG
GGGATGGCTCCGCGGCCCATCAGGTAGACGGCGGGGTGACGGCCCACCGTGCCGACAACGGGTAGCCGGGTTGAGA
GACCGACCGGCCAGATTGGGACTGAGACACGGCCCAGACTCCTACGGGAGGCAGCAGTGGGGAATCTTGCGCAATG
GGGGGAACCCTGACGCAGCGACGCCGCGTGCGGGACGGAGGCCTTCGGGTCGTAAACCGCTTTCAGCAGGGAAGAG
TCAAGACTGTACCTGCAGAAGAAGCCCCGGCTAACTACGTGCCAGCAGCCGCGGTAATACGTAGGGGGCGAGCGTT
ATCCGGATTCATTGGGCGTAAAGCGCGCGTAGGCGGCCCGGCAGGCCGGGGGTCGAAGCGGGGGGCTCAACCCCCC
GAAGCCCCCGGAACCTCCGCGGCTTGGGTCCGGTAGGGGAGGGTGGAACACCCGGTGTAGCGGTGGAATGCGCAGA
TATCGGGTGGAACACCGGTGGCGAAGGCGGCCCTCTGGGCCGAGACCGACGCTGAGGCGCGAAAGCTGGGGGAGCG
AACAGGATTAGATACCCTGGTAGTCCCAGCCGTAAACGATGGACGCTGGGTGTGGGGGGACGATCCCCCCGTGCCG
CAGCCNACGCATTAAGCGTCCCGCCTGGGGAGTACGGCCGCAAGGCTAAAACTCAAAGGAATTGACGGGGGCCCGC
ACAAGCAGCGGAGCATGTGGCTTAATTCGAAGCAACGCGAAGAACCTTACGGCGCATCCCCCCGAGGCCCACGGGG
GGTCCGCCGCGTGGGTCAGAGGAGCGCATACGGGAGGTGCATGGTTGTCGTCAGCTCGTGTCGTGAGATGTTGGGT
TAAGTCCCGCAACGAGCGCAACCCCCGCCGCGTGTTGCCATCGGGTGATGCCGGGAACCCACGCGGGACCGCCGCC
GTCAAGGCGGAGGAGGGCGGGGACGACGTCAAGTCATCATGCCCCTTATGCCCTGGGCTGCACACGTGCTACAATG
GCCGGTACAGAGGGATGCCACCCCGCGAGGGGGAGCGGATCCCGGAAAGCCGGCCCCAGTTCGGATTGGGGGCTGC
AACCCGCCCCCATGAAGTCGGAGTTGCTAGTAATCGCGGATCAGCATGCCGCGGTGAATGCGTTCCCGGGCCTTGT
ACACACCGCCCGTCACACCACCCGAGTCGTCTGCACCCGAAGTCGCCGGCCCAACCGCAAGGGGG (SEQ ID NO:
42)
FJ957551.1.1489
AGAGTTTGATCCTGGCTCAGGACGAACGCTGGCGGCGTGCCTAATACATGCAAGTCGAGCGGAGTTACTTTGAGAG
CTTGCTTTCAAAGTAACTTAGCGGCGGACGGGTGAGTAACACGTAGGCAACCTGCCCCTTAGACTGGGATAACTAC
CGGAAACGGTAGCTAATACCGGATAATTTCTTTTTTCTCCTGAAGGAAGAATGAAAGACGGAGCAATCTGTCACTG
AGGGATGGGCCTGCGGCGCATTAGCTAGTTGGTGGGGTAACGGCCCACCAAGGCGACGATGCGTAGCCGACCTGAG
AGGGTGATCGGCCACATTGGAACTGAGATACGGTCCAGACTCCTACGGGAGGCAGCAGTGGGGAATATTGCACAAT
GGGGGAAACCCTGATGCAGCAACGCCGCGTGAGTGATGAAGGTCTTCGGATTGTAAAGCTCTGTCTTTAGGGACGA
TAATGACGGTACCTAAGGAGGAAGCCACGGCTAACTACGTGCCAGCAGCCGCGGTAATACGTAGGTGGCAAGCGTT
ATCCGGATTTACTGGGCGTAAAGGGAGCGTAGGCGGATATTTAAGTGGGATGTGAAATACCCGAGCTTAACTTGGG
AGCTGCATTCCAAACTGGATATCTAGAGTGCAGGAGAGGAGAATGGAATTCCTAGTGTAGCGGTGAAATGCGTAGA
GATTAGGAAGAACACCAGTGGCGAAGGCGATTCTCTGGACTGTAACTGACGCTGAGGCTCGAAAGCGTGGGGAGCA
AACAGGATTAGATACCCTGGTAGTCCACGCCGTAAACGATGAATACCAGGTGTAGGGGCCCCAAGCCTCTGTGCCG
CCGCTAACGCATTAAGTATTCCGCCTGGGGAGTACGGTCGCAAGATTAAAACTCAAAGGAATTGACGGGGACCCGC
ACAAGCAGCGGAGCATGTGGTTTAATTCGAAGCAACGCGAAGAACCTTACCTAGACTTGACATGTCCTGAATTACC
AGTAATGTGGGAAGTTCCTTCGGGAACAGGAACACAGGTGGTGCATGGTTGTCGTCAGCTCGTGTCGTGAGATGTT
GGGTTAAGTCCCGCAACGAGCGCAACCCTTATTGTTAGTTGGTACCATTAAGTTGACCACTCTAGCGAGACTGCCC
GGGTTAACCGGGAGGAAGGTGGGGATGACGTCAAATCATCATGCCCCTTATGTCTAGGGCTACACACGTGCTACAA
TGGCAAGTACAAAGAGAAGCAATACTGTGAAGTGGAGCAAAACTCAAAAACTTGTCTCAGTTCGGATTGTAGGCTG
AAACTCGCCTACATGAAGCTGGAGTTGCTAGTAATCGCGAATCAGAATGTCGCGGTGAATACGTTCCCGGGTCTTG
TACACACCGCCCGTCACACCATGAGAGTTGGCAATACCCGAAGTCCGTAAGCTAACCGTAAGGAGGCAGCGGCCGA
AGGTAGGGTCAGCGATTGGGGTGAAGTCGTAACAAGGTAACCAA (SEQ ID NO: 43)
FJ957494.1.1454
TGAGTTTGATCATGGCTCAGGACGAACGCTGGCGGCGTGCCTAACACATGCAAGTCGAGCGATGAAATTTTCTTCG
GAAAATGGATTAGCGGCGGACGGGTGAGTAACACGTGGGTAACCTGCCCTATAGAGAGGGATAGCCTTCCGAAAGG
GAGATTAATACCTCATAATATCCTAGTATCGCATGATACATGGATTAAAGGAGCAATCCGCTATAGGATGGACCCG
CGGCGCATTAGCTAGTTGGTGAGGTAACGGCTCACCAAGGCGACGATGCGTAGCCGACCTGAGAGGGTGATCGGCC
ACATTGGGACTGAGACACGGCCCAGACTCCTACGGGAGGCAGCAGTGGGGAATATTGCACAATGGGGGAAACCCTG
ATGCAGCAACGCCGCGTGAGTGATGACGGTCTTCGGATTGTAAAGCTCTGTCTTTAGGGACGATAATGACGGTACC
TAAGGAGGAAGCCACGGCTAACTACGTGCCAGCAGCCGCGGTAATACGTAGGTGGCAAGCGTTGTCCGGATTTACT
GGGCGTAAAGGGAGCGTAGGCGGATCTTTAAGTGGGATGTGAAATACTCGGGCTCAACCTGGGGGCTGCATTCCAA
ACTGGGGATCTAGAGTACAGGAGGGGNGAGTGGAATTCCTAGTGTAGCGGTGAAATGCGTAGAGATTAGGAAGAAC
ACCAGTGGCGAAGGCGACTNTCTGGACTGTAACTGACGCTGAGGCTCGAAAGCGTGGGGAGCAAACAGGATTAGAT
ACCCTGGTAGTCCACGCCGTAAACGATGAATACTAGGTGTAGGGGGTGTCAACTCCCCCTGTGCCGCCGCTAACGC
ATTAAGTATTCCGCCTGGGGAGTACGGTCGCAAGATTAAAACTCAAAGGAATTGACGGGGGCCCGCACAAGTAGCG
GAGCATGTGGTTTAATTCGACGCAACGCGAAGAACCTTACCTAGACTTGACATCTTCTGCATTACCCTTAATCGGG
GAAGTTCCTTCGGGGACAGAATGACAGGTGGTGCATGGTTGTCGTCAGCTCGTGTCGTGAGATGTTGGGTTAAGTC
CCGCAACGAGCGCAACCCTTAAGCTTAGTTGCCATCATTAAGTTGGGCACTCTAAGTTGACTGCCGGTGACAAACC
GGAGGAAGGTGGGGATGACGTCAAATCATCATGCCCCTTATGTCTAGGGCTACACACGTGCTACAATGGCAAGTAC
AAAGAGAAGCAATACTGTGAAGTGGAGCAAAACTCAAAAACTTGTCTCAGTTCGGATTGTAGGCTGAAACTCGCCT
ACATGAAGCTGGAGTTGCTAGTAATCGCGAATCAGAATGTCGCGGTGAATACGTTCCCGGGTCTTGTACACACCGC
CCGTCACACCATGAGAGTTGGCAATACCCGAAGTCCGTAAGCTAACCGTAAGGAGGCAGCGGCCGAAGGTAGGGTC
AGCGATGGGG (SEQ ID NO: 44)
New.ReferenceOTU52
TACGTAGGTGGCGAGCGTTATCCGGATTTACTGGGCGTAAAGGGAGCGTAGGCGGATGATTAAGTGGGATGTGAAA
TACCCGGGCTCAACTTGGGTGCTGCATTCCAAACTGGTTATCTAGAGTGCAGGAGAGGAGAGTGGAATTCCTAGTG
TAGCGGTGAAATGCGTAGAGATTAGGAAGAACACCAGTGGCGAAGGCGACTCTCTGGACTGTAACTGACGCTGAGG
CTCGAAAGCGTGGGGAGCAAACAGG (SEQ ID NO: 45)
FM865905.1.1392
GGGAATCTCCAGGATCTGATTAGCGGCGGACGGGTGAGTACACGTGGGTAACCTGCCTCATAGAGTGGAATAGCCT
TCCGAAAGGAAGATTAATACCGCATAACGTTGAAAGATGGCATCATCATTCAACCAAAGGAGCAATCCGCTATGAG
ATGGACCCGCGGCGCATTAGCTAGTTGGTGGGGTAACGGCCTACCAAGGCGACGATGCGTAGCCGACCTGAGAGGG
TGATCGGCCACATTGGGACTGAGACACGGCCCAGACTCCTACGGGAGGCAGCAGTGGGGAATATTGCACAATGGGG
GAAACCCTGATGCAGCAACGCCGCGTGAGTGATGAAGGTTTTCGGATCGTAAAGCTCTGTCTTTGGGGAAGATAAT
GACGGTACCCAAGGAGGAAGCCACGGCTAACTACGTGCCAGCAGCCGCGGTAATACGTAGGTGGCGAGCGTTATCC
GGATTTACTGGGCGTAAAGGGAGCGTAGGCGGATGATTAAGTGGGATGTGAAATACCCGGGCTCAACTTGGGTGCT
GCATTCCAAACTGGTTATCTAGAGTGCAGGAGAGGAGAGTGGAATTCCTAGTGTAGCGGTGAAATGCGTAGAGATT
AGGAAGAACACCAGTGGCGAAGGCGACTCTCTGGACTGTAACTGACGCTGAGGCTCGAAAGCGTGGGGAGCAAACA
GGATTAGATACCCTGGTAGTCCACGCCGTAAACGATGAATACTAGGTGTGGGGGTTTCAACACCTCCGTGCCGCCG
CTAACGCATTAAGTATTCCGCCTGGGGAGTACGGTCGCAAGATTAAAACTNAAAGGAATTGACGGGGATCNNCACN
AGTAGCGGAGCATGTGGTTTAATTCGAAGCAACGCGAAGAACCTTACCTACACTTGACATCCCTTGCATTACTCTT
AATCGAGGAAATCTCTTCGGGGACAAGGTGACAGGTGGTGCATGGTTGTCGTCAGCTCGTGTCGTGAGATGTTGGG
TTAAGTCCCGNAACGAGGGGAACCNTTGTCGTTAGTTACTACCATTAAGTTGAGGACTNTAGNGAGACNGCTGGGT
TAACNAGGAGGAAGGTGGGGATGACTCAATCTCTGGNCNTTATGTGTAGGGNTACACACGTGCTACAATGGCTGGT
ACAGAGAGATGCATACCGGGAGGTGGANTCAATTTAAAAACAGTNTCNTTCGGATTGTAGGNTGAANTNNCCTACT
GAAGNTGGAGTTANTAGTAATCGCGAATCAGAATGTCGCGGTGAATACGTTCCCGGGTCTTGTACACNCCNCCCGT
CACNCCATGAGAGTTGGCAATACCCGAAGTCCGTGAGCTAACCGCAAGGAGGCAGCGGCCGAAGGTAGGGTCAGCG
ATTGGGGTGAAGTCGTAACAGGNA (SEQ ID NO: 46)
GQ016239.1.1362
GATGAACGCTGGCGGCATGCCTAATACATGCAAGTCGAACGGAGCGAATATGGAAGCTTGCTTCCGTAAGAGCTCA
GTGGCGAACGGGTGAGTAACACGTAGGTAACCTGCCCATGTGCCCGGGATAACTGCTGGAAACGGTAGCTAAAACC
GGATAGGTGAATAGGAGGCATCTCTTATTCATTAAAGGACCTGTAAGGGTGCGAACATGGATGGACCTGCGGCGCA
TTAGCTGGTTGGAGTGGTAACGGCACACCAAGGCGACGATGCGTAGCCGACCTGAGAGGGCGAACGGCCACATTGG
GACTGAGACACGGCCCAAACTCCTACGGGAGGCAGCAGTAGGGAATTTTCGTCAATGGGGGGAACCCTGAACGAGC
AATGCCGCGTGAGTGAAGAAGGTCTTCGGATCGTAAAGCTCTGTTGTAAGTGAAGAACGGTCAGTAGAGGAAATGA
TACTGAAGTGACGGTAGCTTACCAGAAAGCCACGGCTAACTACGTGCCAGCAGCCGCGGTAATACGTAGGTGGCGA
GCGTTATCCGGAATCATTGGGCGTAAAGGGTGCGCAGGTGGTACATTAAGTCCGAAGTAAAAGGCAGCAGCTCAAC
TGCTGTTGGCTTTGGAAACTGGTGAACTGGAGTGCAGGAGAGGGCGATGGAATTCCATGTGTAGCGGTAAAATGCG
TAGATATATGGAGGAACACCAGTGGCGAAGGCGGTCGCCTGGCCTGCAACTGACACTGAGGCACGAAAGCGTGGGG
AGCAAATAGGATTAGATACCCTAGTAGTCCACGCCGTAAACGATGAGAACTAAGTGTTGGGGAGACTCAGTGCTGC
AGTTAACGCAATAAGTTCTCCGCCTGGGGAGTATGCACGCAAGTGTGAAACTCAAAGGAATTGACGGGGGCCCGCA
CAAGCGGTGGAGTATGTGGTTTAATTCGAAGCAACGCGAAGAACCTTACCAGGCCTTGACATGGATGTAAATGTTC
TAGAGATAGAAAGATAGCTATACATCACACAGGTGGTGCATGGTTGTCGTCAGCTCGTGTCGTGAGATGTTGGGTT
AAGTCCCGCAACGAGCGCAACCCTTATCGCATGTTACCAGTATTGAGTTAGGGACTCATGCGAGACTGCCGGTGAC
AAACCGGAGGAAGGTGGGGATGACGTCAAATCATCATGCCCCTTATGGCCTGGGCTACACACGTACTACAATGGCG
GCTACAAAGAGAAGCGAACCTGCGAGGGGGAGCGGAACTCATAAAGGCCGTCTCAGTTCGGATTGGAGTCTGCAAC
TCGACTCCATGAAGTCGGAATCGCTAGTAATCGCAGATCAGCATGCTGCGGTGAATACGTTCTCGGGCCT (SEQ
ID NO: 47)
HG798451.1.1400
CTTGTGTCACCAACCATAGGGAGGGGGAAAACATGGAAACGGGGTTCATACCGCATAACTTTTTTAGCCCAATGCA
TAAGAAGAAAGGCCTTTCGGGTTTCGGTAAAGGAGGCCCCCGCGGCTCTTATAGTGTGTGTGGAAGTAACCGCTTC
CACAAGGCCCAGGTTTCATACCCGACTGGAGAGTGTGTTCGCCACACTGGGGAAAGGACCCCCGGCCCAGTCTCTC
TAGGGGAGGCAGCAGTAGGAATTTTCGGCAAAGGAAAAAATTTCTGACCGAACAACGCCGGTTGAATGAAGAAGTT
TTTCGGATCGAAAAACTCTGTTGTTAGAGAAGAACAAGGACGTTAGTAACTGAACGTCCCCTGACGGTATCTAACC
AGAAAGCCACGGCTAATTACGTGCCAGCAGCCGCGGTAATACGTAGGTGGCAAGCGTTGTCCGGATTTATTGGGCG
TAAAGCGAGCGCAGGCGGTTTCTTAAGTCTGATGTGAAAGCCCCCGGCTCAACCGGGGAGGGTCATTGGAAACTGG
GAGACTTGAGTGCAGAAGAGGAGAGTGGAATTCCATGTGTAGCGGTGAAATGCGTAGATATATGGAGGAACACCAG
TGGCGAAGGCGGCTCTCTGGTCTGTAACTGACGCTGAGGCTCGAAAGCGTGGGGAGCAAACAGGATTAGATACCCT
GGTAGTCCACGCCGTAAACGATGAGTGCTAAGTGTTGGAGGGTTTCCGCCCTTCAGTGCTGCAGCAAACGCATTAA
GCACTCCGCCTGGGGAGTACGACCGCAAGGTTGAAACTCAAAGGAATTGACGGGGGCCCGCACAAGCGGTGGAGCA
TGTGGTTTAATTCGAAGCAACGCGAAGAACCTTACCAGGTCTTGACATCCTTTGACCACTCTAGAGATAGAGCTTT
CCCTTCGGGGACAAAGTGACAGGTGGTGCATGGTTGTCGTCAGCTCGTGTCGTGAGATGTTGGGTTAAGTCCCGCA
ACGAGCGCAACCCTTATTGTTAGTTGCCATCATTTAGTTGGGCACTCTAGCGAGACTGCCGGTGACAAACCGGAGG
AAGGTGGGGATGACGTCAAATCATCATGCCCCTTATGACCTGGGCTACACACGTGCTACAATGGGAAGTACAACGA
GTCGCTAGACCGCGAGGTCATGCAAATCTCTTAAAGCTTCTCTCAGTTCGGATTGCAGGCTGCAACTCGCCTGCAT
GAAGCCGGAATCGCTAGTAATCGCGGATCAGCACGCCGCGGTGAATACGTTCCCGGGCCTTGTACACACCGCCCGT
CACACCACGAGAGTTTGTAACACCCGAAGTCGGTGAGGTAACCTTTTTGGAGCCAGCCGCCTAAGGTGGGATAGAT
GATTGGGGTGAAGTCGTAACCAACGTATGCC (SEQ ID NO: 48)
EU461791.1.1414
AGAGTTTGATCCTGGCTCAGGACTAACGCTGGCGGCGTGCCTAATACATGCAAGTAGAACGCTGAAGACTTTAGCT
TGCTAAAGTTGGAAGAGTTGCGAACGGGTGAGTAACGCGTAGGTAACCTGCCTACTAGCGGGGGATAACTATTGGA
AACGATAGCTAATACCGCATAACAGCATTTAACCCATGTTAGATGCTTGAAAGGAGCAATTGCTTCACTAGTAGAT
GGACCTGCGTTGTATTAGCTAGTTGGTGAGGTAACGGCTCACCAAGGCGACGATACATAGCCGACCTGAGAGGGTG
ATCGGCCACACTGGGACTGAGACACGGCCCAGACTCCTACGGGAGGCAGCAGTAGGGAATCTTCGGCAATGGGGGC
AACCCTGACCGAGCAACGCCGCGTGAGTGAAGAAGGTTTTCGGATCGTAAAGCTCTGTTGTAAGAGAAGAACGTGT
GTGAGAGTGGAAAGTTCACACAGTGACGGTAACTTACCAGAAAGGGACGGCTAACTACGTGCCAGCAGCCGCGGTA
ATACGTAGGTCCCGAGCGTTGTCCGGATTTATTGGGCGTAAAGCGAGCGCAGGCGGTTTAATAAGTCTGAAGTTAA
AGGCAGTGGCTTAACCATTGTTCGCTTTGGAAACTGTTAGACTTGAGTGCAGAAGGGGAGAGTGGAATTCCATGTG
TAGCGGTGAAATGCGTAGATATATGGAGGAACACCGGTGGCGAAAGCGGCTCTCTGGTCTGTAACTGACGCTGAGG
CTCGAAAGCGTGGGGAGCAAACAGGATTAGATACCCTGGTAGTCCACGCCGTAAACGATGAGTGAAAGGTGTTAGG
CCCTTTCCGGGGCTTAGTTGCTGCACGCTAACTGCATTATGACACTCCGCCAGGGGAGTACGACCGCTAGGTTGAA
ACTCAAAGGAGTTGACGGGGGCCAGCACAACCGGTGGAGCATGTGGTTGAATTGGAAGCAACGCGAAGAGCCTTAC
CAGGTCTTGACATCCCGACGCTATTCCTAGAGATAGGAAGTTTCTTCGGGACATTCGGTGGCAGGTGGTGCATGGT
AGTCGTCAGCTCGTGTCGTGAGATGTTGGGTTAAGTCCCGCAACGAGCGCAACCCCTATTGTTAGTTGCCATACAT
TAAGTTGGGCACTCTAGCGAGACTGCCGGTAATAAACCGGAGGAAGGTGGGGATGACGTCAAATCATCATGCCCCT
TATGACCTGGGCTACACACGACGCTACAATGGTTGGTACAACGAGTCGCGAGTCGGTGACGGCAAGCAAATCTCTT
AAAGCCAATCTCAGTTCGGATTGTAGGCTGCAACTCGCCTACATGAAGTCGGAATCGCTAGTAATCGCGGATCAGC
ACGCCGCGGTGAATACGTTCCCGGGCCTTGCACTCACCGCCCGTCA (SEQ ID NO: 49)
GU303759.1.1517
AGAGTTTGATCATGGCTCAGGACGAACGCCGGCGGCGTGCCTAATACATGCAAGTAGAACGCTGAAGACTTTAGCT
TGCTAAAGTTGGAAGAGTTGCGAACGGGTGAGTAACGCGTAGGTAACCTGCCTACTAGCGGGGGATAACTATTGGA
AACGATAGCTAATACCGTATAACAGCATTTAACACATGTTAGATGCTTGAAAGGAGCAATTGCTTCACTAGTAGAT
GGACCTGCGTTGTATTAGCTAGTTGGTGAGGTAACGGCTCACCAAGGCGACGATACATAGCCGACCTGAGAGGGTG
ATCGGCCACACTGGGACTGAGACACGGCCCAGACTCCTACGGGAGGCAGCAGTAGGGAATCTTCGGCAATGGGGGC
AACCCTGACCGAGCAACGCCGCGTGAGTGAAGAAGGTTTTCGGATCGTAAAGCTCTGTTGTAAGAGAAGAACGTGT
GTGAGAGTGGAAAGTTCACACAGTGACGGTAACTTACCAGAAAGGGACGGCTAACTACGTGCCAGCAGCCGCGGTA
ATACGTAGGTCCCGAGCGTTGTCCGGATTTATTGGGCGTAAAGCGAGCGCAGGCGGTTTAATAAGTCTGAAGTTAA
AGGCAGTGGCTTAACCATTGTTCGCTTTGGAAACTGTTAGACTTGAGTGCAGAAGGGGAGAGTGGAATTCCATGTG
TAGCGGTGAAATGCGTAGATATATGGARGGAAACACCGGTGGCGAAAGCGGCTCTCTGGTCTGTAACTGACGCTGA
GGCTCGAGAAGCGTGGGGAGCAAACAGGATTAGATACCCTGGTAGTCCACGCCGTAAGCGATGAGTGCTAGGTGTT
AGGCCCTTTCCGGGGCTTAGTGCCGCAGCTAACGCATTAAGCACTCCGCCTGGGGAGTACGACCGCAAGGTTGAAA
CTCAAAGGAATTGACGGGGGCCCGCACAAGCGGTGGAGCATGTGGTTTAATTCGAAGCAACGCGAAGAACCTTACC
AGGTCTTGACATCCCGATGCTATTCCTAGAGATAGGAAGTTTCTTCGGAACATCGGTGACAGGTGGTGCATGGTTG
TCGTCAGCTCGTGTCGTGAGATGTTGGGTTAAGTCCCGCAACGAGCGCAACCCCTATTGTTAGTTGCCATCATTAA
GTTGGGCACTCTAGCGAGACTGCCGGTAATAAACCGGAGGAAGGTGGGGATGACGTCAAATCATCATGCCCCTTAT
GACCTGGGCTACACACGTGCTACAATGGCGGTCAACAGAGGGAAGCAATACTGTGAAGTGGAGCAAACCCCTAAAA
GCCGTCCCAGTTCGGATTGCAGGCTGCAACCCGCCTGTATGAAGTTGGAATCGCTAGTAATCGCGGATCAGCATGC
CGCGGTGAATACGTTCCCGGGCCTTGTACACACCGCCCGTCACACCATGAGAGTCGGGAACACCCGAAGTCCGTAG
CCTAACTTTCACGAGGGGGCGCGGCCGAAGGTGGGTTCGATAATTGGGGTGAAGTCGTAACAAGGTAACCGTA
(SEQ ID NO: 50)
New.ReferenceOTU114
TACGTAGGTCCCGAGCGTTGTCCGGATTTATTGGGCGTAAAGCGAGCGCAGGCGGTTTAATAAGTCTGAAGTTAAA
GGCAGTGGCTTAACCATTTTTCGCTTTGGAAACTGTTAGACTTGAGTGCAGAAGGGGAGAGTGGAATTCCATGTGT
AGCGGTGAAATGCGTAGATATATGGAGGAACACCGGTGGCGAAAGCGGCTCTCTGGTCTGTAACTGACGCTGAGGC
TCGAAAGCGTGGGGAGCAAACAGG (SEQ ID NO: 51)
AB506154.1.1541
AGAGTTTGATCCTGGCTCAGGACGAACGCTGGCGGCGTGCCTAATACATGCAAGTAGAACGCTGAAGAAAGGAGCT
TGCTTCTTTTGGATGAGTTGCGAACGGGTGAGTAACGCGTAGGTAACCTGCCTTGTAGCGGGGGATAACTATTGGA
AACGATAGCTAATACCGCATAACAGCTTTTGACACATGTTAGAAGCTTGAAAGATGCAATTGCATCACTACGAGAT
GGACCTGCGTTGTATTAGCTAGTAGGTAGGGTAACGGCCTACCTAGGCGACGATACATAGCCGACCTGAGAGGGTG
ATCGGCCACACTGGGACTGAGACACGGCCCAGACTCCTACGGGAGGCAGCAGTAGGGAATCTTCGGCAATGGGGGC
AACCCTGACCGAGCAACGCCGCGTGAGTGAAGAAGGTTTTCGGATCGTAAAGCTCTGTTGTAAGAGAAGAACGTGT
GTGAGAGTGGAAAGTTCACACAGTGACGGTAACTTACCAGAAAGGGACGGCTAACTACGTGCCAGCAGCCGCGGTG
ATACGTAGGTCCCGAGCGTTGTCCGGATTTATTGGGCGTAAAGCGAGCGCAGGCGGTTTAATAAGTCTGAAGTTAA
AGGCAGTGGCTTAACCATTGTTCGCTTTGGAAACTGTTAAACTTGAGTGCAGAAGGGGAGAGTGGAATTCCATGTG
TAGCGGTGAAATGCGTAGATATATGGAGGAACACCGGTGGCGAAAGCGGCTCTCTGGTCTGTAACTGACGCTGAGG
CTCGAAAGCGTGGGGAGCAAACAGGATTAGATACCCTGGTAGTCCACGCCGTAAACGATGAGTGCTAGGTGTTAGG
CCCTTTCCGGGGCTTAGTGCCGCAGCTAACGCATTAAGTATTCCGCCTGGGGAGTACGGTCGCAAGATTAAAACTC
AAAGGAATTGACGGGGGCCCGCACAAGCAGCGGAGCATGTGGTTTAATTCGAAGCAACGCGAAGAACCTTACCTAG
ACTTGACATCTCCTGCATTACTCTTAATCGAGGAAGTCCCTTCGGGGACAGGATGACAGGTGGTGCATGGTTGTCG
TCAGCTCGTGTCGTGAGATGTTGGGTTAAGTCCCGCAACGAGCGCAACCCTTATTGTTAGTTGCCATCATTAAGTT
GGGCACTCTAGCGAGACTGCCCGGGTTAACCGGGAGGAAGGTGGGGATGACGTCAAATCATCATGCCCCTTATGTC
TAGGGCTACACACGTGCTACAATGGTCGGTACAATAAGACGCAAGCCCGCGAGGGGGAGCAAAACTGGAAAACCGA
TCTCAGTTCGGATTGTAGGCTGAAACTCGCCTACATGAAGCTGGAGTTGCTAGTAATCGCGAATCAGCATGTCGCG
GTGAATACGTTCCCGGGCCTTGTACACACCGCCCGTCACACCATGAGAGTTGGCAATACCCAAAGTACGTGATCTA
ACCCGCAAGGGAGGAAGCGTCCTAAGGTAGGGTCAGCGATTGGGGTGAAGTCGTAACAAGGTAGCCGTAGGAGAAC
CTGCGGCTG (SEQ ID NO: 52)
EU774370.1.1398
AGAGTTTGCTCTTGGGTCAGGATGAACGCTGGCGGCGTGCTTAACACATGCAAGTCGAGCGAGAAAAGTTCTTCGG
AGCTTTTCTAGCGGCGGACGGGTGAGTAACACGTGGGCAACCTGCCTCATAGAGGGGAATAGCCTTCCGAAAGGAA
GATTAATACCGCATAACATTGTTGAAAGGCATCTTTTAACAATCAAAGGAGCAATCCGCTATGAGATGGGCCCGCG
GCGCATTAGCTAGTTGGTGAGGTAACGGCTCACCAAGGCGACGATGCGTAGCCGACCTGAGAGGGTGATCGGCCAC
ATTGGAACTGAGACACGGTCCAGACTCCTACGGGAGGCAGCAGTGGGGAATATTGCACAATGGGGGAAACCCTGAT
GCAGCGACGCCGCGTGAGTGAAGAAGTATTTCGGTATGTAAAGCTCTGTTGTAAGAGAAGAACGTGTGTGAGAGTG
GAAAGTTCACACAGTGACGGTAACTTACCAGAGAGGGACGGCTAACTACGTGCCAGCAGCCGCGGTAATACGTAGG
TCCCGAGCGTTGTCCGGATTTATTGGGCGTAAAGCGAGCGCAGGCGGTTTAATAAGTCTGAAGTTAAAGGCAGTGG
CTTAACCATTGTTCGCTTTGGAAACTGTTAGACTTGAGTGCAGAAGGGGAGAGTGGAATTCCATGTGTAGCGGTGA
AATGCGTAGATATATGGAGGAACACCGGTGGCGAAAGCGGCTCTCTGGTCTGTAACTGACGCTGAGGCTCGAAAGC
GTGGGGAGCAAACAGGATTAGATACCCTGGTAGTCCACGCCGTAAACGATGAGTGCTAGGTGTTAGGCCCTTTCCG
GGGCTTAGTGCCGCAGCTAACGCATTAAGCACTCCGCCTGGGGAGTACGACCGCAAGGTTGAAACTCAAAGGAATT
GACGGGGGCCCGCACAAGCGGTGGAGCATGTGGTTTAATTCGAAGCAACGCGAAGAACCTTACCAGGTCTTGACAT
CCCGATGCTATTCCTAGAGATAGGAAGTTTCTTCGGAACATCGGTGACAGGTGGTGCATGGTTGTCGTCAGCTCGT
GTCGTGAGATGTTGGGTTAAGTCCCGCAACGAGCGCAACCCCTATTGTTAGTTGCCATCATTAAGTTGGGCACTCT
AGCGAGACTGCCGGTAATAAACCGGAGGAAGGTGGGGATGACGTCAAATCATCATGCCCCTTATGACCTGGGCTAC
ACACGTGCTACAATGGTTGGTACAACGAGTCGCGAGTCGGTGACGGCAAGCAAATCTCTTAAAGCCAATCTCAGTT
CGGATTGTAGGCTGCAACTCGCCTACATGAAGTCGGAATCGCTAGTAATCGCGGATCAGCACGCCGCGGTGAATAC
GTTCCCGGGCCTTGCACTCACCGCCCGTCA (SEQ ID NO: 53)
HK557089.3.1395
AGACTTTAGCTTGCTAAAGTTGGAAGAGTTGCGAACGGGTGAGTAACGCGTAGGTAACCTGCCTACTAGCGGGGGA
TAACTATTGGAAACGATAGCTAATACCGCATAACAGCATTTAACCCATGTTAGATGCTTGAAAGGAGCAATTGCTT
CACTAGTAGATGGACCTGCGTTGTATTAGCTAGTTGGTGAGGTAACGGCTCACCAAGGCGACGATACATAGCCGAC
CTGAGAGGGTGATCGGCCACACTGGGACTGAGACACGGCCCAGACTCCTACGGGAGGCAGCAGTAGGGAATCTTCG
GCAATGGGGGCAACCCTGACCGAGCAACGCCGCGTGAGTGAAGAAGGTTTTCGGATCGTAAAGCTCTGTTGTAAGA
GAAGAACGTGTGTGAGAGTGGAAAGTTCACACAGTGACGGTAACTTACCAGAAAGGGACGGCTAACTACGTGCCAG
CAGCCGCGGTAATACGTAGGTCCCGAGCGTTGTCCGGATTTATTGGGCGTAAAGCGAGCGCAGGCGGTTTAATAAG
TCTGAAGTTAAAGGCAGTGGCTTAACCATTGTTCGCTTTGGAAACTGTTAGACTTGAGTGCAGAAGGGGAGAGTGG
AATTCCATGTGTAGCGGTGAAATGCGTAGATATATGGAGGAACACCGGTGGCGAAAGCGGCTCTCTGGTCTGTAAC
TGACGCTGAGGCTCGAAAGCGTGGGGAGCAAACAGGATTAGATACCCTGGTAGTCCACGCCGTAAACGATGAGTGC
TAGGTGTTAGGCCCTTTCCGGGGCTTAGTGCCGCAGCTAACGCATTAAGCACTCCGCCTGGGGAGTACGACCGCAA
GGTTGAAACTCAAAGGAATTGACGGGGGCCCGCACAAGCGGTGGAGCATGTGGTTTAATTCGAAGCAACGCGAAGA
ACCTTACCAGGTCTTGACATCCCGATGCTATTCCTAGAGATAGGAAGTTTCTTCGGAACTGTGAGACTTGAGGGCA
GAAGGGTAGAGTGCACTTGTATGGGGAGCTGTGGAATGCGTTCCCGCAACGAGCGCAACCCCTATTGTTAGTTGCC
ATCATTAAGTTGGGCACTCTAGCGAGACTGCCGGTAATAAACCGGAGGAAGGTGGGGATGACGTCAAATCATCATG
CCCCTTATGACCTGGGCTACACACGTGCTACAATGGTTGGTACAACGAGTCGCGAGTCGGTGACGGCAAGCAAATC
TCTTAAAGCCAATCTCAGTTCGGATTGTAGGCTGCAACTCGCCTACATGAAGTCGGAATCGCTAGTAATCGCGGAT
CAGCACGCCGCGGTGAATACGTTCCCGGGCCTTGTACACACCGCCCGTCACACCACGAGAGTTTGTAACACCCGAA
GTCGGTGAGGTANCCTTTTAGGAGC (SEQ ID NO: 54)
HQ807346.1.1456
TTCAGGACGAACGCTGGCGGCGTGCCTAATACATGCAAGTAGAACGCTGAAGACTTTAGCTTGCTAAAGTTGGAAG
AGTTGCGAACGGGTGAGTAACGCGTAGGTAACCTGCCTACTAGCGGGGGATAACTATTGGAAACGATAGCTAATAC
CGCATAACAGCATTTAACCCATGTTAGATGCTTGAAAGGAGCAATTGCTTCACTAGTAGATGGACCTGCGTTGTAT
TAGCTAGTTGGTGAGGTAACGGCTCACCAAGGCGACGATACATAGCCGACCTGAGAGGGTGATCGGCCACACTGGG
ACTGAGACACGGCCCAGACTCCTACGGGAGGCAGCAGTAGGGAATCTTCGGCAATGGGGGCAACCCTGACCGAGCA
ACGCCGCGTGAGTGAAGAAGGTTTTCGGATCGTAAAGCTCTGTTGTAAGAGAAGAACGTGTGTGAGAGTGGAAAGT
TCACACAGTGACGGTAACTTACCAGAAAGGGACGGCTAACTACGTGCCAGCAGCCGCGGTAATACGTAGGTCCCGA
GCGTTGTCCGGATTTATTGGGCGTAAAGCGAGCGCAGGCGGTTTAATAAGTCTGAAGTTAAAGGCAGTGGCTTAAC
CATTGTTCGCTTTGGAAACTGTTAGACTTGAGTGCAGAAGGGGAGAGTGGAATTCCATGTGTAGCGGTGAAATGCG
TAGATATATGGAGGAACACCGGTGGCGAAAGCGGCTCTCTGGTCTGTAACTGACGCTGAGGCTCGAAAGCGTGGGG
AGCAAACAGGATTAGATACCCTGGTAGTCCACGCCGTAAACGATGAGTGCTAGGTGTTAGGCCCTTTCCGGGGCTT
AGTGCCGCAGCTAACGCATTAAGCACTCCGCCTGGGGAGTACGACCGCAAGGTTGAAACTCAAAGGAATTGACGGG
GGCCCGCACAAGCGGTGGAGCATGTGGTTTAATTCGAAGCAACGCGAAGAACCTTACCAGGTCTTGACATCCTTTG
ACCACTCTAGAGATAGAGCTTCCCCTTCGGGGGCAAAGTGACAGGTGGTGCATGGTTGTCGTCAGCTCGTGTCGTG
AGATGTTGGGTTAAGTCCCGCAACGAGCGCAACCCTTATTGTTAGTTGCCATCATTTAGTTGGGCACTCTAGCGAG
ACTGCCGGTGACAAACCGGAGGAAGGTGGGGATGACGTCAAATCATCATGCCCCTTATGACCTGGGCTACACACGT
GCTACAATGGGAAGTACAACGAGTTGCGAAGTCGCGAGGCTAAGCTAATCTCTTAAAGCTTCTCTCAGTTCGGATT
GTAGGCTGCAACTCGCCTACATGAAGCCGGAATCGCTAGTAATCGCGGATCAGCACGCCGCGGTGAATACGTTCCC
GGGCCTTGTACACACCGCCCGTCACACCACGAGAGTTTGTAACACCCGAAGTCGGTGAGGTAACCTTTTAGGAGCC
AGCCGCCTAAGG (SEQ ID NO: 55)
HQ748204.1.1442
CTAATACATGCGAGGAGAACGCTGAAGACTTTCTTTTGCTATAGTTGGGAGAGTTGCTAACGGGTGAGTAACGCGT
AGGTGACCTGCCTACTAGCGGGGGATAACTATTGCAAACGATAGCTAATACCGCATAACAGCCTTTAACCCATGTT
AGATGCTTGAAAGGAGCAATTGCTTCACTAGTAGATGGACCTGCGTTGTATTAGCTAGTTGGTGAGGTAACGGCTC
ACCAAGGCGACGATACATAGCCGACCTGAGAGGGTGATCGGCCACACTGGGACTGAGACACGGCCCATACTCCTAC
GGGAGGCACCAGTAGGGAATCTTCGGGAATGGGGGCAACCCTGACCGAGCAACGCCGCGTGAGTGAAGAAGGTTTT
CGGATCGTAAAGCTCTGTTGTAAGAGAAGAACGTGTGTGAGAGTGGAAAGTTCACACTGTGACGGTAACTTACCAG
AAAGGGACGGCTAACTACGTGCCAGCAGCCGCGGTAATACGTAGGTCCCGAGCGTTGTCCGGATTTATTGGGCGTA
AAGCGAGCGCAGGCGGTTTAATAAGTCTGAAGTTAAAGGCAGTGGCTTAACCATTGTTCGCTTTGGAAACTGTTAG
ACTTGAGTGCATAAGGGGAGAGTGGAATTCCATGTGTAGCGGTGAAATGCGTAGATATATGGAGGAACACCGGTGG
CGAAAGCGGCTCTCTGGTCTGTAACTGACGTTGAGGCTCGAAAGCGTGGGGAGCAAACAGGATTAGATACCCTGGT
AGTCCACGCTGTAAACGATGAGTGGTAGGTGTTAGGCCCTTTCTGGGGTTTAGTGCCGCAGATTACGCATTAAGCC
ATTCGCCTGGGGAGTACGACCGCAAGGTTGAAACTTAAAGGAATTGACGGGGGCCCGCACAAGCGGTGGAGCATGT
GGTTTAATTAGAAGCAACGCGAAGAACCTTACCAGGTCTTGACATCCCGATGCTATTCTTAGAGATAGGAAGTTTC
TTCGGAACATCGGTGACAGGTGGTGCATGGTTGTCGTCAGCTCGTGTCGAGAGATGTTGGGTTAAGTCCCTCAACG
AGCGCAACCCCTATTTTTATTTGCCATCATTAAGTTGGGCAATCTAGCGAGACTGCCGGTAATAAACCGGAGGAAG
GTGGGGATGACGTCAAATCATCATGCTCCTTATGTCATGGGGTACACACGTGGTACAATGGTTGGTACAACGAGTC
GCGAGTTGGTGAAGGCAAGCAAATCTCTTAAAGCCAATATCAGTTCGGATTGTAGGCTGCAAATAGCCTACATGTA
GTCGGAATTGTTAGTAATCGGGGATCAGCACTCCGCGGTGAATACGTTTCCGGGCCTTGTACACCCCGCCCGTCTA
CACCACGAGAGTTTGTAACACCCGAAGTCGGTGAGGTAACTCTTTTAGGAGCCAGCCGCCTAAGGTGGGATAGA
(SEQ ID NO: 56)
GU179917.1.1382
AGAGTTTGATTATGGCTCAGGATGAACGCTGGCGGCGTGCCTAATACATGCAAGTCGAACGCGAGCAGCAATGCTC
GAGTGGCGAACGGGTGAGTAATACATAAGTAACCTGCCCTAGACAGGGGGATAACTGCTGGAAACGGCAGCTAAGA
CCGCATAGGTATGGACACTGCATGGTGACCATATTAAAAGTGCCAAGGCACTGGTAGAGGATGGACTTATGGCGCA
TTAGCTGGTTGGTGAGGTAACGGCTCACCAAGGCGACGATGCGTAGCCGACCTGAGAGGGTGACCGGCCACACTGG
GACTGAGACACGGCCCAGACTCCTACGGGAGGCAGCAGTAGGGAATTTTCGGCAATGGGGGGAACCCTGACCGAGC
AACGCCGCGTGAAGGAAGAAGGAATTCGTTCTGTAAACTTCTGTTATAAAGGAAGAACGGCGGATATAGGGAATGA
TATCCGAGTGACGGTACTTTATGAGAAAGCCACGGCTAACTACGTGCCAGCAGCCGCGGTAATACGTAGGTGGCGA
GCGTTATCCGGAATTATTGGGCGTAAAGAGGGAGCAGGCGGCGGCAGAGGTCTGTGGTGAAAGACTGAAGCTTAAC
TTCAGTAAGCCATAGAAACCGGGCTGCTAGAGTGCAGGAGAGGATCGTGGAATTCCATGTGTAGCGGTGAAATGCG
TAGATATATGGAGGAACACCAGTGGCGAAGGCGACGGTCTGGCCTGTAACTGACGCTCATTCCCGAAAGCGTGGGG
AGCAAATAGGATTAGATACCCTAGTAGTCCACGCCGTAAACGATGAGTACTAAGTGTTGGGAGTCAAATTTCAGTG
CTGCAGTTAACGCAATAAGTACTCCGCCTGAGTAGTACGTTCGCAAGAATGAAACTCAAAGGAATTGACGGGGGCC
CGCACAAGCGGTGGAGCATGTGGTTTAATTCGATGATACGCGAGGAACCTTACCAGGGCTTAAATGTGACTGACAG
GTCCGGAAACGGACTTTTCTTCGGACAGTTACAGGTGCTGCATGGTTGTCGTCAGCTCGTGCCGTGAGGTGTCAGG
TTAAGTCCTATAACGAGCGCAACCCCTGTCGCTAGTTGCCAGCGAGTAATGTCGGGAACTCTAGCGAGACTGCCAG
TGCAAACTGCGAGGAAGGTGGGGATGACGTCAAATCATCACGGCCCTTACGCCCTGGGCTACACACGTGCTACAAT
GGCCGGTACAGAGAGCAGCCACCCCGCGAGGGGGAGCGAATCTACAAAACCGGTCACAGTTCGGATCGGAGTCTGC
AACTCGACTCCGTGAAGCTGGAATCGCTAGTAATCGGATATCAGCCATGATCCGGTGAATACGTTCCCGGGCCTTG
TACACACCCCCGTC (SEQ ID NO: 57)
GQ448336.1.1418
AGAGTTTGATCATGGCTCAGGACGAACGCTGGCGGCGTGCTTAACACATGCAAGTCGAACGAGAAGAGATGAGAAG
CTTGCTTCTTATCTCTTCGAGTGGCAAACGGGTGAGTAACGCGTAAGCAACCTGCCCTTCAGATGGGGACAACAGC
TGGAAACGGCTGCTAATACCGAATACGTTCTTTTTGTCGCATGGCAGAGGGAAGAAAGGGAGGCTCTTCGGAGCTT
TCGCTGAAGGAGGGGCTTGCGTCTGATTAGCTAGTTGGAGGGGTAACGGCCCACCAAGGCGACGATCAGTAGCCGG
TCTGAGAGGATGAACGGCCACATTGGGACTGAGACACGGCCCAGACTCCTACGGGAGGCAGCAGTGGGGAATCTTC
CGCAATGGACGAAAGTCTGACGGAGCAACGCCGCGTGAACGATGACGGCCTTCGGGTTGTAAAGTTCTGTTATACG
GGACGAATGGCGTAGCGGTCAATACCCGTTACGAGTGACGGTACCGTAAGAGAAAGCCACGGCTAACTACGTGCCA
GCAGCCGCGGTAATACGTAGGTGGCAAGCGTTGTCCGGAATTATTGGGCGTAAAGGGCGCGCAGGCGGCGTCGTAA
GTCGGTCTTAAAAGTGCGGGGCTTAACCCCGTGAGGGGACCGAAACTGCGATGCTAGAGTATCGGAGAGGAAAGCG
GAATTCCTAGTGTAGCGGTGAAATGCGTAGATATTAGGAGGAACACCAGTGGCGAAAGCGGCTTTCTGGACGACAA
CTGACGCTGAGGCGCGAAAGCCAGGGGAGCAAACGGGATTAGATACCCCGGTAGTCCTGGCCGTAAACGATGGATA
CTAGGTGTAGGAGGTATCGACCCCTTCTGTGCCGGAGTTAACGCAATAAGTATCCCGCCTGGGGAGTACGGCCGCA
AGGCTGAAACTCAAAGGAATTGACGGGGGCCCGCACAAGCGGTGGAACATGTGGTTTAATTCGATGATACGCGAGG
AACCTTACCCGGGCTTAAATTGCAGTGGAATGATGTGGAAACATGTCAGTGAGCAATCACCGCTGTGAAGGTGCTG
CATGGTTGTCGTCAGCTCGTGCCGTGAGGTGTCGGCTTAAGTGCCATAACGAGCGCAACCCTTATCTTCAGTTACT
AACAGGTCATGCTGAGGACTCTGGAGAGACTGCCGTCGTAAGATGTGAGGAAGGTGGGGATGACGTCAAATCAGCA
CGGCCCTTACGTCCGGGGCTACACACGTGTTACAATGGGGGGTACAGAGGGCCGCTACCACGCGAGTGGATGCCAA
TCCCAAAAACCTCTCTCAGTTCGGACTGGAGTCTGCAACCCGACTCCACGAAGCTGGATTCGCTAGTAATCGCGCA
TCAGCCACGGCGCGGTGAATACGTTCCCGGGCCTTGCACTCACCGCCCGT (SEQ ID NO: 58)
DQ804865.1.1390
AGAGTTTGATCCTGGCTCAGGATGAACGCTGGCGGCGTGCTTAACACATGCAAGTCGAACGGGAAACTTTTCATTG
AAGCTTCGGCAGATTTGGTCTGTTTCTAGTGGCGGACGGGTGAGTAACGCGTGGGTAACCTGCCTTATACAGGGGG
ATAACAACCAGAAATGGTTGCTAATACCGCATAAGCGCACAGGACCGCATGGTCCGGTGTGAAAAACTCCGGTGGT
ATAAGATGGACCCGCGTTGGATTAGCTAGTTGGCAGGGTAACGGCCTACCAAGGCGACGATCCATAGCCGGCCTGA
GAGGGTGAACGGCCACATTGGGACTGAGACACGGCCCAGACTCCTACGGGAGGCAGCAGTGGGGAATATTGCACAA
TGGGGGAAACCCTGATGCAGCGACGCCGCGTGAAGGAAGAAGTATCTCGGTATGTAAACTTCTATCAGCAGGGAAG
ATAGTGACGGTACCTGACTAAGAAGCCCCGGCTAAATACGTGCCAGCAGCCGCGGTAATACGTATGGTTCAAGCGT
TATCCGGATTTACTGGGTGTAAAGGGTGAGTAGGCGGTTATGCAAGTCATATGTGAAATGTCGGGGCTCAACTCCG
GCCTGCATAAGAAACTGTATAACTAGAGTGCAGGAGAGGCAAGCGGAATTCCTAGTGTAGCGGTGAAATGCGTAGA
TATTAGGAAGAACACCGGTGGCGAAGGCGGCTTGCTGGACTGTTACTGACGCTGAGTCACGAAAGCGTGGGGAGCA
AACAGGATTAGATACCCTGGTAGTCCACGCCGTAAACGATGAATACTAGGTGTCGGGTGGCAAAGCCATTCGGTGC
CGCAGCAAACGCAATAAGTATTCCACCTGGGGAGTACGTTCGCAAGAATGAAACTCAAAGGAATTGACGGGGACCC
GCACAAGCGGTGGAGTATGTGGTTTAATTCGAAGCAACGCGAAGAACCTTACCAGGCCTTGACATGGATATAAATG
TTCTAGAGATAGAAAGATAGCTATATATCACACAGGTGGTGCATGGTTGTCGTCAGCTCGTGTCGTGAGATGTTGG
GTTAAGTCCCGCAACGAGCGCAACCCTTGTCTTCTGTTACCAGCATTGAGTTGGGGACTCAGGAGAGACTGCCGGT
GACAAACCGGAGGAAGGTGGGGATGACGTCAAATCATCATGCCCCTTATGGCCTGGGCTACACACGTACTACAATG
GCGCCTACAAAGAGCAGCGACACCGCGAGGTGAAGCGAATCTCATAAAGGGCGTCTCAGTTCGGATTGAAGTCTGC
AACTCGACTTCATGAAGTCGGAATCGCTAGTAATCGCAGATCAGCATGCTGCGGTGAATACGTTCCCGGGTCTTGT
ACTCACCGCCCGTCA (SEQ ID NO: 59)
GQ491757.1.1361
GATGAACGCTGGCGGCGTGCTTAACACATGCAAGTCGAGCGAAGCACTTAAGTGGATCTCTTCGGATTGAAACTTA
TTTGACTGAGCGGCGGACGGGTGAGTAACGCGTGGGTAACCTGCCTCATACAGGGGGATAACAGTTAGAAATGGCT
GCTAATACCGCATAAGCGCACAGGACCGCATGGTCTGGTGTGAAAAACTCCGGTGGTATGAGATGGACCCGCGTCT
GATTAGCTAGTTGGAGGGGTAACGGCCCACCAAGGCGACGATCAGTAGCCGGCCTGAGAGGGTGAACGGCCACATT
GGGACTGAGACACGGCCCAGACTCCTACGGGAGGCAGCAGTGGGGAATATTGCACAATGGGGGAAACCCTGATGCA
GCGACGCCGCGTGAAGGAAGAAGTATCTCGGTATGTAAACTTCTATCAGCAGGGAAGAAAATGACGGTACCTGACT
AAGAAGCCCCGGCTAACTACGTGCCAGCAGCCGCGGTAATACGTAGGGGGCAAGCGTTATCCGGATTTACTGGGTG
TAAAGGGAGCGTAGACGGAAGAGCAAGTCTGATGTGAAAGGCTGGGGCTTAACCCCAGGACTGCATTGGAAACTGT
TTTTCTAGAGTGCCGGAGAGGTAAGCGGAATTCCTAGTGTAGCGGTGAAATGCGTAGATATTAGGAGGAACACCGG
TGGCGAAGGCGGCTTACTGGACGACCACTGACGCTGAGGCTCGAAAGCGTGGGGAGCAAACAGGATTAGATACCCT
GGTAGTCCACGCCGTAAACCGATGAATAATAGGTGTCGGGGAACAATAGTTCTTTGGTGCCGCAGCAAAACGCATT
AAGTATTCCACCTGGGGAGTACGTTCGCAAGAATGAAACTCAAAGGAATTGACGGGGACCCGCACAAGCGGTGGAG
CATGTGGTTTAATTCGATGCAACGCGAAGAACCTTACCTGCTCTTGACATCCCACTGACCGGACAGTAATGTGTCC
TTTTCTTCTGAACAGTGGAGACAGGTGGTGCATGGTTGTCGTCAGCTCGTGTCGTGAGATGTTGGGTTAAGTCCCG
CAACGAGCGCAACCCTCGTCTTTAGTAGCCAGCAGTCCGGCTGGGCACTCTAGAGAGACTGCCAGGGATAACCTGG
AGGAAGGCGGGGAGGACGTCAAATCATCATGCCCCTTACGAGCAGGGCTACACACGTGCTACAATGGCGTAAACAA
AGGGAAGCGACCCCGTGAAGGTGAGCAAATCTCAAAAATAACGTCTCAGTTCGGATTGTAGTCTGCAACTCGACTA
CATGAAGCTGGAATCGCTAGTAATCGCGAATCAGAATGTCGCGGTGAATAAAAGGCCGGGTCTTGCACA (SEQ ID
NO: 60)
New.ReferenceOTU56
TACGGAAGGTCCAGGCGTTATCCGGATTTATTGGGTTTAAAGGGAGCGCAGGCGGACTCTTAAGTCAGTTGTGAAA
TACGGCGGCTCAACCGTCGGACTGCAGTTGATACTGGGAGTCTTGAGTACACGCAGAGATACTGGAATTCATGGTG
TAGCGGTGAAATGCTCAGATATCATGAGGAACTCCGATCGCGAAGGCAGGTATCTGGAGTGTAACTGACGCTGAGG
CTCGAAAGTGCGGGTATCAAACAGG (SEQ ID NO: 61)
KF842598.1.1394
AGAGTTTGATCCTGGCTCAGGATGAACGCTAGCGACAGGCTTAACACATGCAAGTCGAGGGGCAGCATGATTTGTA
GCAATACAGATTGATGGCGACCGGCGCACGGGTGAGTAACGCGTATGCAACTTACCTATCAGAGGGGGATAGCCCG
GCGAAAGTCGGATTAATACCCCATAAAACAGGGGTCCCGCATGGGAATATTTGTTAAAGATTCATCGCTGATAGAT
AGGCATGCGTTCCATTAGGCAGTTGGCGGGGTAACGGCCCACCAAACCGACGATGGATAGGGGTTCTGAGAGGAAG
GTCCCCCACATTGGTACTGAGACACGGACCAAACTCCTACGGGAGGCAGCAGTGAGGAATATTGGTCAATGGCCGA
GAGGCTGAACCAGCCAAGTCGCGTGAAGGAAGAAGGATCTATGGTCTGTAAACTTCTTTTATAGGGGAATAAAGTG
GAGGACGTGTCCTTTTTTGTATGTACCCTATGAATAAGCATCGGCTAACTCCGTGCCAGCAGCCGCGGTAATACGG
AGGATGCGAGCGTTATCCGGATTTATTGGGTTTAAAGGGTGCGTAGGTGGTGATTTAAGTCAGCGGTGAAAGTTTG
TGGCTCAACCATAAAATTGCCGTTGAAACTGGGTTACTTGAGTGTGTTTGAGGTAGGCGGAATGCGTGGTGTAGCG
GTGAAATGCATAGATATCACGCAGAACTCCGATTGCGAAGGCAGCTTACTAAACCATAACTGACACTGAAGCACGA
AAGCGTGGGGATCAAACAGGATTAGATACCCTGGTAGTCCACGCAGTAAACGATGATTACTAGGAGTTTGCGATAC
AATGTAAGCTCTACAGCGAAAGCGTTAAGTAATCCACCTGGGGAGTACGCCGGCAACGGTGAAACTCAAAGGAATT
GACGGGGGCCCGCACAAGCGGAGGAACATGTGGTTTAATTCGATGATACGCGAGGAACCTTACCCGGGTTTGAACG
TAGTCTGACCGGAATGGAAACACTCCTTCTAGCAATAGCAGATTACAAGGTGCTGCATGGTTGCCTCAACTCCGGC
CCGGAAGGTCCGGCTTAATTGCCATAACAAGCGCACCCTTTTACCAAGGTTCAAACAGGTGAAGCTTGAAGACTCT
GTGGAACCTCCCCCCTAACCTGTGAGAAGAAGTGGGGATACACTCAATAAACCACGGCCCTTAATCCCGGGGGGAA
CACTGGTTACAATGGGTTGGGAAAGGGGGCTTCCTGGCGACAGGATGCTAATCTCCAAACCATGTCTCAGTTCGGA
TCGGAGTCTGCAACTCGACTCCGTGAAGCTGGATTCGCTAGTAATCGCGCATCAGCCATGGCGCGGTGAATACGTT
CCCGGGCCTTGTACACACCGCCCGTC (SEQ ID NO: 62)
HQ802052.1.1445
TACAGGCTTAACACATGCAAGTCGAGGGGCAGCATGATTGAAGCTTGCTTCAATTGATGGCGACCGGCGCACGGGT
GAGTAACACGTATCCAACCTTCCGTACACTCAGGGATAGCCTTTCGAAAGAAAGATTAATACCTGATGGTATCTTA
AGCACACATGTAATTAAGATTAAAGATTTATCGGTGTACGATGGGGATGCGTTCCATTAGGTAGTAGGCGGGGTAA
CGGCCCACCTAGCCTACGATGGATGGGGGTTCTGAGAGGAAGGTCCCCCACATTGGAACTGAGACACGGTCCAAAC
TCCTACGGGAGGCAGCAGTGAGGAATATTGGTCAATGGACGAGAGTCTGAACCAGCCAAGTAGCGTGAAGGATGAA
GGTCCTACGGATTGTAAACTTCTTTTATAAGGGAATAAAACCTCCCACGTGTGGGAGCTTGTATGTACCTTATGAA
TAAGCATCGGCTAACTCCGTGCCAGCAGCCGCGGTAATACGGAGGATGCGAGCGTTATCCGGATTTATTGGGTTTA
AAGGGAGCGCAGACGGGTCGTTAAGTCAGCTGTGAAAGTTTGGGGCTCAACCTTAAAATTGCAGTTGATACTGGCG
TCCTTGAGTGCGGTTGAGGTGTGCGGAATTCGTGGTGTAGCGGTGAAATGCTTAGATATCACGAAGAACTCCGATT
GCGAAGGCAGCACACTAAGCCGTAACTGACGTTCATGCTCGAAAGTGTGGGTATCAAACAGGATTAGATACCCTGG
TAGTCCACACAGTAAACGATGAATACTCGCTGTTTGCGATATACAGTAAGCGGCCAAGCGAAAGCATTAAGTATTC
CACCTGGGGAGTACGCCGGCAACGGTGAAACTCAAAGGAATTGACGGGGGCCCGCACAAGCGGAGGAACATGTGGT
TTAATTCGATGATACGCGAGGAACCTTACCCGGGCTTAAATTGCATTTGAATATATTGGAAACAGTATAGCCGTAA
GGCAAATGTGAAGGTGCTGCATGGTTGTCGTCAGCTCGTGCCGTGAGGTGTCGGCTTAAGTGCCATAACGAGCGCA
ACCCTTATCTTCAGTTACTAACAGGTCATGCTGAGGACTCTGGAGAGACTGCCGTCGTAAGATGTGAGGAAGGTGG
GGATGACGTCAAATCAGCACGGCCCTTACGTCCGGGGCTACACACGTGTTACAATGGGGGGTACAGAAGGCCGCTA
CCTGGTGACAGGATGCTAATCCCAAAAGCCTCTCTCAGTTCGGATCGAAGTCTGCAACCCGACTTCGTGAAGCTGG
ATTCGCTAGTAATCGCGCATCAGCCATGGCGCGGTGAATACGTTCCCGGGCCTTGTACACACCGCCCGTCAAGCCA
TGAAAGCCGGGGGTACCTGAAGTACGTAACCGCAAGGAGCGTCCTAGGGTAAAACTGGTAATTGGGGCTAAGTCAT
A (SEQ ID NO: 63)
GX182404.8.1529
AGAGTTTGATCATGGCTCAGATTGAACGCTGGCGGCAGGCCTAACACATGCAAGTCGAACGGTAACAGGAAGAAGC
TTGCTTCTTTGCTGACGAGTGGCGGACGGGTGAGTAATGTCTGGGAAACTGCCTGATGGAGGGGGATAACTACTGG
AAACGGTAGCTAATACCGCATAACGTCGCAAGACCAAAGAGGGGGACCTTCGGGCCTCTTGCCATCGGATGTGCCC
AGATGGGATTAGCTAGTAGGTGGGGTAACGGCTCACCTAGGCGACGATCCCTAGCTGGTCTGAGAGGATGACCAGC
CACACTGGAACTGAGACACGGTCCAGACTCCTACGGGAGGCAGCAGTGGGGAATATTGCACAATGGGCGCAAGCCT
GATGCAGCCATGCCGCGTGTATGAAGAAGGCCTTCGGGTTGTAAAGTACTTTCAGCGGGGAGGAAGGGAGTAAAGT
TAATACCTTTGCTCATTGACGTTACCCGCAGAAGAAGCACCGGCTAACTCCGTGCCAGCAGCCGCGGTAATACGGA
GGGTGCAAGCGTTAATCGGAATTACTGGGCGTAAAGCGCACGCAGGCGGTTTGTTAAGTCAGATGTGAAATCCCCG
GGCTCAACCTGGGAACTGCATCTGATACTGGCAAGCTTGAGTCTCGTAGAGGGGGGTAGAATTCCAGGTGTAGCGG
TGAAATGCGTAGAGATCTGGAGGAATACCGGTGGCGAAGGCGGCCCCCTGGACGAAGACTGACGCTCAGGTGCGAA
AGCGTGGGGAGCAAACAGGATTAGATACCCTGGTAGTCCACGCCGTAAACGATGTCGACTTGGAGGTTGTGCCCTT
GAGGCGTGGCTTCCGGAGCTAACGCGTTAAGTCGACCGCCTGGGGAGTACGGCCGCAAGGTTAAAACTCAAATGAA
TTGACGGGGGCCCGCACAAGCGGCGGAGCATGTGGATTAATTCGATGCAACGCGAAGAACCTTACCTGGGTTTGAC
ATGCACAGGACGCGTCTAGAGATAGGCGTTCCCTTGTGGCCTGTGTGCAGGTGGTGCATGGCTGTCGTCAGCTCGT
GTCGTGAGATGTTGGGTTAAGTCCCGCAACGAGCGCAACCCTTGTCTCATGTTGCCAGCACGTAATGGTGGGGACT
CGTGAGAGACTGCCGGGGTCAACTCGGAGGAAGGTGGGGATGACGTCAAGTCATCATGCCCCTTATGTCCAGGGCT
TCACACATGCTACAATGGCCGGTACAAAGGGCTGCGATGCCGCGAGGTTAAGCGAATCCTTAAAAGCCGGTCTCAG
TTCGGATCGGGGTCTGCAACTCGACCCCGTGAAGTCGGAGTCGCTAGTAATCGCAGATCAGCAACGCTGCGGTGAA
TACGTTCCCGGGCCTTGTACACACCGCCCGTCACGTCATGAAAGTCGGTAACACCCGAAGCCAGTGGCCTAACCCT
CGGGAGGGAGCTGTCGAAGGTGGGATCGGCGATTGGGACGAAGTCGTAACAAGGTAACCGTAGGGGAACCTGCGGT
TG (SEQ ID NO: 64)
FJ950694.1.1472
CGCCCTGATTGACGGCTATACACATGCAAGTCGAACGGTAACAGGAAACAGCTTGCTTCTTTGCTGACGAGTGGCG
GACGGGTGAGTAATGTCTGGGAAACTGCCTGATGGAGGGGGATAACTACTGGAAACGGTAGCTAATACCGCATAAC
GTCGCAAGACCAAAGAGGGGGACCTTCGGGCCTCTTGCCATCGGATGTGCCCAGATGGGATTAGCTAGTAGGTGGG
GTAACGGCTCCATCCCTAGGCGAGCCGAATCCTTAGCCTGGTCTGAGAGGAATGACCAGCCACACTGGGACTGAGA
ACACGGTCCAGACTCCTACGGGAGGCAGCAGTGGGGAATATTGCACAATGGGCGCAAGCCTGATGCAGCCATGCCG
CGTGTATGAAGAAGGCCTTCGGGTTGTAAAGTACTTTCAGCGGGGAGGAAGGGAGTAAAGTTAATACCCTTTGCTC
ATTGACGTTACCCGCAGAAGAAGCACCGGCTAACTCCGTGCCAGCAGCCGCGGTAATACGGAGGGTGCAAGCGTTA
ATCGGAATTACTGGGCGTAAAGCGCACGCAGGCGGTTTGTTAAGTCAGATGTGAAATCCCCGGGCTCAACCTGGGA
ACTGCATCTGATACTGGCAAGCTTGAGTCTCGTAGAGGGGGGTAGAATTCCAGGTGTAGCGGTGAAATGCGTAGAG
ATCTGGAGGAATACCGGTGGCGAAGGCGGCCCCCTGGACGAAGACTGACGCTCAGGTGCGAAAGCGTGGGGAGCAA
ACAGGATTAGATACCCTGGTAGTCCACGCCGTAAACGATGTCGACTTGGAGGTTGTGCCCTTGAGGCGTGGCTTCC
GGAGCTAACGCGTTAAGTCGACCGCCTGGGGAGTACGGCCGCAAGGTTAAAACTCAAATGAATTGACGGGGGCCCG
CACAAGCGGTGGAGCATGTGGTTTAATTCGATGCAACGCGAAGAACCTTACCTGGTCTTGACATCCACGGGAAGTT
TTCAGAGATGAGAATGTGCCTTCGGGAACCGTGAGACAGGTGCTGCATGGCTGTCGTCAGCTCGTGTTGTGAAATG
TTGGGTTAAGTCCCGCAACGAGCGCAACCCTTATCCTTTGTTGCCAGCGGTCCGGCCGGGAACTCAAAGGAGACTG
CCAGTGATAAACTGGAGGAAGGTGGGGATGACGTCCAGGTCATCATGGCCCTTACGAACCAGGGCTACACACGTGC
CTACAATGGACGCATCCAAAGAGAGAGCGAACCCTGCCCGCGAGAGCAAGCGGACCTCATAAAGTGCGTCGTAGTC
CGGATTGGAGTCTGCAACTCGACTCCATGAAGTCGGAATCGCTAGTAATCGTGGATCAGAATGCCACGGTGAATAC
GTTCCCGGGCCTTGTACACACCGCCCGTCACACCATGGGAGTGGGTTGCAAAAGAAGTAGGTAGCTTAACCTTCGG
GAGGGCGCTTACCACTTTGGATGCGAGG (SEQ ID NO: 65)
GQ448506.1.1374
AGAGTTTGATCATGGCTCAGGATGAACGCTAGCTACAGGCTTAACACATGCAAGTCGAGGGGCAGCATGGTCTTAG
CTTGCTAAGGCCGATGGCGACCGGCGCACGGGTGAGTAACACGTATCCAACCTGCCGTCTACTCTTGGACAGCCTT
CTGAAAGGAAGATTAATACAAGATGGCATCATGAGTCCGCATGTTCACATGATTAAAGGTATTCCGGTAGACGATG
GGGATGCGTTCCATTAGATAGTAGGCGGGGTAACGGCCCACCTAGTCTTCGATGGGTAGGGGTTCTGAGAGGAAGG
TCCCCCACATTGGAACTGAGACACGGCCCAAACTCATACGGGAGGCAGCAGTGGGGAATATTGCACAATGGGGGAA
ACCCTGATGCAGCGACGCCGCGTGAAGGATGAAGTATTTCGGTATGTAAACTTCTATCAGCAGGGAAGAAAATGAC
GGTACCTGACTAAGAAGCCCCGGCTAACTACGTGCCAGCAGCCGCGGTAATACGTAGGGGGCAAGCGTTATCCGGA
TTTACTGGGTGTAAAGGGAGCGTAGACGGCAGTGCAAGTCTGAAGTGAAAGCCCGGGGCTCAACCCCGGGACTGCT
TTGGAAACTGTGCAGCTAGAGTGTCGGAGAGGCAAGCGGAATTCCTAGTGTAGCGGTGAAATGCGTAGATATTAGG
AGGAACACCAGTGGCGAAGGCGGCTTGCTGGACGATGACTGACGTTGAGGCTCGAAAGCGTGGGGAGCAAACAGGA
TTAGATACCCTGGTAGTCCACGCCGTAAACGATGACTACTAGGTGTCGGGGAGCAAAGCTCTTCGGTGCCGCAGCC
AACGCAATAAGTAGTCCACCTGGGGAGTACGTTCGCAAGAATGAAACTCAAAGGAATTGACGGGGACCCGCACAAG
CGGTGGAGCATGTGGTTTAATTCGAAGCAACGCGAAGAACCTTACCTGCTCTTGACATCCCTCTGACCGCTCTTTA
ATCGGAGCTTTCCTTCGGGACAGAGGAGACAGGTGGTGCATGGTTGTCGTCAGCTCGTGTCGTGAGATGTTGGGTT
AAGTCCCGCAACGAGCGCAACCCTTATGGTCAGTTACTACGCAAGAGGACTCTGGCCAGACTGCCGTTGACAAAAC
GGAGGAAGGTGGGGATGACGTCAAATCATCATGCCCTTTATGACTTGGGCTACACACGTACTACAATGGCGTTAAA
CAAAGAGAAGCGAGACCGCGAGGTGGAGCAAAACTCGGAAACAACGTCCCAGTTCGGACTGCAGGCTGCAACTCGC
CTGCACGAAGTCGGAATTGCTAGTAATCGCAGATCAGCATGCTGCGGTGAATACGTTCCCGGGCCTTGCACTCACC
GCCCGT (SEQ ID NO: 66)
HQ802983.1.1440
TAAGATGAACGCTGGCGGCGTGCTTAACACATGCAAGTCCTATGAAGCGCTTAAACGGATTTCTTCGGATTGAAGT
TTTTGTGACTGAGTGGCGGACGGGTGAGTAACGCGTGGGTAACTTGCCTCATACAGGGGGATAACAGTTAGAAATG
ACTGCTAATACCGCATAAGCGCACAGTGCTGCATGGCACAGTGTGAAAAACTCCGGTGGTATGAGATGGACCCGCG
TCTGATTAGCTAGTTGGTGGGGTAACGGCCTACCAAGGCGACGATCAGTAGCCGGCCTGAGAGGGTGAACGGCCAC
ATTGGGACTGAGACACGGCCCAAACTCCTACGGGAGGCAGCAGTGGGGAATATTGCACAATGGGGGAAACCCTGAT
GCAGCGACGCCGCGTGAGCGAAGAAGTATTTCGGTATGTAAAGCTCTATCAGCAGGGAAGAAAATGACGGTACCTG
ACTAAGAAGCACCGGCTAAATACGTGCCAGCAGCCGCGGTAATACGTATGGTGCAAGCGTTATCCGGATTTACTGG
GTGTAAAGGGAGCGTAGACGGTTGTGTAAGTCTGATGTGAAAGCCCGGGGCTCAACCCCGGGACTGCATTGGAAAC
TATGTAACTAGAGTGTCGGAGAGGTAAGCGGAATTCCTAGTGTAGCGGTGAAATGCGTAGATATTAGGAGGAACAC
CAGTGGCGAAGGCGGCTTACTGGACGATCACTGACGTTGAGGCTCGAAAGCGTGGGGAGCAAACAGGATTAGATAC
CCTGGTAGTCCACGCCGTAAACGATGACTACTAGGTGTCGGGGCCCATAAGGGCTTCGGTGCCGCAGCAAACGCAA
TAAGTATTCCACCTGGGGAGTACGTTCGCAAGAATGAAACTCAAAGGAATTGACGGGGACCCGCACAAGCGGTGGA
GCATGTGGTTTAATTCGAAGCAACGCGAAGAACCTTACCTGGTCTTGACATCCCACTGACCGGACAGTAATGTGTC
CTTTCCTCCGGGACAGTGGAGACAGGTGGTGCATGGTTGTCGTCAGCTCGTGTCGTGAGATGTTGGGTTAAGTCCC
GCAACGAGCGCAACCCCTATCCTTAGTAGCCAGCAGTAAGATGGGCACTCTAGGGAGACTGCCAGGGATAACCTGG
AGGAAGGTGGGGATGACGTCAAATCATCATGCCCCTTATGACTTGGGCTACACACGTGCTACAATGGCGTAAACAA
AGTGAAGCGAAGTCGTGAGGCCAAGCAAATCACAAAAATAACGTCTCAGTTCGGATTGTAGTCTGCAACTCGACTA
CAAGAAGCTGGAATCGCTAGTAATCGCAGATCAGAATGCTGCGGTGAATACGTTCCCGGGTCTTGTACACACCGCC
CGTCACACCATGGGAGTCGAAAATGCCCGAAGTCGGTGACCTAACGAAAGAAGGAGCCGCCGAAGGCAGGTT
(SEQ ID NO: 67)
DQ793824.1.1370
ATGAACGCTGGCGGCGTGCCTAACACATGCAAGTCGAACGAAGCGCTTGAACGGATATCTTCGGACTGAAGTTCTT
GCGACTGAGTGGCGGACGGGTGAGTAACGCGTGGGTAACCTGCCTCATACAGGGGGATAACAGTTAGAAATGACTG
CTAATACCGCATAAGCGCACAGCTTCGCATGGAGCAGTGTGAAAAACTCCGGTGGTATGAGATGGACCCGCGTCAG
ATTAGCTAGTTGGCAGGGTAACGGCCTACCAAGGCGACGATCTGTAGCCGACCTGAGAGGGTGACCGGCCACATTG
GGACTGAGACACGGCCCAAACTCCTACGGGAGGCAGCAGTGGGGAATATTGCACAATGGGGGAAACCCTGATGCAG
CGACGCCGCGTGAGCGAAGAAGTATTTCGGTATGTAAAGCTCTATCAGCAGGGAAGATAATGACGGTACCTGACTA
AGAAGCTCCGGCTAAATACGTGCCAGCAGCCGCGGTAATACGTATGGAGCAAGCGTTATCCGGATTTACTGGGTGT
AAAGGGAGCGTAGACGGTTTGACAAGTCTGATGTGAAATTCCAGGGCTTAACCCTGGACCTGCATTGGAAACTGTC
GGACTAGAGTGTCGGAGAGGTGAGTGGAATTCCTAGTGTAGCGGTGAAATGCGTAGATATTAGGAGGAACACCAGT
GGCGAAGGCGGCTCACTGGACGATAACTGACGTTGAGGCTCGAAAGCGTGGGGAGCAAACAGGATTAGATACCCTG
GTAGTCCACGCCGTAAACGATGTGTACTAGGTGTTGGGGAGCAAAGCTCTTCGGTGCCGTCGCAAACGCAGTAAGT
ACACCACCTGGGGAGTACGTTCGCAAGAATGAAACTCAAAGGAATTGACGGGGACCCGCACAAGCGGTGGAGCATG
TGGTTTAATTCGAAGCAACGCGAAGAACCTTACCAAATCTTGACATCGGAGTGACCGCTCTTTAATCGGAGCTTTC
CTTCGGGACACTCCAGACAGGTGGTGCATGGTTGTCGTCAGCTCGTGTCGTGAGATGTTGGGTTAAGTCCCGCAAC
GAGCGCAACCCTTATCCTTAGTAGCCAGCAAGTGAAGTTGGGCACTCTAGGGAGACTGCCAGGGATAACCTGGAGG
AAGGTGGGGATGACGTCAAATCATCATGCCCCTTATGATTTGGGCTACACACGTGCTACAATGGCGTAAACAAAGG
GAAGCGATCACGTGAGTGTGAGCAAATCTCAAAAATAACGTCCCAGTTCGGACTGTAGTCTGCAACCCGACTACAC
GAAGCTGGAATCGCTAGTAATCGCAGGTCAGCATACTGCGGTGAATACGTTCCCGGGTCTTGCACACACCGCCCGT
CA (SEQ ID NO: 68)
GQ448468.1.1366
AGAGTTTGATCCTGGCTCAGGATGAACGCTGACAGAATGCTTAACACATGCAAGTATACTTGATCCTTCGGGTGAT
GGTGGCGGACGGGTGAGTAACGCGTAAAGAACTTGCCCTGCAGTCTGGGACAACATTTGGAAACGAATGCTAATCC
CGCATAAGCCCACAGCTCGGCATCGAGCAGAGGGAAAAGGAGTGATCTGCTTTGAGATGGCCTCGCGTCCGATTAG
CTGGTTGGTGAGGTGACGGCCCATCAAGGCAACGATCGGTAGCCGGACTGAGAGGTTGAACGGCCACATTGGGATT
GAGACACGGCCCTTACTCCTACGGGAGGCAGCAGTGGGGAATATTGGACAATGGACCAAAAGTCTGATCCAGCAAT
TCTGTGTGCACGATGAAGTTTTTCGGAATGTAAAGTGCTTTCAGTTGGGACGAAGTAAGTGACGGTACCAACAGAA
GAAGCGACGGCTAAATACGTGCCAGCAGCCGCGGTAATACGTATGTCGCAAGCGTTATCCGGATTTATTGGGCGTA
AAGCGCGTCTAGGCGGTTTGGTAAGTCTGATGTGAAAATGCGGGGCTCAACTCCGTATTGCGTTGGAAACTGCCAA
ACTAGAGTACTGGAGAGGTGGGCGGAACTACAAGTGTAGAGGTGAAATTCGTAGATATTTGTAGGAATGCCGATGG
GGAAGCCAGCCCACTGGACAGATACTGACGCTAAAGCGCGAAAGCGTGGGTAGCAAACAGGATTAGATACCCTGGT
AGTCCACGCCGTAAACGATGATTACTAGGTGTTGGGGGTCGAACCTCAGCGCCCAAGCTAACGCGATAAGTAATCC
GCCTGGGGAGTACGTACGCAAGTATGAAACTCAAAGGAATTGACGGGGACCCGCACAAGCGGTGGAGCATGTGGTT
TAATTCGACGCAACGCGAGGAACCTTACCAGCGTTTGACATCCTAAGAAATTAGCAGAGATGCTTTTGTGCCCCTT
CGGGGGAACTTAGTGACAGGTGGTGCATGGCTGTCGTCAGCTCGTGTCGTGAGATGTTGGGTTAAGTCCCGCAACG
AGCGCAACCCCTTTCGTATGTTGCCATCATTAAGTTGGGCACTCATGCGATACTGCCTGCGATGAGCAGGAGGAAG
GTGGGGATGACGTCAAGTCATCATGCCCCTTATACGCTGGGCTACACACGTGCTACAATGGGTAGTACAGAGAGTC
GCAAACCTGCGAGGGGGAGCTAATCTCAGAAAACTATTCTCAGTTCGGATTGTACTCTGCAACTCGAGTACATGAA
GTTGGAATCGCTAGTAATCGCAAATCAGCTATGTTGCGGTGAATACGTTCTCGGGTCTTGTACACACCGCCCGT
(SEQ ID NO: 69)
EU774020.1.1361
AGAGTTTGATCCTGGCTCAGGACGAACGCTGGCGGCGTGCCTAACACATGCAAGTCGAGCGATTCTCTTCGGAGAA
GAGCGGCGGACGGGTGAGTAACGCGTGGGTAACCTGCCCTGTACACACGGATAACATACCGAAAGGTATGCTAATA
CGGGATAATATATAAGAGTCGCATGACTTTTATATCAAAGATTTTTCGGTACAGGATGGACCCGCGTCTGATTAGC
TTGTTGGCGGGGTAACGGCCCACCAAGGCGACGATCAGTAGCCGACCTGAGAGGGTGATCGGCCACATTGGAACTG
AGACACGGTCCAAACTCCTACGGGAGGCAGCAGTGGGGAATATTGCACAATGGGCGCAAGCCTGATGCAGCAACGC
CGCGTGAGCGATGAAGGCCTTCGGGTCGTAAAGCTCTGTCCTCAAGGAAGATAATGACGGTACTTGAGGAGGAAGC
CCCGGCTAACTACATGCCAGCAGCCGCGGTAATACGTATGTCGCAAGCGTTATCCGGATTTATTGGGCGTAAAGCG
CGTCTAGGTGGTTTGGTAAGTCTGATGTGAAAATGCGGGGCTCAACTCCGTATTGCGTTGGAAACTGCCAAACTAG
AGTACTGGAGAGGTAGGCGGAACTACAAGTGTAGAGGTGAAATTCGTAGATATTTGTAGGAATGCCGATGGGGAAG
CCAGCCTACTGGACAGATACTGACGCTAAAGCGCGAAAGCGTGGGTAGCAAACAGGATTAGATACCCTGGTAGTCC
ACGCCGTAAACGATGATTACTAGGTGTTGGGGGTCGAACCTCAGCGCCCAAGCTAACGCGATAAGTAATCCGCCTG
GGGAGTACGTACGCAAGTATGAAACTCAAAGGAGTTGACGGGGACCCGCACAAGCGGTGGAGCATGTGGTTTAATT
CGACGCAACGCGAGGAACCTTACCAGCGTTTGACATCCTAGGAATGAGAAAGAGATTTCTTAGTGCTCCTTCGGGA
GAACCTAGAGACAGGTGGTGCATGGCTGTCGTCAGCTCGTGTCGTGAGATGTTGGGTTAAGTCCCGCAACGAGCGC
AACCCCTATTGTATGTTGCCATCATTAAGTTGGGCACTCATGCGATACTGCCTGCGATGAGCAGGAGGAAGGTGGG
GATGACGTCAAGTCATCATGCCCCTTATACGCTGGGCTACACACGTGCTACAATGGGCAGTACAGAGAGAAGCAAT
ACCGCGAGGTGGAGCCAAACTTAAAAACCAGTCTCAGTTCGGATTGTAGGCTGAAACTCGCCTACATGAAGCTGGA
GTTACTAGTAATCGCGAATCAGAATGTCGCGGTGAATACGTACCCGGGTCTTGTACACACCGCCCGTCA (SEQ ID
NO: 70)
GQ491183.1.1360
GATGAACCCTTGCGGCGTGCTTAACACATGCAAGTCGAACGGGAAACATTTTATTGAAGCTTCGGCAGATCTAGCT
TGTTTCTAGTGGCGGACGGGTGAGTAACGCGTGGGCAACCTGCCTCACACTGGGGGATAACAGTCAGAAATGGCTG
CTAATACCGCATAAGCGCACAGCATCGCATGATGCAGTGTGAAAAACTCCGGTGGTGTGAGATGGACCCGCGTTGG
ATTAGCTAGTTGGCAGGGCAGCGGCCTACCAAGGCGACGATCCATAGCCGGCCTGAGAGGGTGAACGGCCACATTG
GGACTGAGACACGGCCCAGACTCCCACGGGAGGCAGCAGTGGGGAATATTGCACAATGGGGGAAACCCTGATGCAG
CGACGCCGCGTGAAGGAAGAAGTATCTCGGTATGTAAACTTCTATCAGCAGGGAAGAAAATGACGGTACCTGACTA
AGAAGCCCCGGCTAACTACGTGCCAGCAGCCGCGGTAATACGTAGGGGGCAAGCGTTATCCGGATTTACTGGGTGT
AAAGGGAGCGTAGACGGTGTTGCAAGTCTGATGTGAAAGGCGGGGGCTCAACCCCTGGACTGCATTGGAAACTGTG
ATACTCGAGTGCCGGAGAGGTAAGCGGAATTCCTAGTGTAGCGGTGAAATGCGTAGATATTAGAAGGAATACCAGT
GGCGAAGGCGGCTTACTGGACGGTAACTGACGTTGAGGCTCGAAAGCGTGGGGAGCAAACAAGATTAAGAAACCTC
TGGGTAGTCCACGCCCGTAAACGAAGGAATAAAGGGGTCGGGAGCAGAGCTTTTCGGTGCCGCAGCAAACCCAATA
AGTATTCCACCTTGAGAGGACGTTCGCAAGAATGAAACTCAAAGGAATTGACGGGGGACCCGCACAAGCGGTGGAG
CATGTGGTTTAATTCGAAGCAACGCGAAGAACCTTACCAAGTCTTGACATCCCTCTGACCGCACCTTAACCGGTGC
TTTCCTTCGGGACAGAGGAGACAGGTGGTGCATGGTTGTCGTCAGCTCGTGTCGTGAGATGTTGGGTTAAGTCCCG
CAACGAGCGCAACCCTTATCCTTAGTAGCCAGCGGTCCGGCCGGGCACTCTGGGGAGACTGCCAGGGATAACCTGG
AGGAAGGTGGGGATGACGTCAAATCATCATGCCCCTTATGATTTGGGCTACACACGTGCTACAATGGCGTAAACAA
AGGGAAGCGATCACGTGAGTGCGAGCAAATCTCAAAAATAACGTCCCAGTTCGGACTGTAGTCTGCAACCCGACTA
CACGAAGCTGGAATCGCTAGTAATCGCAGGTCAGCATACTGCGGTGAATACGTTCCCGGGTCTTGTAC (SEQ ID
NO: 71)
GQ491426.1.1332
GCTCAGGATGAACGCTGGCGGCGTGCTTAACACATGCAAGTCGAGCGAAGCACTTGCCATTGACTCTTCGGAAGAT
TTGGCATTTGACTGAGCGGCGGACGGGTGAGTAACGCGTGGGTAACCTGCCTCATACGGGGGAATAACAGTTAGAA
ATGGCTGCTAATGCCGCATAACCGCACAGGACCGCATGGACTGGTGTGAAAAACTGAGGTGGTATGAGATGGGCCC
GCGTCTGATTAGGTTAGTTGGCGGGGTAACGGCCCACCAAGCCGACGATCAGTAGCCGACCTGAGAGGGACCGGCC
ACATTGGGACTGAGACATGGCCCAGACTCCTACGGGAGGCAGCAGTGGGGAATATTGCACAATGGAGGAAACTCTG
ATGCAGCGACGCCGCATGAAGGAAGAAGTATCTCGGTATGTAAACTTCTATCAGCAGGGAAGAAAATGACGGTACC
TGACTAAGAAGCCCCGGCTAACTACGTGCCAGCAGCCGCGGTAATACGTAGGGGGCAAGCGTTATCCGGATTTACT
GGGTGTAAAGGGAGCGTAGACGGACGGGCAAGTCTGATGTGAAAGCCCGGGGCTTAACCCCGGGACTGCATTGGAA
ACTGTCCATCTTGAGTGCCGGAGAGGTAAGCGGAATTCCTAGTGTAGCGGTGAAATGCGTAGATATTAGGAGGAAC
ACCAGTGGCGAAGGCGGCTTACTGGACGGTAACTGACGTTGAGGCTCGAAAGCGTGGGGAGCAAACAGGATTAGAT
ACCCTGGTAGTCCACGCCGTAAACGATCAATAATGGGTGTCGGGTTGCAAAGCAATCCGGTGCCGCAGCAAACGCA
GTAAGTATTCCCCCTCGGGAGTACGTTCGCAAGAATGAAACTCAAAGGAAGGGACGGGGATCCGCACAAGCGGCGG
AGCATGTGGTTTAATTAGAAGCAACGCGAAGAACCTTACCAAGTCTTGACATCTGCCTGACCGTTCCTTAACCGGA
ACTATCTTTCGGGACAGGCAAGACAGGTGGTGCATGGTTGTCGTCAGCTCGTGTCGTGAGATGTTGGGTTAAGTCC
CGCAACGAGCGCAACCCCTGTCCTTAGTAGCCAGCAGTCCGGCTGGGCACTCTAGGGAGACTGCCGGGGGTAACCC
GGAGGAAGGCGGGGAGGAGGTCAAATCATCATGCCCCCCCTGATTTGGGCTACACACGTGGTACAATGGCGTAAAC
AAAGGGAAGCGGAGTGGTGACGCTGAGCAAATCTCAAAAATAACGTCCCACTTCGGACTGCAGTCTGCAACTCGAC
TGCACGAAGCTGGAATCGCTAGTAATCGCGAATCAGAATG (SEQ ID NO: 72)
GQ493039.1.1311
GATGAACGCTGGCGGCGTGCCTAACACATGCAAGTCGAGCGATTTACTTCGGTAAAGAGCGGCGGACGGGTGAGTA
ACGCGTGGGTAACCTGCCCTGTACACACGGATAACATACCGAAAGGTATGCTAATACGAGATAATATGCTTTTATC
GCATGGTAGAAGTATCAAAGCTTTTGCGGTACAGGATGGACCCGCGTCTGATTAGCTAGTTGGTAAGGTAACGGCT
TACCAAGGCAACGATCAGTAGCCGACCTGAGAGGGTGATCGGCCACATTGGAACTGAGACACGGTCCAAACTCCTA
CGGGAGGCAGCAGTGGGGAATATTGCACAATGGGCGAAAGCCTGATGCAGCAACGCCGCGTGAGCGATGAAGGCCT
TCGGGTCGTAAAGCTCTGTCCTCAAGGAAGATAATGACGGTACTTGAGGAGGAAGCCCCGGCTAACTACGTGCCAG
CAGCCGCGGTAATACGTAGGGGGCTAGCGTTATCCGGAATTACTGGGCGTAAAGGGTGCGTAGGTGGTTTCTTAAG
TCAGAGGTGAAAGGCTACGGCTCAACCGTAGTAAGCCTTTGAAACTGGGAAACTTGAGTGCAGGAGAGGAGAGTGG
AATTCCTAGTGTAGCGGTGAAATGCGTAGATATTAGGAGGAACACCAGTTGCGAAGGCGGCTCTCTGGACTGTAAC
TGACACTGAGGCACGAAAGCGTGGGAGCAAACAAGATTAGNTNCCCTGGTAGTCCNCGCCGTNNCCGCCCATAAAG
AGCTGTCGGAGGTTACCCCCTTCGGTGGCGCAGGTAACGCAATAAAGAATTCCGCCTGGGAAGGAACGCTTCGCAA
GAGTGAAATTAAAAGGAATAGACGGGGACCCGCTCAAGTAGTGGAGCATGTGGTTTAATTCGAAGCAACGCGAAGA
ACTTTCTCTAAGCTTGACATCCTTTTGACCGATGCCTAATAGCATCAATCCCTTCTGGGACAGAAGTGACAGGTGG
TGCATGGTTGTTGTCAGCTCGTGTCGTGAGATGTTGGGTTAAGTCCCGCAACGAGCGCAACCCTTGCCTTTAGTTG
CCAGCATTAAGTTGGGCACTCTATAGGGACTGCCAGGGATAACCTGGAGGAAGGTGGGGATGACGTCAAATCATCA
TGCCCCTTATGCTTAGGGCTACACACGTGCTACAATGGGTGGTACAGAGGGCAGCCAAGTCGTGAGGCGGAGCTAA
TCCCTTAAAGCCATTCTCAGTTCGGATTGTAGGCTGAAACTCGCCTACATGAAGCTGGAGTTACTAGTAATCGCAG
ATCAGAATGATGCGGTGAA (SEQ ID NO: 73)
JN387556.1.1324
CGTAAGTAACCTGCCCTGTACACACGGATAACATACCGAAAGGTATGCTAATACGGGATAATATATTTTGATCGCA
TGGTCGAGATATCAAAGCTCCGGCGGTACACCAGGGACCCCCGACAGAGGAGCTAGTTGGTAGTAATGTCACCAAG
GCGACGATCAGAAGCCGAACTGAGAGGGGGATCCGCACATGACTGAGACACGGTCAAACTCCTACGGGAGGCAGCA
GTGGGGAATATGCCAATGGGCGAAAGCTGATGCAGCACGCGCGTGAGCGATGAGGCTCGGGTCGTAAAGCTCGTCT
CAAGGAAGATAATGACGGTACTTGAGGAGGAAGCCCCGGCTAACTACGTGCCAGCAGCCGCGGTAATACGTAGGGG
GCTAGCGTTATCCGGAATTACTGGGCGTAAAGGGTGCGTAGGCGGTCTTTCAAGTCAGGAGTGAAAGGCTACGGCT
CAACCGTAGTAAGCTCTTGAAACTGTAAGACTTGAGTGCAGGAGAGGAGAGTGGAATTCCTAGTGTAGCGGTGAAA
TGCGTAGATATTAGGAGGAACACCAGTTGCGAAGGCGGCTCTCTGGACTGTAACTGACGCTGAGGCACGAAAGCGT
GGGGAGCAAACAGGATTAGATACCCTGGTAGTCCACGCCGTAAACGATGAGTACTAGCTGTCGGAGGTTACCCCCT
TCGGTGGCGCAGCTAACGCATTAAGTACTCCGCCTGGGAAGTACGCTCGCAAGAGTGAAACTCAAAGGAATTGACG
GGGACCCGCACAAGTAGCGGAGCATGTGGTTTAATTCGAAGCAACGCGAAGAACCTTACCTAAGCTTGACATCCCA
CTGACCCTTCCCTAATCGGAAGCTTCCCTTCGGGACAGTGGTGACAGGTGGTGCATGGTTGTCGTCAGCTCGTGTC
GTGAGATGTTGGGTTAAGTCCCGCAACGAGCGCAACCCTTGCCTTTAGTTGCCAGCATTAAGTTGGGCACTCTAGA
GGGACTGCCAGGGATAACCCGGAGGAGTGGGGATGACGTCAAATCATCATGCCCTTATGCTAGGCTACACACGTGC
TACAATGGGTGGTCAGAGGCCAGCCAGTCGTGAGGCCGAGCTATCCCATAAGCCATTCTCGTCCGGATTGTAGGCT
GAACTCGCCTACATGAGCTGGAATTACAAGTATGCGATCGATGCTGCGTGATGCGTCCGGGTCTTGTACACACCGC
CCGTCACACCATGGGAGTTGGGGGCGCCCGAAGCCGGATTGCTAACCTTTTGGAAGCGTCCGTCGAAGGTGAAACC
AATAACTGGGGTGAAGTCGTAACAAGGTAACC (SEQ ID NO: 74)
EU775983.1.1288
GAAAGCGGCGGACGGGTGAGTAACGCGTAGGCAACCTGCCCCATACAGAGGGATAGCATCTGGAAACGGATATTAA
TACCTCATAATACTTAGAGATCACATGGTAACTAAGTCAAAGATTTATCGGTATGGGATGGGCCTGCGTCTGATTA
GCTAGTTGGTGGGGTAACGGCTCACCAAGGCGACGATCAGTAGCCGACCTGAGAGGGTGATCGGCCACATTGGAAC
TGAGACACGGTCCAAACTCCTACGGGAGGCAGCAGTGGGGAATATTGCACAATGGGCGCAAGCCTGATGCAGCAAC
GCCGCGTGAGCGATGAAGGCCTTCGGGTCGTAAAGCTCTGTCCTCAAGGAAGATAATGACGGTACTTGAGGAGGAA
GCCCCGGCTAACTACGTGCCAGCAGCCGCGGTAATACGTAGGGGGCTAGCGTTATCCGGATTTACTGGGCGTAAAG
GGTGCGTAGGCGGTCTTTCAAGTCAGGAGTTAAAGGCTACGGCTCAACCGTAGTAAGCTCCTGATACTGTCTGACT
TGAGTGCAGGAGAGGAAAGCGGAATTCCCAGTGTAGCGGTGAAATGCGTAGATATTGGGAGGAACACCAGTAGCGA
AGGCGGCTTTCTGGACTGTAACTGACGCTGAGGCACGAAAGCGTGGGGAGCAAACAGGATTAGATACCCTGGTAGT
CCACGCTGTAAACGATGAGTACTAGGTGTCGGAGGTTACCCCCTTCGGTGCCGCAGCTAACGCATTAAGTACTCCG
CCTGGGGAGTACGCACGCAAGTGTGAAACTCAAAGGAATTGACGGGGACCCGCACAAGTAGCGGAGCATGTGGTTT
AATTCGAAGCAACGCGAAGAACCTTACCTAGGCTTGACATCCTTCTGACCGAGGACTAATCTCCTCTTTCCCTCCG
GGGACAGAAGTGACAGGTGGTGCATGGTTGTCGTCAGCTCGTGTCGTGAGATGTTGGGTTAAGTCCCGCAACGAGC
GCAACCCTTGTCTTTAGTTGCCATCATTAAGTTGGGCACTCTAGAGAGACTGCCAGGGATAACCTGGAGGAAGGTG
GGGATGACGTCAAATCATCATGCCCCTTATGCCTAGGGCTACACACGTGCTACAATGGGTGGTACAGAGGGCAGCC
AAGCCGTGAGGTGGAGCAAATCCCTTAAAGCCATTCTCAGTTCGGATTGTAGGCTGAAACTCGCCTACATGAAGCT
GGAGTTACTAGTAATCGCAGATCAGAATGCTGCGGTGAATGCGTTCCCGGGTCTTGCACACACCGCCCGTCA
(SEQ ID NO: 75)
OTUs in Table 5
GQ449137.1.1391
CTGGCTCAGGATGAACGCTAGCGACAGGCTTAACACATGCAAGTCGAGGGGCATCACGGGAGGTAGCAATACCTTC
TGGTGGCGACCGGCGCACGGGTGAGTAACACGTATGCAACCTGCCCTGTACAGAGGGACAAGCGGTGGAAACGCCG
TCTAATCCCGCATGCACTCTTCCGGGGGCATCCCCGGGAGAGTAAAGGAGAGATCCGGTACAGGATGGACATGCGG
CGCATTAGTTAGTTGGCGGGGTAACGGCCCACCAAGACGACGATGCGTAGGGGTTCTGAGAGGAAGGTCCCCCACA
TTGGAACTGAGACACGGTCCAAACTCCTACGGGAGGCAGCAGTGAGGAATATTGGTCAATGGGCGGAAGCCTGAAC
CAGCCAAGTCGCGTGAGGGAAGACGGCCCTACGGGTTGTAAACCTCTTTTGTCGGGGAGCAATGCCGCCTTTGCGA
AGGCGGAGGGAGAGTACCCGAAGAAAAAGCACCGGCTAACTACGTGCCAGCAGCCGCGGTAATACGTAGGGTGCAA
GCGTTAATCGGAATTACTGGGCGTAAAGCGTGCGCAGGCGGTTCTGTAAGACAGATGTGAAATCCCCGGGCTCAAC
CTGGGAATTGCATTTGTGACTGCAGGACTAGAGTTCATCAGAGGGGGGTGGAATTCCAAGTGTAGCAGTGAAATGC
GTAGATATTTGGAAGAACACCAATGGCGAAGGCAGCCCCCTGGGATGCGACTGACGCTCATGCACGAAAGCGTGGG
GAGCAAACAGGATTAGATACCCTGGTAGTCCACGCCCTAAACGATGTCTACTGGTTGTTGGGGATTAATATCCTTG
GTAACGAAGCTAACGCGTGAAGTAGACCGCCTGGGGAGTACGGTCGCAAGATTAAAACTCAAAGGAATTGACGGGG
ACCCGCACAAGCGGTGGATGATGTGGATTAATTCGATGATACGCGAGGAACCTTACCCGGGCTCAAACGGCACAGT
GATACTTTTGAAAGGAGGTAGCTCTACGGAGACTGTGCCGAGGTGCTGCATGGTTGTCGTCAGCTCGTGCCGTGAG
GTGTCGGCTTAAGTGCCATAACGAGCGCAACCCCTATTGTCAGTTGCCAGCAGGTAAAGCTGGGGACTCTGACGAG
ACTGCCGGCGCAAGCTGAGAGGAAGGCGGGGATGACGTCAAATCAGCACGGCCCTTACGTCCGGGGCGACACACGT
GTTACAATGGCAGGCACAGCGGGAAGCCACCCGGCGACGGGGAGCGGAACCCGAAAGCCTGTCTCAGTTCGGATCG
GAGTCTGCAACTCGACTCCGTGAAGCTGGATTCGCTAGTAATCGCGCATCAGCCATGGCGCGGTGAATACGTTCCC
GGGCCTTGTACACACCGCCCGTA (SEQ ID NO: 76)
HK555938.1.1357
ACGGCACCCCTCTCCGGAGGGAAGCGAGTGGCGAACGGCTGAGTAACACGTGGAGAACCTGCCCCCTCCCCCGGGA
TAGCCGCCCGAAAGGACGGGTAATACCGGATACCCCCGGGCGCCGCATGGCGCCCGGGCTAAAGCCCCGACGGGAG
GGGATGGCTCCGCGGCCCATCAGGTAGACGGCGGGGTGACGGCCCACCGTGCCGACAACGGGTAGCCGGGTTGAGA
GACCGACCGGCCAGATTGGGACTGAGACACGGCCCAGACTCCTACGGGAGGCAGCAGTGGGGAATCTTGCGCAATG
GGGGGAACCCTGACGCAGCGACGCCGCGTGCGGGACGGAGGCCTTCGGGTCGTAAACCGCTTTCAGCAGGGAAGAG
TCAAGACTGTACCTGCAGAAGAAGCCCCGGCTAACTACGTGCCAGCAGCCGCGGTAATACGTAGGGGGCGAGCGTT
ATCCGGATTCATTGGGCGTAAAGCGCGCGTAGGCGGCCCGGCAGGCCGGGGGTCGAAGCGGGGGGCTCAACCCCCC
GAAGCCCCCGGAACCTCCGCGGCTTGGGTCCGGTAGGGGAGGGTGGAACACCCGGTGTAGCGGTGGAATGCGCAGA
TATCGGGTGGAACACCGGTGGCGAAGGCGGCCCTCTGGGCCGAGACCGACGCTGAGGCGCGAAAGCTGGGGGAGCG
AACAGGATTAGATACCCTGGTAGTCCCAGCCGTAAACGATGGACGCTGGGTGTGGGGGGACGATCCCCCCGTGCCG
CAGCCNACGCATTAAGCGTCCCGCCTGGGGAGTACGGCCGCAAGGCTAAAACTCAAAGGAATTGACGGGGGCCCGC
ACAAGCAGCGGAGCATGTGGCTTAATTCGAAGCAACGCGAAGAACCTTACGGCGCATCCCCCCGAGGCCCACGGGG
GGTCCGCCGCGTGGGTCAGAGGAGCGCATACGGGAGGTGCATGGTTGTCGTCAGCTCGTGTCGTGAGATGTTGGGT
TAAGTCCCGCAACGAGCGCAACCCCCGCCGCGTGTTGCCATCGGGTGATGCCGGGAACCCACGCGGGACCGCCGCC
GTCAAGGCGGAGGAGGGCGGGGACGACGTCAAGTCATCATGCCCCTTATGCCCTGGGCTGCACACGTGCTACAATG
GCCGGTACAGAGGGATGCCACCCCGCGAGGGGGAGCGGATCCCGGAAAGCCGGCCCCAGTTCGGATTGGGGGCTGC
AACCCGCCCCCATGAAGTCGGAGTTGCTAGTAATCGCGGATCAGCATGCCGCGGTGAATGCGTTCCCGGGCCTTGT
ACACACCGCCCGTCACACCACCCGAGTCGTCTGCACCCGAAGTCGCCGGCCCAACCGCAAGGGGG (SEQ ID NO:
77)
GQ358246.1.1466
AGAGTTGATCTGGCTCAGATTGAACGCTGGCGGCAGGCTTAATACATGCAAGTCGAACGGTAACAGCAAAAAAGCT
TGCTTTTTTGGCTGACGAGTGGCGGACGGGTGAGTAATACCTAGGAAGCTGCCTAAACGAGGGGGATAACACCTGG
AAACGGGTGCTAATACCGCATGATACCGCAAGGTCAAAGGTTGGTTTACCAATCGCGTTTAGATGCGCCTAGGAGG
GATTAGCTAGTTGGTGGGGTAACGGCTCACCAAGGCGATGATCAGTAGCCGGTCTGAGAGGATGAACGGCCACATT
GGGACTGAGACACGGCCCAGACTCCTACGGGAGGCAGCAGTGGGGAATCTTCCGCAATGGGCGAAAGCCTGACGGA
GCAATGCCGCGTGAGTGATGAAGGGATTCGTCCCGTAAAGCTCTGTTGTATATGACGAATGTGCAGATTGTGAATA
ATGATTTGTAATGACGGTAGTATACGAGGAAGCCACGGCTAACTACGTGCCAGCAGCCGCGGTAATACGTAGGTGG
CGAGCGTTGTCCGGAATTATTGGGCGTAAAGAGCATGTAGGCGGTTTTTTAAGTCTGGAGTGAAAATGCGGGGCTC
AACCCCGTATGGCTCTGGATACTGGAAGACTTGAGTGCAGGAGAGGAAAGGGGAATTCCCAGTGTAGCGGTGAAAT
GCGTAGATATTGGGAGGAACACCAGTGGCGAAGGCGCCTTTCTGGACTGTGTCTGACGCTGAGATGCGAAAGCCAG
GGTAGCGAACGGGATTAGATACCCCGGTAGTCCTGGCCGTAAACGATGGGTACTAGGTGTGGGAGGTATCGACCCC
TTCCGTGCCGGAGTTAACGCAATAAGTACCCCGCCTGGGGAGTACGTCCGCAAGGATGAAACTCAAAGGAATTGAC
GGGGGCCCGCACAAGCGGTGGAGTATGTGGTTTAATTCGACGCAACGCGAAGAACCTTACCAAGGCTTGACATTGA
TTGAAAGACCTAGAGATAGGTCCCTCTCTTCGGAGACAAGAAAACAGGTGGTGCATGGCTGTCGTCAGCTCGTGTC
GTGAGATGTTGGGTTAAGTCCCGCAACGAGCGCAACCCCTATCCTATGTTACCAGCGGGTAATGCCGGGGACTCAT
AGGAGACTGCCAAGGACAACTTGGAGGAAGGCGGGGATGACGTCAAGTCATCATGCCCCTTATGTCTTGGGCTACA
CACGTACTACAATGGTCGGCAACAGAGGGAAGCAAAGCCGCGAGGCAGAGCAAACCCCAGAAACCCGATCTCAGTT
CGGATTGCAGGCTGCAACTCGCCTGCATGAAGTCGGAATCGCTAGTAATCGCAGGTCAGCATACTGCGGTGATTAC
TATCCCGGGCGTTGTACTCACCGCCCGTCAGGCGGAGTTCGTACTTCAAATGTGCCACACTGGG (SEQ ID NO:
78)
New.ReferenceOTU82
TACGTAGGTGGCAAGCGTTGTCCGGATTTACTGGGCGTAAAGGGAGCGTAGGCGGATTTTTAAGTGGGATGTGAAA
TACCCGGGCTCAACCTGGGTGCTGCATTCCAAACTGGAAATCTAGAGTGCAGGAGGGGAAAGTGGAATTCCTAGTG
TAGCGGTGAAATGCGTAGAGATTAGGAAGAACACCAGTGGCGAAGGCGACTTTCTGGACTGTAACTGACGCTGAGG
CTCGAAAGCGTGGGGAGCAAACAGG (SEQ ID NO: 79)
New.ReferenceOTU52
TACGTAGGTGGCGAGCGTTATCCGGATTTACTGGGCGTAAAGGGAGCGTAGGCGGATGATTAAGTGGGATGTGAAA
TACCCGGGCTCAACTTGGGTGCTGCATTCCAAACTGGTTATCTAGAGTGCAGGAGAGGAGAGTGGAATTCCTAGTG
TAGCGGTGAAATGCGTAGAGATTAGGAAGAACACCAGTGGCGAAGGCGACTCTCTGGACTGTAACTGACGCTGAGG
CTCGAAAGCGTGGGGAGCAAACAGG (SEQ ID NO: 80)
GQ138615.1.1402
AGAGTTTGATCCTGGCTCAGGATGAACGCTAGCGATAGGCCTAACACATGCAAGTCGAGGGGCAGCACATGAGTAG
CAATACGATGGTGGCGACCGGCGCACGGGTGAGTAACACGTATGCAACCTACCTTTAACAGGGGAATAACCCGTTG
AAAAACGGACTAATACTCCATAACACAGGGGTCCCGCATGGGAATATTTGTTAAAGATTTATCGGTTGAAGATGGG
CATGCGTTCCATTAGCTAGTTGGTAGGGTAAAGGCCTACCAAGGCGACGATGCGTAGCCGACCTGAGAGGGTGAAC
GGCCACACTGGGACTGAGACACGGCCCACACTCCTACGGGAGGCAGCAGTAGGGAATCTTCGGCAATGGGCGAAAG
CCTGACCGAGCAACGCCGCGTGAATGATGAAGGCCTTCGGGTTGTAAAATTCTGTTATAAGGGAAGAACGACTTTA
GTAGGAAATGGCTAGAGTGTGACGGTACCTTATGAGAAAGCCACGGCTAACTACGTGCCAGCAGCCGCGGTAATAC
GTAGGTGGCGAGCGTTATCCGGAATTATTGGGCGTAAAGAGCGCGCAGGTGGTTGATTAAGTCTGATGTGAAAGCC
CACGGCTTAACCGTGGAGGGTCATTGGAAACTGGTCGACTTGAGTGCAGAAGAGGGAAGTGGAATTCCATGTGTAG
CGGTGAAATGCGTAGAGATATGGAGGAACACCAGTGGCGAAGGCGGCTTCCTGGTCTGTAACTGACACTGAGGCGC
GAAAGCGTGGGGAGCAAACAGGATTAGATACCCTGGTAGTCCACGCCGTAAACGATGAGTGCTAAGTGTTGGGGGT
CGAACCTCAGTGCTGAAGTTAACGCATTAAGCACTCCGCCTGGGGAGTACGGTCGCAAGACTGAAACTCAAAGGAA
TTGACGGGGACCCGCACAAGCGGTGGAGCATGTGGTTTAATTCGAAGCAACGCGAAGAACCTTACCAGGTCTTGAC
ATACCATTGACCGTTCTAGAGATAGGATTTTCCCTTCGGGGACAATGGATACAGGTGGTGCATGGTTGTCGTCAGC
TCGTGTCGTGAGATGTTGGGTTAGGTCCCGCAACGAGCGCAACCCCTGTCGTTAGTTGCCAGCATTCAGTTGGGGA
CTCTAACGAGACTGCCAGTGACAAACTGGAGGAAGGTGGGGATGACGTCAAATCATCATGCCCCTTATGACCTGGG
CTACACACGTGCTACAATGGTTGGTACAAAGAGAAGCGAAGCGGTGACGTGGAGCAAACCTCATAAAGCCAATCTC
AGTTCGGATTGTAGGCTGCAACTCGCCTACATGAAGTTGGAATCGCTAGTAATCGCGAATCAGAATGTCGCGGTGA
ATACGTTCCCGGGTCTTGTACACACCGCCCGTCA (SEQ ID NO: 81)
JN681884.1.1409
TGCAAGTAGAACGCTGAAGACTGGTGCTTGCACCGGTTGGAAGAGTTGCGAACGGGTGAGTAACGCGTAGGTAACC
TGCCTGATAGCGGGGGATAACTATTGGAAACGATAGCTAATACCGCATAACAGGGAATAACACATGTTATTTTTTT
GAAAGGGGCAATTGCTCCACTATCAGATGGACCTGCGTTGTATTAGCTAGTAGGTGAGGTAACGGCTCACCTAGGC
GACGATACATAGCCGACCTGAGAGGGTGATCGGCCACACTGGGACTGAGACACGGCCCAGACTCCTACGGGAGGCA
GCAGTAGGGAATCTTCGGCAATGGGGGCAACCCTGACCGAGCAACGCCGCGTGAGTGAAGAAGGTTTTCGGATCGT
AAAGCTCTGTTGTAAGAGAAGAACGTTGAGTAGAGTGGAAAGTTACTCAAGTGACGGTATCTTACCAGAAAGGGAC
GGCTAACTACGTGCCAGCAGCCGCGGTAATACGTAGGTCCCGAGCGTTGTCCGGATTTATTGGGCGTAAAGCGAGC
GCAGGCGGTTTAATAAGTCTGAAGTTAAAGGCAGTGGCTCAACCATTGTTCGCTTTGGAAACTGTTAAACTTGAGT
GCAGAAGGGGAGAGTGGAATTCCATGTGTAGCGGTGAAATGCGTAGATATATGGAGGAACACCGGTGGCGAAAGCG
GCTCTCTGGTCTGTAACTGACGCTGAGGCTCGAAAGCGTGGGTAGCGAACAGGATTAGATACCCTGGTAGTCCACG
CCGTAAACGATGAGTGCTAGGTGTTGGGTCCTTTCCGGGACTCAGTGCCGACGCTAACGCATTAAGCACTCCGCCT
GGGGAGTACGACCGCAAGGTTGAAACTCAAAGGAATTGACGGGGGCCCGCACAAGCGGTGGAGCATGTGGTTTAAT
TCGAAGCAACGCGAAGAACCTTACCAGGTCTTGACATCCCGATGCTATCCCTAGAGATAGGGAGTTACTTCGGTAC
ATCGGTGACAGGTGGTGCATGGTTGTCGTCAGCTCGTGTCGTGAGATGTTGGGTTAAGTCCCGCAACGAGCGCAAC
CCCTATTGTTAGTTGCCATCATTCAGTTGGGCACTCTAGCGAGACTGCCGGTAATAAACCGGAGGAAGGTGGGGAT
GACGTCAAATCATCATGCCCCTTATGACCTGGGCTACACACGTGCTACAATGGTTGGTACAACGAGTTGCGAGTCG
GTGACGGCAAGCTAATCTCTTAAAGCCAATCTCAGTTCGGATTGTAGGCTGCAACTCGCCTACATGAAGTCGGAAT
CGCTAGTAATCGCGGATCAGCACGCCGCGGTGAATACGTTCCCGGGCCTTGTACACACCGCCCGTCACACCACGAG
AGTTTGTAACACCCAAAGTCGGTGAGGTAACCTTCGGAGCC (SEQ ID NO: 82)
GU303759.1.1517
AGAGTTTGATCATGGCTCAGGACGAACGCCGGCGGCGTGCCTAATACATGCAAGTAGAACGCTGAAGACTTTAGCT
TGCTAAAGTTGGAAGAGTTGCGAACGGGTGAGTAACGCGTAGGTAACCTGCCTACTAGCGGGGGATAACTATTGGA
AACGATAGCTAATACCGTATAACAGCATTTAACACATGTTAGATGCTTGAAAGGAGCAATTGCTTCACTAGTAGAT
GGACCTGCGTTGTATTAGCTAGTTGGTGAGGTAACGGCTCACCAAGGCGACGATACATAGCCGACCTGAGAGGGTG
ATCGGCCACACTGGGACTGAGACACGGCCCAGACTCCTACGGGAGGCAGCAGTAGGGAATCTTCGGCAATGGGGGC
AACCCTGACCGAGCAACGCCGCGTGAGTGAAGAAGGTTTTCGGATCGTAAAGCTCTGTTGTAAGAGAAGAACGTGT
GTGAGAGTGGAAAGTTCACACAGTGACGGTAACTTACCAGAAAGGGACGGCTAACTACGTGCCAGCAGCCGCGGTA
ATACGTAGGTCCCGAGCGTTGTCCGGATTTATTGGGCGTAAAGCGAGCGCAGGCGGTTTAATAAGTCTGAAGTTAA
AGGCAGTGGCTTAACCATTGTTCGCTTTGGAAACTGTTAGACTTGAGTGCAGAAGGGGAGAGTGGAATTCCATGTG
TAGCGGTGAAATGCGTAGATATATGGARGGAAACACCGGTGGCGAAAGCGGCTCTCTGGTCTGTAACTGACGCTGA
GGCTCGAGAAGCGTGGGGAGCAAACAGGATTAGATACCCTGGTAGTCCACGCCGTAAGCGATGAGTGCTAGGTGTT
AGGCCCTTTCCGGGGCTTAGTGCCGCAGCTAACGCATTAAGCACTCCGCCTGGGGAGTACGACCGCAAGGTTGAAA
CTCAAAGGAATTGACGGGGGCCCGCACAAGCGGTGGAGCATGTGGTTTAATTCGAAGCAACGCGAAGAACCTTACC
AGGTCTTGACATCCCGATGCTATTCCTAGAGATAGGAAGTTTCTTCGGAACATCGGTGACAGGTGGTGCATGGTTG
TCGTCAGCTCGTGTCGTGAGATGTTGGGTTAAGTCCCGCAACGAGCGCAACCCCTATTGTTAGTTGCCATCATTAA
GTTGGGCACTCTAGCGAGACTGCCGGTAATAAACCGGAGGAAGGTGGGGATGACGTCAAATCATCATGCCCCTTAT
GACCTGGGCTACACACGTGCTACAATGGCGGTCAACAGAGGGAAGCAATACTGTGAAGTGGAGCAAACCCCTAAAA
GCCGTCCCAGTTCGGATTGCAGGCTGCAACCCGCCTGTATGAAGTTGGAATCGCTAGTAATCGCGGATCAGCATGC
CGCGGTGAATACGTTCCCGGGCCTTGTACACACCGCCCGTCACACCATGAGAGTCGGGAACACCCGAAGTCCGTAG
CCTAACTTTCACGAGGGGGCGCGGCCGAAGGTGGGTTCGATAATTGGGGTGAAGTCGTAACAAGGTAACCGTA
(SEQ ID NO: 83)
New.ReferenceOTU114
TACGTAGGTCCCGAGCGTTGTCCGGATTTATTGGGCGTAAAGCGAGCGCAGGCGGTTTAATAAGTCTGAAGTTAAA
GGCAGTGGCTTAACCATTTTTCGCTTTGGAAACTGTTAGACTTGAGTGCAGAAGGGGAGAGTGGAATTCCATGTGT
AGCGGTGAAATGCGTAGATATATGGAGGAACACCGGTGGCGAAAGCGGCTCTCTGGTCTGTAACTGACGCTGAGGC
TCGAAAGCGTGGGGAGCAAACAGG (SEQ ID NO: 84)
EU774881.1.1422
AGAGTTTGATCCTGGCTCAGGACGAACGCTGGCGGCGTGCCTAATACATGCAAGTCGAGCGAGCGGAACTAACAGA
TTTACTTCGGTAATGACGTTAGGAAAGCGAGCGGCGGATGGGTGAGTAACACGTGGGGAACCTGCCCCATAGTCTG
GGATACCACTTGGAAACAGGTGCTAATACCGGATAAGAAAGCAGATCGCATGATCAGCTTTTAAAAGGCGGCGTAA
GCTGTCGCTATGGGATGGCCCCGCGGTGCATTAGCTAGTTGGTAAGGTAAAGGCTTACCAAGGCAATGATGCATAG
CCGAGTTGAGAGACTGATCGGCCACATTGGGACTGAGACACGGCCCAAACTCCTACGGGAGGCAGCAGTAGGGAAT
CTTCCACAATGGACGCAAGTCTGATGGAGCAACGCCGCGTGAGTGAAGAAGGTTTTCGGATCGTAAAGCTCTGTTG
TTGGTGAAGAAGGATAGAGGTAGTAACTGGCCTTTATTTGACGGTAATCAACCAGAAAGTCACGGCTAACTACGTG
CCAGCAGCCGCGGTAATACGTAGGTGGCAAGCGTTGTCCGGATTTATTGGGCGTAAAGCGAGCGCAGGCGGTTTAA
TAAGTCTGAAGTTAAAGGCAGTGGCTTAACCATTGTTCGCTTTGGAAACTGTTAGACTTGAGTGCAGAAGGGGAGA
GTGGAATTCCATGTGTAGCGGTGAAATGCGTAGATATATGGAGGAACACCGGTGGCGAAAGCGGCTCTCTGGTCTG
TAACTGACGCTGAGGCTCGAAAGCGTGGGGAGCAAACAGGATTAGATACCCTGGTAGTCCACGCCGTAAACGATGA
GTGCTAGGTGTTAGGCCCTTTCCGGGGCTTAGTGCCGCAGCTAACGCATTAAGCACTCCGCCTGGGGAGTACGACC
GCAAGGTTGAAACTCAAAGGAATTGACGGGGGCCCGCACAAGCGGTGGAGCATGTGGTTTAATTCGAAGCAACGCG
AAGAACCTTACCAGGTCTTGACATCCCGATGCTATTCCTAGAGATAGGAAGTTTCTTCGGAACATCGGTGACAGGT
GGTGCATGGTTGTCGTCAGCTCGTGTCGTGAGATGTTGGGTTAAGTCCCGCAACGAGCGCAACCCCTATTGTTAGT
TGCCATCATTAAGTTGGGCACTCTAGCGAGACTGCCGGTAATAAACCGGAGGAAGGTGGGGATGACGTCAAGTCAT
CATGCCCCTTATGACCTGGGCTACACACGTGCTACAATGGGCAGTACAACGAGAAGCGAGCCTGCGAAGGCAAGCG
AATCTCTGAAAGCTGTTCTCAGTTCGGACTGCAGTCTGCAACTCGACTGCACGAAGCTGGAATCGCTAGTAATCGC
GGATCAGCACGCCGCGGTGAATACGTTCCCGGGCCTTGCACACACCGCCCGTCA (SEQ ID NO: 85)
AB469559.1.1551
AGAGTTTGATCCTGGCTCAGGACGAACGCTGGCGGCGTGCCTAATACATGCAAGTGGAACGCACAGTTAGTATGTA
GTTTACTACAACATTACTTGTGAGTCGCGAACGGGTGAGTAACGCGTAGGTAACCTGCCTTGTAGCGGGGGATAAC
TATTGGAAACGATAGCTAATACCGCATAACAGTTGATAACTCATGTTATTAGCTTGAAAGATGCAACAGCATCACT
ACGAGATGGACCTGCGTTGTATTAGCTAGTTGGTGAGGTAAAGGCTCACCAAGGCCACGATACATAGCCGACCTGA
GAGGGTGATCGGCCACATTGGGACTGAGACACGGCCCAAACTCCTACGGGAGGCAGCAGTAGGGAATCTTCGGCAA
TGGGGGCAACCCTGACCGAGCAACGCCGCGTGAGTGAAGAAGGTTTTCGGATCGTAAAGCTCTGTTGTAAGAGAAG
AACGTTGATGAGAGTGGAAAATTCATCAAGTGACGGTATCTTACCAGAAAGGGACGGCTAACTACGTGCCAGCAGC
CGCGGTAATACGTAGGTCCCGAGCGTTGTCCGGATTTATTGGGCGTAAAGCGAGCGCAGGCGGTTTCGTAAGTCTG
AAGTTAAAGGCAGTGGCTCAACCATTGTTCGCTTTGGAAACTGCGAGACTTGAGTGCAGAAGGGGAGAGTGGAATT
CCATGTGTAGCGGTGAAATGCGTAGATATATGGAGGAACACCGGTGGCGAAAGCGGCTCTCTGGTCTGTAACTGAC
GCTGAGGCTCGAAAGCGTGGGGAGCAAACAGGATTAGATACCCTGGTAGTCCACGCCGTAAACGATGAGTGCTAGG
TGTTAGGCCCTTTCCGGGGCTTAGTGCCGCAGCTAACGCATTAAGCACTCCGCCTGGGGAGTACGACCGCAAGGTT
GAAACTCAAAGGAATTGACGGGGGCCCGCACAAGCGGTGGAGCATGTGGTTTAATTCGAAGCAACGCGAAGAACCT
TACCAGGTCTTGACATCCCGATGCCCGCTCTAGAGATAGAGTTTTACTTTTGTACATCGGTGACAGGTGGTGCATG
GTTGTCGTCAGCTCGTGTCGTGAGATGTTGGGTTAAGTCCCGCAACGAGCGCAACCCCTATTGTTAGTTGCCATCA
TTGAGTTGGGCACTCTAGCGAGACTGCCGGTAATAAACCGGAGGAAGGTGGGGATGACGTCAAATCATCATGCCCC
TTATGACCTGGGCTACACACGTGCTACAATGGCTGGTACAACGAGTCGCAAGCCGGTGACGGCAAGCTAATCTCTT
AAAGCCAGTCTCAGTTCGGATTGTAGGCTGCAACTCGCCTACATGAAGTCGGAATCGCTAGTAATCGCGGATCAGC
ACGCCGCGGTGAATACGTTCCCGGGCCTTGTACACACCGCCCGTCACACCACGAGAGTTTGTAACACCCGAAGTCG
GTGAGGTAACCTTTTAGGAGCCAGCCGCCTAAGGTGGGATAGATGATTGGGGTGAAGTCGTAACAAGGTAGCCGTA
TCGGAAGGTGCGGCTG (SEQ ID NO: 86)
HK557089.3.1395
AGACTTTAGCTTGCTAAAGTTGGAAGAGTTGCGAACGGGTGAGTAACGCGTAGGTAACCTGCCTACTAGCGGGGGA
TAACTATTGGAAACGATAGCTAATACCGCATAACAGCATTTAACCCATGTTAGATGCTTGAAAGGAGCAATTGCTT
CACTAGTAGATGGACCTGCGTTGTATTAGCTAGTTGGTGAGGTAACGGCTCACCAAGGCGACGATACATAGCCGAC
CTGAGAGGGTGATCGGCCACACTGGGACTGAGACACGGCCCAGACTCCTACGGGAGGCAGCAGTAGGGAATCTTCG
GCAATGGGGGCAACCCTGACCGAGCAACGCCGCGTGAGTGAAGAAGGTTTTCGGATCGTAAAGCTCTGTTGTAAGA
GAAGAACGTGTGTGAGAGTGGAAAGTTCACACAGTGACGGTAACTTACCAGAAAGGGACGGCTAACTACGTGCCAG
CAGCCGCGGTAATACGTAGGTCCCGAGCGTTGTCCGGATTTATTGGGCGTAAAGCGAGCGCAGGCGGTTTAATAAG
TCTGAAGTTAAAGGCAGTGGCTTAACCATTGTTCGCTTTGGAAACTGTTAGACTTGAGTGCAGAAGGGGAGAGTGG
AATTCCATGTGTAGCGGTGAAATGCGTAGATATATGGAGGAACACCGGTGGCGAAAGCGGCTCTCTGGTCTGTAAC
TGACGCTGAGGCTCGAAAGCGTGGGGAGCAAACAGGATTAGATACCCTGGTAGTCCACGCCGTAAACGATGAGTGC
TAGGTGTTAGGCCCTTTCCGGGGCTTAGTGCCGCAGCTAACGCATTAAGCACTCCGCCTGGGGAGTACGACCGCAA
GGTTGAAACTCAAAGGAATTGACGGGGGCCCGCACAAGCGGTGGAGCATGTGGTTTAATTCGAAGCAACGCGAAGA
ACCTTACCAGGTCTTGACATCCCGATGCTATTCCTAGAGATAGGAAGTTTCTTCGGAACTGTGAGACTTGAGGGCA
GAAGGGTAGAGTGCACTTGTATGGGGAGCTGTGGAATGCGTTCCCGCAACGAGCGCAACCCCTATTGTTAGTTGCC
ATCATTAAGTTGGGCACTCTAGCGAGACTGCCGGTAATAAACCGGAGGAAGGTGGGGATGACGTCAAATCATCATG
CCCCTTATGACCTGGGCTACACACGTGCTACAATGGTTGGTACAACGAGTCGCGAGTCGGTGACGGCAAGCAAATC
TCTTAAAGCCAATCTCAGTTCGGATTGTAGGCTGCAACTCGCCTACATGAAGTCGGAATCGCTAGTAATCGCGGAT
CAGCACGCCGCGGTGAATACGTTCCCGGGCCTTGTACACACCGCCCGTCACACCACGAGAGTTTGTAACACCCGAA
GTCGGTGAGGTANCCTTTTAGGAGC (SEQ ID NO: 87)
EU358719.1.1513
AGAGTTTGATCCTGGCTCAGGACGAACGCTGGCGGCGTGCCTAATACATGCAAGTAGAACGCTGAAGAAAGGAGCT
TGCTTCTTTTGGATGAGTTGCGAACGGGTGAGTAACGCGTAGGTAACCTGCCTTGTAGCGGGGGATAACTATTGGA
AACGATAGCTAATACCGCATAACAGCTTTTGACACATGTTAGAAGCTTGAAAGATGCAATTGCATCACTACGAGAT
GGACCTGCGTTGTATTAGCTAGTAGGTAGGGTAACGGCCTACCTAGGCGACGATACATAGCCGACCTGAGAGGGTG
ATCGGCCACACTGGGACTGAGACACGGCCCAGACTCCTACGGGAGGCAGCAGTAGGGAATCTTCGGCAATGGGGGC
AACCCTGACCGAGCAACGCCGCGTGAGTGAAGAAGGTTTTCGGATCGTAAAGCTCTGTTGTAAGAGAAGAACGTGT
GTGAGAGTGGAAAGTTCACACAGTGACGGTAACTTACCAGAAAGGGACGGCTAACTACGTGCCAGCAGCCGCGGTA
ATACGTAGGTCCCGAGCGTTGTCCGGATTTATTGGGCGTAAAGCGAGCGCAGGCGGTTTAATAAGTCTGAAGTTAA
AGGCAGTGGCTTAACCATTGTTCGCTTTGGAAACTGTTAAACTTGAGTGCAGAAGGGGAGAGTGGAATTCCATGTG
TAGCGGTGAAATGCGTAGATATATGGAGGAACACCGGTGGCGAAAGCGGCTCTCTGGTCTGTAACTGACGCTGAGG
CTCGAAAGCGTGGGGAGCAAACAGGATTAGATACCCTGGTAGTCCACGCCGTAAACGATGAGTGCTAGGTGTTAGG
CCCTTTCCGGGGCTTAGTGCCGCAGCTAACGCATTAAGCACTCCGCCTGGGGAGTACGACCGCAAGGTTGAAACTC
AAAGGAATTGACGGGGGCCCGCACAAGCGGTGGAGCATGTGGTTTAATTCGAAGCAACGCGAAGAACCTTACCAGG
TCTTGACATCCCAGTGACCGCTCTAGAGATAGAGTTTTTCTTCGGAACACTGGTGACAGGTGGTGCATGGTTGTCG
TCAGCTCGTGTCGTGAGATGTTGGGTTAAGTCCCGCAACGAGCGCAACCCTTATTGTTAGTTGCCATCATTCAGTT
GGGCACTCTAGCGAGACTGCCGGTAATAAACCGGAGGAAGGTGGGGATGACGTCAAATCATCATGCCCCTTATGAC
CTGGGCTACACACGTGCTACAATGGTTGGTACAACGAGTCGCAAGTCGGTGACGGCAAGCAAATCTCTTAAAGCCA
ATCTCAGTTCGGATTGTAGGCTGCAACTCGCCTACATGAAGCAGGAATTGCTAGTAATGGCAGGTCAGCATACTGC
CGTGAATACGTTCCCGGGCCTTGTACACACCGCCCGTCACACCATGAGAGTCTGTAACACCCGAAGTCGGTAGTCT
AACTACGGAGGACGCCGCCGAAGGTGGGACAGATAATTGGGGTGAAGTCGTAACAAGGTAGCCGTA (SEQ ID
NO: 88)
HQ748204.1.1442
CTAATACATGCGAGGAGAACGCTGAAGACTTTCTTTTGCTATAGTTGGGAGAGTTGCTAACGGGTGAGTAACGCGT
AGGTGACCTGCCTACTAGCGGGGGATAACTATTGCAAACGATAGCTAATACCGCATAACAGCCTTTAACCCATGTT
AGATGCTTGAAAGGAGCAATTGCTTCACTAGTAGATGGACCTGCGTTGTATTAGCTAGTTGGTGAGGTAACGGCTC
ACCAAGGCGACGATACATAGCCGACCTGAGAGGGTGATCGGCCACACTGGGACTGAGACACGGCCCATACTCCTAC
GGGAGGCACCAGTAGGGAATCTTCGGGAATGGGGGCAACCCTGACCGAGCAACGCCGCGTGAGTGAAGAAGGTTTT
CGGATCGTAAAGCTCTGTTGTAAGAGAAGAACGTGTGTGAGAGTGGAAAGTTCACACTGTGACGGTAACTTACCAG
AAAGGGACGGCTAACTACGTGCCAGCAGCCGCGGTAATACGTAGGTCCCGAGCGTTGTCCGGATTTATTGGGCGTA
AAGCGAGCGCAGGCGGTTTAATAAGTCTGAAGTTAAAGGCAGTGGCTTAACCATTGTTCGCTTTGGAAACTGTTAG
ACTTGAGTGCATAAGGGGAGAGTGGAATTCCATGTGTAGCGGTGAAATGCGTAGATATATGGAGGAACACCGGTGG
CGAAAGCGGCTCTCTGGTCTGTAACTGACGTTGAGGCTCGAAAGCGTGGGGAGCAAACAGGATTAGATACCCTGGT
AGTCCACGCTGTAAACGATGAGTGGTAGGTGTTAGGCCCTTTCTGGGGTTTAGTGCCGCAGATTACGCATTAAGCC
ATTCGCCTGGGGAGTACGACCGCAAGGTTGAAACTTAAAGGAATTGACGGGGGCCCGCACAAGCGGTGGAGCATGT
GGTTTAATTAGAAGCAACGCGAAGAACCTTACCAGGTCTTGACATCCCGATGCTATTCTTAGAGATAGGAAGTTTC
TTCGGAACATCGGTGACAGGTGGTGCATGGTTGTCGTCAGCTCGTGTCGAGAGATGTTGGGTTAAGTCCCTCAACG
AGCGCAACCCCTATTTTTATTTGCCATCATTAAGTTGGGCAATCTAGCGAGACTGCCGGTAATAAACCGGAGGAAG
GTGGGGATGACGTCAAATCATCATGCTCCTTATGTCATGGGGTACACACGTGGTACAATGGTTGGTACAACGAGTC
GCGAGTTGGTGAAGGCAAGCAAATCTCTTAAAGCCAATATCAGTTCGGATTGTAGGCTGCAAATAGCCTACATGTA
GTCGGAATTGTTAGTAATCGGGGATCAGCACTCCGCGGTGAATACGTTTCCGGGCCTTGTACACCCCGCCCGTCTA
CACCACGAGAGTTTGTAACACCCGAAGTCGGTGAGGTAACTCTTTTAGGAGCCAGCCGCCTAAGGTGGGATAGA
(SEQ ID NO: 89)
GQ338727.1.1397
CTACCTGCAGTCGACGAACACCTTATTTGATTTTCTTCGGAACTGAAGATTTGGTGATTGAGTGGCGGACGGGTGA
GTAACGCGTGGGTAACCTGCCCTGTACAGGGGGATAACAGTCAGAAATGACTGCTAATACCGCATAAGACCACAGC
ACCGCATGGTGCAGGGGTAAAAACTCCGGTGGTACAGGATGGACCCGCGTCTGATTAGCTGGTTGGTGAGGTAACG
GCTCACCAAGGCGACGATCAGTAGCCGGCTTGAGAAAGTGAACGGCCACATTGGGACTGAGACACGGCCCAAACTC
CTACGGGAGGCAGCAGTGGGGAATATTGCACAATGGGGGAAACCCTGATGCAGCGACGCCGCGTGAGTGAAGAAGT
ATCTCGGTATGTAAAGCTCTATCAGCAGGGAAGAAAATGACGGTACCTGACTAAGAAGCCCCGGCTAACTACGTGC
CAGCAGCCGCGGTAATACGTAGGGGGCAAGCGTTATCCGGAATTACTGGGTGTAAAGGGTGCGTAGGTGGTATGGC
AAGTCAGAAGTGAAAACCCAGGGCTTAACTCTGGGACTGCTTTTGAAACTGTCAGACTGGAGTGCAGGAGAGGTAA
GCGGAATTCCTAGTGTAGCGGTGAAATGCGTAGATATTAGGAGGAACATCAGTGGCGAAGGCGGCTTACTGGACTG
AAACTGACACTGAGGCACGAAAGCGTGGGGAGCAAACAGGATTAGATACCCTGGTAGTCCACGCCGTAAACGATGA
ATACTAGGTGTCGGGGCCGTAGAGGCTTCGGTGCCGCAGCCAACGCAGTAAGTATTCCACCTGGGGAGTACGTTCG
CAAGAATGAAACTCAAAGGAATTGACGGGGACCCGCACAAGTAGCGGAGCATGTGGTTTAATTCGAAGCAACGCGA
AGAACCTTACCTAAGCTTGACATCCTTTTGACCGATGCCTAATCGCATCTTTCCCTTCGGGGACAGAAGTGACAGG
TGGTGCATGGTTGTCGTCAGCTCGTGTCGTGAGATGTTGGGTTAAGTCCCGCAACGAGCGCAACCCTTGCCTTTAG
TTGCCATCATTAAGTTGGGCACTCTAGAGGGACTGCCAGGGATAACCTGGAGGAAGGTGGGGATGACGTCAAATCA
TCATGCCCCTTATGCTTAGGGCTACACACGTGCTACAATGGGTGGTACAGAGGGCAGCGAAGTCGTGAGGCCAAGC
TAATCCCTTAAAGCCATTCTCAGTTCGGATTGTAGGCTGAAACCCGCCTACATGAAGCTGGAGTTACTAGTAATCG
CAGATCAGAATGCTGCGGTGAATGCGTTCCCGGGTCTTGTACACACCGCCCGTCACACCATGGGAGTTGGGGGCGC
CCGAAGCCGGCTAGCTACTTTGGAAGCGT (SEQ ID NO: 90)
HQ803964.1.1435
GGGGGGCTTAACACATGCAAGTCGAACGAAGCGCTTTCGCTTTAATCTTCGGAGGAAAGAGGAAGTGACTGAGTGG
CGGACGGGTGAGTAACGCGTGGGTAACCTGCCTCATACAGGGGGATAACAGTTAGAAATGGCTGCTAATACCGCAT
AAGCATACAGCACCGCATGGTGCAGTGTGAAAAACTCCGGTGGTATAAGATGGACCCGCGTCTGATTAGGTAGTTG
GTGGGGTAACGGCCTACCAAGCCGACGATCAGTAGCCGACCTGAGAGGGTGACCGGCCACATTGGGACTGAGACAC
GGCCCAAACTCCTACGGGAGGCAGCAGTGGGGAATATTGCACAATGGGGGAAACCCTGATGCAGCGACGCCGCGTG
AAGGAAGAAGTATTTCGGTATGTAAACTTCTATCAGCAGGGAAGAAAATGACGGTACCTGACTAAGAAGCCCCGGC
TAACTACGTGCCAGCAGCCGCGGTAATACGTAGGGGGCAAGCGTTATCCGGATTTACTGGGTGTAAAGGGAGCGTA
GACGGAAGTGCAAGTCTGAAGTGAAAGCCCGGGGCTCAACCCCGTGACTGCTTTGGAAACTGTGCTTCTAGAGTGT
CGGAGAGGTAAGCGGAATTCCTAGTGTAGCGGTGAAATGCGTAGATATTAGGAGGAACATCAGTGGCGAAGGCGGC
TTACTGGGCGATAACTGACGTTGAGGCTCGAAAGCGTGGGGAGCAAACAGGATTAGATACCCTGGTAGTCCACGCC
GTAAACGATGAATACTAGGTGTCGGGAAGCACAGCTTTTCGGTGCCGCCGCAAACGCATTAAGTATTCCACCTGGG
GAGTACGTTCGCAAGAATGAAACTCAAAGGAATTGACGGGGACCCGCACAAGCGGTGGAGCATGTGGTTTAATTCG
AAGCAACGCGAAGAACCTTACCAAGTCTTGACATCCCGGTGACCGGACAGTAATGTGTCCTTTTCTTCGGAACACC
GGTGACAGGTGGTGCATGGTTGTCGTCAGCTCGTGTCGTGAGATGTTGGGTTAAGTCCCGCAACGAGCGCAACCCT
TATCCCCAGTAGCCAGCGGTTCGGCCGGGCACTCTGAGGAGACTGCCAGGGATAACCTGGAGGAAGGTGGGGATGA
CGTCAAATCATCATGCCCCTTATGACTTGGGCTACACACGTGCTACAATGGCGTAAACAAAGGGAAGCGAGACCGT
GAGGTGGAGCAAATCCCAAAAATAACGTCTCAGTTCGGACTGTAGTCTGCAACCCGACTACACGAAGCTGGAATCG
CTAGTAATCGCAGATCAGAATGCTGCGGTGAATACGTTCCCGGGTCTTGTACACACCGCCCGTCACACCATGGGAG
TTGGAAATGCCCGAAGTCAGTGACCCAACCGCAAGGAGGGAGCTGCCGAAGGCAGGTTCGATAACTG (SEQ ID
NO: 91)
FJ951866.1.1493
AGAGTTTGATCCTGGCTCAGGATGAACGCTGGCGGCGTGCTTAACACATGCAAGTCGAACGAAGCACTTTAACTTG
ATTTTTTCGGAATGATTGTTCTTGTGACTGAGTGGCGGACGGGTGAGTAACGCGTGGGTAACCTGCCTCATACAGG
GGGATAACAGTTAGAAATGACTGCTAATACCGCATAAGCGCACGGTATCGCATGATACAGTGTGAAAAACTCCGGT
GGTATGAGATGGACCCGCGTCTGATTAGCTAGTTGGCGGGGTAACGGCCCACCAAGGCGACGATCAGTAGCCGACC
TGAGAGGGTGACCGGCCACATTGGGACTGAGACACGGCCCAAACTCCTACGGGAGGCAGCAGTGGGGAATATTGCA
CAATGGGCGAAAGCCTGATGCAGCGACGCCGCGTGAACGAAAAAGTATTTCGGTATGTAAAGTTCTATCAGCAGGG
AAGATAATGACGGTACCTGACTAAGAAGCACCGGCTAAATACGTGCCAGCAGCCGCGGTAATACGTATGGTGCAAG
CGTTATCCGGATTTACTGGGTGTAAAGGGAGCGCAGGCGGTACGGCAAGTCTGATGTGAAAGCCCGGGGCTCAACC
CCGGTACTGCATTGGAAACTGTCGAACTAGAGTGTCGGAGGGGTAAGCGGAATTCCTAGTGTAGCGGTGAAATGCG
TAGATATTAGGAGGAACACCAGTGGCGAAGGCGGCTTACTGGACGACAACTGACGCTGAGGCGCGAAAGCGTGGGG
AGCAAACAGGATTAGATACCCTGGTAGTCCACGCTGTAAACGATGAATACTAGGTGTGGGAGGACTGACCCCTTCC
GTGCCGCAGTTAACACAATAAGTATTCCACCTGGGGAGTACGGCCGCAAGGCTGAAACTCAAAGGAATTGACGGGG
GCCCGCACAAGCAGTGGATTATGTGGTTTAATTCGACGCAACGCGAAGAACCTTACCAGGACTTGACATCCAACTA
ACGAAGTAGAGATACATTAGGTGCCCTTCGGGGAAAGTTGAGACAGGTGGTGCATGGTTGTCGTCAGCTCGTGTCG
TGAGATGTTGGGTTAAGTCCCGCAACGAGCGCAACCCCTATTGTTAGTTGCTACGCAAGAGCACTCTAGCGAGACT
GCCGTTGACAAAACGGAGGAAGGCGGGGACGACGTCAAATCATCATGCCCCTTATGTCCTGGGCTACACACGTAAT
ACAATGGCCGTCAACAAAGGGAAGCAAAGCCGCGAGGTGGAGCAAATCCCCAAAAACGGTCTCAGTTCGGATTGCA
GGCTGCAACTCGCCTGCATGAAGCTGGAATTGCTAGTAATCGTGGATCAGCATGCCACGGTGAATACGTTCCCGGG
CCTTGTACACACCGCCCGTCACACCATGAGAGTCGGGAACACCCGAAGTCCGTAGTCTAACCGCAAGGAGGGCGCG
GCCGAAGGTGGGTCCGGTAATTGGGGTGAAGTCGTAACAAGGTAACCGT (SEQ ID NO: 92)
EU772870.1.1289
AGTGGCGAACGGGTGAGTAACGCGTGAGGAACCTGCCTTTCAGTGGGGGACAACAGTTGGAAACGACTGCTAATAC
CGCATGATACTTTTTGGAGGCATCTCTGAAAAGTCAAAGCTTTATGTGCTGAAAGATGGTCTCGCGTCTGATTAGC
TAGTTGGTGAGGTAACGGCTCACCAAGGCGACGATCAGTAGCCGGTCTGAGAGGATGAACGGCCACATTGGGACTG
AGATACGGCCCAGACTCCTACGGGAGGCAGCAGTGGGGAATATTGGGCAATGGGGGAAACCCTGACCCAGCAACGC
CGCGTGAAGGAAGAAGGCCTTCGGGTTGTAAACTTCTTTTACCAGGGACGAAGAACGTGACGGTACCTGGAGAAAA
AGCAACGGCTAACTACGTGCCAGCAGCCGCGGTAATACGTAGGTTGCAAGCGTTATCCGGATTTATTGGGCGTAAA
GCGCGTCTAGGCGGTTTGGTAAGTCTGATGTGAAAATGCGGGGCTCAACTCCGTATTGCGTTGGAAACTGCTAAAC
TAGAGTACTGGAGAGGTAGGCGGAACTACAAGTGTAGAGGTGAAATTCGTAGATATTTGTAGGAATGCCGATGGGG
AAGCCAGCCTACTGGACAGATACTGACGCTAAAGCGCGAAAGCGTGGGGAGCAAACAGGATTAGATACCCTGGTAG
TCCACGCTGTAAACGATGAGTACTAGGTGTCGGAGGTTACCCCCTTCGGTGCCGCAGCTAACGCATTAAGTACTCC
GCCTGGGGAGTACGCACGCAAGTGTGGAACTCAAAGGAATTGACGGGGACCCGCACAAGTAGCGGAGCATGTGGTT
TAATTCGAAGCAACGCGAAGAACCTTACCTAGGCTTGACATCCTTCTGACCGAGGACTAATCTCCTCTTTCCCTCC
GGGGACAGAAGTGACAGGTGGTGCATGGTTGTCGTCAGCTCGTGTCGTGAGATGTTGGGTTAAGTCCCGCAACGAG
CGCAACCCTTGTCTTTAGTTGCCATCATTTAGTTGGGCACTCTGGAGAGACTGCCAGGGATAACCTGGAGGAAGGT
GGGGATGACGTCAAATCATCATGCCCCTTATGCCTAGGGCTACACACGTGCTACAATGGGTGGTACAGAGGGCAGC
TAAGCCGTGAGGTGGAGCAAATCCCTTAAAGCCATTCTCAGTTCGGATTGTAGGCTGAAACTCGCCTACATGAAGC
TGGAGTTACTAGTAATCGCAGATCAGAATGCTGCGGTGAATGCGTTCCCGGGTCTTGTACACACCGCCCGTCA
(SEQ ID NO: 93)
GQ448468.1.1366
AGAGTTTGATCCTGGCTCAGGATGAACGCTGACAGAATGCTTAACACATGCAAGTATACTTGATCCTTCGGGTGAT
GGTGGCGGACGGGTGAGTAACGCGTAAAGAACTTGCCCTGCAGTCTGGGACAACATTTGGAAACGAATGCTAATCC
CGCATAAGCCCACAGCTCGGCATCGAGCAGAGGGAAAAGGAGTGATCTGCTTTGAGATGGCCTCGCGTCCGATTAG
CTGGTTGGTGAGGTGACGGCCCATCAAGGCAACGATCGGTAGCCGGACTGAGAGGTTGAACGGCCACATTGGGATT
GAGACACGGCCCTTACTCCTACGGGAGGCAGCAGTGGGGAATATTGGACAATGGACCAAAAGTCTGATCCAGCAAT
TCTGTGTGCACGATGAAGTTTTTCGGAATGTAAAGTGCTTTCAGTTGGGACGAAGTAAGTGACGGTACCAACAGAA
GAAGCGACGGCTAAATACGTGCCAGCAGCCGCGGTAATACGTATGTCGCAAGCGTTATCCGGATTTATTGGGCGTA
AAGCGCGTCTAGGCGGTTTGGTAAGTCTGATGTGAAAATGCGGGGCTCAACTCCGTATTGCGTTGGAAACTGCCAA
ACTAGAGTACTGGAGAGGTGGGCGGAACTACAAGTGTAGAGGTGAAATTCGTAGATATTTGTAGGAATGCCGATGG
GGAAGCCAGCCCACTGGACAGATACTGACGCTAAAGCGCGAAAGCGTGGGTAGCAAACAGGATTAGATACCCTGGT
AGTCCACGCCGTAAACGATGATTACTAGGTGTTGGGGGTCGAACCTCAGCGCCCAAGCTAACGCGATAAGTAATCC
GCCTGGGGAGTACGTACGCAAGTATGAAACTCAAAGGAATTGACGGGGACCCGCACAAGCGGTGGAGCATGTGGTT
TAATTCGACGCAACGCGAGGAACCTTACCAGCGTTTGACATCCTAAGAAATTAGCAGAGATGCTTTTGTGCCCCTT
CGGGGGAACTTAGTGACAGGTGGTGCATGGCTGTCGTCAGCTCGTGTCGTGAGATGTTGGGTTAAGTCCCGCAACG
AGCGCAACCCCTTTCGTATGTTGCCATCATTAAGTTGGGCACTCATGCGATACTGCCTGCGATGAGCAGGAGGAAG
GTGGGGATGACGTCAAGTCATCATGCCCCTTATACGCTGGGCTACACACGTGCTACAATGGGTAGTACAGAGAGTC
GCAAACCTGCGAGGGGGAGCTAATCTCAGAAAACTATTCTCAGTTCGGATTGTACTCTGCAACTCGAGTACATGAA
GTTGGAATCGCTAGTAATCGCAAATCAGCTATGTTGCGGTGAATACGTTCTCGGGTCTTGTACACACCGCCCGT
(SEQ ID NO: 94)
EU774020.1.1361
AGAGTTTGATCCTGGCTCAGGACGAACGCTGGCGGCGTGCCTAACACATGCAAGTCGAGCGATTCTCTTCGGAGAA
GAGCGGCGGACGGGTGAGTAACGCGTGGGTAACCTGCCCTGTACACACGGATAACATACCGAAAGGTATGCTAATA
CGGGATAATATATAAGAGTCGCATGACTTTTATATCAAAGATTTTTCGGTACAGGATGGACCCGCGTCTGATTAGC
TTGTTGGCGGGGTAACGGCCCACCAAGGCGACGATCAGTAGCCGACCTGAGAGGGTGATCGGCCACATTGGAACTG
AGACACGGTCCAAACTCCTACGGGAGGCAGCAGTGGGGAATATTGCACAATGGGCGCAAGCCTGATGCAGCAACGC
CGCGTGAGCGATGAAGGCCTTCGGGTCGTAAAGCTCTGTCCTCAAGGAAGATAATGACGGTACTTGAGGAGGAAGC
CCCGGCTAACTACATGCCAGCAGCCGCGGTAATACGTATGTCGCAAGCGTTATCCGGATTTATTGGGCGTAAAGCG
CGTCTAGGTGGTTTGGTAAGTCTGATGTGAAAATGCGGGGCTCAACTCCGTATTGCGTTGGAAACTGCCAAACTAG
AGTACTGGAGAGGTAGGCGGAACTACAAGTGTAGAGGTGAAATTCGTAGATATTTGTAGGAATGCCGATGGGGAAG
CCAGCCTACTGGACAGATACTGACGCTAAAGCGCGAAAGCGTGGGTAGCAAACAGGATTAGATACCCTGGTAGTCC
ACGCCGTAAACGATGATTACTAGGTGTTGGGGGTCGAACCTCAGCGCCCAAGCTAACGCGATAAGTAATCCGCCTG
GGGAGTACGTACGCAAGTATGAAACTCAAAGGAGTTGACGGGGACCCGCACAAGCGGTGGAGCATGTGGTTTAATT
CGACGCAACGCGAGGAACCTTACCAGCGTTTGACATCCTAGGAATGAGAAAGAGATTTCTTAGTGCTCCTTCGGGA
GAACCTAGAGACAGGTGGTGCATGGCTGTCGTCAGCTCGTGTCGTGAGATGTTGGGTTAAGTCCCGCAACGAGCGC
AACCCCTATTGTATGTTGCCATCATTAAGTTGGGCACTCATGCGATACTGCCTGCGATGAGCAGGAGGAAGGTGGG
GATGACGTCAAGTCATCATGCCCCTTATACGCTGGGCTACACACGTGCTACAATGGGCAGTACAGAGAGAAGCAAT
ACCGCGAGGTGGAGCCAAACTTAAAAACCAGTCTCAGTTCGGATTGTAGGCTGAAACTCGCCTACATGAAGCTGGA
GTTACTAGTAATCGCGAATCAGAATGTCGCGGTGAATACGTACCCGGGTCTTGTACACACCGCCCGTCA (SEQ ID
NO: 95)
HQ782658.1.1415
AATGCTTAACACATGCAAGTCTACTTGATCCTTCGGGTGATGGTGGCGGACGGGTGAGTAACGCGTAAAGAACTTG
CCTTGCAGTCTGGGACAACGTCTGGAAACGGACGCTAATACCGGATATTATGCGAGAGTCGCATGGCTCTTTCATG
AAAGCTATATGCGCTGCAGGAGAGCTTTGCGTCCCATTAGTTAGTTGGTGAGGTAACGGCTCACCAAGACCGCGAT
GGGTAGCCGGCCTGAGAGGGTGAACGGCCACAAGGGGACTGAGACACGGCCCTTACTCCTACGGGAGGCAGCAGTG
GGGAATATTGGACAATGGACCAAAAGTCTGATCCAGCAATTCTGTGTGCACGATGACGGTCTTAGGATTGTAAAGT
GCTTTCAATCGGGAAAAAGAAAGTGATGGTACCGATAGAAGAAGCGACGGCTAAATACGTGCCAGCAGCCGCGGTA
ATACGTATGTCGCAAGCGTTATCCGGATTTATTGGGCGTAAAGCGCGTCTAGGCGGTCTGGTAAGTCTGATGTGGA
AATGCGGGGCTCAACTCCGTATTGCGTTGGAAACTGCCAGACTAGAGTACTGGAGAGGTGGGCGGAACTACAAGTG
TAGAGGTGAAATTCGTAGATATTTGTAGGAATGCCGATAGAGAAGTCAGCTCACTGGACAGATACTGACGCTGAAG
CGCGAAAGCATGGGGAGCAAACAGGATTAGATACCCTGGTAGTCCATGCCGTAAACGATGATTACTAAGCGTCGGG
GGTCGAACCTCGGCACTCAAGCTAACGCGATAAGTAATCCGCCTGGGGAGTACGTACGCAAGTATGAAACTCAAAG
GAATTGACGGGGACCCGCACAAGTGGTGGAGCATGTGGTTTAATTGGACGCAACGCGAGGAACTTTACCAGCGTGT
GACATCCTAGGAATGAGAAAGAGATTTTTCAGTGCTCCTTCGGGAGAACCCAGAGACAGGTGGTGCATGGCTGTGG
TCAGCTCGTGTCGTGAGATGTTGGGTTAAGTCCCGCAACGAGCGCAACCCCTATTGTATGTTGCCATCATTAAGTT
GGGCAATCATGCGATGCTGCCTGCGACGAGCAGGAGGAAGGTGGGGATGAGGTCAAGTCATCATGCCCGTTATATG
CTGGGCTACACACGTGCTACAATGGGCAGTACAGAGAGAAGCAAATATGCGAGGAGGAGCAAATGTCAGAAAGCTG
TTCGTAGTTCGGATTGTACTCTGCAACTGGAGTACATGAAGTTGGAATCAGTAGTAATCGCAAATCAGCAATGTTG
CGGTGAATACGTTCTCGGGTCTGGTACACACCGCCCGTCACACCACGAGAGTTGATTGCACCTGAAGTAGCAGGCC
TAACCGTAAGGAAGGGTGGTCCGAGGGTGTGGTTAGCGATTGGGGTG (SEQ ID NO: 96)
DQ794633.1.1395
AGAGTTTGATCCTGGCTCAGGATGAACGCTGGCGGCGTGCTTAACACATGCAAGTCGAGCGAAGCGCTTTTACGGA
TTTCTTCGGATTGAAGTGATTGTGACTGAGCGGCGGACGGGTGAGTAACGCGTGGGTAACCTGCCTCATACAGGGG
GATAACAGTTAGAAATGACTGCTAATACCGCATAAGCGCACAGTACCGCATGGGTACGGTGTGAAAAACTCCGGTG
GTATGAGATGGACCCGCGTCTGATTAGGTAGTTGGTGGGGTAACGGCCTACCAAGCCAACGATCAGTAGCCGACCT
GAGAGGGCGACCGGCCACATTGGGACTGAGACACGGCCCAAACTCCTACGGGAGGCAGCAGTGGGGAATATTGCAC
AATGGGGGAAACCCTGATGCAGCGACGCCGCGTGAAGGATGAAGTATTTCGGTATGTAAACTTCTATCAGCAGGGA
AGAAAATAACGGTACCTGAGTAAGAAGCCCCGGCTAACTACGTGCCAGCAGCCGCGGTAATACGTAGGGGGCAAGC
GTTATCCGGATTTACTGGGTGTAAAGGGAGCGTAGACGGAAGTGCAAGTCTGATGTGAAAACCCGAGGCTCAACCA
CGGGACTGCATTGGAAACTGTGCTTCTAGAGTGCCGGAGAGGTAAGCGGAATTCCTAGTGTAGCGGTGAAATGCGT
AGATATTAGGAGGAACACCAGTGGCGAAGGCGGCTTACTGGACGGTAACTGACGTTGAGGCTCGAAAGCGTGGGGA
GCAAACAGGATTAGATACCCTGGTAGTCCACGCCGTAAACGATGACTTACTAGGGTGTCGGGCAGCAAAGCTGTTC
GGTTGCCGCAGCCATCGCAATAAGTAGTCCACCTGGGGGAGTACGTTCGCAAGAATGAAACTCAAAGGAATTGACG
GGGACCCGCACAAGCGGTGGAGCATGTGGTTTAATTCGAAGCAACGCGAAGAACCTTACCTGCTCTTGACATCCCT
CTGACCGGCAAGTAATGTTGCCTTTCCTTCGGGACAGAGGAGACAGGTGGTGCATGGTTGTCGTCAGCTCGTGTCG
TGAGATGTTGGGTTAAGTCCCGCAACGAGCGCAACCCCTATCTTCAGTAGCCAGCATTTAAGGTGGGCACTCAGGA
GAGACTGCCAGGGATAACCTGGAGGAAGGTGGGGATGACGTCAAATCATCATGCCCCTTATGAGCAGGGCTACACA
CGTGCTACAATGGCGTAAACAAAGGGAAGCGAAAGGGTGACCTGGAGCAAATCTCAGAAATAACGTCTCAGTTCGG
ATTGTAGTCTGCAACTCGACTACATGAAGCTGGAATCGCTAGTAATCGCGAATCAGCATGTCGCGGTGAATACGTT
CCCGGGTCTTGTACTCACCGCCCGTCA (SEQ ID NO: 97)
FN668375.4306350.4307737
AGAGTTTGATCCTGGCTCAGGATGAACGCTGGCGGCGTGCCTAACACATGCAAGTTGAGCGATTTACTTCGGTAAA
GAGCGGCGGACGGGTGAGTAACGCGTGGGTAACCTACCCTGTACACACGGATAACATACCGAAAGGTATGCTAATA
CGGGATAATATATTTGAGAGGCATCTCTTGAATATCAAAGGTGAGCCAGTACAGGATGGACCCGCGTCTGATTAGC
TAGTTGGTAAGGTAACGGCTTACCAAGGCGACGATCAGTAGCCGACCTGAGAGGGTGATCGGCCACATTGGAACTG
AGACACGGTCCAAACTCCTACGGGAGGCAGCAGTGGGGAATATTGCACAATGGGCGAAAGCCTGATGCAGCAACGC
CGCGTGAGTGATGAAGGCCTTCGGGTCGTAAAACTCTGTCCTCAAGGAAGATAATGACGGTACTTGAGGAGGAAGC
CCCGGCTAACTACGTGCCAGCAGCCGCGGTAATACGTAGGGGGCTAGCGTTATCCGGATTTACTGGGCGTAAAGGG
TGCGTAGGCGGTCTTTCAAGTCAGGAGTGAAAGGCTACGGCTCAACCGTAGTAAGCTCTTGAAACTGGGAGACTTG
AGTGCAGGAGAGGAGAGTGGAATTCCTAGTGTAGCGGTGAAATGCGTAGATATTAGGAGGAACACCAGTTGCGAAG
GCGGCTCTCTGGACTGTAACTGACGCTGAGGCACGAAAGCGTGGGGAGCAAACAGGATTAGATACCCTGGTAGTCC
ACGCTGTAAACGATGAGTACTAGGTGTCGGGGGTTACCCCCTTCGGTGCCGCAGCTAACGCATTAAGTACTCCGCC
TGGGAAGTACGCTCGCAAGAGTGAAACTCAAAGGAATTGACGGGGACCCGCACAAGTAGCGGAGCATGTGGTTTAA
TTCGAAGCAACGCGAAGAACCTTACCTAAGCTTGACATCCCAATGACATCTCCTTAATCGGAGAGTTCCCTTCGGG
GACATTGGTGACAGGTGGTGCATGGTTGTCGTCAGCTCGTGTCGTGAGATGTTGGGTTAAGTCCCGCAACGAGCGC
AACCCTTGTCTTTAGTTGCCATCATTAAGTTGGGCACTCTAGAGAGACTGCCAGGGATAACCTGGAGGAAGGTGGG
GATGACGTCAAATCATCATGCCCCTTATGCTTAGGGCTACACACGTGCTGATTATGCTAAGGAAATAGGATTTACT
GGACAATTCTTAATAGAGCCTAAGCCAAAAGAGCCTACTAAACATCAATATGATTTTGATACTGCTACTGTTTTAG
GATTTTTAAGAAAGTATAATCTGGATAAATACTTCAAAGTGAATATAGAAGCAAACCATGCAACACTTGCAGGACA
TACTTTCCAACATGAATTAA (SEQ ID NO: 98)
GQ867445.1.1457
CGCTGGCGGCGTGCTTAACACATGCAAGTCGAACGAAGCGATTTGGAGGAAGTTTTCGGATGAAATCTGAATTGAC
TGAGTGGCGGACGGGTGAGTAACGCGTGGGTAACCTGCCTCACACAGGGGGACAACAGTTAGAAATGGCTGCTAAT
ACCGCATAAGCGCACAGCTTCGCATGAAGCAGTGTGAAAAACTCCGGTGGTGTGAGATGGACCCGCGTCTGATTAG
GTAGTTGGTGGGGTAACGGCCTACCAAGCCGACGATCAGTAGCCGACCTGAGAGGGTGACCGGCCACATTGGGACT
GAGACACGGCCCAAACTCCTACGGGAGGCAGCAGTGGGGAATATTGCACAATGGGGGAAACCCTGATGCAGCGACG
CCGCGTGAGTGAAGAAGTATTTCGGTATGTAAAGCTCTATCAGCAGGGAAGAAAATGACGGTACCTGACTAAGAAG
CCCCGGCTAACTACGTGCCAGCAGCCGCGGTAATACGTAGGGGGCAAGCGTTATCCGGATTTACTGGGTGTAAAGG
GAGCGTAGACGGCTTGGCAAGTCTGAAGTGAAAGCCCGGGGCTCAACCCCGGGACTGCTTTGGAAACTGTCAGGCT
AGAGTGCTGGAGAGGTAAGTGGAATTCCTAGTGTAGCGGTGAAATGCATAGATATTAGGAGGAACACCAGTGGCGA
AGGCGGCTTACTGGACAGTAACTGACGTTGAGGCTCGAAAGCGTGGGGAGCAAACAGGATTAGATACCCTGGTAGT
CCACGCCGTAAACGATGAATACTAGGTGTTGGGGAGCAAAGCTCTTCGGTGCCGTCGCAAACGCAATAAGTATTCC
ACCTGGGAAGTACGTTCGCAAGAATGAAACTCAAAGGAATTGACGGGGACCCGCACAAGCGGTGGAGCATGTGGTT
TAATTCGAAGCAACGCGAAGAACCTTACCAAGTCTTGACATCCCATTGAAAAGCCCGTAACGGGGTTCCCTCTTCG
GAGCAATGGAGACAGGTGGTGCATGGTTGTCGTCAGCTCGTGTCGTGAGATGTTGGGTTAAGTCCCGCAACGAGCG
CAACCCTTATCCTAAGTAGCCAGCAGGTAGAGCTGGGCACTCTTGGGAGACTGCCAGGGACAACCTGGAGGAAGGT
GGGGATGACGTCAAATCATCATGCCCCTTATGATTTGGGCTACACACGTGCTACAATGGCGTAAACAAAGGGAAGC
GAAGCTGTGAAGCTAAGCAAATCTCAAAAATAACGTCTCAGTTCGGATTGTAGTCTGCAACTCGACTACATGAAGC
TGGAATCGCTAGTAATCGCGGATCAGAATGCCGCGGTGAACACGTTCCCGGGTCTTGTACACACCGCCCGTCACAC
CATGGGAGTCAGTAACGCCCGAAGCCAGTGACCTAACCGCAAGGAAGGAGCTGTCGAAGGCGGGACCGATAACTGG
GGTGAAGTCGTAA (SEQ ID NO: 99)

### REFERENCES

1. Nitzan, O., Elias, M., Peretz, A. & Saliba, W. Role of antibiotics for treatment of inflammatory bowel disease. World J. Gastroenterol. 22, 1078-1087 (2016).
2. Khan, K. J. et al. Antibiotic therapy in inflammatory bowel disease: a systematic review and meta-analysis. Am. J. Gastroenterol. 106, 661-673 (2011).
3. Knights, D., Lassen, K. G. & Xavier, R. J. Advances in inflammatory bowel disease pathogenesis: linking host genetics and the microbiome. Gut 62, 1505-1510 (2013).
4. Garrett, W. S. et al. Communicable ulcerative colitis induced by T-bet deficiency in the innate immune system. Cell 131, 33-45 (2007).
5. Couturier-Maillard, A. et al. NOD2-mediated dysbiosis predisposes mice to transmissible colitis and colorectal cancer. J. Clin. Invest. 123, 700-711 (2013).
6. Moon, C. et al. Vertically transmitted faecal IgA levels determine extra-chromosomal phenotypic variation. Nature 521, 90-93 (2015).
7. David, L. A. et al. Diet rapidly and reproducibly alters the human gut microbiome. Nature 505, 559-563 (2014).
8. Muegge, B. D. et al. Diet drives convergence in gut microbiome functions across mammalian phylogeny and within humans. Science 332, 970-974 (2011).
9. Lee, D. et al. Diet in the pathogenesis and treatment of inflammatory bowel diseases. Gastroenterology 148, 1087-1106 (2015).
10. Levine, A., Sigall Boneh, R. & Wine, E. Evolving role of diet in the pathogenesis and treatment of inflammatory bowel diseases. Gut 67, 1726-1738 (2018).
11. Chassaing, B. et al. Dietary emulsifiers impact the mouse gut microbiota promoting colitis and metabolic syndrome. Nature 519, 92-96 (2015).
12. Desai, M. S. et al. A Dietary Fiber-Deprived Gut Microbiota Degrades the Colonic Mucus Barrier and Enhances Pathogen Susceptibility. Cell 167, 1339-1353.e21 (2016).
13. Hou, J. K., Abraham, B. & El-Serag, H. Dietary intake and risk of developing inflammatory bowel disease: a systematic review of the literature. Am. J Gastroenterol. 106, 563-573 (2011).
14. Ruemmele, F. M. et al. Consensus guidelines of ECCO/ESPGHAN on the medical management of pediatric Crohn's disease. J Crohns Colitis 8, 1179-1207 (2014).
15. Cohen-Dolev, N. et al. Differences in outcomes over time with exclusive enteral nutrition compared with steroids in children with mild to moderate crohn's disease: results from the GROWTH CD study. J Crohns Colitis 12, 306-312 (2018).
16. Sigall-Boneh, R. et al. Partial enteral nutrition with a Crohn's disease exclusion diet is effective for induction of remission in children and young adults with Crohn's disease. Inflamm. Bowel Dis. 20, 1353-1360 (2014).
17. Lee, D. et al. Comparative Effectiveness of Nutritional and Biological Therapy in North American Children with Active Crohn's Disease. Inflamm. Bowel Dis. 21, 1786-1793 (2015).
18. Borrelli, O. et al. Polymeric diet alone versus corticosteroids in the treatment of active pediatric Crohn's disease: a randomized controlled open-label trial. Clin. Gastroenterol. Hepatol. 4, 744-753 (2006).
19. Kaakoush, N. O. et al. Effect of exclusive enteral nutrition on the microbiota of children with newly diagnosed Crohn's disease. Clin. Transl. Gastroenterol. 6, e71 (2015).
20. Quince, C. et al. Extensive modulation of the fecal metagenome in children with crohn's disease during exclusive enteral nutrition. Am. J. Gastroenterol. 110, 1718-29; quiz 1730 (2015).
21. Gerasimidis, K. et al. Decline in presumptively protective gut bacterial species and metabolites are paradoxically associated with disease improvement in pediatric Crohn's disease during enteral nutrition. Inflamm. Bowel Dis. 20, 861-871 (2014).
22. Schwerd, T. et al. Exclusive enteral nutrition in active pediatric Crohn disease: Effects on intestinal microbiota and immune regulation. J. Allergy Clin. Immunol. 138, 592-596 (2016).
23. Lewis, J. D. et al. Inflammation, antibiotics, and diet as environmental stressors of the gut microbiome in pediatric crohn's disease. Cell Host Microbe 18, 489-500 (2015).
24. D'Argenio, V. et al. An altered gut microbiome profile in a child affected by crohn's disease normalized after nutritional therapy. Am. J Gastroenterol. 108, 851-852 (2013).
25. Wu, G. D. et al. Linking long-term dietary patterns with gut microbial enterotypes. Science 334, 105-108 (2011).
26. Gurry, T. et al. Predictability and persistence of prebiotic dietary supplementation in a healthy human cohort. Sci. Rep. 8, 12699 (2018).
27. Wu, G. D. et al. Comparative metabolomics in vegans and omnivores reveal constraints on diet-dependent gut microbiota metabolite production. Gut 65, 63-72 (2016).
28. Obregon-Tito, A. J. et al. Subsistence strategies in traditional societies distinguish gut microbiomes. Nat. Commun. 6, 6505 (2015).
29. Smits, S. A. et al. Seasonal cycling in the gut microbiome of the Hadza hunter-gatherers of Tanzania. Science 357, 802-806 (2017).
30. Cerquetella, M. et al. Inflammatory bowel disease in the dog: differences and similarities with humans. World J. Gastroenterol. 16, 1050-1056 (2010).
31. Jergens, A. E. & Simpson, K. W. Inflammatory bowel disease in veterinary medicine. Front Biosci (Elite Ed) 4, 1404-1419 (2012).
32. Peiravan, A. et al. Genome-wide association studies of inflammatory bowel disease in German shepherd dogs. PLoS ONE 13, e0200685 (2018).
33. Vizquez-Baeza, Y., Hyde, E. R., Suchodolski, J. S. & Knight, R. Dog and human inflammatory bowel disease rely on overlapping yet distinct dysbiosis networks. Nat. Microbiol. 1, 16177 (2016).
34. Suchodolski, J. S., Dowd, S. E., Wilke, V., Steiner, J. M. & Jergens, A. E. 16S rRNA gene pyrosequencing reveals bacterial dysbiosis in the duodenum of dogs with idiopathic inflammatory bowel disease. PLoS ONE 7, e39333 (2012).
35. Simpson, K. W. et al. Adherent and invasive Escherichia coli is associated with granulomatous colitis in boxer dogs. Infect. Immun. 74, 4778-4792 (2006).
36. Allenspach, K., Wieland, B., Grone, A. & Gaschen, F. Chronic enteropathies in dogs: evaluation of risk factors for negative outcome. J Vet Intern Med 21, 700-8 (2007).
37. Coelho, L. P. et al. Similarity of the dog and human gut microbiomes in gene content and response to diet. Microbiome 6, 72 (2018).
38. Kalenyak, K., Isaiah, A., Heilmann, R. M., Suchodolski, J. S. & Burgener, I. A. Comparison of the intestinal mucosal microbiota in dogs diagnosed with idiopathic inflammatory bowel disease and dogs with food-responsive diarrhea before and after treatment. FEMS Microbiol. Ecol. 94, (2018).
39. Minamoto, Y., Dhanani, N., Markel, M. E., Steiner, J. M. & Suchodolski, J. S. Prevalence of Clostridium perfringens, Clostridium perfringens enterotoxin and dysbiosis in fecal samples of dogs with diarrhea. Vet. Microbiol. 174, 463-473 (2014).
40. Ziese, A.-L. et al. Effect of probiotic treatment on the clinical course, intestinal microbiome, and toxigenic Clostridium perfringens in dogs with acute hemorrhagic diarrhea. PLoS ONE 13, e0204691 (2018).
41. Gevers, D. et al. The treatment-naive microbiome in new-onset Crohn's disease. Cell Host Microbe 15, 382-392 (2014).
42. Islam, K. B. M. S. et al. Bile acid is a host factor that regulates the composition of the cecal microbiota in rats. Gastroenterology 141, 1773-1781 (2011).
43. Wells, J. E. & Hylemon, P. B. Identification and characterization of a bile acid 7alpha-dehydroxylation operon in Clostridium sp. strain TO-931, a highly active 7alpha-dehydroxylating strain isolated from human feces. Appl. Environ. Microbiol. 66, 1107-1113 (2000).
44. Kitahara, M., Takamine, F., Imamura, T. & Benno, Y. Clostridium hiranonis sp. nov., a human intestinal bacterium with bile acid 7alpha-dehydroxylating activity. Int. J Syst. Evol. Microbiol. 51, 39-44 (2001).
45. Banaszkiewicz, A. et al. Enterotoxigenic Clostridium perfringens infection and pediatric patients with inflammatory bowel disease. J Crohns Colitis 8, 276-281 (2014).
46. Segata, N. et al. Metagenomic microbial community profiling using unique clade-specific marker genes. Nat. Methods 9, 811-814 (2012).
47. Kitahara, M., Takamine, F., Imamura, T. & Benno, Y. Assignment of Eubacterium sp. VPI 12708 and related strains with high bile acid 7alpha-dehydroxylating activity to Clostridium scindens and proposal of Clostridium hylemonae sp. nov., isolated from human faeces. Int. J. Syst. Evol. Microbiol. 50 Pt 3, 971-978 (2000).
48. Zaneveld, J. R., McMinds, R. & Vega Thurber, R. Stress and stability: applying the Anna Karenina principle to animal microbiomes. Nat. Microbiol. 2, 17121 (2017).
49. Nagy-Szakal, D. et al. Monotonous diets protect against acute colitis in mice: epidemiologic and therapeutic implications. J. Pediatr. Gastroenterol. Nutr. 56, 544-550 (2013).
50. Mandigers, P. J. J., Biourge, V., van den Ingh, T. S. G. A. M., Ankringa, N. & German, A. J. A randomized, open-label, positively-controlled field trial of a hydrolyzed protein diet in dogs with chronic small bowel enteropathy. J. Vet. Intern. Med. 24, 1350-1357 (2010).
51. Marks, S. L., Laflamme, D. P. & McAloose, D. Dietary trial using a commercial hypoallergenic diet containing hydrolyzed protein for dogs with inflammatory bowel disease. Vet. Ther. 3, 109-118 (2002).
52. Buffie, C. G. et al. Precision microbiome reconstitution restores bile acid mediated resistance to Clostridium difficile. Nature 517, 205-208 (2015).
53. Sorg, J. A. & Sonenshein, A. L. Bile salts and glycine as cogerminants for Clostridium difficile spores. J. Bacteriol. 190, 2505-2512 (2008).
54. Dunn, K. A. et al. Early Changes in Microbial Community Structure Are Associated with Sustained Remission After Nutritional Treatment of Pediatric Crohn's Disease. Inflamm. Bowel Dis. 22, 2853-2862 (2016).
55. Duvallet, C., Gibbons, S. M., Gurry, T., Irizarry, R. A. & Alm, E. J. Meta-analysis of gut microbiome studies identifies disease-specific and shared responses. Nat. Commun. 8, 1784 (2017).
56. Oliveira, F. S. et al. MicrobiomeDB: a systems biology platform for integrating, mining and analyzing microbiome experiments. Nucleic Acids Res. 46, D684-D691 (2018).
57. Gonzalez, A. et al. Qiita: rapid, web-enabled microbiome meta-analysis. Nat. Methods 15, 796-798 (2018).
58. Theriot, C. M., Bowman, A. A. & Young, V. B. Antibiotic-Induced Alterations of the Gut Microbiota Alter Secondary Bile Acid Production and Allow for Clostridium difficile Spore Germination and Outgrowth in the Large Intestine. mSphere 1, (2016).
59. van Nood, E. et al. Duodenal infusion of donor feces for recurrent Clostridium difficile. N. Engl. J. Med. 368, 407-415 (2013).
60. Wang, Y. et al. Fecal microbiota transplantation for refractory immune checkpoint inhibitor-associated colitis. Nat. Med. (2018). doi:10.1038/s41591-018-0238-9
61. Kozich, J. J., Westcott, S. L., Baxter, N. T., Highlander, S. K. & Schloss, P. D. Development of a dual-index sequencing strategy and curation pipeline for analyzing amplicon sequence data on the MiSeq Illumina sequencing platform. Appl. Environ. Microbiol. 79, 5112-5120 (2013).
62. Caporaso, J. G. et al. QIIIE allows analysis of high-throughput community sequencing data. Nat. Methods 7, 335-336 (2010).
63. Schloss, P. D. et al. Introducing mothur: open-source, platform-independent, community-supported software for describing and comparing microbial communities. Appl. Environ. Microbiol. 75, 7537-7541 (2009).
64. Edgar, R. C. Search and clustering orders of magnitude faster than BLAST. Bioinformatics 26, 2460-2461 (2010).
65. Quast, C. et al. The SILVA ribosomal RNA gene database project: improved data processing and web-based tools. Nucleic Acids Res. 41, D590-6 (2013).
66. Pruesse, E. et al. SILVA: a comprehensive online resource for quality checked and aligned ribosomal RNA sequence data compatible with ARB. Nucleic Acids Res. 35, 7188-7196 (2007).
67. Core Team, R. R: A Language and Environment for Statistical Computing. (2017).
68. Huber, W. et al. Orchestrating high-throughput genomic analysis with Bioconductor. Nat. Methods 12, 115-121 (2015).
69. McMurdie, P. J. & Holmes, S. phyloseq: an R package for reproducible interactive analysis and graphics of microbiome census data. PLoS ONE 8, e61217 (2013).
70. Pielou, E. C. The measurement of diversity in different types of biological collections. J. Theor. Biol. 13, 131-144 (1966).
71. Alßhauer, K. P., Wemheuer, B., Daniel, R. & Meinicke, P. Tax4Fun: predicting functional profiles from metagenomic 16S rRNA data. Bioinformatics 31, 2882-2884 (2015).
72. Love, M. I., Huber, W. & Anders, S. Moderated estimation of fold change and dispersion for RNA-seq data with DESeq2. Genome Biol. 15, 550-550 (2014).
73. Li, H. et al. The Sequence Alignment/Map format and SAMtools. Bioinformatics 25, 2078-2079 (2009).
74. Li, H. & Durbin, R. Fast and accurate long-read alignment with Burrows-Wheeler transform. Bioinformatics 26, 589-595 (2010).
75. Browne, H. P. et al. Culturing of “unculturable” human microbiota reveals novel taxa and extensive sporulation. Nature 533, 543-546 (2016).
76. Goodman, A. L. et al. Extensive personal human gut microbiota culture collections characterized and manipulated in gnotobiotic mice. Proc Natl Acad Sci USA 108, 6252-6257 (2011).
77. De MAN, J. C., Rogosa, M. & Sharpe, M. E. A MEDIUM FOR THE CULTIVATION OF LACTOBACILLI. Journal of Applied Bacteriology 23, 130-135 (1960).
78. Lunter, G. & Goodson, M. Stampy: a statistical algorithm for sensitive and fast mapping of Illumina sequence reads. Genome Res. 21, 936-939 (2011).
79. Friedman, E. S. et al. FXR-Dependent Modulation of the Human Small Intestinal Microbiome by the Bile Acid Derivative Obeticholic Acid. Gastroenterology (2018). doi:10.1053/j.gastro.2018.08.022
80. Leinonen, R., Sugawara, H., Shumway, M. & International Nucleotide Sequence Database Collaboration. The sequence read archive. Nucleic Acids Res. 39, D19-21 (2011).

Although the presently disclosed subject matter and its advantages have been described in detail, it should be understood that various changes, substitutions and alterations can be made herein without departing from the spirit and scope of the present disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

1. A method for determining susceptibility of an intestinal disorder in a companion animal, comprising:
a) measuring a first amount of a first intestinal microorganism and/or a second amount of a second intestinal microorganism in the companion animal;
b) comparing the first amount of the first intestinal microorganism with a first reference amount of the first intestinal microorganism, and/or comparing the second amount of the second intestinal microorganism with a second reference amount of the second intestinal microorganism, wherein the reference amounts of the intestinal microorganisms are determined based on the amounts of the intestinal microorganisms in a plurality of healthy companion animals; and
c) determining that the companion animal is susceptible of an intestinal disorder, when the first amount of the intestinal microorganism is higher than the first reference amount of the first intestinal microorganism, and/or when the second amount of the second intestinal microorganism is lower than the second reference amount of the second intestinal microorganism.

2. The method of claim 1, wherein the first intestinal microorganism comprises one or more bacterium comprising a 16S rRNA comprising a nucleotide sequence that is at least about 90% homologous or identical to the 16S rRNA nucleotide sequence of HQ802983.1.1440, GQ449092.1.1375, GQ448744.1.1393, KF842598.1.1394, HG798451.1.1400, New.ReferenceOTU52, HK555938.1.1357, FJ957494.1.1454, FN667392.1.1495, New.ReferenceOTU54, HQ760911.1.1437, GQ006324.1.1342, FJ950694.1.1472, FM865905.1.1392, FJ506371.1.1371, FJ957528.1.1445, JF712675.1.1540, New.ReferenceOTU82, AB009242.1.1451, HQ751549.1.1448, AB506370.1.1516, DQ057365.1.1393, FN667422.1.1495, AJ270486.1.1241, FN668375.4306350.4307737, GQ867426.1.1494, GX182404.8.1529, JF224013.1.1362, GQ448246.1.1389, KC245406.1.1465, FN667084.1.1493, EU470512.1.1400, EU768569.1.1352, AY239462.1.1500, KC504009.1.1465, FMl 79752.1.1686, New.ReferenceOTU114, HK.557089.3.1395, JQ208181.1.1352, HQ803964.1.1435, AM276759.1.1484, JN387556.1.1324, GQ448486.1.1387, HK.694029.9.1487, HQ754680.1.1441, FN563300.1.1447, FP929060.3837.5503, GQ448506.1.1374, Enterococcus durans, C. perfringens, or E. coli.

3. The method of claim 2, wherein the first intestinal microorganism is C. perfringens, E. coli and any combination thereof.

4. The method of claim 1, wherein the second intestinal microorganism comprises one or more bacterium comprising a 16S rRNA comprising a nucleotide sequence that is at least about 90% homologous or identical to the 16S rRNA nucleotide sequence of EU774020.1.1361, HQ793763.1.1451, HQ792787.1.1438, New.ReferenceOTU109, HQ792778.1.1436, or DQ113765.1.1450.

5. The method of claim 1, further comprising providing a customized recommendation of a treatment regimen, and/or further monitoring the intestinal microorganism, when the first amount of the first intestinal microorganism is lower than the first reference amount of the first intestinal microorganism, and/or when the second amount of the second intestinal microorganism is higher than the second reference amount of the second intestinal microorganism.
